SEQUENCE LISTING (80 sequences)

SEQ ID NO: 1 - protein, 118 aa, synthetic construct
  source 1..118: note = VH domain
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 2 - protein, 111 aa, synthetic construct
  source 1..111: note = VL
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 3 - protein, 8 aa, synthetic construct
  source 1..8: note = IMGT HCDR1
GYIFTAYT

SEQ ID NO: 4 - protein, 8 aa, synthetic construct
  source 1..8: note = IMGT HCDR2
IKPNNGLA

SEQ ID NO: 5 - protein, 11 aa, synthetic construct
  source 1..11: note = IMGT HCDR3
ARSEITTEFDY

SEQ ID NO: 6 - protein, 10 aa, synthetic construct
  source 1..10: note = IMGT LCDR1
ESVDSYANSF

SEQ ID NO: 7 - skipped

SEQ ID NO: 8 - protein, 9 aa, synthetic construct
  source 1..9: note = IMGT, Chothia, Kabat, AbM LCDR3
QQSKEDPLT

SEQ ID NO: 9 - protein, 7 aa, synthetic construct
  source 1..7: note = Chothia HCDR1
GYIFTAY

SEQ ID NO: 10 - protein, 6 aa, synthetic construct
  source 1..6: note = Chothia HCDR2
KPNNGL

SEQ ID NO: 11 - protein, 9 aa, synthetic construct
  source 1..9: note = Chothia, Kabat, AbM HCDR3
SEITTEFDY

SEQ ID NO: 12 - protein, 15 aa, synthetic construct
  source 1..15: note = Chothia, Kabat, AbM LCDR1
KSSESVDSYANSFLH

SEQ ID NO: 13 - protein, 7 aa, synthetic construct
  source 1..7: note = Chothia, Kabat, AbM LCDR2
RASTRES

SEQ ID NO: 14 - protein, 5 aa, synthetic construct
  source 1..5: note = Kabat HCDR1
AYTMH

SEQ ID NO: 15 - protein, 17 aa, synthetic construct
  source 1..17: note = Kabat HCDR2
WIKPNNGLANYAQKFQG

SEQ ID NO: 16 - protein, 6 aa, synthetic construct
  source 1..6: note = Contact HCDR1
TAYTMH

SEQ ID NO: 17 - protein, 13 aa, synthetic construct
  source 1..13: note = Contact HCDR2
WMGWIKPNNGLAN

SEQ ID NO: 18 - protein, 10 aa, synthetic construct
  source 1..10: note = Contact HCDR3
ARSEITTEFD

SEQ ID NO: 19 - protein, 11 aa, synthetic construct
  source 1..11: note = Contact LCDR1
DSYANSFLHWY

SEQ ID NO: 20 - protein, 10 aa, synthetic construct
  source 1..10: note = Contact LCDR2
LLIYRASTRE

SEQ ID NO: 21 - protein, 8 aa, synthetic construct
  source 1..8: note = Contact LCDR3
QQSKEDPL

SEQ ID NO: 22 - protein, 10 aa, synthetic construct
  source 1..10: note = AbM HCDR1
GYIFTAYTMH

SEQ ID NO: 23 - protein, 10 aa, synthetic construct
  source 1..10: note = AbM HCDR2
WIKPNNGLAN

SEQ ID NO: 24 - protein, 23 aa, synthetic construct
  source 1..23: note = IgG1 Full Hinge
EPKSCDKTHTCPPCPAPELLGGP

SEQ ID NO: 25 - protein, 10 aa, synthetic construct
  source 1..10: note = IgG1 Upper Hinge
EPKSCDKTHT

SEQ ID NO: 26 - protein, 19 aa, synthetic construct
  source 1..19: note = IgG2 Full Hinge
ERKCCVECPPCPAPPVAGP

SEQ ID NO: 27 - skipped

SEQ ID NO: 28 - protein, 20 aa, synthetic construct
  source 1..20: note = IgG4 Full Hinge
ESKYGPPCPSCPAPEFLGGP

SEQ ID NO: 29 - protein, 7 aa, synthetic construct
  source 1..7: note = IgG4 Upper Hinge
ESKYGPP

SEQ ID NO: 30 - protein, 217 aa, Homo sapiens
  source 1..217: note = IgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 329 aa, Homo sapiens
  source 1..329: note = CH1-hinge-CH2-CH3 (IGHG1*01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 107 aa, Homo sapiens [sequence omitted]

SEQ ID NO: 33 - protein, 329 aa, synthetic construct
  source 1..329: note = CH1-hinge-CH2-CH3 (HetFc-A) (IGHG1*01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 34 - protein, 329 aa, synthetic construct
  source 1..329: note = CH1-hinge-CH2-CH3 (HetFc-B) (IGHG1*01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 35 - protein, 15 aa, synthetic construct
  source 1..15: note = Wild-Type Hinge
EPKSCDKTHTCPPCP

SEQ ID NO: 36 - protein, 13 aa, synthetic construct
  source 1..13: note = Modified Hinge
EPKSCDCHCPPCP

SEQ ID NO: 37 - protein, 23 aa, synthetic construct
  source 1..23: note = signal peptide
MRPTWAWWLFLVLLLALWAPARG

SEQ ID NO: 38 - protein, 218 aa, synthetic construct
  source 1..218: note = Clone ID 11462 and 23998 Full
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 654 bp, synthetic construct
  source 1..654: mol_type = other DNA; note = Clone ID 11462 and 23998 Full
gacatcgtgatgacccagtcccctgattctctggccgtgtccctgggcgagagggcaacaatcaactgcaagagctccgagagcgtggactcctacgccaattctttcctgcactggtatcagcagaagccaggccagccccctaagctgctgatctacagggcctctaccagagagagcggagtgccagaccgcttctccggctctggcagcggcacagacttcaccctgacaatctctagcctgcaggccgaggacgtggccgtgtactattgtcagcagtccaaggaggaccccctgacctttggcggaggaacaaaggtggagatcaagcggaccgtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 40 - protein, 111 aa, synthetic construct; identical to SEQ ID NO: 2
  source 1..111: note = Clone ID 11462, 23998, 17423 VL

SEQ ID NO: 41 - protein, 447 aa, synthetic construct
  source 1..447: note = Clone ID 11466 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 42 - DNA, 1341 bp, synthetic construct
  source 1..1341: mol_type = other DNA; note = Clone ID 11466 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 43 - protein, 118 aa, synthetic construct; identical to SEQ ID NO: 1
  source 1..118: note = Clone IDs 11466, 11467, 11509, 17430, 17431, 21292, 21293, 23993, 25169, 26704 VH

SEQ ID NO: 44 - protein, 447 aa, synthetic construct
  source 1..447: note = Clone ID 11467 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 45 - DNA, 1341 bp, synthetic construct
  source 1..1341: mol_type = other DNA; note = Clone ID 11467 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 46 - protein, 445 aa, synthetic construct
  source 1..445: note = Clone ID 11509 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 1335 bp, synthetic construct
  source 1..1335: mol_type = other DNA; note = Clone ID 11509 Full
caggtccagctggtccagagcggagcagaggtgaagaaacccggagcaagcgtcaaggtctcatgcaaagcaagcggctacattttcaccgcctacacaatgcactgggtgcggcaggcccctggacagggcctggagtggatgggctggatcaagccaaacaatggcctggccaactacgcccagaagttccagggcagggtgaccatgacacgcgacacctctatcagcacagcctatatggagctgtctcggctgagaagcgacgataccgccgtgtactattgcgccaggtctgagatcaccacagagttcgattactggggccagggcaccctggtgacagtgagctccgctagcacaaagggaccaagcgtgttcccactggccccctctagcaagtccacctctggaggaacagccgccctgggctgtctggtgaaggactatttccccgagcctgtgaccgtgagctggaacagcggggccctgaccagcggagtgcacacatttcccgccgtgctgcagtcctctggcctgtactccctgagctccgtggtgaccgtgccctctagctccctgggcacccagacatatatctgcaacgtgaatcacaagcctagcaatactaaggtcgacaaaagagtggagcccaagtcctgcgattgtcattgccccccctgcccggcgccagagctgctgggcggaccatccgtgttcctgtttccacccaagcctaaggacaccctgatgatctccaggaccccagaggtgacatgcgtggtggtggacgtgtctcacgaggaccccgaggtgaagtttaactggtacgtggatggcgtggaggtgcacaatgccaagaccaagcccagggaggagcagtacaacagcacctatcgcgtggtgtccgtgctgacagtgctgcaccaggactggctgaacggcaaggagtataagtgcaaggtgagcaataaggccctgcccgcccctatcgagaagaccatctccaaggccaagggccagcctcgcgaaccacaggtgtacacactgcctccaagcagagaggagatgaccaagaaccaggtgtccctgacatgtctggtgaagggcttctatccttccgacatcgccgtggagtgggagtctaatggccagccagagaacaattacaagaccacaccccctgtgctggactctgatggcagcttctttctgtattctaagctgaccgtggataagagcagatggcagcagggcaacgtgttcagctgttctgtgatgcacgaagcactgcacaatcactacactcagaaaagcctgagcttaagccctggg

SEQ ID NO: 48 - protein, 228 aa, synthetic construct
  source 1..228: note = Clone ID 17423 Full
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKCSGTASVVCLLNNFYPREAKVQWKVDNALQSGNSQESVTEQDSKDSTYSLSSTLTLSKADYEKHKVYACEVTHQGLSSPVTKSFNRGECESSCDVKLV

SEQ ID NO: 49 - DNA, 684 bp, synthetic construct
  source 1..684: mol_type = other DNA; note = Clone ID 17423 Full
gacatcgtgatgacccagtcccctgattctctggccgtgtccctgggcgagagggcaacaatcaactgcaagagctccgagagcgtggactcctacgccaattctttcctgcactggtatcagcagaagccaggccagccccctaagctgctgatctacagggcctctaccagagagagcggagtgccagaccgcttctccggctctggcagcggcacagacttcaccctgacaatctctagcctgcaggccgaggacgtggccgtgtactattgtcagcagtccaaggaggaccccctgacctttggcggaggaacaaaggtggagatcaagcggaccgtggcggcgcccagcgtgttcatctttccccctagcgacgagcagctgaagtgcagcggaaccgcctccgtggtgtgcctgctgaacaatttctacccacgggaggccaaggtgcagtggaaggtggataacgccctgcagagcggcaattcccaggagtctgtgaccgagcaggacagcaaggattccacatattctctgagctccaccctgacactgtccaaggccgactacgagaagcacaaggtgtatgcctgcgaggtgacccaccagggcctgtctagccccgtgacaaagtcttttaacagaggcgagtgcgagtcctcttgtgatgtgaagctggtg

SEQ ID NO: 50 - protein, 448 aa, synthetic construct
  source 1..448: note = Clone ID 17430 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGCPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 51 - DNA, 1344 bp, synthetic construct
  source 1..1344: mol_type = other DNA; note = Clone ID 17430 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggctgccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggccaagggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 52 - protein, 448 aa, synthetic construct
  source 1..448: note = Clone ID 17431 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGCPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 53 - DNA, 1344 bp, synthetic construct
  source 1..1344: mol_type = other DNA; note = Clone ID 17431 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggctgccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggccaagggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 54 - protein, 219 aa, synthetic construct
  source 1..219: note = Clone ID 21226 Full
DIVMTQSPDSLAVSLGERATINCKSSESVDSYANSFLHWYQQKPCGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 657 bp, synthetic construct
  source 1..657: mol_type = other DNA; note = Clone ID 21226 Full
gacatcgtgatgacccagtcccctgattctctggccgtgtccctgggcgagagggcaacaatcaactgcaagagctccgagagcgtggactcctacgccaattctttcctgcactggtatcagcagaagccatgtggccagccccctaagctgctgatctacagggcctctaccagagagagcggagtgccagacaggttctccggctctggcagcggcacagacttcaccctgacaatctctagcctgcaggccgaggacgtggccgtgtactattgccagcagtccaaggaggaccccctgacctttggcggaggaacaaaggtggagatcaagcgcaccgtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 56 - protein, 112 aa, synthetic construct
  source 1..112: note = Clone ID 21226 VL
DIVMTQSPDSLAVSLGERATINCKSSESVDSYANSFLHWYQQKPCGQPPKLLIYRASTRESGVPDRFSGSGSGTDFTLTISSLQAEDVAVYYCQQSKEDPLTFGGGTKVEIK

SEQ ID NO: 57 - protein, 448 aa, synthetic construct
  source 1..448: note = Clone ID 21233 Full
QVQLVQSGACEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 58 - DNA, 1344 bp, synthetic construct
  source 1..1344: mol_type = other DNA; note = Clone ID 21233 Full
caggtgcagctggtgcagagcggagcctgcgaggtgaagaagccaggggccagcgtgaaggtgtcttgtaaggccagcggctacatcttcaccgcctatacaatgcactgggtgcggcaggcccccggacagggactggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgcgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 59 - protein, 119 aa, synthetic construct
  source 1..119: note = Clone ID 21233, 21234, 25161, 25165 VH
QVQLVQSGACEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 60 - protein, 448 aa, synthetic construct
  source 1..448: note = Clone ID 21234 Full
QVQLVQSGACEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 61 - DNA, 1344 bp, synthetic construct
  source 1..1344: mol_type = other DNA; note = Clone ID 21234 Full
caggtgcagctggtgcagagcggagcctgcgaggtgaagaagccaggggccagcgtgaaggtgtcttgtaaggccagcggctacatcttcaccgcctatacaatgcactgggtgcggcaggcccccggacagggactggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgcgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 62 - protein, 448 aa, synthetic construct
  source 1..448: note = Clone ID 21292
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 63 - DNA, 1344 bp, synthetic construct; identical to SEQ ID NO: 61
  source 1..1344: mol_type = other DNA; note = Clone ID 21292 Full

SEQ ID NO: 64 - protein, 448 aa, synthetic construct
  source 1..448: note = Clone ID 21293 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 65 - DNA, 1344 bp, synthetic construct
  source 1..1344: mol_type = other DNA; note = Clone ID 21293 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctgctatagagtggtgtccgtgctgacagtgctgcaccaggactggctgaacggcaaggagtataagtgtaaggtgtccaataaggccctgccagcccccatcgagaagaccatctctaaggccaagggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 66 - protein, 447 aa, synthetic construct
  source 1..447: note = Clone ID 23993 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - DNA, 1341 bp, synthetic construct
  source 1..1341: mol_type = other DNA; note = Clone ID 23993 Full
caggtgcagctggtgcagagcggagccgaggtgaagaagccaggggccagcgtgaaggtgtcttgcaaggccagcggctacatcttcaccgcctatacaatgcactgggtgaggcaggcccccggacagggactggagtggatgggctggatcaagcctaacaatggcctggccaactacgcccagaagtttcagggcagggtgaccatgacacgcgacacctctatcagcacagcctatatggagctgtcccggctgagatctgacgatacagccgtgtactattgtgccagatccgagatcaccacagagtttgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 68 - protein, 449 aa, synthetic construct
  source 1..449: note = Clone ID 25161 Full
QVQLVQSGACEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGCPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 69 - DNA, 1347 bp, synthetic construct
  source 1..1347: mol_type = other DNA; note = Clone ID 25161 Full
caggtgcagctggtgcagagcggagcctgcgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggctgccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggccaagggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 70 - protein, 449 aa, synthetic construct
  source 1..449: note = Clone ID 25165 Full
QVQLVQSGACEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 71 - DNA, 1347 bp, synthetic construct
  source 1..1347: mol_type = other DNA; note = Clone ID 25165 Full
caggtgcagctggtgcagagcggagcctgcgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctgctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 72 - protein, 331 aa, synthetic construct
  source 1..331: note = Clone ID 25169 Full
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGCPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 73 - DNA, 1347 bp, synthetic construct
  source 1..1347: mol_type = other DNA; note = Clone ID 25169 Full
caggtgcagctggtgcagagcggagccgaggtgaagaagccaggggccagcgtgaaggtgtcttgcaaggccagcggctacatcttcaccgcctatacaatgcactgggtgcggcaggcccccggacagggcctggagtggatgggctggatcaagcctaacaatggcctggccaactacgcccagaagtttcagggcagggtgaccatgacacgcgacacctctatcagcacagcctatatggagctgtcccggctgagatctgacgataccgccgtgtactattgtgccaggtccgagatcaccacagagttcgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggctgccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctgctatagagtggtgtccgtgctgacagtgctgcaccaggactggctgaacggcaaggagtataagtgcaaggtgtccaataaggccctgccagcccccatcgagaagaccatctctaaggccaagggccagcctcgcgaaccacaggtctacgtctaccccccatcaagagatgaactgacaaaaaatcaggtctctctgacatgcctggtcaaaggattctacccttccgacatcgccgtggagtgggaaagtaacggccagcccgagaacaattacaagaccacaccccctgtcctggactctgatgggagtttcgctctggtgtcaaagctgaccgtcgataaaagccggtggcagcagggcaatgtgtttagctgctccgtcatgcacgaagccctgcacaatcactacacacagaagtccctgagcctgagccctggc

SEQ ID NO: 74 - protein, 448 aa, synthetic construct
  source 1..448: note = Clone ID 26704 Full
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - DNA, 1344 bp, synthetic construct
  source 1..1344: mol_type = other DNA; note = Clone ID 26704 Full
caggtgcagctggtgcagagcggagccgaggtgaagaagccaggggccagcgtgaaggtgtcttgcaaggccagcggctacatcttcaccgcctatacaatgcactgggtgaggcaggcccccggacagggactggagtggatgggctggatcaagcctaacaatggcctggccaactacgcccagaagtttcagggcagggtgaccatgacacgcgacacctctatcagcacagcctatatggagctgtcccggctgagatctgacgatacagccgtgtactattgtgccagatccgagatcaccacagagtttgattactggggccagggcaccctggtgacagtgagctccgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgcccgagctgctgggcggccctagcgtgttcctgtttccccctaagccaaaggacaccctgatgatctctcggacccccgaggtgacatgcgtggtggtggacgtgagccacgaggaccccgaggtgaagttcaactggtacgtggatggcgtggaggtgcacaatgccaagacaaagccccgggaggagcagtacaacagcacctgctatagagtggtgtccgtgctgacagtgctgcaccaggactggctgaacggcaaggagtataagtgtaaggtgtccaataaggccctgccagcccccatcgagaagaccatctctaaggccaagggccagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 76 - protein, 111 aa, synthetic construct
  source 1..111: note = CH2 Domain with cysteine insertion between T299 and Y300
APELLGGPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAK

SEQ ID NO: 77 - protein, 111 aa, synthetic construct
  source 1..111: note = CH2 Domain with cysteine insertion between G237 and P238
APELLGGCPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 78 - protein, 112 aa, synthetic construct
  source 1..112: note = CH2 Domain with cysteine insertions between T299 and Y300, and between G237 and P238
APELLGGCPSVFLFPPKPKDTLMISRTPEVTCVVVDVSHEDPEVKFNWYVDGVEVHNAKTKPREEQYNSTCYRVVSVLTVLHQDWLNGKEYKCKVSNKALPAPIEKTISKAK

SEQ ID NO: 79 - protein, 108 aa, synthetic construct
  source 1..108: note = CL Domain with cysteine insertion between K126 and S127
RTVAAPSVFIFPPSDEQLKCSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 9 aa, synthetic construct
  source 1..9: note = c-terminal peptide
ESSCDVKLV

(19) United States
(12) Patent Application Publication
(10) Pub. No.: US 2025/0222128 A1
(43) Pub. Date: Jul. 10, 2025

(54) ANTIBODY DRUG-CONJUGATES TARGETING C-MET AND METHODS OF USE THEREOF

(71) Applicant: Zymeworks BC Inc., Vancouver (CA)

(72) Inventors: Rupert H. Davies, Seattle, WA (US); James R. Rich, Vancouver (CA); Graham Albert Edwin Garnett, Vancouver (CA); Mario Sanches, Vancouver (CA); Samir Das, Vancouver (CA); Patrick Farber, Vancouver (CA); Stuart Daniel Barnscher, Vancouver (CA)

(21) Appl. No.: 19/091,242

(22) Filed: Mar. 26, 2025

Related U.S. Application Data
(63) Continuation of application No. PCT/CA2023/051289, filed on Sep. 28, 2023.
(60) Provisional application No. 63/411,039, filed on Sep. 28, 2022.

(51) Int. Cl. A61K 47/68; A61P 35/00; C07K 16/28

(52) U.S. Cl. CPC A61K 47/6849; A61K 47/68031; A61K 47/6889; A61P 35/00; C07K 16/2863; C07K 2317/524; C07K 2317/526; C07K 2317/53; C07K 2317/56

(57) ABSTRACT

Antibody-drug conjugates (ADCs) comprising an antibody construct that specifically binds c-Met conjugated to one or more drugs, such as the auristatin analogue, compound 1, via a linker. Also described are anti-cMet antibody constructs that have been engineered to include one or more cysteine insertion mutations with each inserted cysteine residue providing a conjugation “handle” allowing for conjugation of a drug-linker to provide an ADC. Further described are multivalent drug-linkers comprising a plurality of the auristatin analogues suitable for use in the ADCs described herein.

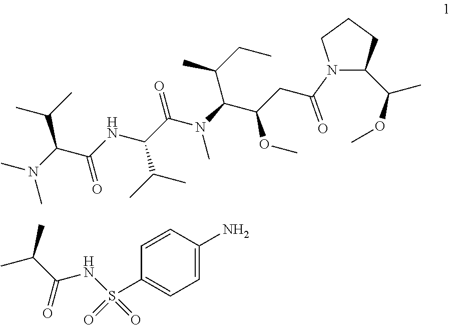

### CROSS REFERENCE TO RELATED APPLICATIONS

This application is a continuation of International Application No. PCT/CA2023/051289 filed on Sep. 28, 2023 which claims the benefit of U.S. Provisional Application No. 63/411,039, filed Sep. 28, 2022, which is hereby incorporated in its entirety by reference.

### SEQUENCE LISTING

The instant application contains a Sequence Listing which is hereby incorporated by reference in its entirety. Said XML copy, created on Nov. 6, 2023, is named ZYME092WO_ST26_sequence_listing.xml, and is 113,121 bytes in size.

### FIELD

The present disclosure relates to the field of immunotherapeutics and, in particular, to antibody-drug conjugates comprising an anti-cMet antibody and an auristatin analogue and their use in therapy.

### BACKGROUND

c-Met (also known as hepatocyte growth factor receptor (HGFR)) is a receptor tyrosine kinase encoded by the MET proto-oncogene. c-Met is frequently overexpressed in cancers and activation of c-Met signaling is related to drug resistance and the processes of carcinogenesis, invasion and metastasis (Gherardi et al, 2012, Nat Rev Cancer, 12:89-103). As such, disruption of MET signalling is considered to be a promising approach for cancer therapy. Various therapeutic strategies targeting c-Met are being explored including MET kinase inhibitors, HGF inhibitors and antibodies and antibody-drug conjugates (ADCs) that bind to c-Met.

Several ADCs targeting c-Met are being developed including telisotuzumab vedotin (formerly ABBV-399) (AbbVie Inc.), BYON3521 (Byondis B.V.), RC108 (RemeGen Co., Ltd) and REGN5093-M114 (Regeneron Pharmaceuticals). Of these, telisotuzumab vedotin, which comprises the anti-cMet antibody telisotuzumab (see, U.S. Pat. No. 8,545,839) conjugated to monomethyl auristatin E (MMAE), is the most advanced and is currently in Phase III clinical trials. In Phase II trials, telisotuzumab vedotin was reported to demonstrate anti-tumour activity in cMet-positive non-small cell lung carcinoma (NSCLC) with 65% of patients reporting grade ≥3 treatment-emergent adverse events (Camidge, et al., 2021, Clin Cancer Res, 27 (21): 5781-5792).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are antibody-drug conjugates (ADCs) targeting c-Met and methods of using these ADCs in therapy. One aspect of the disclosure relates to an antibody-drug conjugate having Formula I:

A-(L-(D)ₙ)ₚ (I)
wherein:
A is an antibody construct comprising an antigen-binding domain and an immunoglobulin (Ig) hinge region, the antigen-binding domain specifically binding to c-Met and comprising the heavy chain CDR sequences (HCDR1, HCDR2 and HCDR3) of the VH domain sequence set forth in SEQ ID NO:1, and the light chain CDR sequences (LCDR1, LCDR2 and LCDR3) of the VL domain sequence set forth in SEQ ID NO:2, and the Ig hinge region comprising an upper hinge sequence having an amino acid sequence of a native IgG1, IgG2 or IgG4 upper hinge sequence;
L is a cleavable linker;
D is:

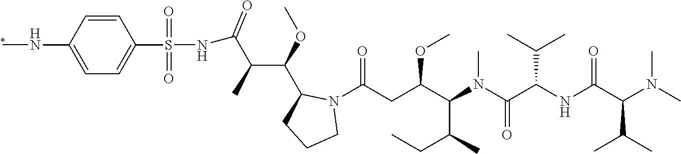 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

where * is the point of attachment to L,
n is between 1 and 4, and
p is between 1 and 8.

In certain embodiments, the antibody-drug conjugate has the structure:

### ADC 002

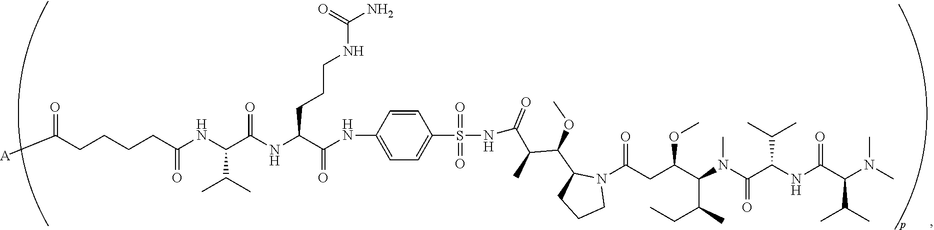

wherein A is the antibody construct that specifically binds to c-Met, and p is 6, or

### ADC 004

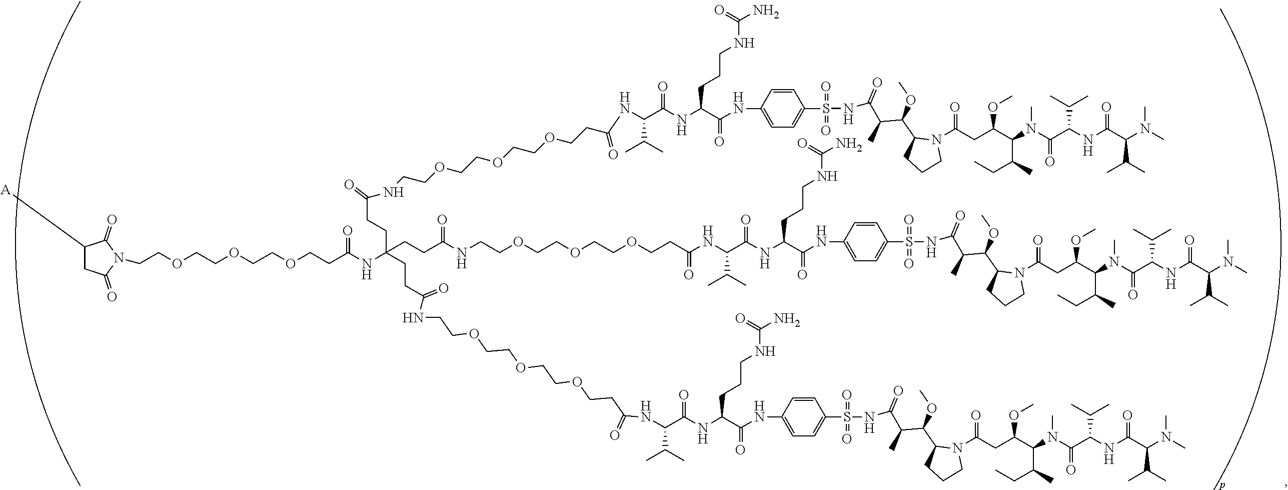

wherein A is the antibody construct that specifically binds to c-Met, and p is 2.

Another aspect of the disclosure relates to an antibody construct comprising:
an antigen-binding domain comprising a VL domain and a VH domain, and optionally a CH1 domain and a CL domain, wherein the antigen-binding domain specifically bind c-Met,
an Fc region comprising a CH2 domain having two CH2 domain sequences and a CH3 domain having two CH3 domain sequences,
wherein the antigen-binding domain comprises the heavy chain CDR sequences (HCDR1, HCDR2 and HCDR3) of the VH domain sequence set forth in SEQ ID NO:1, and the light chain CDR sequences (LCDR1, LCDR2 and LCDR3) of the VL domain sequence set forth in SEQ ID NO: 2, and
wherein the antibody construct comprises one or more cysteine insertion mutations independently selected from:
(a) a cysteine residue inserted between positions 40 and 41 in the VL domain;
(b) a cysteine residue inserted between positions 126 and 127 in the CL domain;
(c) a cysteine residue inserted between positions 9 and 10 in the VH domain;
(d) a cysteine residue inserted between positions 237 and 238 in a CH2 domain sequence, and
(e) a cysteine residue inserted between positions 299 and 300 in a CH2 domain sequence,
wherein the numbering of amino acids in the VL, CL and VH domains is Kabat numbering and the numbering of amino acids in the CH2 domain is EU numbering.

Another aspect of the disclosure relates to the use of an antibody construct comprising one or more cysteine insertion mutations as described herein to prepare an antibody drug conjugate.

Another aspect of the present disclosure relates to an antibody-drug conjugate comprising an antibody construct having one or more cysteine insertion mutations as described herein conjugated to a cytotoxin via a linker.

In certain embodiments, the antibody-drug conjugate comprising an antibody construct having one or more cysteine insertion mutations conjugated to a cytotoxin via a linker has Formula I:

A-(L-(D)ₙ)ₚ (I)
wherein:
A is the antibody construct;
L is a cleavable linker;
D is:

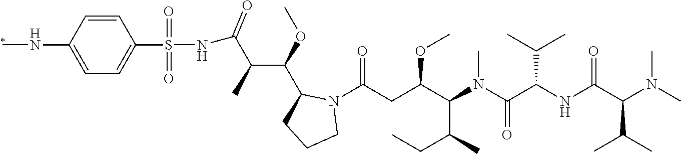 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

where * is the point of attachment to L,
n is between 1 and 4, and
p is between 1 and 8, and
wherein each L is conjugated to a sulfhydryl group of an inserted cysteine residue.

Another aspect of the disclosure relates to an antibody-drug conjugate having the structure:

### ADC 004

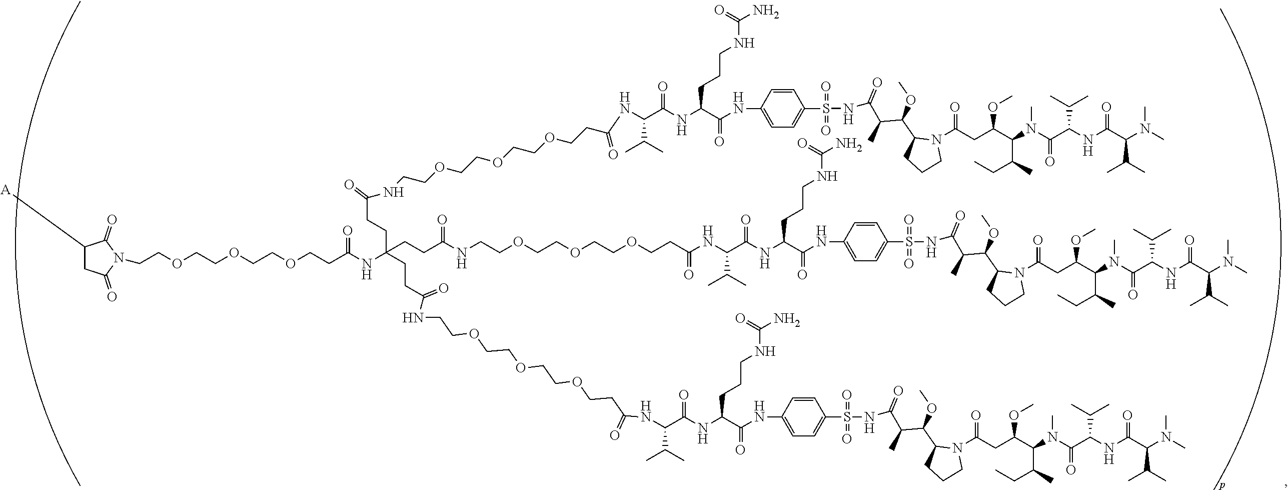

wherein p is 2, and A is an antibody construct comprising:
an antigen-binding domain comprising a VL domain and a VH domain, and optionally a CH1 domain and a CL domain, wherein the antigen-binding domain specifically bind c-Met,
an Fc region comprising a CH2 domain having two CH2 domain sequences and a CH3 domain having two CH3 domain sequences,
wherein the antigen-binding domain comprises the heavy chain CDR sequences (HCDR1, HCDR2 and HCDR3) of the VH domain sequence set forth in SEQ ID NO:1, and the light chain CDR sequences (LCDR1, LCDR2 and LCDR3) of the VL domain sequence set forth in SEQ ID NO: 2, and
wherein the antibody construct comprises a cysteine residue inserted between positions 299 and 300 in each CH2 domain sequence, and
wherein the antibody construct is a bivalent antibody comprising two antigen-binding domains, each antigen-binding domain specifically binding to c-Met.

Another aspect of the disclosure relates to a polynucleotide or set of polynucleotides encoding an antibody construct comprising one or more cysteine insertion mutations as described herein.

Another aspect of the disclosure relates to a vector or set of vectors comprising a polynucleotide or set of polynucleotides encoding an antibody construct comprising one or more cysteine insertion mutations as described herein.

Another aspect of the disclosure relates to a host cell comprising a vector or set of vectors comprising a polynucleotide or set of polynucleotides encoding an antibody comstruct comprising one or more cysteine insertion mutations as described herein.

Another aspect of the disclosure relates to a pharmaceutical composition comprising an antibody-drug conjugate as described herein and a pharmaceutically acceptable carrier or diluent.

Another aspect of the disclosure relates to a method of treating cancer in a subject comprising administering to the subject an effective amount of an antibody-drug conjugate as described herein.

Another aspect of the disclosure relates to an antibody-drug conjugate as described herein for use in therapy, for example, in the treatment of cancer in a subject in need thereof.

Another aspect of the disclosure relates to a use of an antibody-drug conjugate as described herein in the manufacture of a medicament for the treatment of cancer.

Another aspect of the disclosure relates to a multivalent drug-linker selected from:

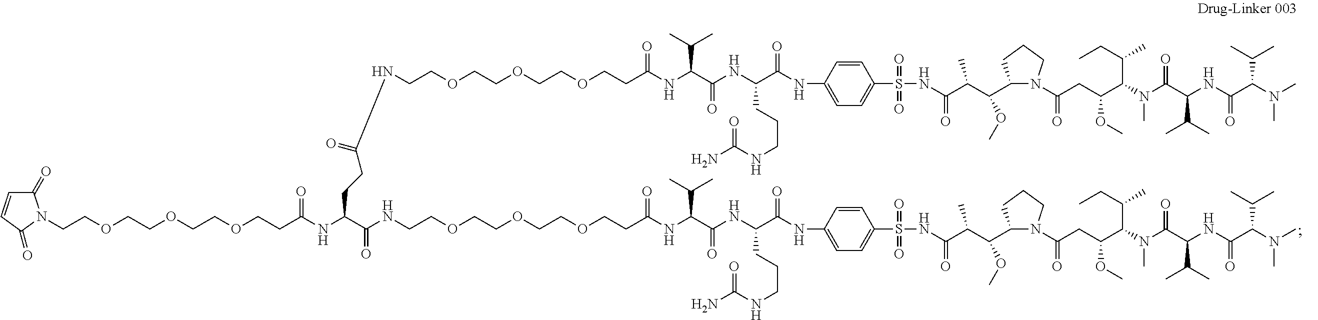 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCOCCOCCOCCN2C(=O)C=CC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C]

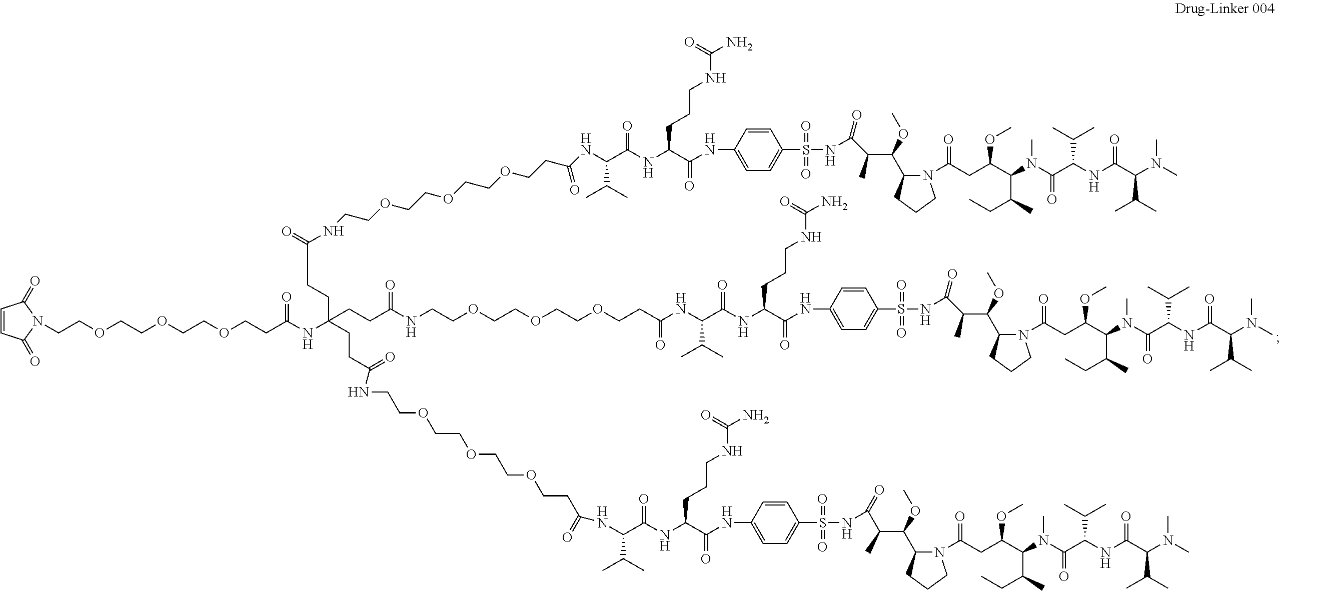

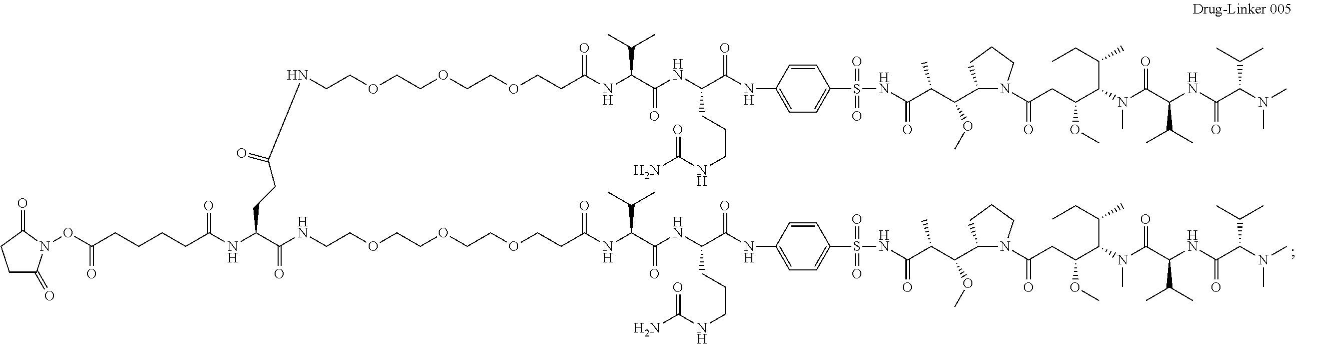 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)ON2C(=O)CCC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C]

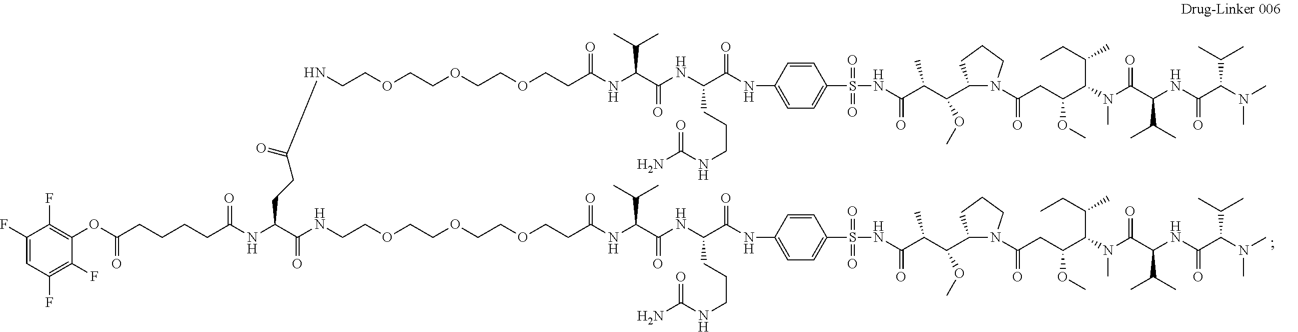 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)Oc2c(F)c(F)cc(F)c2F)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C]

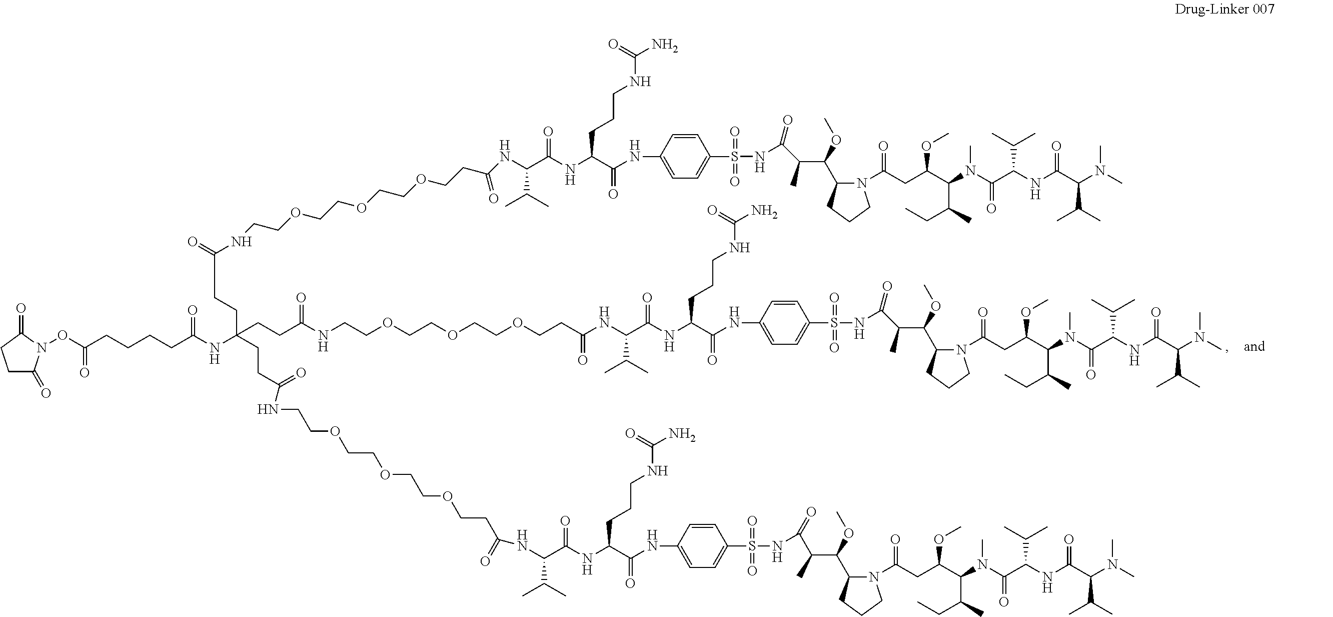

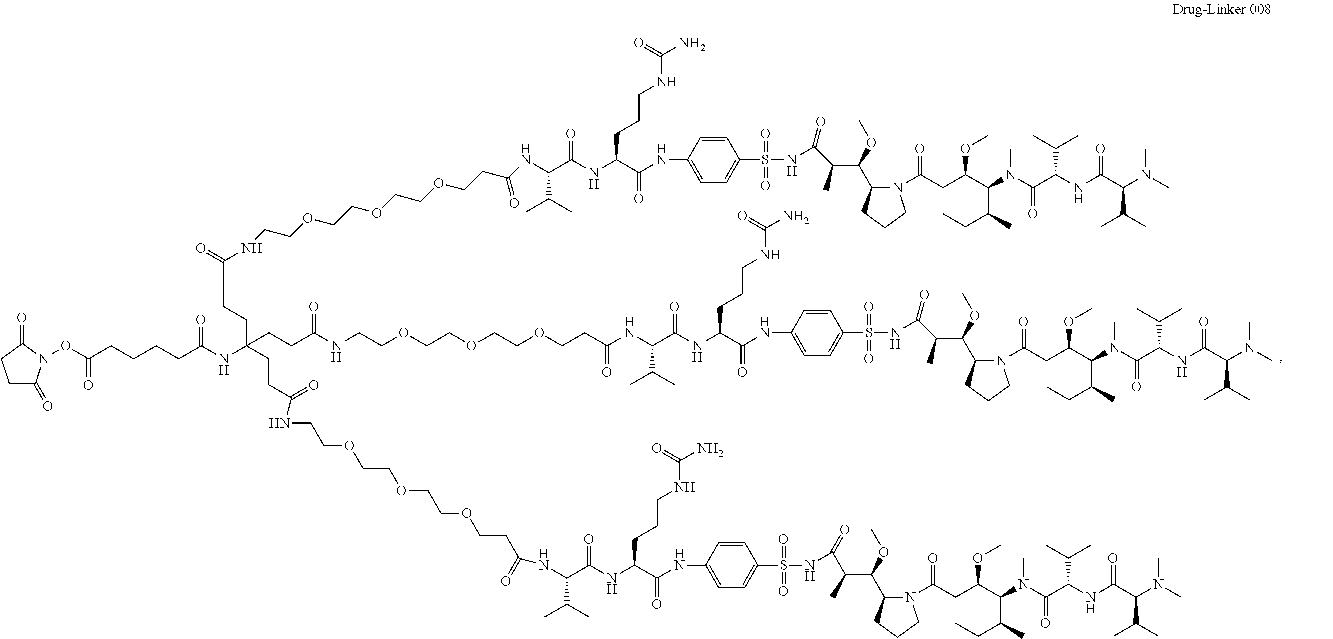

### BRIEF DESCRIPTION OF THE DRAWINGS

FIG. 1 presents an alignment of the hinge sequences of human IgG1 (SEQ ID NO: 24), IgG2 (SEQ ID NO: 26) and IgG4 (SEQ ID NO: 28). Upper, central and lower hinge regions are noted.

FIG. 2 presents (A) SDS-PAGE results for a representative anti-cMet cysteine insertion variant, v29001, under non-reducing (NR) and reducing (R) conditions (M=molecular weight marker), (B) the UPLC-SEC chromatogram for a representative anti-cMet cysteine insertion variant, v29001, and (C) an expanded view of the peak at 7.827 min. shown in upper panel (B).

FIG. 3 presents an exemplary reaction pathway for preparation of the trivalent drug-linkers, Drug-Linker 007 and Drug-Linker 008.

FIG. 4 presents the results of an assessment of cMet pathway agonism by proliferation of H596 lung cancer cells treated with anti-cMet antibodies (v17429, v17606 and v17427) and ADCs (v17427-Drug Linker 001 and v17427-MCvcPABC-MMAE). Data presented as the mean (±SEM) of 3 independent experimental replicates.

FIG. 5 presents the results of an assessment of cMet pathway agonism by measurement of AKT phosphorylation by ELISA for (A) H596 lung cancer cells, and (B) H441 lung cancer cells, treated with anti-cMet antibodies (v17429, v17606 and v17427) and ADCs (v17427-Drug Linker 001 and v17427-MCvcPABC-MMAE). Data presented as the mean (±SEM) of 3 independent experimental replicates.

FIG. 6 presents the results of an assessment of cMet pathway agonism by proliferation of H596 lung cancer cells treated with anti-cMet antibodies (v17429 and v17427) and ADCs comprising various anti-cMet cysteine insertion variants conjugated to Drug-Linker 001 at DAR 2. Data presented as the mean (±SEM) of 4 independent experimental replicates.

FIG. 7 presents the results of an assessment of cMet pathway agonism by measurement of AKT phosphorylation by ELISA for H441 lung cancer cells treated with anti-cMet antibodies (v17429, v17606 and v17427), cysteine-conjugated v17427-Drug-Linker 001 at DAR 4, lysine-conjugated v17427-Drug-Linker 002 at DAR 2, and ADCs comprising anti-cMet antibodies site-specifically conjugated to Drug-Linker 001 at (A) DAR 1, (B) DAR 2, and (C) DAR 3. Data presented as the mean (±SEM) of 2 independent experimental replicates.

FIG. 8 presents the results of an in vivo assessment of anti-tumour activity for ADCs comprising anti-cMet antibodies conjugated at DAR 4 to Drug-Linker 001 or MCvcPABC-MMAE in (A) cMet high HCC827 lung cancer model, (B) cMet high EBC1 lung cancer model, (C) cMet high H1975 lung cancer model, (D) cMet mid/high HT29 colorectal cancer model, (E) cMet low H292 lung cancer model, and (F) cMet low SW48 colorectal cancer model.

FIG. 9 presents the results of an in vivo assessment of anti-tumour activity for ADCs comprising anti-cMet antibodies conjugated to Drug-Linker 001 at DAR1, 2, 3 or 4, or Drug-Linker 002 at DAR2 in a cMet high H1975 lung cancer model, (A) at toxin-matched doses of 24, 12, 8 and 6 mg/kg, and (B) at toxin-matched doses of 4, 2, 1.3 and 1 mg/kg.

FIG. 10 presents the results of an in vivo assessment of anti-tumour activity for ADCs comprising anti-cMet antibodies conjugated to Drug-Linker 001 at DAR1, 2, 3 or 4, or Drug-Linker 002 at DAR2 in a cMet mid/high HT29 colorectal cancer model, (A) at toxin-matched doses of 12, 6, 4 and 3 mg/kg, and (B) at toxin-matched doses of 6, 3, 2 and 1.5 mg/kg.

FIG. 11 presents the results of an in vivo assessment of anti-tumour activity for ADCs comprising anti-cMet antibodies conjugated to Drug-Linker 002, Drug-Linker 003, Drug-Linker 004 or MCvcPABC-MMAE at various DARs and doses (as shown) in (A) cMet high H1975 lung cancer model, (B) cMet mid/high HT29 colorectal cancer model, (C) cMet low H292 lung cancer model, (D) cMet mid/high Hs746t gastric cancer model, and (E) cMet mid HCT116 colorectal cancer model.

FIG. 12 presents the results of an in vivo assessment of anti-tumour activity for ADCs comprising anti-cMet antibodies conjugated to Drug-Linker 003 at DAR4, Drug-Linker 004 at DAR6 or MCvcPABC-MMAE at DAR3 in various PDX models at the doses shown. *Models where v29001-Drug-Linker 004 DAR6 was not tested.

FIG. 13 presents results from an assessment of pharmacokinetics in Tg32 mice for ADCs comprising anti-cMet antibodies conjugated to Drug-Linker 002 at DAR4 or DAR 6, Drug-Linker 003 at DAR4, or Drug-Linker 004 at DAR6 and the corresponding free antibodies, (A) total IgG concentration in serum over time, and (B) total ADC concentration in serum over time.

FIG. 14 presents results from an assessment of the in vivo stability of ADCs comprising anti-cMet antibodies conjugated to Drug-Linker 002 at DAR4 or DAR 6, Drug-Linker 003 at DAR4, or Drug-Linker 004 at DAR6 as assessed by (A) % DAR remaining (Drug-Linker 002 ADCs) and (B) % thiosuccinimide ring opening (RO) and % DAR remaining (Drug-Linker 003 ADCs and Drug-Linker 004 ADCs).

### DETAILED DESCRIPTION

The present disclosure relates to antibody-drug conjugates (ADCs) comprising an antibody construct that specifically binds c-Met (an “anti-cMet antibody construct”) conjugated to a drug, such as a cytotoxin, via a linker. In the ADCs, the anti-cMet antibody construct may be conjugated to one drug molecule or it may be conjugated to more than one drug molecule.

Certain embodiments of the present disclosure relate to ADCs comprising an anti-cMet antibody construct conjugated to an auristatin analogue, compound 1, via a linker.

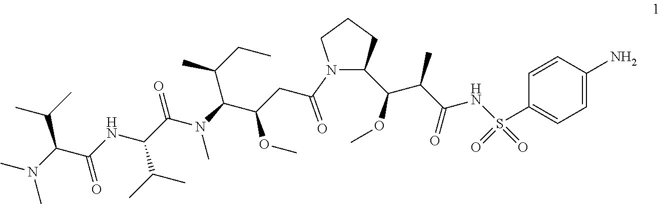 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

In such embodiments, the anti-cMet antibody construct may be conjugated to one of the auristatin analogues or it may be conjugated to more than one of the auristatin analogues.

The present disclosure also relates to anti-cMet antibody constructs that have been engineered to include one or more cysteine insertion mutations. Each inserted cysteine residue provides a conjugation “handle” allowing for conjugation of a drug-linker to provide an ADC. Certain embodiments of the present disclosure relate to ADCs comprising an anti-cMet antibody construct that has been engineered to include one or more cysteine insertion mutations that is conjugated to one or more drug molecules via the one or more inserted cysteines.

The present disclosure further relates to multivalent drug-linkers comprising a plurality of the auristatin analogues suitable for use in the ADCs described herein. Certain embodiments of the present disclosure relate to ADCs comprising an anti-cMet antibody construct conjugated to a multivalent drug-linker comprising a plurality of the auristatin analogues.

The ADCs of the present disclosure may find use as therapeutics, for example, for the treatment of cancer.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. From N-terminus to C-terminus, both the light chain variable region (VL) and the heavy chain variable region (VH) of an antibody typically comprise the domains FR1, CDR1, FR2, CDR2, FR3, CDR3, and FR4. The three heavy chain CDRs are referred to herein as HCDR1, HCDR2, and HCDR3, and the three light chain CDRs are referred to herein as LCDR1, LCDR2, and LCDR3. CDRs provide the majority of contact residues for the binding of the antibody to the antigen or epitope. Often, the three heavy chain CDRs and the three light chain CDRs are required to bind antigen. However, in some instances, even a single variable domain can confer binding specificity to the antigen. Furthermore, as is known in the art, in some cases, antigen-binding may also occur through a combination of one or more CDRs selected from the VH and/or VL domains, for example HCDR3.

A number of different definitions of the CDR sequences are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM (University of Bath) and Contact (MacCallum, et al., 1996, J Mol Biol, 262 (5): 732-745) definitions. By way of example, CDR definitions according to Kabat, Chothia, IMGT, AbM and Contact are provided in Table 1 below. Accordingly, as would be readily apparent to one skilled in the art, the exact numbering and placement of CDRs may differ based on the numbering system employed. However, it is to be understood that the disclosure herein of a VH includes the disclosure of the associated (inherent) heavy chain CDRs (HCDRs) as defined by any of the known numbering systems. Similarly, disclosure herein of a VL includes the disclosure of the associated (inherent) light chain CDRs (LCDRs) as defined by any of the known numbering systems.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, H33 | H52-H56 | H95-H102 | L24-L34 | L50-L56 | L89-L97 |
| | or H34 | | | | | |
| IMGT | H26-H33, H34, | H51-H57 | H93-H102 | L27-L32 | L50-L52 | L89-L97 |
| | H35, H35A or | | | | | |
| | H35B | | | | | |
| AbM | H26-H35B | H50-H58 | H95-H102 | L24-L34 | L50-L56 | L89-L97 |
| Contact | H30-H35B | H47-H58 | H93-H101 | L30-L36 | L46-L55 | L89-L96 |

¹Either the Kabat or Chothia numbering system may be used for HCDR2, HCDR3 and the light chain CDRs for all definitions except Contact, which uses Chothia numbering
²Using Kabat numbering. The position in the Kabat numbering scheme that demarcates the end of the Chothia and IMGT CDR-H1 loop varies depending on the length of the loop because Kabat places insertions outside those CDR definitions at positions 35A and 35B. However, the IMGT and Chothia CDR-H1 loop can be unambiguously defined using Chothia numbering. CDR-H1 definitions using Chothia numbering: Kabat H31-H35, Chothia H26-H32, AbM H26-H35, IMGT H26-H33, Contact H30-H35.

The terms “subject” and “patient,” as used herein, refers to an animal, in some embodiments a mammal, which is the object of treatment, observation or experiment. The animal may be a human, a non-human primate, a companion animal (for example, dog, cat, or the like), farm animal (for example, cow, sheep, pig, horse, or the like) or a laboratory animal (for example, rat, mouse, guinea pig, non-human primate, or the like). In certain embodiments, the subject is a human.

It is contemplated that any embodiment discussed herein can be implemented with respect to any method, use or composition disclosed herein, and vice versa.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Antibody-Drug Conjugates

Certain embodiments of the present disclosure relate to antibody-drug conjugates (ADCs) having Formula I:

A-(L-(D)ₙ)ₚ I
in which,
A is an antibody construct that specifically binds c-Met;
L is a linker;
D is an auristatin analogue having a structure:

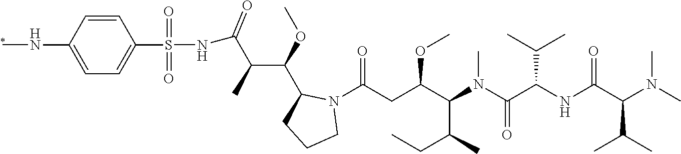 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

where * is the point of attachment to L;
n is between 1 and 4, and
p is between 1 and 8.

Linker, L, may be monovalent (bound to a single D, where n=1), or it may be multivalent (bound to multiple D, where n=2, 3 or 4).

In Formula I above, the parameters n and p define the number of auristatin analogue molecules, D, conjugated to the antibody construct, A. More specifically, the product of n×p defines the drug-to-antibody ratio, or “DAR,” for the ADC. One skilled in the art will appreciate that a given DAR may be achieved by various combinations of n and p. For example, an ADC with a DAR of 4 may comprise an antibody construct conjugated to four drug-linkers each drug-linker including a single D (i.e. n=1 and p=4), or an antibody construct conjugated to two drug linkers each drug-linker including two Ds (i.e. n=2 and p=2). Similarly, in another example, an ADC with a DAR of 6 may comprise an antibody construct conjugated to six drug-linkers each drug-linker including a single D (i.e. n=1 and p=6), or an antibody construct conjugated to three drug-linkers each drug-linker including two Ds (i.e. n=2 and p=3), or an antibody construct conjugated to two drug-linkers each drug-linker including three Ds (i.e. n=3 and p=2).

Those skilled in the art will also appreciate that within an ADC preparation comprising a plurality of ADCs, each anti-cMet antibody construct, A, is conjugated to an integer number of auristatin analogues, D, however, the DAR determined for the ADC preparation may give a non-integer result reflecting a statistical average of the individual DARs for the plurality of ADCs comprised by the preparation. Accordingly, ADC preparations having both integer and non-integer DARs are intended to be encompassed by Formula I.

In some embodiments, the ADCs of Formula I have a DAR between about 1 and about 6. In some embodiments, the ADCs of Formula I have a DAR between about 2 and about 6. In some embodiments, the ADCs of Formula I have a DAR between about 4 and about 6.

In some embodiments, in the ADCs of Formula I, n is 1 and p is 4, and a preparation of the ADCs has a DAR of about 4. In some embodiments, in the ADCs of Formula I, n is 2 and p is 2, and a preparation of the ADCs has a DAR of about 4. In some embodiments, in the ADCs of Formula I, n is 3 and p is 2, and a preparation of the ADCs has a DAR of about 6. In some embodiments, in the ADCs of Formula I, n is 1 and p is 6, and a preparation of the ADCs has a DAR of about 6.

### Anti-cMet Antibody Constructs

The ADCs of the present disclosure comprise an anti-cMet antibody construct. In this context, the term “antibody construct” refers to a polypeptide or a set of polypeptides that comprises one or more antigen-binding domains, where each of the one or more antigen-binding domains specifically binds to an epitope or antigen. Where the antibody construct comprises two or more antigen-binding domains, each of the antigen-binding domains may bind the same epitope or antigen (i.e. the antibody construct is monospecific) or they may bind to different epitopes or antigens (i.e. the antibody construct is bispecific or multispecific). In accordance with the present disclosure, the anti-cMet antibody construct comprises at least one antigen-binding domain that specifically binds to c-Met. In certain embodiments, the anti-cMet antibody construct may further comprise a scaffold and at least one of the one or more antigen-binding domains can be fused or covalently attached to the scaffold, optionally via a linker.

In certain embodiments, the anti-cMet antibody construct comprises two antigen-binding domains, each of which specifically binds to c-Met. In some embodiments, the anti-cMet antibody construct comprises two antigen-binding domains, each of which specifically binds to c-Met, and a scaffold. In some embodiments, the anti-cMet antibody construct may comprise three or four antigen-binding domains and a scaffold. In these formats, at least a first antigen-binding domain is operably linked to the scaffold and the remaining antigen-binding domain(s) may each independently be operably linked to the scaffold or to the first antigen-binding domain or, when more than two antigen-binding domains are present, to another antigen-binding domain.

In certain embodiments, the anti-cMet antibody construct may be in an antibody format that is based on an immunoglobulin (Ig). In certain embodiments, the anti-cMet antibody construct may be based on an IgG class immunoglobulin, for example, an IgG1, IgG2, IgG3 or IgG4 immunoglobulin. In some embodiments, the anti-cMet antibody construct may be based on an IgG1 immunoglobulin. In the context of the present disclosure, when an anti-cMet antibody construct is based on a specified immunoglobulin isotype, it is meant that the anti-cMet antibody construct comprises all or a portion of the constant region of the specified immunoglobulin isotype. For example, an anti-cMet antibody construct based on a given Ig isotype may comprise at least one antigen-binding domain operably linked to an Ig scaffold, where the scaffold comprises an Fc region from the given isotype and optionally an Ig hinge region from the same or a different isotype. It is to be understood that the anti-cMet antibody constructs may also comprise hybrids of isotypes and/or subclasses in some embodiments. It is also to be understood that the Fc region and/or hinge region may optionally be modified to impart one or more desirable functional properties as is known in the art.

In some embodiments, the anti-cMet antibody constructs may be derived from two or more immunoglobulins that are from different species, for example, the anti-cMet antibody construct may be a chimeric antibody or a humanized antibody. The terms “chimeric antibody” and “humanized antibody” both refer generally to antibodies that combine immunoglobulin regions or domains from more than one species.

A “chimeric antibody” typically comprises at least one variable domain from a non-human antibody, such as a rabbit or rodent (for example, murine) antibody, and at least one constant domain from a human antibody. The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567.

A “humanized antibody” is a type of chimeric antibody that contains minimal sequence derived from a non-human antibody. Generally, humanized antibodies are human immunoglobulins (recipient antibody) in which residues from a hypervariable region or CDR of the recipient are replaced by residues from a hypervariable region or CDR of a non-human species (donor antibody), such as mouse, rat, rabbit or non-human primate, having the desired specificity and affinity for a target antigen. This technique for creating humanized antibodies is often referred to as “CDR grafting.”

In some instances, additional modifications are made to further refine antibody performance. For example, framework region (FR) residues of the human immunoglobulin are replaced by corresponding non-human residues, or the humanized antibodies may comprise residues that are not found in either the recipient antibody or the donor antibody. In general, a variable domain in a humanized antibody will comprise all or substantially all of the hypervariable regions or CDRs from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329, and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterised human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting may in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57 (20): 4593-4599).

Alternatively, or in addition to, these traditional approaches, more recent technologies may be employed to further reduce the immunogenicity of a CDR-grafted humanized antibody. For example, frameworks based on human germline sequences or consensus sequences may be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e. the similarity to human germline sequence) of the humanized antibody and thus may help reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

In certain embodiments, the anti-cMet antibody construct comprises an antigen-binding domain that specifically binds c-Met and an immunoglobulin (Ig) hinge region. In some embodiments, the antibody construct comprises two antigen-binding domains, each of which specifically bind c-Met, and an Ig hinge region. In some embodiments, the anti-cMet antibody construct comprises at least one antigen-binding domain that specifically binds c-Met, an Ig hinge region and a scaffold.

In certain embodiments, the anti-cMet antibody construct comprises an antigen-binding domain that specifically binds c-Met, an Ig hinge region, and a scaffold that is an Fc region. In some embodiments, the antibody construct comprises two antigen-binding domains, each of which specifically bind c-Met; an Ig hinge region, and an Fc region.

In certain embodiments, the anti-cMet antibody construct is an antibody or an antigen-binding antibody fragment. In some embodiments, the anti-cMet antibody construct is a bivalent antibody. In some embodiments, the anti-cMet antibody is a monospecific or bispecific antibody. In some embodiments, the anti-cMet antibody is a monospecific antibody. In some embodiments, the anti-cMet antibody is a bivalent, monospecific antibody.

### Antigen-Binding Domains

The anti-cMet antibody constructs of the present disclosure comprise at least one antigen-binding domain that specifically binds to cMet. By “specifically binds” to c-Met, it is meant that the antibody construct binds to c-Met and does not exhibit significant binding to non-c-Met proteins. In certain embodiments, the at least one antigen-binding domain that specifically binds to c-Met is capable of binding to human c-Met. In some embodiments, the at least one antigen-binding domain that specifically binds to c-Met is capable of binding to human c-Met and to cynomolgus c-Met. In some embodiments, the at least one antigen-binding domain that specifically binds to c-Met is capable of binding to human c-Met and cynomolgus c-Met, and does not exhibit significant binding to c-Met from other species.

The at least one antigen-binding domain may be an immunoglobulin-based antigen-binding domain, such as an antigen-binding antibody fragment. Examples of an antigen-binding antibody fragment include, but are not limited to, a Fab fragment, a Fab′ fragment, a single chain Fab (scFab), a single chain Fv (scFv) and a single domain antibody (sdAb).

A “Fab fragment” contains the constant domain of the light chain (CL) and the first constant domain of the heavy chain (CH1) along with the variable domains of the light and heavy chains (VL and VH, respectively). Fab′ fragments differ from Fab fragments by the addition of a few amino acid residues at the C-terminus of the heavy chain CH1 domain, including one or more cysteines from the antibody hinge region. A Fab fragment may also be a single-chain Fab molecule, i.e. a Fab molecule in which the Fab light chain and the Fab heavy chain are connected by a peptide linker to form a single peptide chain. For example, the C-terminus of the Fab light chain may be connected to the N-terminus of the Fab heavy chain in the single-chain Fab molecule.

An “scFv” includes a heavy chain variable domain (VH) and a light chain variable domain (VL) of an antibody in a single polypeptide chain. The scFv may optionally further comprise a polypeptide linker between the VH and VL domains which enables the scFv to form a desired structure for antigen binding. For example, an scFv may include a VL connected from its C-terminus to the N-terminus of a VH by a polypeptide linker. Alternately, an scFv may comprise a VH connected through its C-terminus to the N-terminus of a VL by a polypeptide linker (see review in Pluckthun in The Pharmacology of Monoclonal Antibodies, vol. 113, Rosenburg and Moore eds., Springer-Verlag, New York, pp. 269-315 (1994)).

An “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc of an antibody (see, for example, Harmsen & De Haard, 2007, Appl. Microbiol Biotechnol., 77 (1): 13-22).

In those embodiments in which the anti-cMet antibody constructs comprise two or more antigen-binding domains, each additional antigen-binding domain may independently be an immunoglobulin-based antigen-binding domain, such as an antigen-binding antibody fragment, or a non-immunoglobulin-based antigen-binding domain, such as a non-immunoglobulin-based antibody mimetic, or other polypeptide or small molecule capable of specifically binding to its target, for example, a natural or engineered ligand. Non-immunoglobulin-based antibody mimetic formats include, for example, anticalins, fynomers, affimers, alphabodies, DARPins and avimers. The additional antigen-binding domains may bind to the same epitope within c-Met, may bind to a different epitope within c-Met, or may bind to a different antigen.

In certain embodiments, the at least one antigen-binding domain that specifically binds to c-Met comprised by the anti-cMet antibody construct comprises the heavy chain CDR sequences (HCDR1, HCDR2 and HCDR3) of the VH domain sequence set forth in SEQ ID NO: 1, and the light chain CDR sequences (LCDR1, LCDR2 and LCDR3) of the VL domain sequence set forth in SEQ ID NO: 2 (see Table 2).

In certain embodiments, the at least one antigen-binding domain that specifically binds to c-Met comprised by the anti-cMet antibody construct comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23, and an HCDR3 sequence selected from the sequences as set forth in SEQ ID NOs: 5, 11 and 18 (see Table 2). In certain embodiments, the at least one antigen-binding domain that specifically binds to c-Met comprised by the anti-cMet antibody construct comprises an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21 (see Table 2).

In certain embodiments, the at least one antigen-binding domain that specifically binds to c-Met comprised by the anti-cMet antibody construct comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23; an HCDR3 sequence selected from the sequences as set forth in SEQ ID NOs: 5, 11 and 18; an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21 (see Table 2).

In certain embodiments, the at least one antigen-binding domain that specifically binds to c-Met comprised by the anti-cMet antibody construct comprises heavy chain CDR (HCDR1, HCDR2 and HCDR3) sequences as set forth in SEQ ID NOs: 3, 4 and 5, respectively, and the light chain CDR (LCDR1, LCDR2 and LCDR3) sequences as set forth in SEQ ID NOs: 6, 7 and 8, respectively (see Table 2).

TABLE 2
VH, VL and CDR Sequences

| | | SEQ ID |
|---|---|---|
| Name | Sequence | NO |
| VH | QVQLVQSGAEVKKPGASVKVSCKASGYIFTAYTMHWVRQAPG | 1 |
| | QGLEWMGWIKPNNGLANYAQKFQGRVTMTRDTSISTAYMELS | |
| | RLRSDDTAVYYCARSEITTEFDYWGQGTLVTVSS | |
| VL | DIVMTQSPDSLAVSLGERATINCKSSESVDSYANSFLHWYQQKP | 2 |
| | GQPPKLLIYRASTRESGVPDRFSGSGSGTDFTLTISSLQAEDVAV | |
| | YYCQQSKEDPLTFGGGTKVEIK | |

IMGT

| | | |
|---|---|---|
| HCDR1 | GYIFTAYT | 3 |
| HCDR2 | IKPNNGLA | 4 |
| HCDR3 | ARSEITTEFDY | 5 |
| LCDR1 | ESVDSYANSF | 6 |
| LCDR2 | RAS | 7 |
| LCDR3 | QQSKEDPLT | 8 |

Chothia

| | | |
|---|---|---|
| HCDR1 | GYIFTAY | 9 |
| HCDR2 | KPNNGL | 10 |
| HCDR3 | SEITTEFDY | 11 |
| LCDR1 | KSSESVDSYANSFLH | 12 |
| LCDR2 | RASTRES | 13 |
| LCDR3 | QQSKEDPLT | 8 |

Kabat

| | | |
|---|---|---|
| HCDR1 | AYTMH | 14 |
| HCDR2 | WIKPNNGLANYAQKFQG | 15 |
| HCDR3 | SEITTEFDY | 11 |
| LCDR1 | KSSESVDSYANSFLH | 12 |
| LCDR2 | RASTRES | 13 |
| LCDR3 | QQSKEDPLT | 8 |

Contact

| | | |
|---|---|---|
| HCDR1 | TAYTMH | 16 |
| HCDR2 | WMGWIKPNNGLAN | 17 |
| HCDR3 | ARSEITTEFD | 18 |
| LCDR1 | DSYANSFLHWY | 19 |
| LCDR2 | LLIYRASTRE | 20 |
| LCDR3 | QQSKEDPL | 21 |

AbM

| | | |
|---|---|---|
| HCDR1 | GYIFTAYTMH | 22 |
| HCDR2 | WIKPNNGLAN | 23 |
| HCDR3 | SEITTEFDY | 11 |
| LCDR1 | KSSESVDSYANSFLH | 12 |
| LCDR2 | RASTRES | 13 |
| LCDR3 | QQSKEDPLT | 8 |

In certain embodiments, the anti-cMet antibody construct comprises two antigen-binding domains, each of which specifically binds to c-Met. In some embodiments, each of the two antigen-binding domains that specifically binds to c-Met comprised by the anti-cMet antibody construct comprises the heavy chain CDR sequences (HCDR1, HCDR2 and HCDR3) of the VH domain sequence set forth in SEQ ID NO: 1, and the light chain CDR sequences (LCDR1, LCDR2 and LCDR3) of the VL domain sequence set forth in SEQ ID NO: 2 (see Table 2).

In certain embodiments, the anti-cMet antibody construct comprises two antigen-binding domains, each of which comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23, and an HCDR3 sequence selected from the sequences as set forth in SEQ ID NOs: 5, 11 and 18 (see Table 2). In certain embodiments, the anti-cMet antibody construct comprises two antigen-binding domains, each of which comprises an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21 (see Table 2).

In certain embodiments, the anti-cMet antibody construct comprises two antigen-binding domains, each of which comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23; an HCDR3 sequence selected from the sequences as set forth in SEQ ID NOs: 5, 11 and 18; an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21 (see Table 2).

In certain embodiments, the anti-cMet antibody construct comprises two antigen-binding domains, each of which comprises heavy chain CDR (HCDR1, HCDR2 and HCDR3) sequences as set forth in SEQ ID NOs: 3, 4 and 5, respectively, and the light chain CDR (LCDR1, LCDR2 and LCDR3) sequences as set forth in SEQ ID NOs: 6, 7 and 8, respectively (see Table 2).

### Hinge Region

In certain embodiments, the anti-cMet antibody construct comprises an immunoglobulin (Ig) hinge region. The Ig hinge region may be based on a native human IgG1, IgG2 or IgG4 hinge region sequence or it may be a modified version of a native human IgG1, IgG2 or IgG4 hinge region sequence.

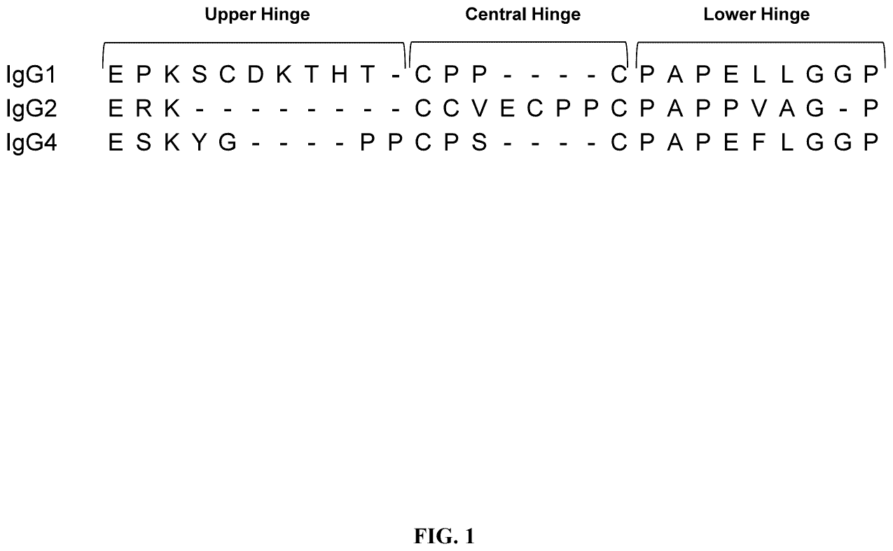

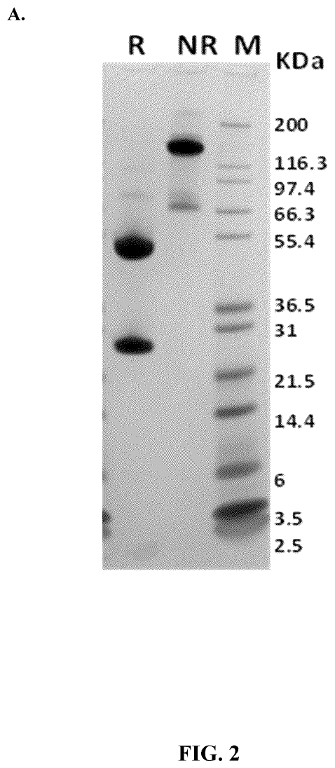

As is known in the art, the hinge region of an immunoglobulin is a flexible, hydrophilic region that connects the CH1 and CH2 domains and is generally defined as extending from position 216 to position 238 in IgG1 (Burton, 1985, Molec. Immunol., 22:161-206). Hinge regions of other IgG isotypes may be aligned with the IgG1 sequence by aligning the first and last cysteine residues that form inter-heavy chain disulfide bonds as shown in FIG. 1. An Ig hinge region can be considered to comprise three sub-parts: the upper hinge, central hinge and lower hinge (see FIG. 1) (Burton, 1985, ibid; see also Deveuve, et al., 2019, Med Sci (Paris), 35 (12): 1098-1105). The sequences of the native full and upper hinge regions of human IgG1, IgG2 and IgG4 are provided in Table 3.

TABLE 3
Hinge Region Sequences

| IgG | | | SEQ |
|---|---|---|---|
| Subclass | Region | Sequence | ID NO |
| IgG1 | Full | EPKSCDKTHTCPPCPAPELLGGP | 24 |
| | Upper | EPKSCDKTHT | 25 |
| IgG2 | Full | ERKCCVECPPCPAPPVAGP | 26 |
| | Upper | ERK | 27 |
| IgG4 | Full | ESKYGPPCPSCPAPEFLGGP | 28 |
| | Upper | ESKYGPP | 29 |

In certain embodiments, the anti-cMet antibody construct comprises an Ig hinge region comprising an upper hinge sequence having an amino acid sequence of a native IgG1, IgG2 or IgG4 upper hinge sequence. In some embodiments, the anti-cMet antibody construct comprises an Ig hinge region that comprises the amino acid sequence as set forth in any one of SEQ ID NOs: 25, 27 or 29. In some embodiments, the anti-cMet antibody construct comprises an Ig hinge region comprising an upper hinge sequence having an amino acid sequence of a native IgG1 upper hinge sequence. In some embodiments, the anti-cMet antibody construct comprises an Ig hinge region that comprises the amino acid sequence as set forth in SEQ ID NO: 25.

In certain embodiments, the anti-cMet antibody construct comprises an Ig hinge region comprising at least a portion of a native IgG1, IgG2 or IgG4 hinge sequence, for example, the upper hinge sequence or the upper and central (or “core”) hinge sequences. In some embodiments, the anti-cMet antibody construct comprises an Ig hinge region comprising at least a portion of a native IgG1 hinge sequence. In some embodiments, the anti-cMet antibody construct comprises an Ig hinge region comprising at least a portion of a native IgG1 hinge sequence, where the portion has the amino acid sequence: EPKSCDKTHTCPPCP (SEQ ID NO: 35).

In certain embodiments, the anti-cMet antibody construct comprises an Ig hinge region comprising an amino acid sequence of a native IgG1, IgG2 or IgG4 hinge region. In some embodiments, the anti-cMet antibody construct comprises an Ig hinge region that comprises the amino acid sequence as set forth in any one of SEQ ID NOs: 24, 26 or 28. In some embodiments, the anti-cMet antibody construct comprises an Ig hinge region comprising an amino acid sequence of a native IgG1 hinge region. In some embodiments, the anti-cMet antibody construct comprises an Ig hinge region that comprises the amino acid sequence as set forth in SEQ ID NO: 24.

In certain embodiments, the anti-cMet antibody construct comprises an Ig hinge region that is a modified version of a native human IgG1, IgG2 or IgG4 hinge region sequence. In some embodiments, the anti-cMet antibody construct comprises an Ig hinge region that is a modified version of a native human IgG1 hinge region sequence. For example, in some embodiments, the anti-cMet antibody construct may comprise one of the modified hinge sequences described in U.S. Pat. No. 8,545,839 or U.S. Pat. No. 8,741,290. In some embodiments, the anti-cMet antibody construct may comprise the modified hinge sequence: EPKSCDCHCPPCP (SEQ ID NO: 36).

### Scaffolds

In certain embodiments, at least one of the one or more antigen-binding domains comprised by the anti-cMet antibody constructs of the present disclosure are operably linked to a scaffold. The term “operably linked,” as used herein, means that the components described are in a relationship permitting them to function in their intended manner. Examples of suitable scaffolds are described in more detail below and include, but are not limited to, immunoglobulin Fc regions, albumin, albumin analogues and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include antibodies based on the DOCK-AND-LOCK™ (DNL™) technology developed by IBC Pharmaceuticals, Inc. and Immunomedics, Inc. (see, for example, Chang, et al., 2007, Clin. Cancer Res., 13: 5586s-5591s).

A scaffold may be a peptide, polypeptide, polymer, nanoparticle or other chemical entity. Where the scaffold is a polypeptide, the antigen-binding domain may be linked to either the N- or C-terminus of the polypeptide scaffold. Anti-cMet antibody constructs comprising a polypeptide scaffold in which one or more of the antigen-binding domains are linked to a region other than the N- or C-terminus, for example, via the side chain of an amino acid with or without a linker, are also contemplated in certain embodiments.

The antigen-binding domain(s) of the anti-cMet antibody construct may be linked to the scaffold by genetic fusion or chemical conjugation. In certain embodiments, when the scaffold is a peptide or polypeptide, the antigen-binding domain(s) are linked to the scaffold by genetic fusion. In some embodiments, where the scaffold is a polymer or nanoparticle, the antigen-binding domain(s) may be linked to the scaffold by chemical conjugation.

In certain embodiments, the anti-cMet antibody construct may comprise a protein scaffold. The use of protein scaffolds in combination with antigen-binding moieties has been described (see, for example, Müller et al., 2007, J. Biol. Chem., 282:12650-12660; McDonaugh et al., 2012, Mol. Cancer Ther., 11:582-593; Vallera et al., 2005, Clin. Cancer Res., 11:3879-3888; Song et al., 2006, Biotech. Appl. Biochem., 45:147-154, and U.S. Patent Application Publication No. 2009/0285816).

In certain embodiments, the anti-cMet antibody construct may comprise a protein scaffold that is based on an immunoglobulin Fc region, an albumin or an albumin analogue or derivative. For example, fusing antigen-binding moieties such as scFvs, diabodies or single chain diabodies to albumin has been shown to improve the serum half-life of the antigen-binding moieties (Müller et al., ibid.). Antigen-binding moieties may be fused at the N- and/or C-terminus of albumin, optionally via a linker. Derivatives of albumin in the form of heteromultimers that comprise two transporter polypeptides obtained by segmentation of an albumin protein such that the transporter polypeptides self-assemble to form quasi-native albumin have been described (see International Patent Application Publication Nos. WO 2012/116453 and WO 2014/012082). As a result of the segmentation of albumin, the heteromultimer includes four termini and thus can be fused to up to four different antigen-binding moieties, optionally via linkers.

In some embodiments, the anti-cMet antibody construct may comprise a protein scaffold that is based on an immunoglobulin Fc region, for example, an IgG Fc region.

### Fc Regions

The terms “Fc region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

In certain embodiments, the anti-cMet antibody constructs of the present disclosure may comprise a scaffold that is based on an immunoglobulin (Ig) Fc region. The Fc region may be dimeric and composed of two Fc polypeptides or alternatively, the Fc region may be composed of a single polypeptide.

An “Fc polypeptide” in the context of a dimeric Fc refers to one of the two polypeptides forming the dimeric Fc domain, i.e. a polypeptide comprising one or more C-terminal constant regions of an immunoglobulin heavy chain that is capable of stable self-association. When referring to a dimeric Fc region, the terms “first Fc polypeptide” and “second Fc polypeptide” may be used interchangeably provided that the Fc region comprises one first Fc polypeptide and one second Fc polypeptide.

An Fc region may comprise a CH3 domain or it may comprise both a CH3 and a CH2 domain. For example, in certain embodiments, an Fc polypeptide of a dimeric IgG Fc region may comprise an IgG CH2 domain sequence and an IgG CH3 domain sequence. In such embodiments, the CH3 domain comprises two CH3 domain sequences, one from each of the two Fc polypeptides of the dimeric Fc region, and the CH2 domain comprises two CH2 domain sequences, one from each of the two Fc polypeptides of the dimeric Fc region.

In some embodiments, the anti-cMet antibody construct may comprise a scaffold that is based on an IgG Fc region and comprises an IgG CH2 domain and an IgG CH3 domain. In some embodiments, the anti-cMet antibody construct may comprise a scaffold that is based on a human IgG Fc region. In some embodiments, the anti-cMet antibody construct may comprise a scaffold based on an IgG1 Fc region. In some embodiments, the anti-cMet antibody construct may comprise a scaffold based on a human IgG1 Fc region.

In certain embodiments, the anti-cMet antibody construct may comprise a scaffold based on an IgG Fc region, which is a homodimeric Fc region, comprising a first Fc polypeptide and a second Fc polypeptide, each comprising a CH3 domain sequence, and optionally a CH2 domain sequence and in which the first and second Fc polypeptides are the same. In certain embodiments, the anti-cMet antibody construct may comprise a scaffold based on an IgG Fc region, which is a heterodimeric Fc region, comprising a first Fc polypeptide and a second Fc polypeptide, each comprising a CH3 domain sequence, and optionally a CH2 domain sequence and in which the first and second Fc polypeptides are different. In some embodiments, the anti-cMet antibody construct may comprise a scaffold based on an Fc region which comprises two CH3 domain sequences, at least one of which comprises one or more amino acid modifications. In some embodiments, the anti-cMet antibody construct may comprise a scaffold based on an Fc region which comprises two CH3 domain sequences and two CH2 domain sequences, at least one of the CH2 domain sequences comprising one or more amino acid modifications.

In some embodiments, the anti-cMet antibody construct may comprise a heterodimeric Fc region comprising a modified CH3 domain, where the modified CH3 domain is an asymmetrically modified CH3 domain comprising one or more asymmetric amino acid modifications. As used herein, an “asymmetric amino acid modification” refers to a modification, such as a substitution or an insertion, in which an amino acid at a specific position on a first CH3 domain sequence or CH2 domain sequence is different to the amino acid on a second CH3 domain sequence or CH2 domain sequence at the same position. These asymmetric amino acid modifications can be a result of modification of only one of the two amino acids at the same respective amino acid position on each sequence, or different modifications of both amino acids at the same respective position on each of the first and second CH3 or CH2 domain sequences. Each of the first and second CH3 or CH2 domain sequences of a heterodimeric Fc may comprise one or more than one asymmetric amino acid modification.

In some embodiments, the anti-cMet antibody construct may comprise a heterodimeric Fc comprising a modified CH3 domain, where the modified CH3 domain comprises one or more amino acid modifications that promote formation of the heterodimeric Fc over formation of a homodimeric Fc. In some embodiments, one or more of the amino acid modifications that promote formation of the heterodimeric Fc are asymmetric amino acid modifications.

Amino acid modifications that may be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23 (4): 195-202 (strand exchange engineered domain (SEED) technology) and Labrijn et al., 2013, Proc Natl Acad Sci USA, 110 (13): 5145-50 (Fab-arm exchange). Other examples include approaches combining positive and negative design strategies to produce stable asymmetrically modified Fc regions as described in International Publication Nos. WO 2012/058768 and WO 2013/063702. In certain embodiments, the anti-cMet antibody construct may comprise a scaffold based on a modified Fc region as described in International Publication No. WO 2012/058768 or WO 2013/063702.

Table 4 provides the amino acid sequence of the human IgG1 Fc sequence (SEQ ID NO: 30), corresponding to amino acids 231 to 447 of the full-length human IgG1 heavy chain. The CH3 sequence comprises amino acids 341-447 of the full-length human IgG1 heavy chain. Also shown in Table 4 are CH3 domain amino acid modifications that promote formation of a heterodimeric Fc as described in in International Patent Application Publication Nos. WO 2012/058768 and WO 2013/063702.

In certain embodiments, the anti-cMet antibody construct may comprise a heterodimeric Fc scaffold having a modified CH3 domain comprising the modifications of any one of Variant 1, Variant 2, Variant 3, Variant 4 or Variant 5, as shown in Table 4.

In certain embodiments, the anti-cMet antibody construct may comprise a heterodimeric Fc scaffold having a modified CH3 domain comprising a first Fc polypeptide and a second Fc polypeptide, in which
a) the first Fc polypeptide comprises the amino acid modifications L351Y, F405A and Y407V, and the second Fc polypeptide comprises the amino acid modifications T366L, K392M and T394W, or
b) the first Fc polypeptide comprises the amino acid modifications L351Y, F405A and Y407V, and the second Fc polypeptide comprises the amino acid modifications T366L, K392L and T394W, or
c) the first Fc polypeptide comprises the amino acid modifications T350V, L351Y, F405A and Y407V, and the second Fc polypeptide comprises the amino acid modifications T350V, T366L, K392M and T394W, or
d) the first Fc polypeptide comprises the amino acid modifications T350V, L351Y, F405A and Y407V, and the second Fc polypeptide comprises the amino acid modifications T350V, T366L, K392L and T394W, or
e) the first Fc polypeptide comprises the amino acid modifications T350V, L351Y, S400E, F405A and Y407V, and the second Fc polypeptide comprises the amino acid modifications T350V, T366L, N390R, K392M and T394W.

TABLE 4
Human IgG1 Fc Sequence¹ and CH3 Domain Amino Acid Modifications
Promoting Heterodimer Formation

APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK (SEQ ID NO: 30)

| Variant No | Chain | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

¹Sequence from positions 231-447 (EU numbering)

In some embodiments, the anti-cMet antibody construct may comprise a scaffold based on an Fc region comprising two CH3 domain sequences and two CH2 domain sequences, at least one of the CH2 domain sequences comprising one or more amino acid modifications. Modifications in the CH2 domain can affect the binding of Fc receptors (FcRs) to the Fc, such as receptors of the FcγRI, FcγRII and FcγRIII subclasses. In some embodiments, the anti-cMet antibody construct comprises a scaffold based on an IgG Fc having a modified CH2 domain, wherein the modification of the CH2 domain results in altered binding to one or more of the FcγRI, FcγRII and FcγRIII receptors.

A number of amino acid modifications to the CH2 domain that selectively alter the affinity of the Fc for different Fcγ receptors are known in the art (see, for example, Lu, et al., 2011, J Immunol Methods, 365 (1-2): 132-41; Stavenhagen, et al. 2007, Cancer Res 67 (18): 8882-90; Nordstrom, et al., 2011, Breast Cancer Res, 13 (6): R123; Stewart, et al., 2011, Protein Eng Des Sel., 24 (9): 671-8; Shields, et al., 2001, J Biol Chem, 276 (9): 6591-604; Lazar, et al., 2006, Proc Natl Acad Sci USA, 103 (11): 4005-10, Chu, et al., 2008, Mol Immunol, 45 (15): 3926-33; International Publication No. WO 2021/232162, and Therapeutic Antibody Engineering (Strohl & Strohl, Woodhead Publishing series in Biomedicine No 11, ISBN 1 907568 37 9, October 2012, page 283)).

Amino acid modifications that result in increased FcγR binding and amino acid modifications that result in decreased FcγR binding can each be useful in certain indications. For example, increasing binding affinity of an Fc for FcγRIIIa (an activating receptor) may result in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249; U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420:204-219). Other examples of mutations that may be introduced into the hinge or CH2 domain to produce a “knock-out” variant include the amino acid modifications L234A/L235A, and L234A/L235A/D265S.

In certain embodiments, the anti-cMet antibody constructs described herein may comprise a scaffold based on an IgG Fc in which native glycosylation has been modified. As is known in the art, glycosylation of an Fc may be modified to increase or decrease effector function. For example, mutation of the conserved asparagine residue at position 297 to alanine, glutamine, lysine or histidine (i.e. N297A, Q, K or H) results in an aglycoslated Fc that lacks all effector function (Bolt et al., 1993, Eur. J. Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibodies may be produced, for example in knockout Chinese hamster ovary (CHO) cells lacking fucosyltransferase (FUT8) (Yamane-Ohnuki et al., 2004, Biotechnol. Bioeng., 87:614-622); in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogues to culture medium during antibody production to inhibit incorporation of fucose into the carbohydrate on the antibody. Other methods of producing antibodies with little or no fucose on the Fc glycosylation site (N297) are well known in the art. For example, the GlymaX® technology (ProBioGen AG) (see von Horsten et al., 2010, Glycobiology, 20 (12): 1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants may have reduced fucosylation and/or improved ADCC function (see, for example, International Publication No. WO 2003/011878, U.S. Pat. No. 6,602,684 and US Patent Application Publication No. US 2005/0123546). Useful glycosylation variants also include those having at least one galactose residue in the oligosaccharide attached to the Fc region, which may have improved CDC function (see, for example, International Publication Nos. WO 1997/030087, WO 1998/58964 and WO 1999/22764).

### Anti-cMet Antibody Constructs Comprising Cysteine Mutations

Certain embodiments of the present disclosure relate to anti-cMet antibody constructs as described above that further comprise one or more non-native cysteine residues. The one or more non-native cysteine residues provide a “conjugation handle” allowing for conjugation of a drug-linker as described herein.

Modification of antibodies to include non-native cysteine residues may be achieved by substitution of a native residue with a cysteine residue (see, for example, U.S. Pat. Nos. 7,521,541; 8,455,622 and 9,000,130) or by insertion of a cysteine residue between two native residues in the antibody sequence (see, for example, U.S. Pat. No. 10,744,206).

The anti-cMet antibody constructs may comprise one or more cysteine substitution mutations, one or more cysteine insertion mutations, or a combination thereof. In some embodiments, the anti-cMet antibody constructs may comprise between one and four cysteine substitution mutations, cysteine insertion mutations, or a combination thereof. In some embodiments, the anti-cMet antibody constructs may comprise one or more cysteine substitution mutations, for example, between one and four cysteine substitution mutations. In some embodiments, the anti-cMet antibody constructs may comprise one or more cysteine insertion mutations, for example, between one and four cysteine insertion mutations.

In certain embodiments, the anti-cMet antibody construct comprises at least one antigen-binding domain comprising a VL domain and a VH domain, and optionally a CH1 domain and CL domain, and an Fc region comprising a CH2 domain and a CH3 domain. In some such embodiments, the anti-cMet antibody constructs may comprise one or more cysteine insertion mutations each independently selected from:
(a) a cysteine residue inserted between positions 39 and 40 in the VL domain;
(b) a cysteine residue inserted between positions 40 and 41 in the VL domain;
(c) a cysteine residue inserted between positions 126 and 127 in the CL domain;
(d) a cysteine residue inserted between positions 148 and 149 in the CL domain;
(e) a cysteine residue inserted between positions 149 and 150 in the CL domain;
(f) a cysteine residue inserted between positions 9 and 10 in the VH domain;
(g) a cysteine residue inserted between positions 169 and 170 in the CH1 domain;
(h) a cysteine residue inserted between positions 237 and 238 in the CH2 domain;
(i) a cysteine residue inserted between positions 295 and 296 in the CH2 domain, and
(j) a cysteine residue inserted between positions 299 and 300 in the CH2 domain.

Numbering of amino acids in the VL, CL and VH domains used herein when describing the cysteine insertion mutations is Kabat numbering and the numbering of amino acids in the CH2 domain is EU numbering.

In some embodiments, the anti-cMet antibody constructs may comprise one or more cysteine insertion mutations each independently selected from:
(i) a cysteine residue inserted between positions 40 and 41 in the VL domain;
(ii) a cysteine residue inserted between positions 126 and 127 in the CL domain;
(iii) a cysteine residue inserted between positions 9 and 10 in the VH domain;
(iv) a cysteine residue inserted between positions 237 and 238 in the CH2 domain, and
(v) a cysteine residue inserted between positions 299 and 300 in the CH2 domain.

In some embodiments, the anti-cMet antibody construct may be monovalent and comprises one antigen-binding domain comprising a VL domain and a VH domain, and optionally a CH1 domain and a CL domain. In some embodiments, a monovalent anti-cMet antibody construct may comprise a cysteine residue inserted between positions 39 and 40 in the VL domain; a cysteine residue inserted between positions 40 and 41 in the VL domain; a cysteine residue inserted between positions 126 and 127 in the CL domain; a cysteine residue inserted between positions 148 and 149 in the CL domain; a cysteine residue inserted between positions 149 and 150 in the CL domain; a cysteine residue inserted between positions 9 and 10 in the VH domain, and/or a cysteine residue inserted between positions 169 and 170 in the CH1 domain.

In some embodiments, the anti-cMet antibody construct may be bivalent and comprise two antigen-binding domains, each comprising a VL domain and a VH domain, and optionally a CH1 domain and a CL domain. The antigen-binding domains may both bind the same antigen, or they may each bind a different antigen. A bivalent anti-cMet antibody construct may comprise one or more cysteine insertion mutations in one antigen-binding domain, or it may comprise one or more cysteine insertion mutations in each antigen-binding domain. When the anti-cMet antibody construct comprises one or more cysteine insertion mutations in each antigen-binding domain, each antigen-binding domain may comprise the same cysteine insertion mutation(s) or they may comprise different cysteine insertion mutation(s).

In some embodiments, a bivalent anti-cMet antibody construct may comprise a cysteine residue inserted between positions 39 and 40 in one VL domain. In some embodiments, a bivalent anti-cMet antibody construct may comprise a cysteine residue inserted between positions 39 and 40 in each VL domain. In some embodiments, a bivalent anti-cMet antibody construct may comprise a cysteine residue inserted between positions 40 and 41 in one VL domain. In some embodiments, a bivalent anti-cMet antibody construct may comprise a cysteine residue inserted between positions 40 and 41 in each VL domain. In some embodiments, a bivalent anti-cMet antibody construct may comprise a cysteine residue inserted between positions 126 and 127 in one CL domain. In some embodiments, a bivalent anti-cMet antibody construct may comprise a cysteine residue inserted between positions 126 and 127 in each CL domain. In some embodiments, a bivalent anti-cMet antibody construct may comprise a cysteine residue inserted between positions 148 and 149 in one CL domain. In some embodiments, a bivalent anti-cMet antibody construct may comprise a cysteine residue inserted between positions 148 and 149 in each CL domain. In some embodiments, a bivalent anti-cMet antibody construct may comprise a cysteine residue inserted between positions 149 and 150 in one CL domain. In some embodiments, a bivalent anti-cMet antibody construct may comprise a cysteine residue inserted between positions 149 and 150 in each CL domain. In some embodiments, a bivalent anti-cMet antibody construct may comprise a cysteine residue inserted between positions 9 and 10 in one VH domain. In some embodiments, a bivalent anti-cMet antibody construct may comprise a cysteine residue inserted between positions 9 and 10 in each VH domain. In some embodiments, a bivalent anti-cMet antibody construct may comprise a cysteine residue inserted between positions 169 and 170 in one CH1 domain. In some embodiments, a bivalent anti-cMet antibody construct may comprise a cysteine residue inserted between positions 169 and 170 in each CH1 domain.

In certain embodiments, the anti-cMet antibody construct may comprise an Fc region. In some embodiments, the anti-cMet antibody construct may comprise a dimeric Fc region composed of two Fc polypeptides as described above, in which the CH3 domain of the Fc region comprises two CH3 domain sequences, one from each of the two Fc polypeptides of the dimeric Fc region, and the CH2 domain of the Fc region comprises two CH2 domain sequences, one from each of the two Fc polypeptides of the dimeric Fc region. In some embodiments, the anti-cMet antibody construct may comprise an Fc region composed of a single Fc polypeptide as described above, in which the CH3 domain of the Fc region comprises two CH3 domain sequences, and the CH2 domain of the Fc region comprises two CH2 domain sequences, with both CH3 domain sequences and both CH2 domain sequences being comprised by the single Fc polypeptide.

In some embodiments, the anti-cMet antibody constructs may comprise an Fc region and a cysteine residue inserted between positions 237 and 238 in one CH2 domain sequence. In some embodiments, the anti-cMet antibody construct may comprise an Fc region and a cysteine residue inserted between positions 237 and 238 in each CH2 domain sequence. In some embodiments, the anti-cMet antibody constructs may comprise an Fc region and a cysteine residue inserted between positions 295 and 296 in one CH2 domain sequence. In some embodiments, the anti-cMet antibody construct may comprise an Fc region and a cysteine residue inserted between positions 295 and 296 in each CH2 domain sequence. In some embodiments, the anti-cMet antibody constructs may comprise an Fc region and a cysteine residue inserted between positions 299 and 300 in one CH2 domain sequence. In some embodiments, the anti-cMet antibody construct may comprise an Fc region and a cysteine residue inserted between positions 299 and 300 in each CH2 domain sequence.

In certain embodiments, the anti-cMet antibody construct comprises an antigen-binding domain that specifically binds c-Met and comprises a VL domain and a VH domain, and optionally a CH1 domain and a CL domain, and an Fc region comprising a CH2 domain having two CH2 domain sequences and a CH3 domain having two CH3 domain sequences, where the antibody construct comprises one or more cysteine insertion mutations independently selected from:
(a) a cysteine residue inserted between positions 40 and 41 in the VL domain;
(b) a cysteine residue inserted between positions 126 and 127 in the CL domain;
(c) a cysteine residue inserted between positions 9 and 10 in the VH domain;
(d) a cysteine residue inserted between positions 237 and 238 in a CH2 domain sequence, and
(e) a cysteine residue inserted between positions 299 and 300 in a CH2 domain sequence.

Various combinations of the cysteine insertion mutations described above are contemplated and may be selected based on whether the antibody construct is monovalent, bivalent or multivalent, and on the nature of the antigen-binding domain (for example, whether the antigen-binding domain is a Fab or an scFv). In certain embodiments, the anti-cMet antibody construct may comprise a combination of cysteine insertions. In some embodiments, the anti-cMet antibody construct may comprise a combination of cysteine insertions where the combination comprises:
(a) a cysteine residue inserted between positions 299 and 300 and between positions 237 and 238 in the CH2 domain, or
(b) a cysteine residue inserted between positions 299 and 300 in the CH2 domain, and between positions 9 and 10 in the VH domain, or
(c) a cysteine residue inserted between positions 299 and 300 in the CH2 domain, and between positions 40 and 41 in the VL domain, or
(d) a cysteine residue inserted between positions 237 and 238 in the CH2 domain, and between positions 9 and 10 in the VH domain, or
(e) a cysteine residue inserted between positions 9 and 10 in the VH domain, and between positions 40 and 41 in the VL domain.

In some embodiments, the anti-cMet antibody construct comprises at least one antigen-binding domain comprising a VL domain, a VH domain, and optionally a CH1 domain and a CL domain, and an Fc region, where the anti-cMet antibody construct comprises (i) a cysteine insertion between positions 299 and 300 in one CH2 domain sequence, and (ii) a cysteine residue inserted between positions 299 and 300, and a cysteine residue inserted between positions 237 and 238 in the other CH2 domain sequence. In some embodiments, the anti-cMet antibody construct comprises at least one antigen-binding domain comprising a VL domain, a VH domain, and optionally a CH1 domain and a CL domain, and an Fc region, where the anti-cMet antibody construct comprises (i) a cysteine insertion between positions 299 and 300 in each CH2 domain sequence, and (ii) a cysteine residue inserted between positions 9 and 10 in one VH domain.

In some embodiments, the anti-cMet antibody construct comprises two antigen-binding domains each comprising a VL domain, a VH domain, and optionally a CH1 domain and a CL domain, and an Fc region, where the anti-cMet antibody construct comprises (i) a cysteine insertion between positions 299 and 300 in one CH2 domain sequence, and (ii) a cysteine residue inserted between positions 299 and 300, and a cysteine residue inserted between positions 237 and 238 in the other CH2 domain sequence. In some embodiments, the anti-cMet antibody construct comprises two one antigen-binding domains each comprising a VL domain, a VH domain, and optionally a CH1 domain and a CL domain, and an Fc region, where the anti-cMet antibody construct comprises (i) a cysteine insertion between positions 299 and 300 in each CH2 domain sequence, and (ii) a cysteine residue inserted between positions 9 and 10 in one VH domain.

In some embodiments, the anti-cMet antibody construct comprises two antigen-binding domains each comprising a VL domain, a VH domain, and optionally a CH1 domain and a CL domain, and an Fc region, where the anti-cMet antibody construct comprises a cysteine residue inserted between positions 40 and 41 in each VL domain, and either (i) a cysteine residue inserted between positions 299 and 300 in one CH2 domain sequence, or (ii) a cysteine residue inserted between positions 9 and 10 in one VH domain. In some embodiments, the anti-cMet antibody construct comprises two antigen-binding domains each comprising a VL domain, a VH domain, and optionally a CH1 domain and a CL domain, and an Fc region, where the anti-cMet antibody construct comprises a cysteine residue inserted between positions 9 and 10 in each VH domain, and a cysteine insertion between positions 237 and 238 in one CH2 domain sequence.

In certain embodiments, the anti-cMet antibody construct comprises at least one VH domain, at least one VL domain and an Fc region comprising two CH2 domain sequences and one of the following combinations of cysteine insertion mutations:
(a) a cysteine residue inserted between positions 299 and 300 and between positions 237 and 238 in one or both CH2 domain sequences, or
(b) a cysteine residue inserted between positions 299 and 300 in one or both CH2 domain sequences, and a cysteine residue inserted between positions 9 and 10 in a VH domain, or
(c) a cysteine residue inserted between positions 299 and 300 in one or both CH2 domain sequences, and a cysteine residue inserted between positions 40 and 41 in a VL domain, or
(d) a cysteine residue inserted between positions 237 and 238 in one or both CH2 domain sequences, and a cysteine residue inserted between positions 9 and 10 in a VH domain, or
(e) a cysteine residue inserted between positions 9 and 10 in a VH domain, and a cysteine residue inserted between positions 40 and 41 in a VL domain.

In certain embodiments, the anti-cMet antibody construct comprises one or two VH domains, one or two VL domains and an Fc region comprising two CH2 domain sequences and further comprises:
(i) a cysteine residue inserted between positions 299 and 300 in one CH2 domain sequence, or
(ii) a cysteine residue inserted between positions 299 and 300 in each CH2 domain sequence, or
(iii) a cysteine residue inserted between positions 237 and 238 in one CH2 domain sequence, or
(iv) a cysteine residue inserted between positions 237 and 238 in each CH2 domain sequence, or
(v) a cysteine residue inserted between positions 9 and 10 in one VH domain, or
(vi) a cysteine residue inserted between positions 9 and 10 in each VH domain, or
(vii) a cysteine residue inserted between positions 40 and 41 in each VL domain, or
(viii) a cysteine residue inserted between positions 126 and 127 in each CL domain, or
(ix) a cysteine insertion between positions 299 and 300 in a first CH2 domain sequence, a cysteine residue inserted between positions 299 and 300 in a second CH2 domain sequence, and a cysteine residue inserted between positions 237 and 238 in the second CH2 domain sequence, or
(x) a cysteine residue inserted between positions 9 and 10 in one VH domain, and a cysteine insertion between positions 299 and 300 in each CH2 domain sequence, or
(xi) a cysteine residue inserted between positions 40 and 41 in each VL domain, and a cysteine residue inserted between positions 299 and 300 in one CH2 domain sequence, or
(xii) a cysteine residue inserted between positions 40 and 41 in each VL domain, and a cysteine residue inserted between positions 9 and 10 in one VH domain, or
(xiii) a cysteine residue inserted between positions 9 and 10 in each VH domain, and a cysteine insertion between positions 237 and 238 in one CH2 domain sequence.

In certain embodiments, the anti-cMet antibody construct comprises a VH domain comprising an amino acid sequence as set forth in SEQ ID NO: 59. In certain embodiments, the anti-cMet antibody construct comprises two VH domains, each comprising an amino acid sequence as set forth in SEQ ID NO: 59.

In certain embodiments, the anti-cMet antibody construct comprises a VL domain comprising an amino acid sequence as set forth in SEQ ID NO: 56. In certain embodiments, the anti-cMet antibody construct comprises two VL domains, each comprising an amino acid sequence as set forth in SEQ ID NO: 56.

In certain embodiments, the anti-cMet antibody construct comprises a first heavy chain and a second heavy chain, where the first and second heavy chains each comprise a CH2 domain, where one of the CH2 domains comprises an amino acid sequence selected from the sequences set forth in SEQ ID NOs: 76, 77 and 78. In certain embodiments, the anti-cMet antibody construct comprises a first heavy chain and a second heavy chain, where the first and second heavy chains each comprise a CH2 domain, where both of the CH2 domains comprise an amino acid sequence independently selected from the sequences set forth in SEQ ID NOs: 76, 77 and 78.

In certain embodiments, the anti-cMet antibody construct comprises a first light chain and a second light chain, where the first and second light chains each comprise a CL domain, where one of the CL domains comprises an amino acid sequence as set forth in SEQ ID NO. 79. In certain embodiments, the anti-cMet antibody construct comprises a first light chain and a second light chain, where the first and second light chains each comprise a CL domain, where both of the CL domains comprise an amino acid sequence as set forth in SEQ ID NO. 79.

### Auristatin-Analogue Drug-Linkers

In certain embodiments, the ADCs of the present disclosure comprise an anti-cMet antibody construct as described above conjugated to an auristatin analogue, compound 1, via a linker. Certain embodiments thus relate to drug-linkers (auristatin analogue-linkers) having Formula II:

L-(D)ₙ II
in which
L is a linker;
D has the structure:

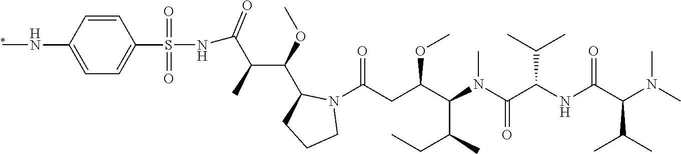 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]

where * is the point of attachment to L, and
n is between 1 and 4.

Linker, L, comprised by the drug-linkers of Formula II and the ADCs of Formula I functions to link one or more auristatin analogues to the anti-cMet antibody construct and may be monovalent or multivalent. A monovalent linker, L, functions to link a single auristatin analogue to a single site on the anti-cMet antibody construct, whereas a multivalent (or polyvalent) linker, L, functions to link more than one auristatin analogue to a single site on the anti-cMet antibody construct. A linker that links one auristatin analogue to more than one site on the anti-cMet antibody construct may also be considered to be multivalent in some embodiments.

In certain embodiments, linker, L, is linked to the anti-cMet antibody construct, A, via a functional group capable of reacting with the target group or groups on the antibody construct and is linked to the auristatin analogue(s) via a functional group capable of reacting with the target amino group on the auristatin analogue. Suitable functional groups are known in the art and include those described, for example, in Bioconjugate Techniques (G. T. Hermanson, 2013, Academic Press). Groups on the anti-cMet antibody construct that may serve as target groups for linker attachment include, but are not limited to, thiol, hydroxyl, carboxyl, amine, aldehyde and ketone groups.

Non-limiting examples of functional groups capable of reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups for reacting with free amines include activated esters (such as N-hydroxysuccinamide (NHS) esters and sulfo-NHS esters), imido esters (such as Traut's reagent), tetrafluorophenyl (TFP) esters, sulfodichlorophenyl esters, isothiocyanates, aldehydes and acid anhydrides (such as diethylenetriaminepentaacetic anhydride (DTPA)). Other examples include the use of succinimido-1,1,3,3-tetra-methyluronium tetrafluoroborate (TSTU) or benzotriazol-1-yl-oxytripyrrolidinophosphonium hexafluorophosphate (PyBOP) to convert a carboxylic acid to an activated ester, which may then be reacted with an amine.

Non-limiting examples of functional groups capable of reacting with an electrophilic group such as an aldehyde or ketone carbonyl group include hydrazide, oxime, amino, hydrazine, thiosemicarbazone, hydrazine carboxylate and arylhydrazide.

In certain embodiments, linker, L, may include a functional group that allows for bridging of two interchain cysteines on the anti-cMet antibody construct, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

Alternatively, the anti-cMet antibody construct may be modified to include a non-natural reactive group, such as an azide, that allows for conjugation to the linker via a complementary reactive group on the linker. For example, conjugation of the linker to the targeting moiety may make use of click chemistry reactions (see, for example, Chio & Bane, 2020, Methods Mol. Biol., 2078:83-97), such as the azide-alkyne cycloaddition (AAC) reaction, which has been used successfully in the development of antibody-drug conjugates. The AAC reaction may be a copper-catalyzed AAC (CuAAC) reaction, which involves coupling of an azide with a linear alkyne, or a strain-promoted AAC (SPAAC) reaction, which involves coupling of an azide with a cyclooctyne.

Linker, L, may be a cleavable or a non-cleavable linker. A cleavable linker is a linker that is susceptible to cleavage under specific conditions, for example, intracellular conditions (such as in an endosome or lysosome) or within the vicinity of a target cell (such as in the tumour microenvironment). Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

Examples of cleavable linkers include, for example, linkers comprising an amino acid sequence that is a cleavage recognition sequence for a protease. Many such cleavage recognition sequences are known in the art. For conjugates that are not intended to be internalized by a cell, for example, an amino acid sequence that is recognized and cleaved by a protease present in the extracellular matrix in the vicinity of a target cell, such as a cancer cell, may be employed. Examples of extracellular tumour-associated proteases include, for example, plasmin, matrix metalloproteases (MMPs), elastase and kallikrein-related peptidases. For conjugates intended to be internalized by a cell, linker, L, may comprise an amino acid sequence that is recognized and cleaved by an endosomal or lysosomal protease. Examples of such proteases include, for example, cathepsins B, C, D, H, L and S, and legumain.

Cleavage recognition sequences may be, for example, dipeptides, tripeptides or tetrapeptides. Non-limiting examples of dipeptide recognition sequences that may be included in cleavable linkers include, but are not limited to, Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

Additional examples of cleavable linkers include disulfide-containing linkers such as N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Other cleavable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumour interstitium (see, for example, De Graaf et al., 2002, Curr. Pharm. Des. 8:1391-1403, and International Patent Publication No. WO 2007/011968). β-glucuronide may also function to improve the hydrophilicity of linker, L.

Another example of a linker that is cleaved internally within a cell and improves hydrophilicity is a linker comprising a pyrophosphate diester moiety (see, for example, Kern et al., 2016, J Am Chem Soc., 138:2430-1445).

In certain embodiments, the linker, L, comprised by an ADC of Formula I and a drug-linker of Formula II is a cleavable linker. In some embodiments, linker, L, comprises a cleavage recognition sequence. In some embodiments, linker, L, may comprise an amino acid sequence that is recognized and cleaved by a lysosomal protease.

Cleavable linkers may optionally further comprise one or more additional functionalities such as self-immolative and self-elimination groups, stretchers or hydrophilic moieties.

Self-immolative and self-elimination groups that find use in linkers include, for example, p-aminobenzyl (PAB) and p-aminobenzyloxycarbonyl (PABC) groups, and methylated ethylene diamine (MED). Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the payload drug. Self-immolative/self-elimination groups, alone or in combination are often included in peptide-based linkers, but may also be included in other types of linkers.

Stretchers that find use in linkers for drug conjugates include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers.

PEG and mPEG stretchers can also function as hydrophilic moieties within a linker. For example, PEG or mPEG may be included in a linker either “in-line” or as pendant groups to increase the hydrophilicity of the linker (see, for example, U.S. Patent Application Publication No. US 2016/0310612). Various PEG-containing linkers are also commercially available from companies such as Quanta BioDesign, Ltd (Plain City, OH). Other hydrophilic groups that may optionally be incorporated into linker, L, include, for example, β-glucuronide, sulfonate groups, carboxylate groups and pyrophosphate diesters.

Selection of an appropriate linker for a given ADC may be readily made by the skilled person having knowledge of the art and taking into account relevant factors, such as the site of attachment to the antibody construct, any structural constraints of the payload drug and the hydrophobicity of the payload drug (see, for example, review in Nolting, Chapter 5, Antibody-Drug Conjugates: Methods in Molecular Biology, 2013, Ducry (Ed.), Springer).

In certain embodiments, drug-linkers of Formula II and ADCs of Formula I may comprise a cleavable linker. In some embodiments, drug-linkers of Formula II and ADCs of Formula I may comprise a peptide-containing linker. In some embodiments, drug-linkers of Formula II and ADCs of Formula I may comprise a protease-cleavable linker.

In certain embodiments, in drug-linkers of Formula II, n is 1, and the drug-linker has Formula III:

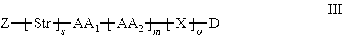

wherein:
Z is a functional group capable of reacting with a target group on the anti-cMet antibody construct, A;
Str is a stretcher;
AA₁ and AA₂ are each independently an amino acid, wherein AA₁-[AA₂]ₘ forms a protease cleavage site;
X is a self-immolative group;
s is 0 or 1;
m is 1, 2 or 3;
o is 0, 1 or 2, and
D has the structure shown in Formula II.

When incorporated into an ADC of the present disclosure, drug-linkers of Formula III have Formula IV:

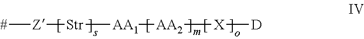

wherein:
Z′ is a linking group that joins the linker to the target group on the anti-cMet antibody construct, A;
Str, AA₁, AA₂, X, s, m, o and D are as defined for Formula III, and #is the point of attachment to the anti-cMet antibody construct, A.

In some embodiments, in Formula III and IV, s is 1.

In some embodiments, in Formula III and IV, o is 0 (i.e. X is absent).

In some embodiments, in Formula III, Z is a functional group capable of reacting with a thiol or amino group on the anti-cMet antibody construct, A.

In some embodiments, in Formula III:
Z is

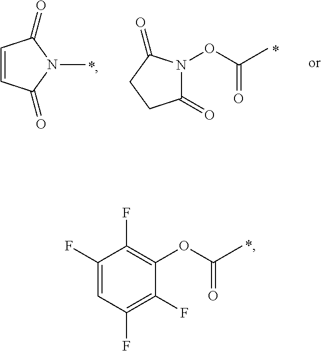

where * is the point of attachment to the remainder of the linker.

In some embodiments, in Formula IV:
Z′ is a carbonyl group (—C(O)—) or

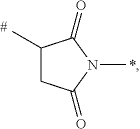

where #is the point of attachment to the anti-cMet antibody construct, A, and * is the point of attachment to the remainder of the linker.

In some embodiments, in Formula III and IV, Str is selected from:

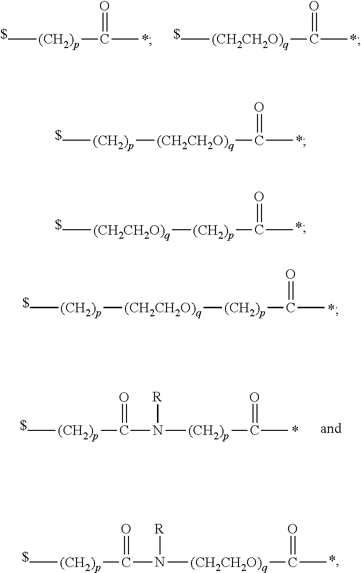

wherein:
each R is independently H or C₁-C₆ alkyl;
each p is independently an integer between 2 and 10;
each q is independently an integer between 1 and 10,
$ is the point of attachment to Z or Z′, and
is the point of attachment to the remainder of the linker.

In some embodiments, in Formula III and IV, Str is:

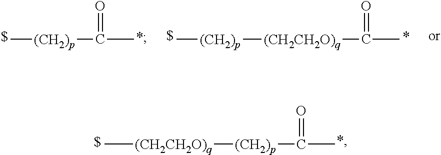

where p, q, $ and * are as defined above.

In some embodiments, in Formula III and IV, Str is:

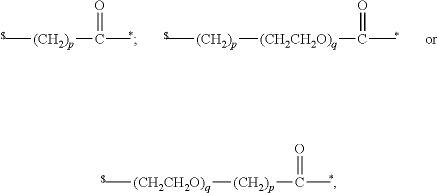

where $ and * are as defined above, p is an integer between 2 and 6, and q is an integer between 2 and 8.

In some embodiments, in Formula III and IV, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D) Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, Ala-Leu-Ala-Leu and Gly-Gly-Phe-Gly.

In some embodiments, in Formula III and IV, m is 1 (i.e. AA₁-[AA₂]ₘ is a dipeptide).

In some embodiments, in Formula III and IV, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in Formula III:
Z is

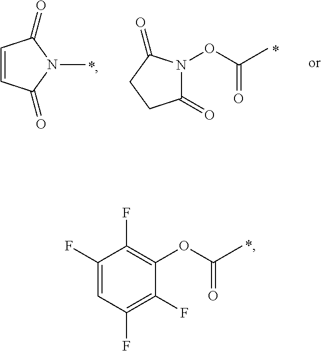

where * is the point of attachment to the remainder of the linker;
Str is

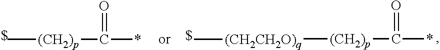

where $ is the point of attachment to Z, * is the point of attachment to the remainder of the linker, p is an integer between 2 and 6, and q is an integer between 2 and 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1, and
is 0.

In some embodiments, in Formula IV:
Z′ is a carbonyl group (—C(O)—) or

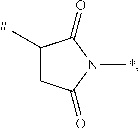

where #is the point of attachment to the anti-cMet antibody construct, A, and * is the point of attachment to the remainder of the linker;
Str is

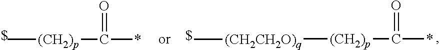

where $ is the point of attachment to Z′, * is the point of attachment to the remainder of the linker, p is an integer between 2 and 6, and q is an integer between 2 and 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1, and
is 0.

In certain embodiments, in drug-linkers of Formula II, n is 1, and the drug-linker has Formula V:

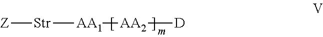

wherein:
Z is a functional group capable of reacting with a target group on the anti-cMet antibody construct, A;
Str is a stretcher;
AA₁ and AA₂ are each independently an amino acid, wherein AA₁-[AA₂]ₘ forms a protease cleavage site;
m is 1, 2 or 3, and
D has the structure shown in Formula II.

When incorporated into an ADC of the present disclosure, drug-linkers of Formula V have Formula VI:

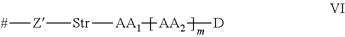

wherein:
Z′ is a linking group that joins the linker to the target group on the anti-cMet antibody construct, A;
Str, AA₁, AA₂, X, m and D are as defined for Formula V, and
#is the point of attachment to the anti-cMet antibody construct, A.

In some embodiments, in Formula V, Z is a functional group capable of reacting with a thiol or amino group on the anti-cMet antibody construct, A.

In some embodiments, in Formula V:
Z is

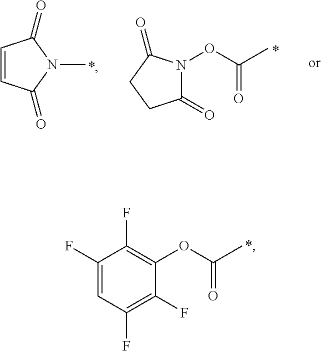

where * is the point of attachment to the remainder of the linker.

In some embodiments, in Formula VI:
Z′ is a carbonyl group (—C(O)—) or

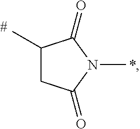

where #is the point of attachment to the anti-cMet antibody construct, A, and * is the point of attachment to the remainder of the linker.

In some embodiments, in Formula V and VI, Str is selected from:

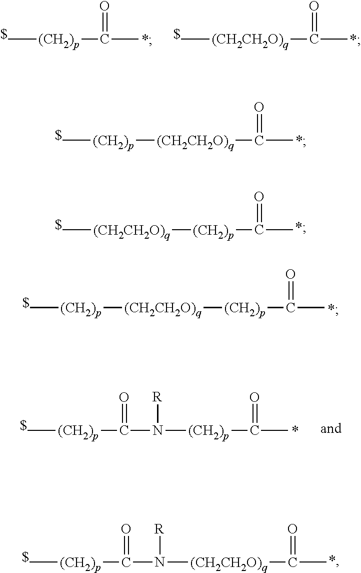

wherein:
each R is independently H or C₁-C₆ alkyl;
each p is independently an integer between 2 and 10;
each q is independently an integer between 1 and 10,
$ is the point of attachment to Z or Z′, and
is the point of attachment to the remainder of the linker.

In some embodiments, in Formula V and VI, Str is:

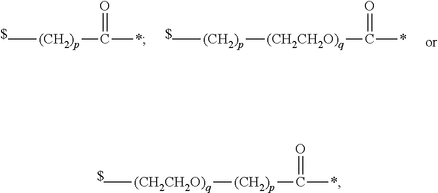

where p, q, $ and * are as defined above.

In some embodiments, in Formula V and VI, Str is:

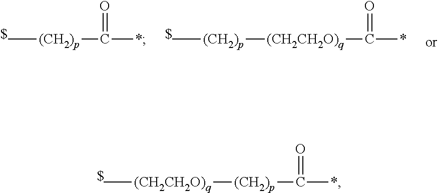

where $ and * are as defined above, p is an integer between 2 and 6, and q is an integer between 2 and 8.

In some embodiments, in Formula V and VI, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D) Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, Ala-Leu-Ala-Leu and Gly-Gly-Phe-Gly.

In some embodiments, in Formula V and VI, m is 1 (i.e. AA₁-[AA₂]ₘ is a dipeptide).

In some embodiments, in Formula V and VI, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in Formula V:
Z is

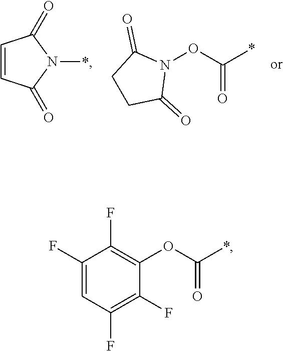

where * is the point of attachment to the remainder of the linker;
Str is

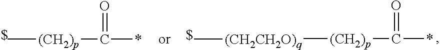

where $ is the point of attachment to Z, * is the point of attachment to the remainder of the linker, p is an integer between 2 and 6, and q is an integer between 2 and 8, and
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in Formula VI:
Z′ is a carbonyl group (—C(O)—) or

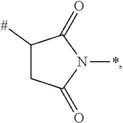

where #is the point of attachment to the anti-cMet antibody construct, and * is the point of attachment to the remainder of the linker;
Str is

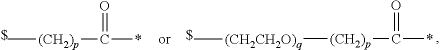

where $ is the point of attachment to Z′, * is the point of attachment to the remainder of the linker, p is an integer between 2 and 6, and q is an integer between 2 and 8, and
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit.

In certain embodiments, in drug-linkers of Formula II, n is 1, and the drug-linker has Formula VII or Formula VIII:

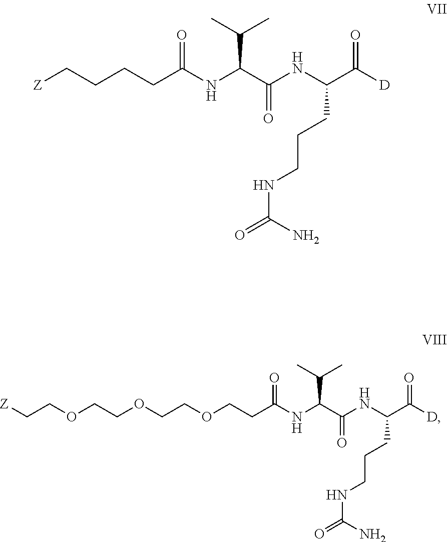

wherein:
Z is a functional group capable of reacting with a target group on the anti-cMet antibody construct, A, and
D has the structure shown in Formula II.

When incorporated into an ADC of the present disclosure, drug-linkers of Formula VII and Formula VIII have Formula IX and Formula X, respectively:

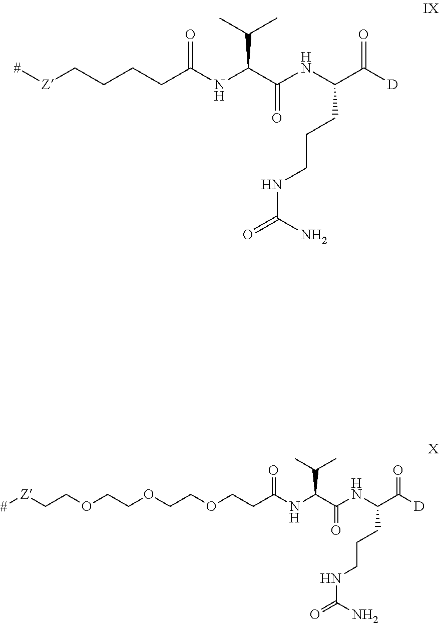

wherein:
Z′ is a linking group that joins the linker to the target group on the anti-cMet antibody construct, A;
#is the point of attachment to the anti-cMet antibody construct, A, and
D has the structure shown in Formula II.

In certain embodiments, in drug-linkers of Formula II, n is >1, and the drug-linker has Formula XI:

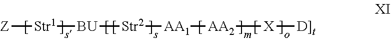

wherein:
Z is a functional group capable of reacting with a target group on the anti-cMet antibody construct, A;
Str¹ and Str² are each independently a stretcher;
BU is a branch unit;
AA₁ and AA₂ are each independently an amino acid, wherein AA₁-[AA₂]ₘ forms a protease cleavage site;
X is a self-immolative group;
s and s′ are each independently 0 or 1;
m is 1, 2 or 3;
o is 0, 1 or 2;
tis 2 or 3, and
D has the structure shown in Formula II.

When incorporated into an ADC of the present disclosure, drug-linkers of Formula XI have Formula XII:

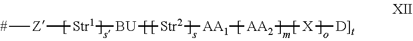

wherein:
Z′ is a linking group that joins the linker to the target group on the anti-cMet antibody construct, A;
Str¹, Str², BU, AA₁, AA₂, X, s, s′, m, o, t and D are as defined for Formula XI, and
#is the point of attachment to the anti-cMet antibody construct, A.

In Formula XI and XII, BU is a multifunctional (tri- or tetra-functional) group that allows multiple components of the drug linker to be joined together. Examples of multifunctional groups that may be employed as a branch unit (BU) include, but are not limited to, tris, amino acids with functional side groups (such as glutamate, aspartate, tyrosine, lysine, cysteine, serine or threonine), tri-substituted aromatic compounds (such as 5-amino isophthalic acid), Behera's amine (di-tert-butyl 4-amino-4-(3-(tert-butoxy)-3-oxopropyl) heptanedioate) and various dendron cores (see, for example, Newkome & Shreiner, 2010, Chem. Reviews, 110 (10): 6338-6442).

In some embodiments, in Formula XI and XII, BU is an amino acid or Behera's amine. In some embodiments, in Formula XI and XII, BU is glutamate or Behera's amine.

In some embodiments, in Formula XI and XII, s is 1. In some embodiments, in Formula XI and XII, s′ is 1. In some embodiments, in Formula XI and XII, s and s′ are each 1.

In some embodiments, in Formula XI and XII, o is 0 (i.e. X is absent).

In some embodiments, in Formula XI, Z is a functional group capable of reacting with a thiol or amino group on the anti-cMet antibody construct, A.

In some embodiments, in Formula XI:
Z is

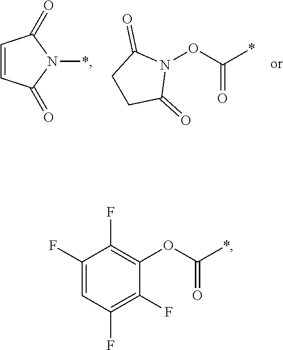

where * is the point of attachment to the remainder of the linker.

In some embodiments, in Formula XI:
Z is

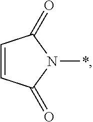

where * is the point of attachment to the remainder of the linker.

In some embodiments, in Formula XII:
Z′ is a carbonyl group (—C(O)—) or

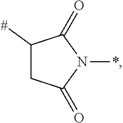

where #is the point of attachment to the anti-cMet antibody construct, A, and * is the point of attachment to the remainder of the linker.

In some embodiments, in Formula XII:
Z′ is

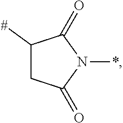

where #is the point of attachment to the anti-cMet antibody construct, A, and * is the point of attachment to the remainder of the linker.

In some embodiments, in Formula XI and XII, Str¹ is selected from:

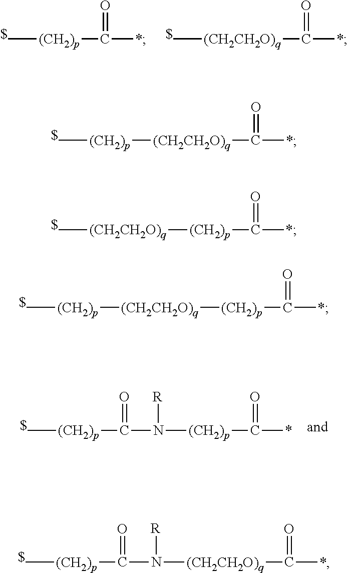

wherein:
each R is independently H or C₁-C₆ alkyl;
each p is independently an integer between 2 and 10;
each q is independently an integer between 1 and 10,
$ is the point of attachment to Z or Z′, and
is the point of attachment to the remainder of the linker.

In some embodiments, in Formula XI and XII, Str² is selected from:

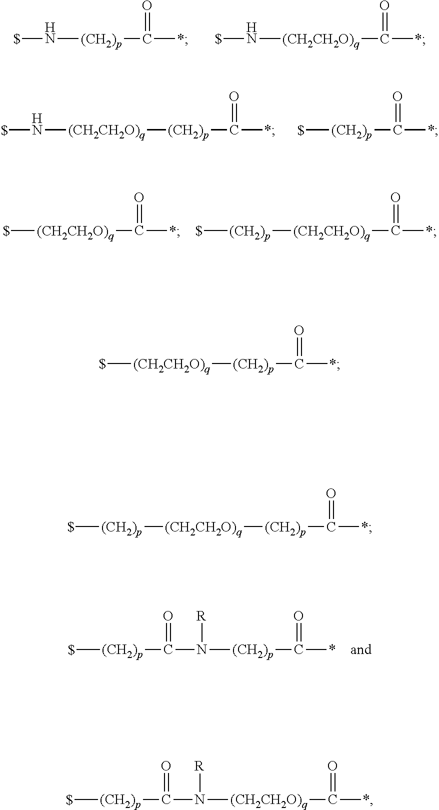

wherein:
each R is independently H or C₁-C₆ alkyl;
each p is independently an integer between 2 and 10;
each q is independently an integer between 1 and 10,
$ is the point of attachment to BU, and
is the point of attachment to the remainder of the linker.

In some embodiments, in Formula XI and XII, Str¹ is:

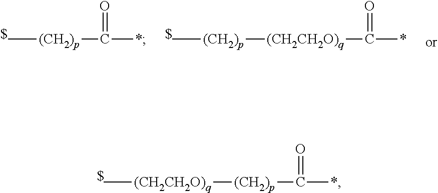

where p, q, $ and * are as defined above for Str¹.

In some embodiments, in Formula XI and XII, Str² is:

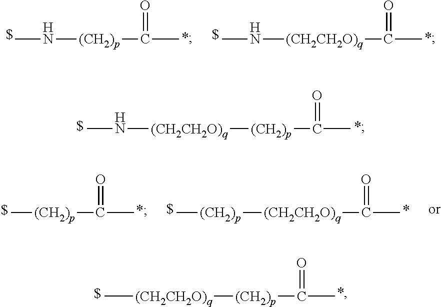

where p, q, $ and * are as defined above for Str².

In some embodiments, in Formula XI and XII, Str¹ is:

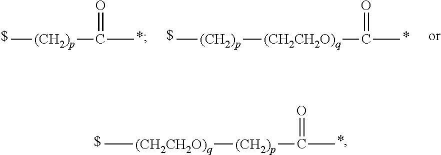

where $ and * are as defined above for Str¹, p is an integer between 2 and 6, and q is an integer between 2 and 8.

In some embodiments, in Formula XI and XII, Str² is:

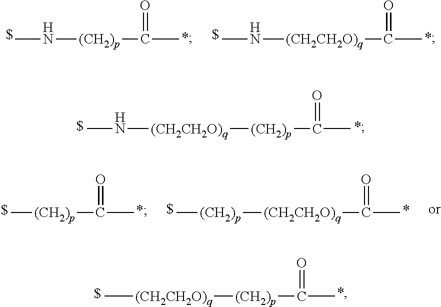

where $ and * are as defined above for Str², p is an integer between 2 and 6, and q is an integer between 2 and 8.

In some embodiments, in Formula XI and XII, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D) Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly,

### Ala-Leu-Ala-Leu and Gly-Gly-Phe-Gly.

In some embodiments, in Formula XI and XII, m is 1 (i.e. AA₁-[AA₂]ₘ is a dipeptide).

In some embodiments, in Formula XI and XII, AA₁-[AA₂]ₘ is a dipeptide selected from Val-Lys, Ala-Lys, Phe-Lys, Val-Cit, Phe-Cit, Leu-Cit, Ile-Cit and Trp-Cit.

In some embodiments, in Formula XI:
Z is

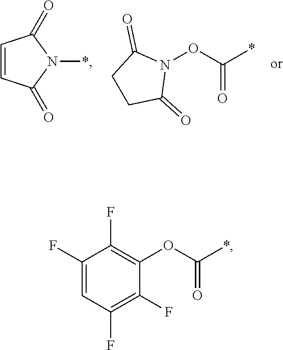

where * is the point of attachment to the remainder of the linker;
BU is an amino acid (for example, glutamate) or Behera's amine,
Str¹ is

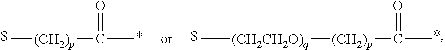

where $ is the point of attachment to Z, * is the point of attachment to the remainder of the linker, p is an integer between 2 and 6, and q is an integer between 2 and 8;
Str² is

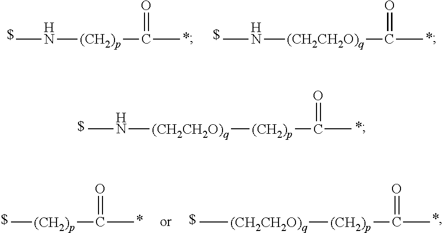

where $ is the point of attachment to BU, * is the point of attachment to the remainder of the linker, p is an integer between 2 and 6, and q is an integer between 2 and 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1;
s′ is 1, and
is 0.

In some embodiments, in Formula XII:
Z′ is a carbonyl group (—C(O)—) or

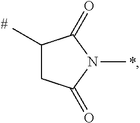

where #is the point of attachment to the anti-cMet antibody construct, A, and * is the point of attachment to the remainder of the linker;
BU is an amino acid (for example, glutamate) or Behera's amine,
Str¹ is

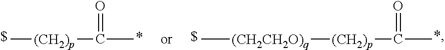

where $ is the point of attachment to Z′, * is the point of attachment to the remainder of the linker, p is an integer between 2 and 6, and q is an integer between 2 and 8;
Str² is

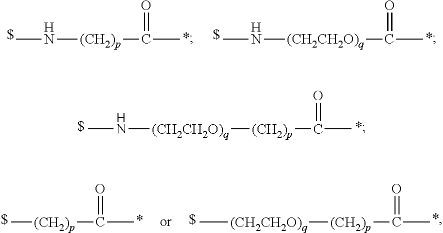

where $ is the point of attachment to BU, * is the point of attachment to the remainder of the linker, p is an integer between 2 and 6, and q is an integer between 2 and 8;
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit;
s is 1;
s′ is 1, and
is 0.

In certain embodiments, drug-linkers of Formula II comprise drug-linkers of Formula XIII:

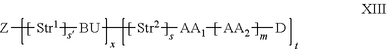

wherein:
Z is a functional group capable of reacting with a target group on the anti-cMet antibody construct, A;
Str¹ and Str² are each independently a stretcher;
BU is a branch unit;
AA₁ and AA₂ are each independently an amino acid, wherein AA₁-[AA₂]ₘ forms a protease cleavage site;
s and s′ are each independently 0 or 1;
m is 1, 2 or 3;
tis 1, 2 or 3;
x is 0 or 1, where when x is 0, tis 1 and when x is 1, t is 2 or 3, and
D has the structure shown in Formula II.

When incorporated into an ADC of the present disclosure, drug-linkers of Formula XIII have Formula XIV:

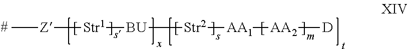

wherein:
Z′ is a linking group that joins the linker to the target group on the anti-cMet antibody construct, A;
Str¹, Str², BU, AA₁, AA₂, s, s′, m, t, x and D are as defined for Formula XIII, and
#is the point of attachment to the anti-cMet antibody construct, A.

In some embodiments, in Formula XIII and XIV, x is 1 and BU is an amino acid or Behera's amine. In some embodiments, in Formula XIII and XIV, x is 1 and BU is glutamate or Behera's amine.

In some embodiments, in Formula XIII and XIV, s is 1. In some embodiments, in Formula XI and XII, x is 1 and s′ is 1. In some embodiments, in Formula XI and XII, x is 1, and s and s′ are each 1.

In some embodiments, in Formula XIII, Z is a functional group capable of reacting with a thiol or amino group on the anti-cMet antibody construct, A.

In some embodiments, in Formula XIII:
Z is

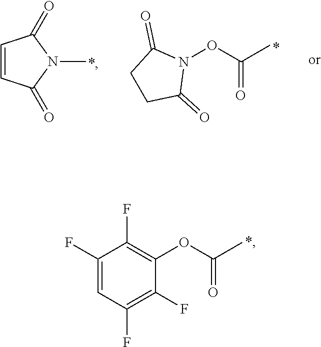

where * is the point of attachment to the remainder of the linker.

In some embodiments, in Formula XIV:
Z′ is a carbonyl group (—C(O)—) or

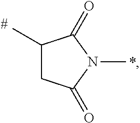

where #is the point of attachment to the anti-cMet antibody construct, A, and * is the point of attachment to the remainder of the linker.

In some embodiments, in Formula XIII and XIV, Str¹ is selected from:

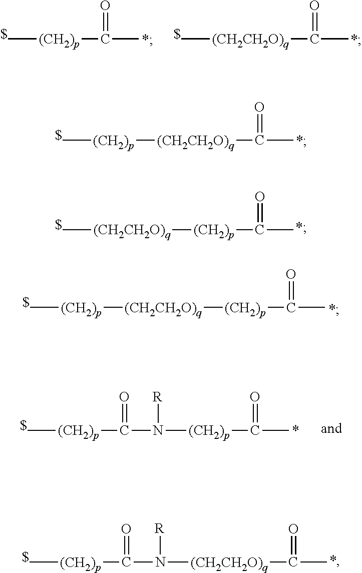

wherein:
each R is independently H or C₁-C₆ alkyl;
each p is independently an integer between 2 and 10;
each q is independently an integer between 1 and 10,
$ is the point of attachment to Z or Z′, and
is the point of attachment to the remainder of the linker.

In some embodiments, in Formula XIII and XIV, Str² is selected from:

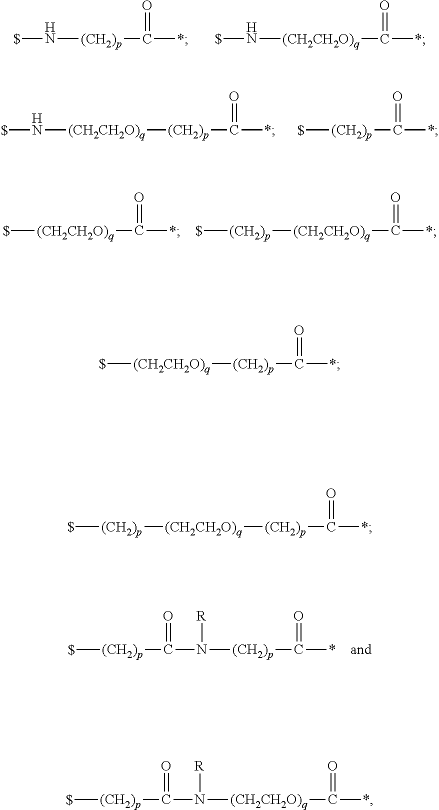

wherein:
each R is independently H or C₁-C₆ alkyl;
each p is independently an integer between 2 and 10;
each q is independently an integer between 1 and 10,
$ is the point of attachment to BU, and
is the point of attachment to the remainder of the linker.

In some embodiments, in Formula XIII and XIV, Str¹ is:

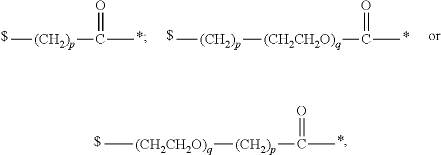

where p, q, $ and * are as defined above for Str¹.

In some embodiments, in Formula XIII and XIV, Str² is:

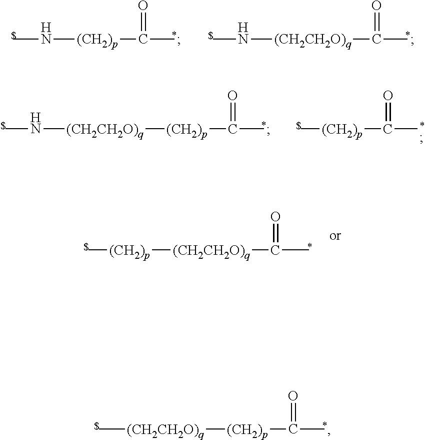

where p, q, $ and * are as defined above for Str².

In some embodiments, in Formula XIII and XIV, Str¹ is:

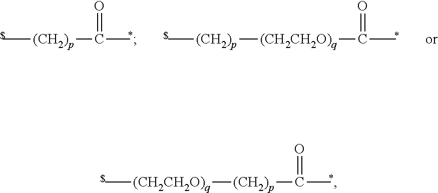

where $ and * are as defined above for Str¹, p is an integer between 2 and 6, and q is an integer between 2 and 8.

In some embodiments, in Formula XIII and XIV, Str² is:

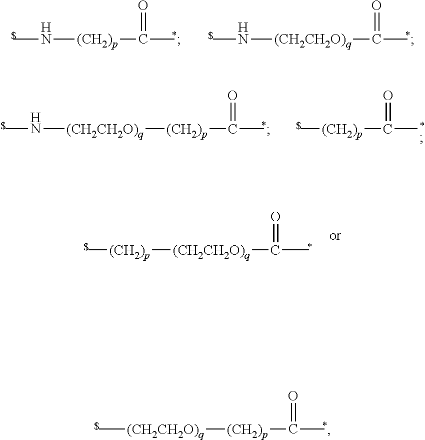

where $ and * are as defined above for Str², p is an integer between 2 and 6, and q is an integer between 2 and 8.

In some embodiments, in Formula XIII and XIV, AA₁-[AA₂]ₘ is selected from Val-Lys, Ala-Lys, Phe-Lys, Val-Cit, Phe-Cit, Leu-Cit, Ile-Cit, Trp-Cit, Phe-Arg, Ala-Phe, Val-Ala, Met-Lys, Asn-Lys, Ile-Pro, Ile-Val, Asp-Val, His-Val, Met-(D)Lys, Asn-(D)Lys, Val-(D)Asp, NorVal-(D) Asp, Ala-(D)Asp, Me₃Lys-Pro, PhenylGly-(D)Lys, Met-(D)Lys, Asn-(D)Lys, Pro-(D)Lys, Met-(D)Lys, Met-Cit-Val, Gly-Cit-Val, (D)Phe-Phe-Lys, (D)Ala-Phe-Lys, Gly-Phe-Leu-Gly, Ala-Leu-Ala-Leu and Gly-Gly-Phe-Gly.

In some embodiments, in Formula XIII and XIV, m is 1 (i.e. AA₁-[AA₂]ₘ is a dipeptide).

In some embodiments, in Formula XIII and XIV, AA₁-[AA₂]ₘ is a dipeptide selected from Val-Lys, Ala-Lys, Phe-Lys, Val-Cit, Phe-Cit, Leu-Cit, Ile-Cit and Trp-Cit.

Non-limiting examples of drug-linkers of Formula II are shown in Table 5 and non-limiting examples of ADCs comprising these drug-linkers are shown in Table 6. In certain embodiments, the ADC of Formula I comprises a drug-linker selected from the drug-linkers shown in Table 5. In certain embodiments, the drug-linker of Formula II is selected from the drug-linkers shown in Table 5. In certain embodiments, the ADC of Formula I is selected from the ADCs shown in Table 6, where A is the anti-cMet antibody construct and p is an integer between 1 and 8. In some embodiments, the ADC of Formula I is selected from the ADCs shown in Table 6, where A is the anti-cMet antibody construct and p is an integer between 2 and 6. In some embodiments, the ADC of Formula I is ADC 001 or ADC 002 shown in Table 6, where A is the anti-cMet antibody construct and p is an integer between 2 and 6. In some embodiments, the ADC of Formula I is selected from ADC 003, ADC 004, ADC 005 and ADC 006 shown in Table 6, where A is the anti-cMet antibody construct and p is an integer between 2 and 4. In some embodiments, the ADC of Formula I is ADC 003 or ADC 005 shown in Table 6, where A is the anti-cMet antibody construct and p is 2 or 3. In some embodiments, the ADC of Formula I is ADC 004 or ADC 006 shown in Table 6, where A is the anti-cMet antibody construct and p is 2.

TABLE 5
Exemplary Drug-Linkers

| Name | Structure |
|---|---|
| Drug-Linker 001 | 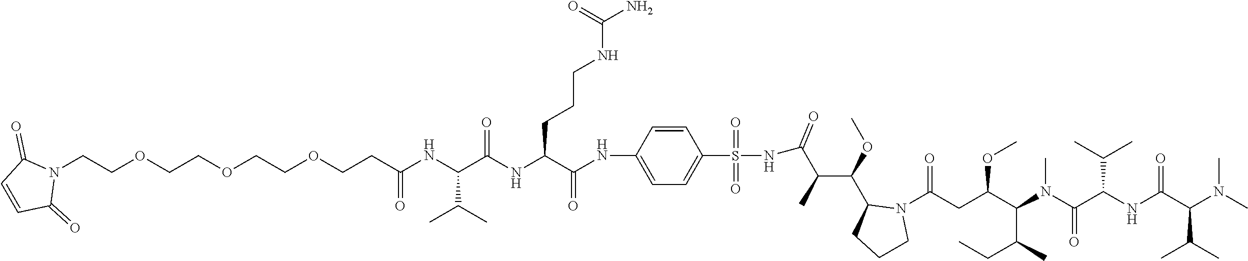 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCN2C(=O)C=CC2=O)C(C)C)cc1)OC)N(C)C(=O)[C@@H](NC(=O)[C@H](C(C)C)N(C)C)C(C)C] |
| Drug-Linker 002 | 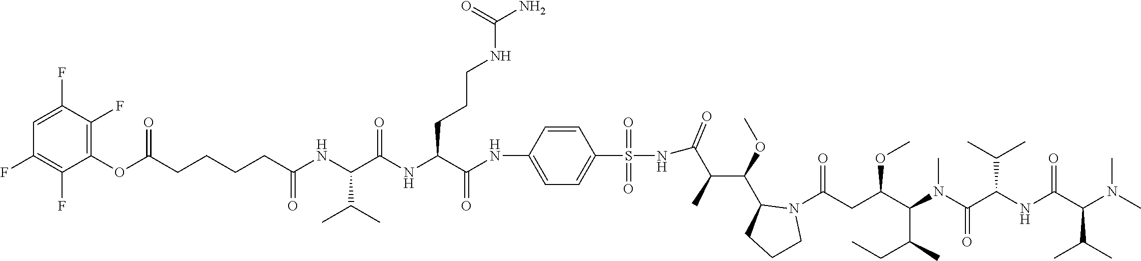 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCCCC(=O)Oc2c(F)c(F)cc(F)c2F)C(C)C)cc1)OC)N(C)C(=O)[C@@H](NC(=O)[C@H](C(C)C)N(C)C)C(C)C] |
| Drug-Linker 003 | 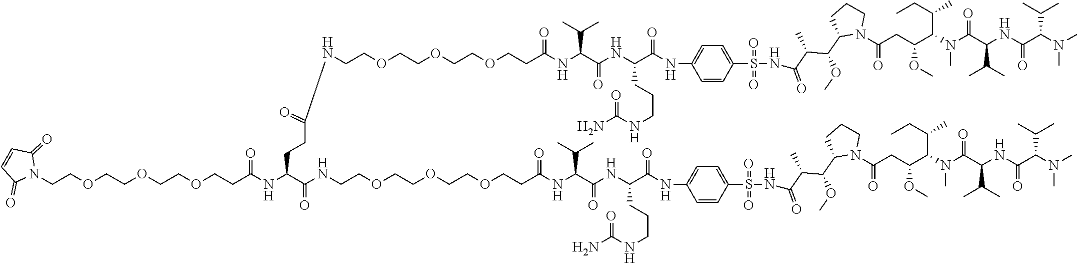 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCOCCOCCOCCN2C(=O)C=CC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] |
| Drug-Linker 004 | 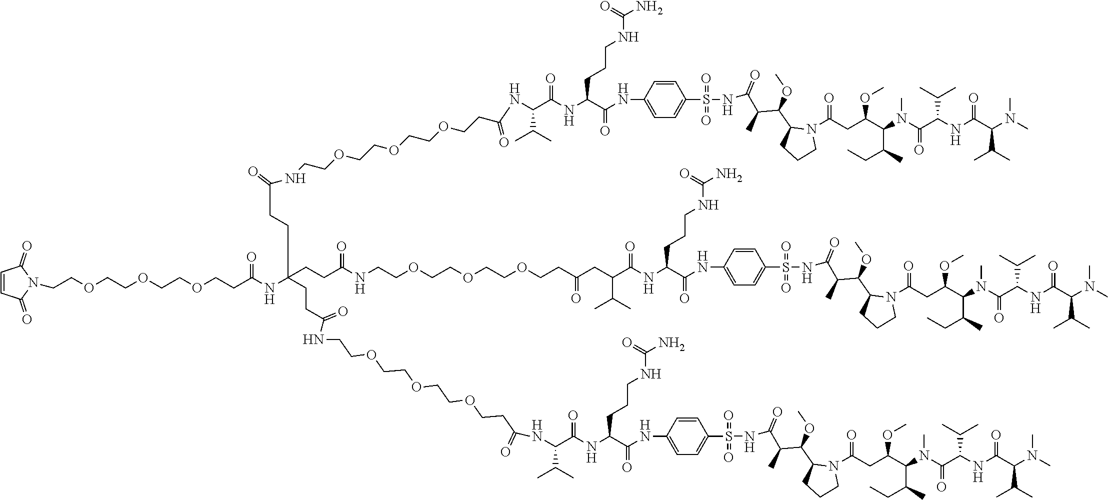 |
| Drug-Linker 005 | 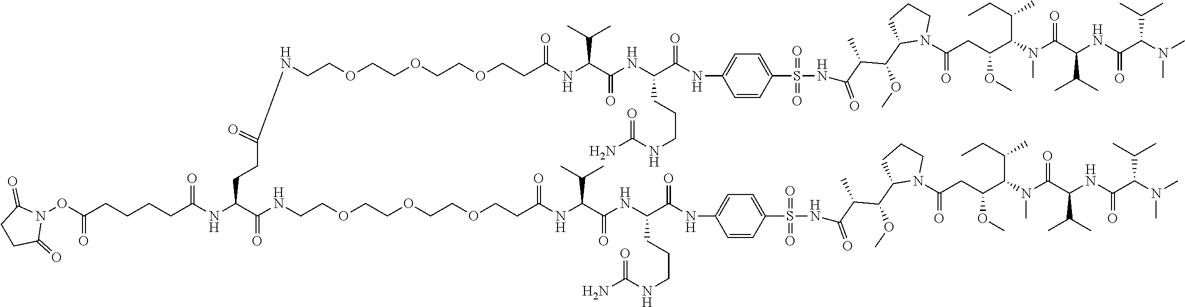 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)ON2C(=O)CCC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] |
| Drug-Linker 006 | 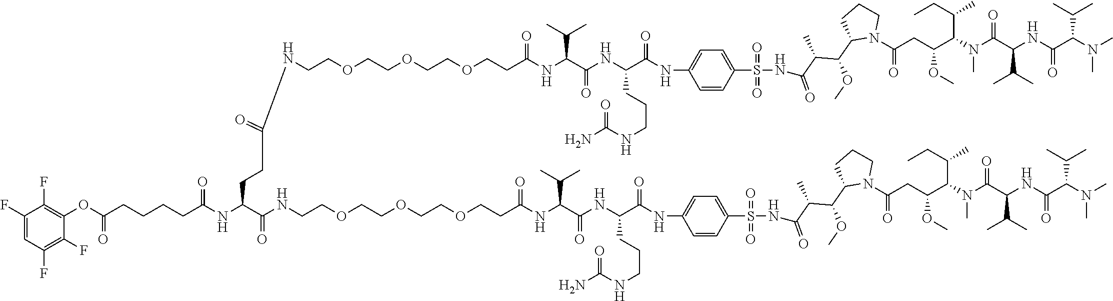 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)Oc2c(F)c(F)cc(F)c2F)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] |
| Drug-Linker 007 | 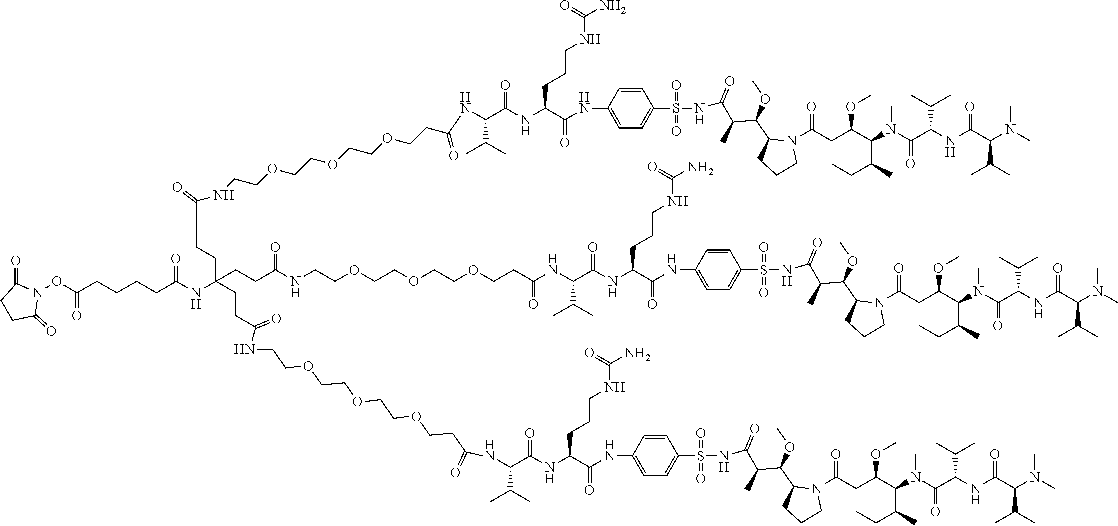 |
| Drug-Linker 008 | 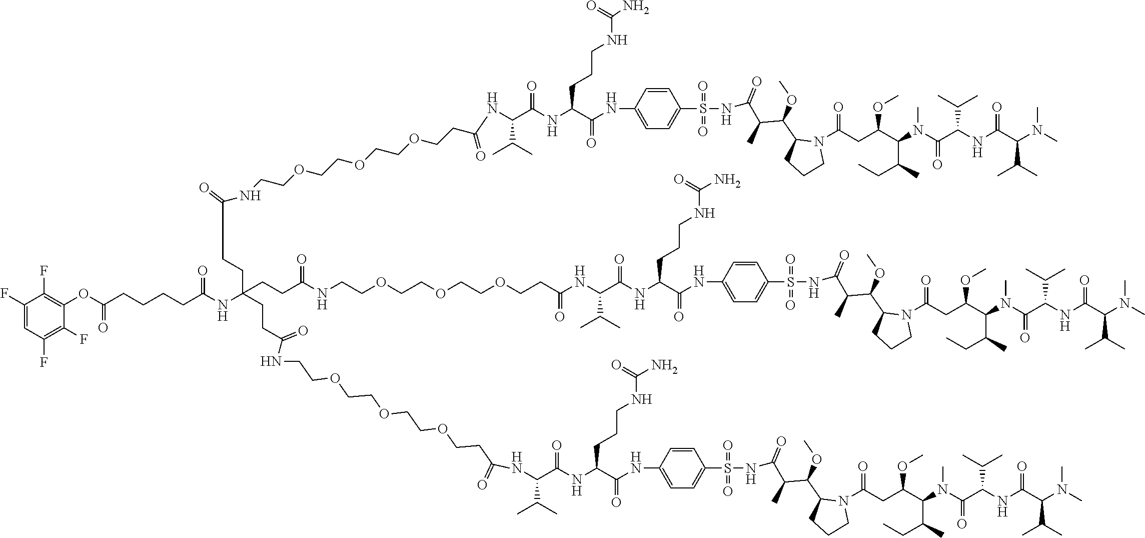 |

TABLE 6
Exemplary ADCs

| Name | Structure |
|---|---|
| ADC 001 | 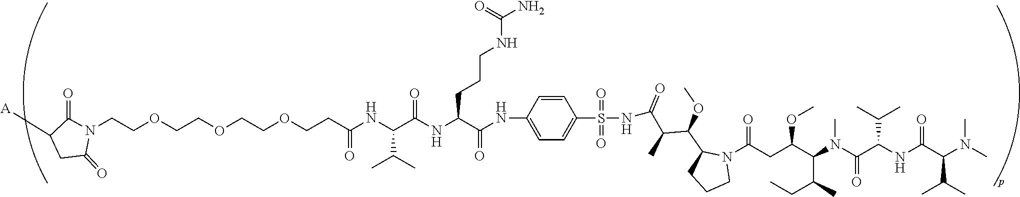 |
| ADC 002 | 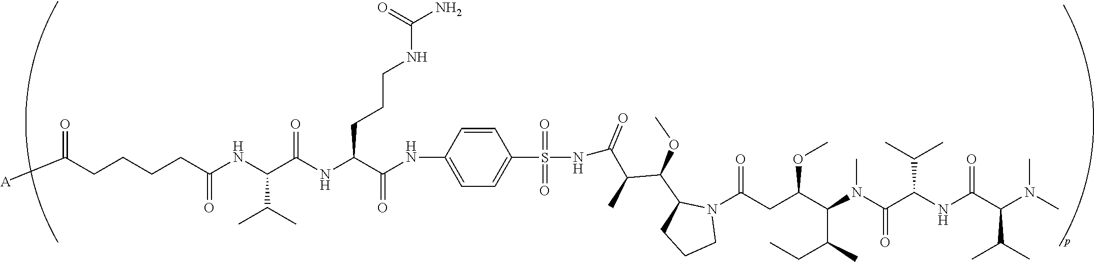 |
| ADC 003 | 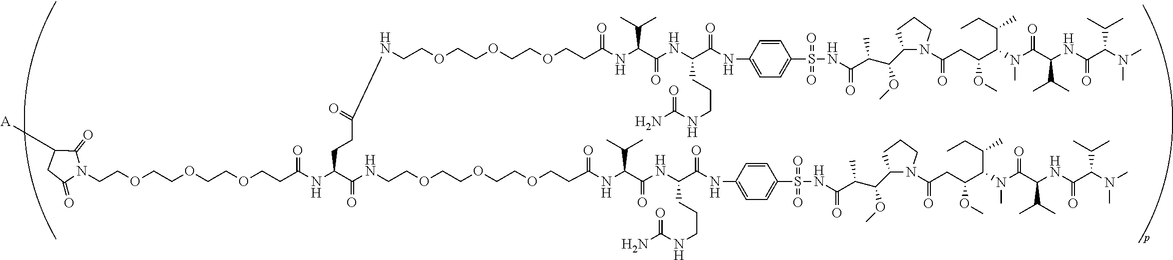 |
| ADC 004 | 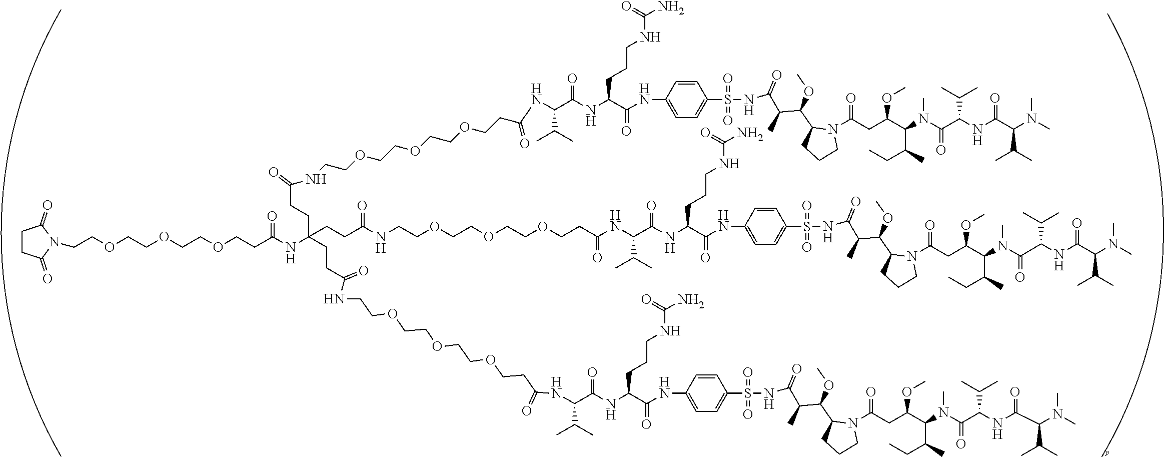 |
| ADC 005 | 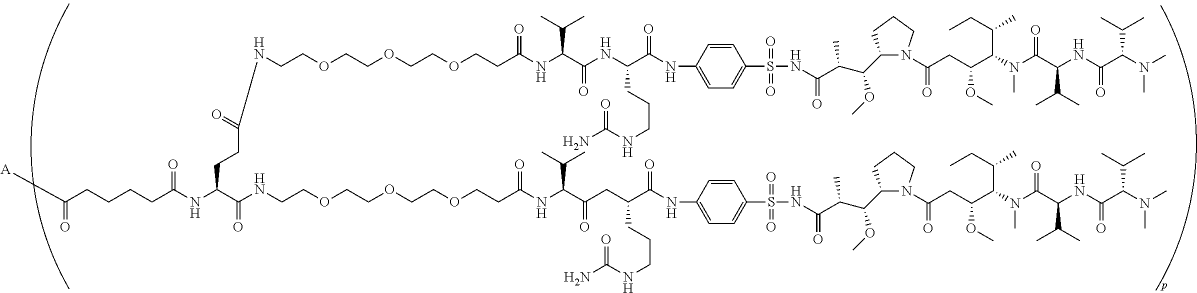 |
| ADC 006 | 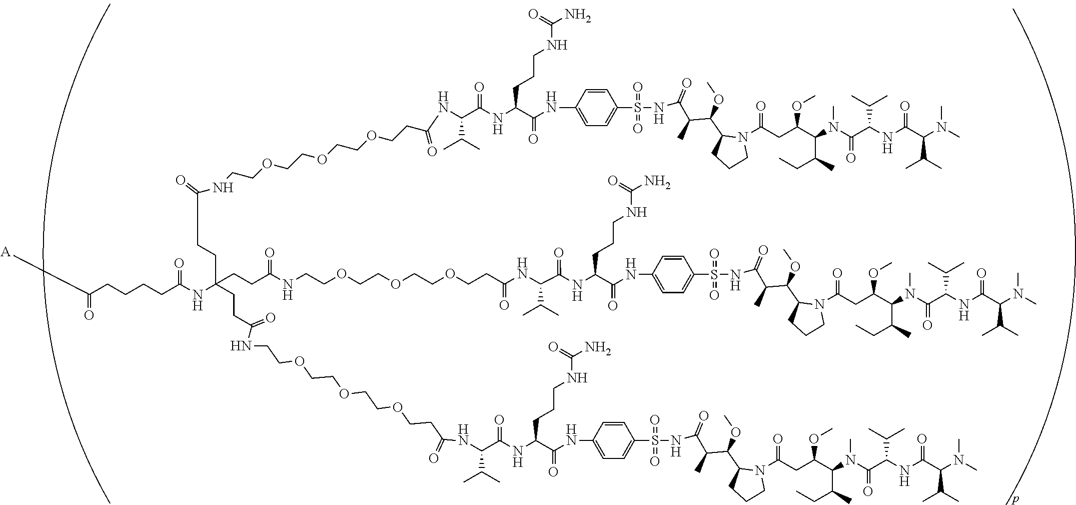 |

### Preparation

### Anti-cMet Antibody Constructs

The anti-cMet antibody constructs described herein may be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

Typically, for recombinant production of an antibody construct, a polynucleotide or set of polynucleotides encoding the anti-cMet antibody construct is generated and inserted into one or more vectors for further cloning and/or expression in a host cell. Polynucleotide(s) encoding the anti-cMet antibody construct may be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the anti-cMet antibody construct will be dependent on the format of the construct, including whether or not the antibody construct comprises a scaffold. For example, when an anti-cMet antibody construct is in a full-size antibody format with a homodimeric Fc, three polynucleotides each encoding one polypeptide chain will be required. When multiple polynucleotides are required, they may be incorporated into one vector or into more than one vector.

Generally, for expression, the polynucleotide or set of polynucleotides is incorporated into an expression vector or vectors together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the antibody construct and that such regulatory elements may be derived from a variety of sources, including bacterial, fungal, viral, mammalian or insect genes. The expression vector may optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector may be an extrachromosomal vector or an integrating vector.

Suitable host cells for cloning or expression of the anti-cMet antibody constructs include various prokaryotic or eukaryotic cells as known in the art. Prokaryotic host cells include, for example, E. coli, A. salmonicida and B. subtilis cells. Eukaryotic host cells include, for example, mammalian cells, plant cells, insect cells and yeast cells (such as Saccharomyces or Pichia cells). The selected host cells comprising the expression vector(s) encoding the anti-cMet antibody construct may be cultured using routine methods.

In certain embodiments, the anti-cMet antibody construct may be produced in eukaryotic cells. In some embodiments, the anti-cMet antibody construct may be produced in mammalian cells. Mammalian cell lines adapted to grow in suspension may be particularly useful for expression of antibody constructs. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251), monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour cells (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Various examples of mammalian host cell lines suitable for production of antibody constructs are reviewed in Yazaki & Wu, Methods in Molecular Biology, Vol. 248, pp. 255-268 (B. K. C. Lo, ed., Humana Press, Totowa, N.J., 2003).

In certain embodiments, the host cell may be a transient or stable higher eukaryotic cell line, such as a mammalian cell line. In some embodiments, the host cell may be a mammalian HEK293T, CHO, HeLa, NS0 or COS cell line, or a cell line derived from any one of these cell lines. In some embodiments, the host cell may be a stable cell line that allows for mature glycosylation of the antibody construct.

Certain embodiments of the present disclosure relate to an isolated polynucleotide or a set of polynucleotides encoding an anti-cMet antibody construct described herein. A polynucleotide in this context may encode all or part of an anti-cMet antibody construct.

The terms “polynucleotide,” “nucleic acid” and “nucleic acid molecule” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA, isolated RNA, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

Certain embodiments of the present disclosure relate to vectors (such as expression vectors) comprising one or more polynucleotides encoding an anti-cMet antibody construct described herein. The polynucleotide(s) may be comprised by a single vector or by more than one vector. In some embodiments, the polynucleotides are comprised by a multicistronic vector.

Certain embodiments of the present disclosure relate to host cells comprising polynucleotide(s) encoding an anti-cMet antibody construct described herein or one or more vectors comprising the polynucleotide(s). In some embodiments, the host cell is eukaryotic, for example, a Chinese Hamster Ovary (CHO) cell, a human embryonic kidney (HEK) cell or a lymphoid cell (for example, Y0, NS0, Sp20 cell).

Typically, the anti-cMet antibody constructs are purified after expression. Proteins may be isolated or purified in a variety of ways known to those skilled in the art (see, for example, Protein Purification: Principles and Practice, 3^{rd} Ed., Scopes, Springer-Verlag, NY, 1994). Standard purification methods include one or more chromatographic techniques, including ion exchange, hydrophobic interaction, affinity, sizing (or gel) filtration and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, may also be useful. As is well known in the art, a variety of natural proteins bind to antibodies and these proteins may be used for purification of certain antibody constructs. For example, the bacterial proteins A and G bind to the Fc region. Likewise, the bacterial protein L binds to the Fab region of some antibodies. Purification may also be enabled by a particular fusion partner. For example, antibodies may be purified using glutathione resin if a GST fusion is employed, Ni⁺² affinity chromatography if a His-tag is employed or immobilized anti-flag antibody if a flag-tag is used. The degree of purification necessary will vary depending on the intended use of the anti-cMet antibody constructs. In some instances, no purification may be necessary.

In certain embodiments, the anti-cMet antibody constructs are substantially pure. The term “substantially pure” (or “substantially purified”) when used in reference to an anti-cMet antibody construct described herein, means that the antibody construct is substantially or essentially free of components that normally accompany or interact with the protein as found in its naturally occurring environment, such as a native cell, or a host cell in the case of recombinantly produced construct. In certain embodiments, an anti-cMet antibody construct that is substantially pure is a protein preparation having less than about 30%, less than about 25%, less than about 20%, less than about 15%, less than about 10%, or less than about 5% (by dry weight) of contaminating protein.

Certain embodiments of the present disclosure relate to a method of making an anti-cMet antibody construct comprising culturing a host cell into which one or more polynucleotides encoding the anti-cMet antibody construct, or one or more expression vectors encoding the anti-cMet antibody construct, have been introduced, under conditions suitable for expression of the anti-cMet antibody construct, and optionally recovering the anti-cMet antibody construct from the host cell (or from host cell culture medium). In some embodiments, the method further comprises submitting the anti-cMet antibody construct to one or more purification steps.

### Post-Translational Modifications

In certain embodiments, the anti-cMet antibody constructs described herein may comprise one or more post-translational modifications. Such post-translational modifications may occur in vivo, or they may be conducted in vitro after isolation of the anti-cMet antibody construct from the host cell.

Post-translational modifications include various modifications as are known in the art, such as glycosylation, acetylation, phosphorylation, amidation, deamidation, derivatization by known protecting/blocking groups, formylation, oxidation, reduction, proteolytic cleavage or specific chemical cleavage by cyanogen bromide, trypsin, chymotrypsin, papain, V8 protease, NaBH₄, or the like (see, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the anti-cMet antibody construct comprises one or more post-translational modifications, the construct may comprise the same type of modification at one or several sites, or it may comprise different modifications at different sites.

Additional examples of post-translational modifications include, but are not limited to, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications may also include modification with a detectable label, such as an enzymatic, fluorescent, luminescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. Examples of luminescent materials include luminol, and bioluminescent materials such as luciferase, luciferin and aequorin. Examples of suitable radioactive materials include iodine, carbon, sulfur, tritium, indium, technetium, thallium, gallium, palladium, molybdenum, xenon and fluorine.

Further examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### ADCs

ADCs of Formula I comprising the anti-cMet antibody construct may be prepared by standard methods known in the art (see, for example, Bioconjugate Techniques (G. T. Hermanson, 2013, Academic Press)). Exemplary methods are provided herein. Various linkers and linker components are commercially available or may be prepared using standard synthetic organic chemistry techniques (see, for example, March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramal Pharma Solutions (Grangemouth, UK) and may be used to prepare the ADCs.

Typically, preparation of the ADCs comprises first preparing a drug-linker, D-L, comprising one or more auristatin analogues and linker L (for example, a drug-linker of Formula II), and then conjugating the drug-linker, D-L, to an appropriate group on the anti-cMet antibody construct, A. Ligation of linker, L, to the anti-cMet antibody construct, A, and subsequent ligation of the anti-cMet antibody construct-linker, A-L, to one or more auristatin analogues, D, remains however an alternative approach that may be employed in some embodiments.

Suitable groups on the anti-cMet antibody construct, A, for attachment of linker, L, include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups.

In certain embodiments, one or more naturally occurring cysteine residues on the anti-cMet antibody construct, A, may be employed to bond to linker, L, via the sulfhydryl group of the cysteine. In certain embodiments, one or more naturally occurring lysine residues on the anti-cMet antibody construct, A, may be employed to bond to linker, L, via the amino group of the lysine.

Alternatively, one or more lysine residues on the anti-cMet antibody construct, A, may be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio) propionate (“SPDP”) and 2-iminothiolane hydrochloride (Traut's Reagent). Alternatively, one or more carbohydrate groups on the anti-cMet antibody construct, A, may be chemically modified to include one or more sulfhydryl groups.

Carbohydrate groups on the anti-cMet antibody construct, A, may also be oxidized to provide an aldehyde (—CHO) group (see, for example, Laguzza et al., 1989, J. Med. Chem. 32 (3): 548-55), which could subsequently be reacted with linker, L, for example, via a hydrazine or hydroxylamine group on linker, L.

The anti-cMet antibody construct, A, may also be modified to include additional cysteine residues as described above or, for example, in U.S. Pat. Nos. 7,521,541; 8,455,622 and 9,000,130. Alternatively, the anti-cMet antibody may be modified to include one or more non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361) to allow for site-specific conjugation. The anti-cMet antibody construct, A, may also be modified to include a non-natural reactive group, such as an azide, that allows for conjugation to the linker via a complementary reactive group on the linker, for example, by click chemistry (see, for example, Chio & Bane, 2020, Methods Mol. Biol., 2078:83-97). A further option is the use of GlycoConnect™ technology (Synaffix BV, Nijmegen, Netherlands), which involves enzymatic remodelling of the antibody glycans to allow for attachment of a linker by metal-free click chemistry (see, for example, European Patent No. EP 2 911 699).

Other protocols for the modification of proteins for the attachment or association of linker, L, are known in the art (see, for example, Coligan et al., Current Protocols in Protein Science, vol. 2, John Wiley & Sons (2002)).

Alternatively, ADCs may be prepared using the enzyme transglutaminase, in particular, bacterial transglutaminase (BTG) from Streptomyces mobaraensis (see, for example, Jeger et al., 2010, Angew. Chem. Int. Ed., 49:9995-9997). BTG forms an amide bond between the side chain carboxamide of a glutamine (the amine acceptor, typically on the antibody) and an alkyleneamino group (the amine donor, typically on the drug-linker), which can be, for example, the ¿-amino group of a lysine or a 5-amino-n-pentyl group. Antibodies may also be modified to include a glutamine containing peptide, or “tag,” which allows BTG conjugation to be used to conjugate the antibody to a drug-linker (see, for example, U.S. Patent Application Publication No. US 2013/0230543 and International (PCT) Publication No. WO 2016/144608).

A similar conjugation approach utilizes the enzyme sortase A. In this approach, the antibody is typically modified to include the sortase A recognition motif (LPXTG, where X is any natural amino acid) and the drug-linker is designed to include an oligoglycine motif (typically GGG) to allow for sortase A-mediated transpeptidation (see, for example, Beerli, et al., 2015, PLos One, 10: e0131177; Chen et al., 2016, Nature: Scientific Reports, 6:31899).

Once conjugation is complete, the average number of auristatin analogue molecules conjugated to the anti-cMet antibody construct, A, (i.e. the “drug-to-antibody ratio” or DAR) may be determined by standard techniques such as UV/VIS spectroscopic analysis, ELISA-based techniques, chromatography techniques such as hydrophobic interaction chromatography (HIC), UV-MALDI mass spectrometry (MS) or MALDI-TOF MS. In addition, distribution of drug-linked forms (for example, the fraction of the anti-cMet antibody construct, A, containing zero, one, two, three, etc. conjugated auristatin analogue molecules) optionally may be analyzed, for example by MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

Certain embodiments of the present disclosure relate to methods of preparing an ADC of Formula I comprising conjugating a drug-linker of Formula II to an anti-cMet antibody construct. In some embodiments, the method comprises conjugating the drug-linker to a cysteine residue on the anti-cMet antibody construct. In some embodiments, the anti-cMet antibody construct comprises one or more cysteine insertion mutations and the method comprises conjugating the drug-linker to an inserted cysteine residue. In some embodiments, the method comprises conjugating the drug-linker to a lysine residue on the anti-cMet antibody construct.

### Pharmaceutical Compositions

For therapeutic uses, the ADCs of the present disclosure are typically formulated as pharmaceutical compositions. Certain embodiments of the present disclosure thus relate to pharmaceutical compositions comprising an ADC as described herein and a pharmaceutically acceptable carrier, diluent, or excipient. Such pharmaceutical compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term “parenteral” as used herein includes subcutaneous injection, and intradermal, intra-articular, intravenous, intramuscular, intravascular, intrasternal, intrathecal injection or infusion. The pharmaceutical composition will typically be formulated in a format suitable for the selected route of administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, the pharmaceutical compositions comprising the ADCs are formulated for parenteral administration, for example as lyophilized formulations or aqueous solutions. Such pharmaceutical compositions may be provided, for example, in a unit dosage injectable form.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions comprising the ADCs may be in the form of a sterile injectable aqueous or oleaginous solution or suspension. Such suspensions may be formulated using suitable dispersing or wetting agents and/or suspending agents that are known in the art. The sterile injectable solution or suspension may comprise the ADC in a non-toxic parentally acceptable diluent or carrier. Acceptable diluents and carriers that may be employed include, for example, 1,3-butanediol, water, Ringer's solution or isotonic sodium chloride solution. In addition, sterile, fixed oils may be employed as a carrier. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents may also be included in the injectable solution or suspension.

In certain embodiments, the composition comprising the ADC may be formulated for intravenous administration to humans. Typically, compositions for intravenous administration are solutions in sterile isotonic aqueous buffer. Where necessary, the composition may also include a solubilizing agent and/or a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule comprising sterile water for injection or saline, for example, can be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Certain embodiments of the present disclosure relate to the therapeutic use of the ADCs described herein. Some embodiments relate to the use of the ADCs as therapeutic agents, for example, as anti-cancer agents.

In certain embodiments, the ADCs described herein may be used in the treatment of cancer. Certain embodiments relate to methods of inhibiting cancer cell or tumour cell growth; inhibiting cancer cell or tumour cell proliferation, or treating cancer in a subject, comprising administering an ADC of Formula I.

Certain embodiments of the present disclosure relate to methods of inhibiting the proliferation of cancer or tumour cells comprising contacting the cells in vitro or in vivo with an ADC of Formula I. Some embodiments relate to a method of killing cancer or tumour cells comprising contacting the cells in vitro or in vivo with an ADC of Formula I. Certain embodiments relate to the use of an ADC of Formula I in a method of inhibiting tumour growth in a subject.

Some embodiments relate to methods of treating a subject having a cancer by administering to the subject an ADC of Formula I. In this context, treating the subject may result in one or more of a reduction in the size of a tumour, the slowing or prevention of an increase in the size of a tumour, an increase in the disease-free survival time between the disappearance or removal of a tumour and its reappearance, prevention of a subsequent occurrence of a tumour (for example, metastasis), an increase in the time to progression, reduction of one or more adverse symptom associated with a tumour, and/or an increase in the overall survival time of a subject having cancer.

Examples of cancers which may be treated with the ADCs described herein in certain embodiments include carcinomas (including adenocarcinomas and squamous cell carcinomas), melanomas and sarcomas. Carcinomas and sarcomas are also frequently referred to as “solid tumours.” Examples of commonly occurring solid tumours that may be treated with the ADCs described herein in certain embodiments include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumour and, therefore, may also be considered to be solid tumours in certain situations. Typically, the cancer to be treated is a cMet-expressing cancer.

Certain embodiments relate to methods of inhibiting the growth of cMet-positive tumour cells comprising contacting the cells with an ADC of Formula I. The cells may be in vitro or in vivo. In certain embodiments, the ADCs may be used in methods of treating a cMet-positive or cMet-overexpressing cancer or tumour in a subject.

Cancers that overexpress c-Met are typically solid tumours. Examples include, but are not limited to, ovarian cancer, lung cancers, breast cancer, gastric cancer, colorectal cancer, head & neck cancer, kidney cancer and pancreatic cancer. In certain embodiments, the ADCs of Formula I may be used in methods of treating a subject having a cMet-positive or cMet-overexpressing ovarian cancer, lung cancer, breast cancer, gastric cancer, colorectal cancer, head & neck cancer, kidney cancer or pancreatic cancer.

### Pharmaceutical Kits

Certain embodiments relate to pharmaceutical kits comprising an ADC of Formula I.

The kit typically will comprise a container holding the ADC and a label and/or package insert on or associated with the container. The label or package insert contains instructions customarily included in commercial packages of therapeutic products, providing information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. In some embodiments, the container may have a sterile access port. For example, the container may be an intravenous solution bag or a vial having a stopper that may be pierced by a hypodermic injection needle.

In addition to the container holding the ADC, the kit may optionally comprise one or more additional containers comprising other components of the kit. For example, a pharmaceutically acceptable buffer (such as bacteriostatic water for injection (BWFI), phosphate-buffered saline, Ringer's solution or dextrose solution), other buffers or diluents.

Suitable containers include, for example, bottles, vials, syringes, intravenous solution bags, and the like. The containers may be formed from a variety of materials such as glass or plastic. If appropriate, one or more components of the kit may be lyophilized or provided in a dry form, such as a powder or granules, and the kit can additionally contain a suitable solvent for reconstitution of the lyophilized or dried component(s).

The kit may further include other materials desirable from a commercial or user standpoint, such as filters, needles, and syringes.

The following Examples are provided for illustrative purposes and are not intended to limit the scope of the invention in any way.

### EXAMPLES

### Example 1: Anti-cMet Antibody Preparation

### 1.1 Generation of Antibodies

A cMet-targeting antibody, variant v32634, was prepared as described below. This antibody is based on telisotuzumab (ABT-700) (see Wang et al., 2016, BMC Cancer, 16:105) but lacks any modifications to the hinge region. In addition to variant v32634, antibody variants comprising various cysteine insertion mutations as described below and a control variant, v17606, which included the hinge sequence modifications comprised by telisotuzumab, were also prepared. An additional control antibody, variant v17429, which is a bivalent version of the anti-cMet antibody, MetMab (onartuzumab), was also prepared. The variants are summarized in Table 1.3. Sequences for each variant are provided in the Sequence Tables. Specifically, Sequence Table A provides the clone numbers for each antibody variant made, e.g., v32634 has a light chain and variable light domain of clone number 23998 and a heavy chain and variable heavy chain domain of clone number 23993. Sequence Table B provides the full heavy or light chain amino acid and nucleic acid sequences, as well as the variable light or heavy chain amino acid sequences for the clones provided in Table A. Sequence Table C provides additional sequences for the cysteine insertion variants. In addition, the VH, VL and CDR sequences are provided in Table 2 above.

Variants were produced in full-size antibody (FSA) format containing either two identical full-length heavy chains resulting in a homodimeric Fc region (HomoFc), or heterodimeric full-length heavy chains comprising complementary mutations in the CH3 region to drive heterodimeric heavy chain pairing resulting in a heterodimeric Fc region (HetFc).

The two identical full-length heavy chains comprised by the HomoFc region contained the human CH1-hinge-CH2-CH3 domain sequence of IGHG1*01 (SEQ ID NO:31; see Table 1.1). The heterodimeric full-length heavy chains comprised by the HetFc region (HetFc-A and HetFc-B) contained the human CH1-hinge-CH2-CH3 domain sequence of IGHG1*01 with the following mutations in the Fc region:
HetFc-A: T350V_L351Y_F405A_Y407V
HetFc-B: T350V_T366L_K392L_T394W

The sequences of HetFc-A (SEQ ID NO:33) and HetFc-B (SEQ ID NO:34) are provided in Table 1.1. The human kappa CL sequence of IGKC*01 (SEQ ID NO:32; see Table 1.1) was used for all constructs.

TABLE 1.1
Antibody Sequences

| | | SEQ ID |
|---|---|---|
| | Sequence | NO |
| CH1-hinge- | ASTKGPSVFPLAPSSKSTSGGTAALGCLVKDYFPEPVTVSWNS | 31 |
| CH2-CH3 | GALTSGVHTFPAVLQSSGLYSLSSVVTVPSSSLGTQTYICNVNH | |
| (IGHG1*01) | KPSNTKVDKKVEPKSCDKTHTCPPCPAPELLGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVYTLPPSRDELTKNQVSLTCLVKGFYPSD | |
| | IAVEWESNGQPENNYKTTPPVLDSDGSFFLYSKLTVDKSRWQQ | |
| | GNVFSCSVMHEALHNHYTQKSLSLSPG | |
| CL (kappa) | RTVAAPSVFIFPPSDEQLKSGTASVVCLLNNFYPREAKVQWKV | 32 |
| (IGKC*01) | DNALQSGNSQESVTEQDSKDSTYSLSSTLTLSKADYEKHKVYA | |
| | CEVTHQGLSSPVTKSFNRGEC | |
| CH1-hinge- | ASTKGPSVFPLAPSSKSTSGGTAALGCLVKDYFPEPVTVSWNS | 33 |
| CH2-CH3 | GALTSGVHTFPAVLQSSGLYSLSSVVTVPSSSLGTQTYICNVNH | |
| (HetFc-A) | KPSNTKVDKKVEPKSCDKTHTCPPCPAPELLGGPSVFLFPPKPK | |
| (IGHG1*01) | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVYVYPPSRDELTKNQVSLTCLVKGFYPS | |
| | DIAVEWESNGQPENNYKTTPPVLDSDGSFALVSKLTVDKSRW | |
| | QQGNVFSCSVMHEALHNHYTQKSLSLSPG | |
| CH1-hinge- | ASTKGPSVFPLAPSSKSTSGGTAALGCLVKDYFPEPVTVSWNS | 34 |
| CH2-CH3 | GALTSGVHTFPAVLQSSGLYSLSSVVTVPSSSLGTQTYICNVNH | |
| (HetFc-B) | KPSNTKVDKKVEPKSCDKTHTCPPCPAPELLGGPSVFLFPPKPK | |
| (IGHG1*01) | DTLMISRTPEVTCVVVDVSHEDPEVKFNWYVDGVEVHNAKTK | |
| | PREEQYNSTYRVVSVLTVLHQDWLNGKEYKCKVSNKALPAPI | |
| | EKTISKAKGQPREPQVYVLPPSRDELTKNQVSLLCLVKGFYPS | |
| | DIAVEWESNGQPENNYLTWPPVLDSDGSFFLYSKLTVDKSRW | |
| | QQGNVFSCSVMHEALHNHYTQKSLSLSPG | |

The wild-type (WT) hinge sequence comprised by variant v32634 and the cysteine insertion variants and the modified hinge sequence comprised by variant v17606 and telisotuzumab are provided in Table 1.2.

TABLE 1.2
WT and Modified Hinge Sequences

| | Sequence | SEQ ID NO |
|---|---|---|
| WT Hinge | EPKSCDKTHTCPPCP | 35 |
| Modified Hinge | EPKSCDCHCPPCP | 36 |

TABLE 1.3
Summary of Variants

| | | | | No. of | Positions of Cysteine Insertions¹ | | |
|---|---|---|---|---|---|---|---|
| Variant | | Fc | | Cysteine | Heavy | Heavy | Light |
| No. | Paratope | Region | Hinge | Insertions | Chain A | Chain B | Chain (×2) |
| v17606 | Teliso- | HomoFc | Modified | 0 | — | — | — |
| | tuzumab | | | | | | |
| v17427 | Teliso- | HetFc | Unmodi- | 0 | — | — | — |
| | tuzumab | | fied | | | | |
| v32634 | Teliso- | HomoFc | Unmodi- | 0 | — | — | — |
| | tuzumab | | fied | | | | |
| v33967 | Teliso- | HetFc | Unmodi- | 1 | None | Between residues | None |
| | tuzumab | | fied | | | T299 and Y300 | |
| v33968 | Teliso- | HetFc | Unmodi- | 1 | Between G237 | None | None |
| | tuzumab | | fied | | and P238 | | |
| v33969 | Teliso- | HetFc | Unmodi- | 1 | Between A9 | None | None |
| | tuzumab | | fied | | and E10 | | |
| v22761² | Teliso- | HetFc | Unmodi- | 2 | None | None | Between residues |
| | tuzumab | | fied | | | | K126 and S127 |
| v22765 | Teliso- | HetFc | Unmodi- | 2 | Between residues | Between residues | None |
| | tuzumab | | fied | | G237 and P238 | G237 and P238 | |
| v28983 | Teliso- | HetFc | Unmodi- | 2 | None | None | Between residues |
| | tuzumab | | fied | | | | P40 and G41 |
| v28989 | Teliso- | HetFc | Unmodi- | 2 | Between residues | Between residues | None |
| | tuzumab | | fied | | A9 and E10 | A9 and E10 | |
| v29001 | Teliso- | HetFc | Unmodi- | 2 | Between residues | Between residues | None |
| | tuzumab | | fied | | T299 and Y300 | T299 and Y300 | |
| v35527 | Teliso- | HomoFc | Unmodi- | 2 | Between residues | Between residues | None |
| | tuzumab | | fied | | T299 and Y300 | T299 and Y300 | |
| v33970 | Teliso- | HetFc | Unmodi- | 3 | Between residues | None | Between residues |
| | tuzumab | | fied | | T299 and Y300 | | P40 and G41 |
| v33971 | Teliso- | HetFc | Unmodi- | 3 | Between residues | Between residues | None |
| | tuzumab | | fied | | T299 and Y300, | T299 and Y300 | |
| | | | | | and between | | |
| | | | | | residues G237 | | |
| | | | | | and P238 | | |
| v33974 | Teliso- | HetFc | Unmodi- | 3 | Between residues | Between residues | None |
| | tuzumab | | fied | | A9 and E10, and | T299 and Y300 | |
| | | | | | between residues | | |
| | | | | | T299 and Y300 | | |
| v33977 | Teliso- | HetFc | Unmodi- | 3 | Between residues | None | Between residues |
| | tuzumab | | fied | | A9 and E10 | | P40 and G41 |
| v33979 | Teliso- | HetFc | Unmodi- | 3 | Between residues | Between residues | None |
| | tuzumab | | fied | | A9 and E10, and | A9 and E10 | |
| | | | | | between residues | | |
| | | | | | G237 and P238 | | |
| v17429 | Bivalent | HetFc | Unmodi- | 0 | None | None | None |
| | MetMab | | fied | | | | |

¹Numbering for mutations in the VH, VL and CL domains is Kabat numbering. Numbering for mutations in the CH2 domain is EU numbering.
²Variant included the peptide ESSCDVKLV [SEQ ID NO: 80] fused to the C-terminal residue of the light chain

### 1.2 Production of Antibodies

The antibody variants listed in Table 1.3 were produced using heavy chain expression vectors comprising heavy chain vector inserts including the signal peptide MRPTWAWWLFLVLLLALWAPARG (SEQ ID NO:37) (Barash et al., 2002, Biochem and Biophys Res. Comm., 294:835-842) and the heavy chain clone terminating at residue G446 (EU numbering) of the CH3 domain ligated into a pTT5 vector, and light chain expression vectors comprising light chain vector inserts comprising the same signal peptide ligated into a pTT5 vector. The resulting heavy and light chain expression vectors were sequenced to confirm correct reading frame and sequence of the coding DNA. A representative example of a protocol by which the antibodies were produced is as follows:

The heavy and light chains were expressed in 200 ml cultures of CHO-3E7 cells. Briefly, CHO-3E7 cells, at a density of 1.7-2×10⁶ cells/ml, viability >95%, were cultured at 37° C. in FreeStyle™ F17 medium (Thermo Fisher Scientific, Waltham, MA) supplemented with 4 mM glutamine (GE Life Sciences, Marlborough, MA) and 0.1% Pluronic® F-68 (Gibco/Thermo Fisher Scientific, Waltham, MA). A total volume of 200 ml CHO-3E7 cells+1× antibiotic/antimycotics (GE Life Sciences, Marlborough, MA) was transfected with a total of 200 ug DNA (100 ug of antibody DNA and 100 μg of GFP/AKT/stuffer DNA) using PEI-MAX® (Polyscience, Inc., Philadelphia, PA) at a DNA:PEI ratio of 1:4 (w/w). Twenty-four hours after the addition of the DNA-PEI mixture, 0.5 mM valproic acid (final concentration)+1% w/v Tryptone (final concentration) were added to the cells, which were then transferred to 32° C. and incubated for 6 more days prior to harvesting.

Protein-A purification was performed in batch mode or using 1 mL HiTrap™ MabSelect™ SuRe™ columns (Cytiva, Marlborough, MA). In batch mode, clarified supernatant samples were incubated in batch with mAb Select SuRe™ resin (GE Healthcare, Chicago, IL), cleaned-in-place (CIP′d) with NaOH and equilibrated in Dulbecco's PBS (DPBS). Resin was poured into CIP′d columns and the columns were washed with DPBS. In both purification modes, protein was eluted with 100 mM sodium citrate buffer pH 3.0. The eluted fractions were pH adjusted by adding 10% (v/v) 1 M HEPES (pH˜10.6-10.7) to yield a final pH of 6-7. Samples were buffer exchanged into DPBS. Protein was quantitated based on absorbance at 280 nm (A280 nm). Based on the purity, variants were further purified by preparatory SEC chromatography on a Superdex™ 200 Increase 10/30 column (GE Healthcare, Chicago, IL) in DPBS mobile phase following protein-A purification.

Following purification, purity of samples was assessed by electrophoresis under non-reducing and reducing conditions using the High Throughput Protein Express assay and Caliper LabChip® GXII or GXII Touch HT (Perkin Elmer, Waltham, MA). Procedures were carried out according to HT Protein Express LabChip® User Guide version 2 with the following modifications. Antibody samples, at either 2 μl or 5 μl (concentration range 5-2000 ng/μl) were added to separate wells in 96 well plates (BioRad, Hercules, CA) along with 7 μl of HT Protein Express Sample Buffer (Perkin Elmer, Cat #760328). Antibody samples were then denatured at 70° C. for 15 mins. The LabChip® instrument was operated using the HT Protein Express Chip (Perkin Elmer, Waltham, MA) and the Ab-200 assay setting. In some cases, purity of samples was also monitored by SDS-PAGE under reducing and non-reducing conditions.

The yield (post protein-A purification) for the variants ranged from mg to gram and is summarized in Table 1.4.

### 1.3 Assessment of Purity by Analytical Size Exclusion Chromatography (SEC)

Purity of the variants was determined by UPLC-SEC. For analytical SEC runs, an Agilent Advance Bio SEC column (300 Å, 2.7 μm, 7.8×150 mm) (Agilent Technologies, Inc., Santa Clara, CA; serial #6377910-24) was equilibrated with 5 column volumes of Buffer A (150 mM NaₓPO₄, pH 6.95) at room temperature. Usually, 20-30 ug of sample at 2-3 mg/mL concentration was loaded onto the column and run for 7 mins at 1 mL/min in an isocratic manner and absorbance at 280 nm was reported. For each sample, the chromatogram was integrated to provide complete, baseline-to-baseline integration of each peak, with reasonably placed separation between partially resolved peaks. The peak corresponding to the major component for IgG (approximate retention time 3.3 min) was reported as the monomer based on the SEC profile of the control IgG1 antibody, trastuzumab. Any peak occurring prior to 3.3 min was designated as HMWS, and any peak occurring after 3.3 min was designated as LMWS, excluding solvent peaks (over 5.2 min).

Purity for each of the cysteine insertion variants is summarized in Table 1.4.

TABLE 1.4
Production Summary for Representative
Cysteine Insertion Variants

| | No. of | | |
|---|---|---|---|
| Variant | Cysteine | Yield | Purity (%) |
| No. | Insertions | (mg/2 L) | (UPLC-SEC) |
| v28983 | 2 | 244.2 | 99.1 |
| v29001 | 2 | 135.1 | 98.8 |
| v35527 | 2 | 1422.7 | 99.7 |
| v33967 | 1 | 525.2 | 99.3 |
| v33968 | 1 | 403.9 | 99.3 |
| v33970 | 3 | 260.5 | 98.8 |
| v33971 | 3 | 46.2 | 99.3 |
| v33977 | 3 | 134.7 | 97.8 |
| v33979 | 3 | 104.4 | 97.8 |

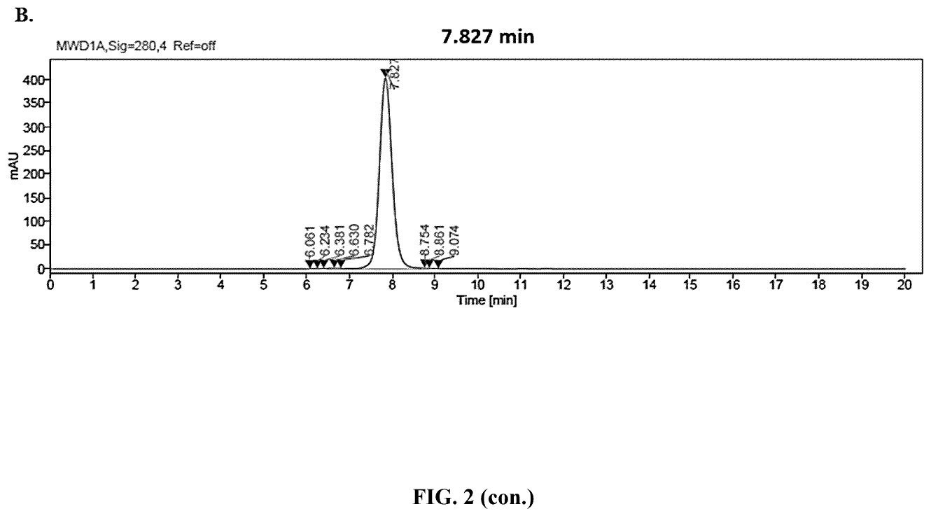

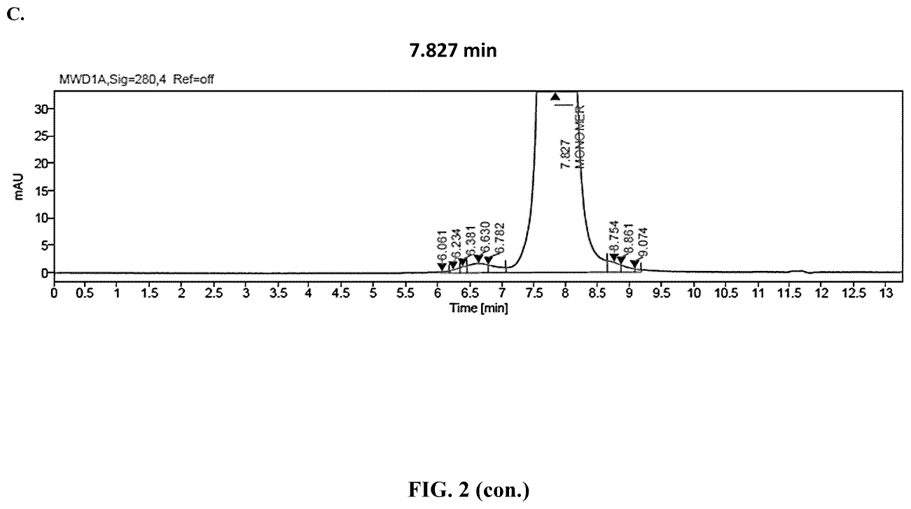

SDS-PAGE results for a representative cysteine insertion variant, v29001, under non-reducing (NR) and reducing (R) conditions corresponding to full-size antibody and intact heavy and light chains are shown in FIG. 2A, and the UPLC-SEC chromatogram for variant v29001 is shown in FIG. 2B. Based on the UPLC-SEC chromatogram, the sample purity for this variant was ˜99%, reflecting high species homogeneity.

### Example 2: Characterization of Cysteine Insertion Variants

Molecular weights of the cysteine insertion variants described in Example 1 were assessed by Liquid Chromatography-Mass Spectrometry (LC-MS). Differential Scanning calorimetry (DSC) was employed to evaluate thermal stability for two of the variants.

### 2.1 Liquid Chromatography-Mass Spectrometry (LC-MS)

The accurate mass of purified variants was measured by LC-MS. Variants were diluted to 1 mg/mL in PBS, pH 7.4, then deglycosylated. For deglycosylation, typically 1 ug EndoS was employed for every 10 μg of antibody and the reaction mix was incubated at room temperature for an hour. In some cases, samples were also reduced by adding 1 uL 500 mM tris(2-carboxyethyl) phosphine (TCEP) to each 10 uL sample followed by incubation at 70° C. for an hour. Finally, samples were run on an LC-MS quadrapole time-of-flight (QTOF) system (Agilent 1290 HPLC coupled to Agilent 6545 QTOF; Agilent Technologies, Inc., Santa Clara, CA), 1 uL injection each. The detailed procedure is described below.
Column: PLRP-S 1000 Å, 8 uM, 50×2.1 mm (Agilent Technologies, Inc., Santa Clara, CA)
Mobile phase C: 0.1% formic acid, 0.025% trifluoroacetic acid and 10% isopropyl alcohol in H₂O
Mobile Phase D: 0.1% formic acid and 10% isopropyl alcohol in acetonitrile
Detection: signal A (280 nm, 4.0 band width), signal B (220 nm, 4.0 band width)·
Gradient:

| Time | Buffer | Buffer |
|---|---|---|
| (Min) | C | D |
| 0 | 80 | 20 |
| 20 | 60 | 40 |
| 22 | 10 | 90 |
| 22.5 | 1 | 99 |
| 24 | 1 | 99 |

Post Run Time: 2 minutes

The measured deglycosylated masses of the full-size variants were consistent with theoretical masses (see Table 2.1).

TABLE 2.1
Mass Verification of Representative Cysteine Insertion Variants by LC-MS

| Variant No. | Expected Mass (Da) | Observed Mass (Da) | Mass Difference (Da) |
|---|---|---|---|
| v28983 | 146269 | 146272 | 3 |
| v29001 | 146269 | 146273 | 4 |
| v35527 | 147019 | 147027 | 8 |
| v33967 | 146166 | 146174 | 8 |
| v33968 | 146166 | 146170 | 4 |
| v33970 | 146371 | 146376 | 5 |
| v33971 | 146371 | 146378 | 7 |
| v33979 | 146371 | 146381 | 10 |

### 2.2 Differential Scanning Calorimetry (DSC)

The difference in melting temperature (Tm) between two of the cysteine insertion variants, v28983 and v29001, and the corresponding parental antibody, v17427, was determined by DSC as follows: 400 μL of purified sample at concentrations of 0.2 mg/mL or 0.4 mg/mL in PBS were used for DSC analysis with a MicroCal VP-Capillary DSC (GE Healthcare, Chicago, IL). At the start of each DSC run, 5 buffer blank injections were performed to stabilize the baseline, and a buffer injection was placed before each sample injection for referencing. Each sample was scanned from 20° C. to 100° C. at a 60° C./hr rate, with low feedback, 8 sec filter, 5 min preTstat, and 70 psi nitrogen pressure. The resulting thermograms were referenced and analyzed using Origin 7 software (OriginLab Corporation, Northampton, MA). The DSC measurements are summarized in Table 2.2.

TABLE 2.2
Thermal stability of Cysteine Insertion
Variants Compared to Parental Antibody

| | | Tm1 | Tm2 | Tm3 |
|---|---|---|---|---|
| Variant | No of Cysteine | (CH2 Domain) | (Fab) | (CH3 Domain) |
| No. | Insertions | (° C.) | (° C.) | (° C.) |
| v17427 | None | 71.9 | 76.0 | 83.0 |
| v28983 | 2 | No change | 72.0 | No change |
| v29001 | 2 | 65.0 | No change | No change |

### Example 3: Preparation of Drug-Linkers

The following abbreviations are used in this Example: ACN=acetonitrile; DCM=dichloromethane; DMF=dimethylformamide; DMSO=dimethylsulphoxide; LC/MS=Liquid Chromatography/Mass Spectrometry; LC-MSD=Liquid Chromatography-Mass Selective Detector; SEC=size-exclusion chromatography; HIC=hydrophobic interaction chromatography; RP-UPLC=reverse-phase ultra performance liquid chromatography; HPLC=high-performance liquid chromatography; MT=maleimidotriethylene glycolate; TCEP=tris(2-carboxyethyl) phosphine; TFA=trifluoroacetic acid; VC=valine-citrulline; UHPLC=ultra high-performance liquid chromatograph.

The following general methods were employed:

Flash Chromatography: Crude reaction products were purified with Biotage® Snap Ultra columns (10, 25, 50, or 100 g) (Biotage, Charlotte, NC), eluting with linear gradients of ethyl acetate/hexanes or methanol/dichloromethane on a Biotage® Isolera™ automated flash system (Biotage, Charlotte, NC). Alternatively, reverse-phase flash purification was conducted using Biotage® Snap Ultra C18 columns (12, 30, 60, or 120 g), eluting with linear gradients of CH3CN+0.1% TFA/H₂O+0.1% TFA. Purified compounds were isolated by either removal of organic solvents by rotavap or lyophilization of acetonitrile/water mixtures.

Preparative HPLC: Reverse-phase HPLC of crude compounds was performed using a Kinetex® 5-μm EVO C18 100 Å (250×21.2 mm) column (Phenomenex, Torrance, CA) on an Agilent 1260 Infinity II preparative LC-MSD system (Agilent Technologies, Inc., Santa Clara, CA), eluting with linear gradients of CH3CN+0.1% TFA/H₂O+0.1% TFA. Purified compounds were isolated by lyophilization of acetonitrile/water mixtures.

NMR: ¹H NMR spectra were collected with either a Bruker AVANCE III 300 Spectrometer (300 MHz) or Bruker AVANCE III 400 Spectrometer (400 MHZ) (Bruker Corporation, Billerica, MA). Chemical shifts are in parts per million (ppm).

2,2-dimethyl-4-oxo-3,8,11,14-tetraoxa-5-azaheptadecan-17-oic acid:

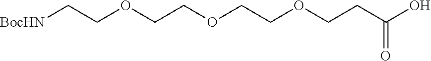 [SMILES: CC(C)(C)OC(=O)NCCOCCOCCOCCC(=O)O]

was obtained from ChemPep Inc. (Wellington, FL).

3.1 (S)-N-((3R,4S,5S)-1-((S)-2-((1R,2R)-3-((4-aminophenyl) sulfonamido)-1-methoxy-2-methyl-3-oxopropyl) pyrrolidin-1-yl)-3-methoxy-5-methyl-1-oxoheptan-4-yl)-2-((S)-2-(dimethylamino)-3-methylbutanamido)-N,3-dimethylbutanamide (Compound 1)

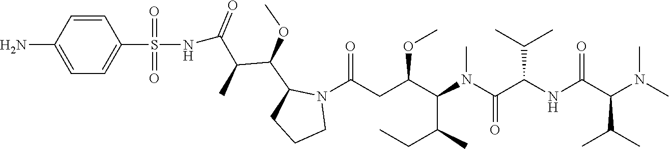 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

Prepared as described in International Publication No. WO 2016/041082.

3.2 (S)-2-((S)-2-amino-3-methylbutanamido)-N-(4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl)-5-ureidopentanamide (Compound 15)

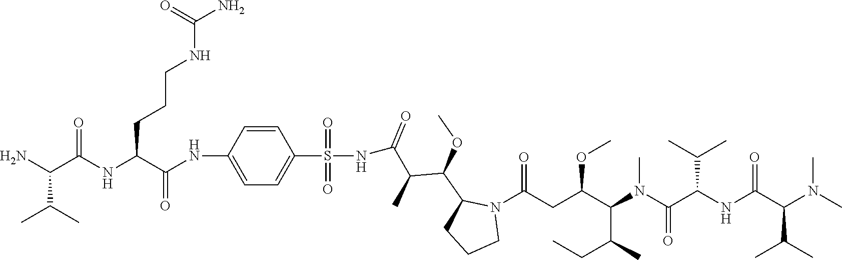 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](N)C(C)C)cc1)OC)N(C)C(=O)[C@@H](NC(=O)[C@H](C(C)C)N(C)C)C(C)C]

Prepared as described in International Publication No. WO 2019/173911.

### 3.3 2,3,5,6-tetrafluorophenyl 3-(2-(2-(2-(2,5-dioxo-2,5-dihydro-1H-pyrrol-1-yl) ethoxy) ethoxy) ethoxy)propanoate (Compound 2)

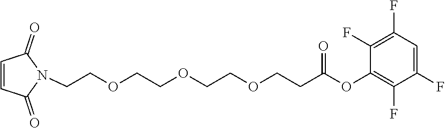 [SMILES: O=C(CCOCCOCCOCCN1C(=O)C=CC1=O)Oc1c(F)c(F)cc(F)c1F]

Flask 1:3-{2-[2-(2-aminoethoxy) ethoxy]ethoxy}propanoic acid (10.0 g, 45.2 mmol, 1 equiv.) and maleic anhydride (4.43 g, 45.2 mmol, 1 equiv.) were dissolved in anhydrous DMF (15 mL) in a dried 250 mL RB flask, and then stirred at room temperature overnight under nitrogen. The following morning, the reaction was determined to be complete by LC/MS. To the reaction was added 2,3,5-collidine (21.9 g, 24.1 mL, 181 mmol, 4 equiv.) and the mixture cooled to 0° C. Flask 2: In a separate flask 2,3,4,5-tetrafluorophenol (30.0 g, 181 mmol, 4 equiv.) was dissolved in 45 mL anhydrous DMF and cooled to 0° C., upon which trifluoroacetic anhydride (38.0 g, 25.4 mL, 1.49 g/mL, 181 mmol, 4 equiv.) was added dropwise over 2 min. The resulting solution was stirred at 0° C. for 10 min before 2,3,5-collidine (21.9 g, 24.1 mL, 0.91 g/mL, 181 mmol, 4 equiv.) was added over 3 min. The final mixture was allowed to stir at 0° C. for 15 min and then then added to the Flask 1 solution over 3 min, after which the final mixture was brought to room temperature and stirred for 48 h. A small amount of intermediate remained as determined by LC/MS and the reaction was stirred for additional 72 h before conversion was determined to be totally complete by LC/MS. The reaction was acidified with 1 M HCl (75 mL) and extracted with Et₂O (3×100 mL). The combined organic layers were then washed with 5% LiCl (60 mL) and brine (30 mL), then dried over MgSO₄ and concentrated in vacuo. The crude product was purified via reverse phase flash chromatography over a 10-50% ACN/H₂O+0.1% TFA gradient. Product containing fractions were pooled for lyophilization and the title compound was recovered as a light orange oil (14.5 g, 32.4 mmol, 71.4%).

LC/MS: calc'd m/z=449.11 for C₁₉H₁₉F₄NO₇, detected [M+H]⁺=450.2 m/z. ¹H NMR (300 MHz, MeOD) δ 7.42 (tt, J=10.5, 7.2 Hz, 1H), 6.81 (s, 2H), 3.87 (t, J=6.0 Hz, 2H), 3.71-3.55 (m, 12H), 2.98 (t, J=6.0 Hz, 2H).

### 3.4 di-tert-butyl 4-(3-(tert-butoxy)-3-oxopropyl)-4-(3-(2-(2-(2-(2,5-dioxo-2,5-dihydro-1H-pyrrol-1-yl) ethoxy) ethoxy) ethoxy) propanamido) heptanedioate (Compound 3)

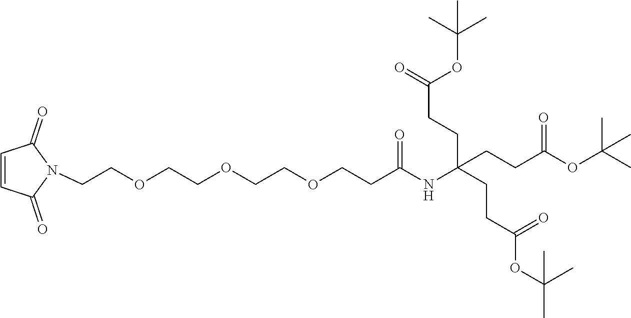 [SMILES: CC(C)(C)OC(=O)CCC(CCC(=O)OC(C)(C)C)(CCC(=O)OC(C)(C)C)NC(=O)CCOCCOCCOCCN1C(=O)C=CC1=O]

1,7-di-tert-butyl 4-amino-4-[3-(tert-butoxy)-3-oxopropyl]heptanedioate (2.89 g, 6.95 mmol, 1.1 equiv.) and Compound 2 (2.84 g, 6.32 mmol, 1 equiv.) were dissolved in 10 mL of anhydrous DMF along with 1-hydroxybenzotriazole monohydrate (0.968 g, 6.32 mmol, 1 equiv.). N-Ethyldiisopropylamine (1.63 g, 2.21 mL, 0.74 g/mL, 12.6 mmol, 2 equiv.) was added over 1 min with rapid stirring at room temperature. The reaction was determined to be complete by LC/MS after 1 h and was diluted with 1 M HCl (5 mL) and ACN (3 mL), then purified via reverse phase chromatography over a 10-100% ACN/H₂O+0.1% TFA gradient. Product was pooled and concentrated in vacuo, then brine (5 mL) was added and the aqueous layer extracted with Et₂O (3×50 mL). Organics were pooled and dried over brine (5 mL) and MgSO₄, then filtered and evaporated in vacuo to recover the title compound as an oily off-white solid (4.18 g, 6.00 mmol, 94.9%).

LC/MS: calc'd m/z=698.40 for C₃₅H₅₈N₂O₁₂, detected [M+H]⁺=699.6 m/z. ¹H NMR (400 MHZ, CDCl₃) δ 6.72 (s, 2H), 6.13 (s, 1H), 3.78-3.69 (m, 4H), 3.67-3.63 (m, 2H), 3.63-3.61 (m, 8H), 2.40 (t, J=5.8 Hz, 2H), 2.27-2.15 (m, 6H), 2.03-1.92 (m, 6H), 1.44 (s, 27H).

### 3.5 4-(2-carboxyethyl)-4-(3-(2-(2-(2-(2,5-dioxo-2,5-dihydro-1H-pyrrol-1-yl) ethoxy) ethoxy) ethoxy) propanamido) heptanedioic acid (Compound 4)

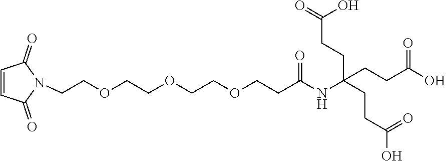 [SMILES: O=C(O)CCC(CCC(=O)O)(CCC(=O)O)NC(=O)CCOCCOCCOCCN1C(=O)C=CC1=O]

Compound 3 (4.18 g, 6.00 mmol, 1 equiv.) was dissolved in 33% TFA/DCM (15 mL) in a 500 mL round bottom flask. Stirred at room temperature for 18 h, at which point reaction was determined to be complete by LC/MS. The reaction was concentrated to dryness in vacuo and the residue co-evaporated with ACN (3×10 mL). The residue was taken up in 10 mL 2:1 H₂O/ACN and lyophilized. The title compound was recovered as an oil which was ˜25% overweight and assumed to contain residual TFA and H₂O. (4.0 g, assumed quant yield 6.00 mmol, 100%).

LC/MS: calc'd m/z=530.21 for C₂₃H₃₄N₂O₁₂, detected [M+H]⁺=531.4 m/z. ¹H NMR (300 MHz, MeOD) δ 6.84 (s, 2H), 3.74-3.68 (m, 4H), 3.68-3.63 (m, 4H), 3.63-3.57 (m, 8H), 2.46-2.39 (m, 2H), 2.39-2.25 (m, 4H), 2.10-1.97 (m, 6H).

### 3.6 bis(2,3,5,6-tetrafluorophenyl) 4-(3-(2-(2-(2-(2,5-dioxo-2,5-dihydro-1H-pyrrol-1-yl) ethoxy) ethoxy) ethoxy) propanamido)-4-(3-oxo-3-(2,3,5,6-tetrafluorophenoxy) propyl) heptanedioate (Compound 5)

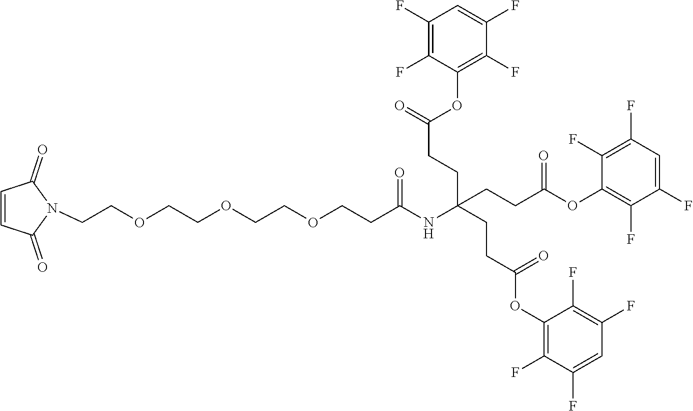 [SMILES: O=C(CCOCCOCCOCCN1C(=O)C=CC1=O)NC(CCC(=O)Oc1c(F)c(F)cc(F)c1F)(CCC(=O)Oc1c(F)c(F)cc(F)c1F)CCC(=O)Oc1c(F)c(F)cc(F)c1F]

Compound 4 (3.34 g, 6.30 mmol, 1 equiv.) was dissolved with 2,3,5,6-tetrafluorophenol (4.18 g, 25.2 mmol, 4 equiv.) in ACN (50 mL) and stirred at 0° C. in a 250 mL round bottom flask. 1-ethyl-3-(3-dimethylaminopropyl) carbodiimide hydrochloride (4.83 g, 25.2 mmol, 4 equiv.) was added. After 30 min, LC/MS indicated conversion was around 60%, with the major impurity being partially esterified intermediate. Additional 1-ethyl-3-(3-dimethylaminopropyl) carbodiimide hydrochloride (500 mg) was added and allowed to stir for 1 h, at which point conversion was nearly total. The reaction was concentrated in vacuo to ˜8 mL volume, then diluted with 1 M HCl (5 mL) and H₂O (5 mL) and purified via reverse phase chromatography over a 10-75% ACN/H₂O+0.1% TFA gradient. Product fractions were pooled and concentrated in vacuo and then extracted with Et₂O (2×40 mL). Organics were pooled and washed with brine (30 mL), then dried over MgSO₄ and filtered to recover the title compound as a clear and colourless oil (2.60 g, 2.67 mmol, 42.3% yield).

LC/MS: calc'd m/z=974.2 for C₄₁H₃₄F₁₂N₂O₁₂, detected [M+H]⁺=975.4 m/z. ¹H NMR (300 MHz, CDCl₃) δ 7.03 (tt, J=9.8, 7.0 Hz, 3H), 6.71 (s, 2H), 6.62 (s, 1H), 3.82-3.76 (m, 2H), 3.76-3.69 (m, 2H), 3.69-3.59 (m, 10H), 2.81 (dd, J=9.0, 6.8 Hz, 6H), 2.54 (t, J=5.5 Hz, 2H), 2.37-2.26 (m, 6H).

### 3.7 bis(2,3,5,6-tetrafluorophenyl) (tert-butoxycarbonyl)-L-glutamate (Compound 6)

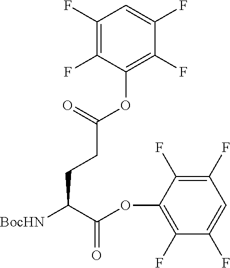 [SMILES: CC(C)(C)OC(=O)N[C@@H](CCC(=O)Oc1c(F)c(F)cc(F)c1F)C(=O)Oc1c(F)c(F)cc(F)c1F]

To a 250 mL round bottom flask containing glutamate (1.80 g, 7.28 mmol, 1 equiv.), ACN (30 mL) and 2,3,5,6-tetrafluorophenol (2.54 g, 15.3 mmol, 2.1 equiv.) and 1-ethyl-3-(3-dimethylaminopropyl) carbodiimide hydrochloride (2.93 g, 15.3 mmol, 2.1 equiv.) were added. The reaction was stirred for 18 h at room temperature at which time reaction was determined to be complete by LC/MS. The reaction mixture was concentrated to a crude oil in vacuo and then redissolved into EtOAc (50 mL) and 1 M HCl (20 mL). The organic layer was washed with 1 M HCl (2×20 mL), saturated NaHCO₃ (20 mL) and 1× brine (20 mL), then dried over MgSO₄, filtered and evaporated in vacuo to yield the title compound as a white solid. (3.27 g, 6.02 mmol, 82.7% yield).

LC/MS: calc'd m/z=543.09 for C₂₂H₁₇F₈NO₆, detected [M+Na]⁺=556.4 m/z. ¹H NMR (400 MHZ, CDCl₃) δ 7.13-6.97 (m, 2H), 5.17 (s, 1H), 4.83 (s, 1H), 2.94 (q, J=7.2 Hz, 2H), 2.63-2.50 (m, 1H), 2.39-2.24 (m, 1H), 1.50 (s, 9H).

### 3.8 (S)-15-((tert-butoxycarbonyl)amino)-14,18-dioxo-4,7,10,22,25,28-hexaoxa-13,19-diazahentriacontanedioic acid (Compound 7)

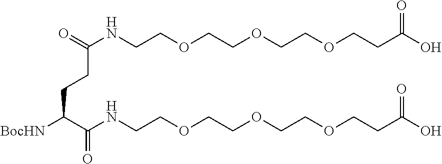 [SMILES: CC(C)(C)OC(=O)N[C@@H](CCC(=O)NCCOCCOCCOCCC(=O)O)C(=O)NCCOCCOCCOCCC(=O)O]

To a flask containing Compound 6 (1.00 g, 1.84 mmol, 1 equiv.) and 3-(2-(2-(2-aminoethoxy) ethoxy) ethoxy) propanoic acid (0.855 g, 3.87 mmol, 2.1 equiv.) was added ACN (15 mL) and saturated NaHCO₃ solution (1.14 M, 9.59 mL, 11.0 mmol, 6 equiv.). The reaction was stirred at room temperature for 18 h, at which point the reaction was determined to be complete by LC/MS. Reaction was used without purification assuming quantitative yield.

LC/MS: calc'd m/z=653.34 for C₂₈H₅₁N₃O₁₄, detected [M+H]⁺=654.7 m/z.

### 3.9 bis(2,3,5,6-tetrafluorophenyl) (S)-15-((tert-butoxycarbonyl)amino)-14,18-dioxo-4,7,10,22,25,28-hexaoxa-13,19-diazahentriacontanedioate (Compound 8)

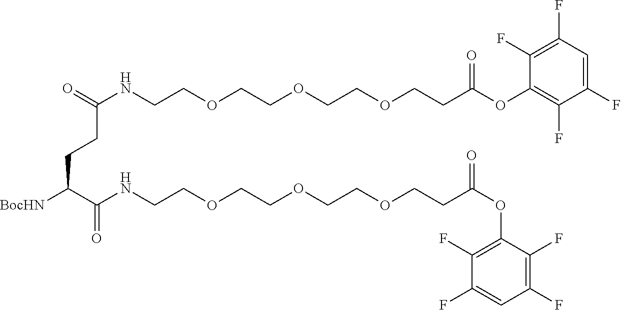 [SMILES: CC(C)(C)OC(=O)N[C@@H](CCC(=O)NCCOCCOCCOCCC(=O)Oc1c(F)c(F)cc(F)c1F)C(=O)NCCOCCOCCOCCC(=O)Oc1c(F)c(F)cc(F)c1F]

To a reaction solution containing Compound 7 (1.20 g, 1.84 mmol, 1 equiv.) was added 1 M NaH₂PO₄ (5 mL) and 1 M HCl (5 mL) to lower pH to ˜5. 2,3,5,6-tetrafluorophenol (0.641 g, 3.87 mmol, 2.1 equiv.) and 1-ethyl-3-(3-dimethylaminopropyl) carbodiimide hydrochloride (1.06 g, 5.52 mmol, 3.0 equiv.) were added and the reaction was stirred for 18 h at room temperature at which point LC/MS indicated the presence of intermediate species as well as product. Additional 1-ethyl-3-(3-dimethylaminopropyl) carbodiimide hydrochloride (300 mg) was added to bring reaction to completion. The reaction mixture was concentrated in vacuo to remove the bulk of the ACN and then purified via reverse phase chromatography over a 10-100% ACN/H₂O+0.1% TFA gradient. Product fractions were pooled and concentrated in vacuo to remove ACN, then extracted with Et₂O (50 mL) and 3×DCM (50 mL). Organics were pooled and dried over MgSO₄, then filtered and evaporated in vacuo to recover the title compound as a clear oil (1.05 g, 1.11 mmol, 60.1%).

LC/MS: calc'd m/z=949.32 for C₄₀H₅₁F₃N₃O₁₄, detected [M+H]⁺=950.8 m/z. ¹H NMR (400 MHZ, CDCl₃) δ 7.12 (s, 1H), 7.07-6.97 (m, 2H), 6.76 (s, 1H), 5.66 (d, J=7.6 Hz, 1H), 4.12 (d, J=7.2 Hz, 1H), 3.89 (t, J=6.2 Hz, 4H), 3.71-3.61 (m, 16H), 3.61-3.57 (m, 2H), 3.54-3.36 (m, 3H), 2.96 (td, J=6.2, 1.8 Hz, 4H), 2.40-2.19 (m, 2H), 2.11-1.92 (m, 2H), 1.43 (s, 9H).

3.10 tert-butyl ((6S,9S,25S,43S,46S)-1,51-diamino-6,46-bis((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9,43-diisopropyl-1,8,11,24,28,41,44,51-octaoxo-14,17,20,32,35,38-hexaoxa-2,7,10,23,29,42,45,50-octaazahenpentacontan-25-yl) carbamate (Compound 9)

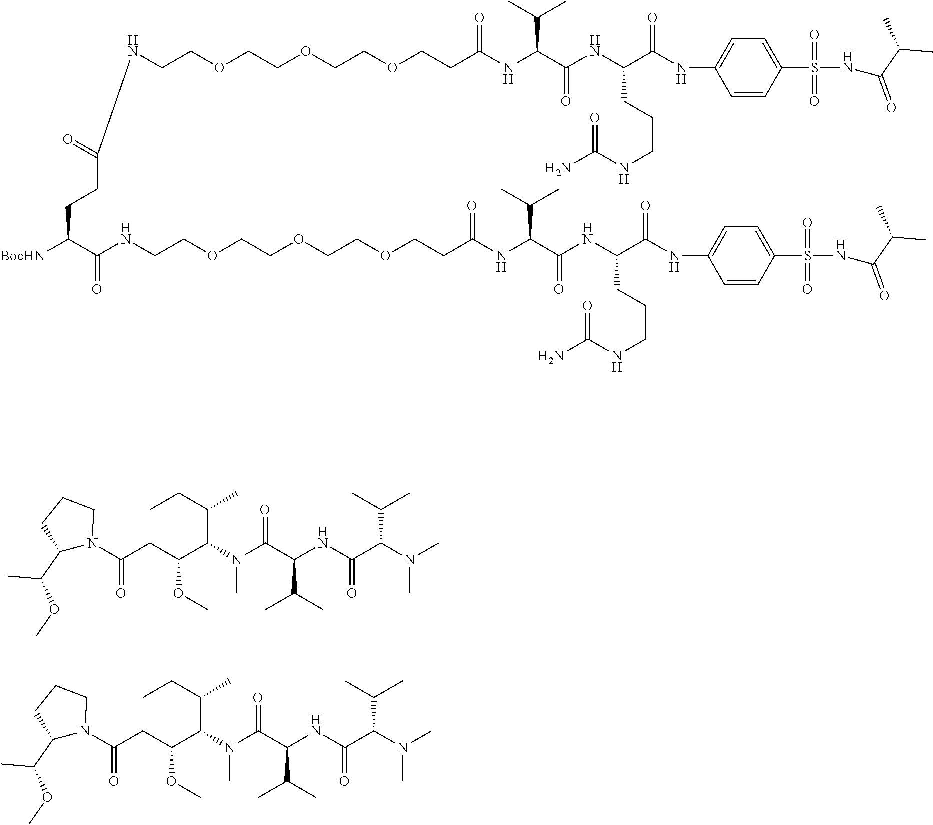

Compound 8 (570 mg, 0.6 mmol, 1 equiv.) was dissolved in 2 mL DMF and Compound 15 (1.66 g, 80 w/w %, 1.32 mmol, 2.2 equiv.) was added as a DMF solution (3 mL). The resulting solution was stirred at room temperature and N-ethyldiisopropylamine (0.310 g, 0.419 mL, 0.741 g/mL, 2.40 mmol, 4 equiv.) was added. The reaction was allowed to stir at room temperature for 42 h, at which point LC/MS indicated reaction was complete. The reaction was diluted with 1 M NaH₂PO₄ (3 mL) and 1 M HCl (1 mL) and purified via reverse phase chromatography over a 10-55% ACN/H₂O+0.1% TFA gradient. Product fractions were pooled and evaporated in vacuo to recover the title compound as a white solid (1.00 g, 0.379 mmol, 63.2%).

LC/MS: calc'd m/z=2635.53 for C₁₂₄H₂₁₅N₂₃O₃₄S₂, detected [M+3H]³⁺=879.8 m/z. 1H NMR (400 MHZ, MeOD) δ 7.90 (d, J=8.9 Hz, 3H), 7.77 (d, J=20.8 Hz, 5H), 4.70 (t, J=8.7 Hz, 1H), 4.60-4.52 (m, 3H), 4.28 (dd, J=17.8, 7.2 Hz, 2H), 4.11 (d, J=18.5 Hz, 3H), 3.77 (s, 3H), 3.66-3.57 (m, 15H), 3.54 (d, J=5.5 Hz, 2H), 3.33 (dt, J=3.3, 1.7 Hz, 61H), 3.22-3.11 (m, 5H), 2.66-2.47 (m, 8H), 2.41 (d, J=5.4 Hz, 8H), 2.32 (d, J=7.9 Hz, 2H), 2.21-1.98 (m, 4H), 1.94 (d, J=19.9 Hz, 2H), 1.68-1.53 (m, 2H), 1.45 (s, 11H), 1.21-1.09 (m, 6H), 1.03 (dq, J=17.9, 6.7 Hz, 37H), 0.93-0.83 (m, 10H).

3.11 (S)-2-amino-N¹,N⁵-bis((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl) pentanediamide (Compound 10)

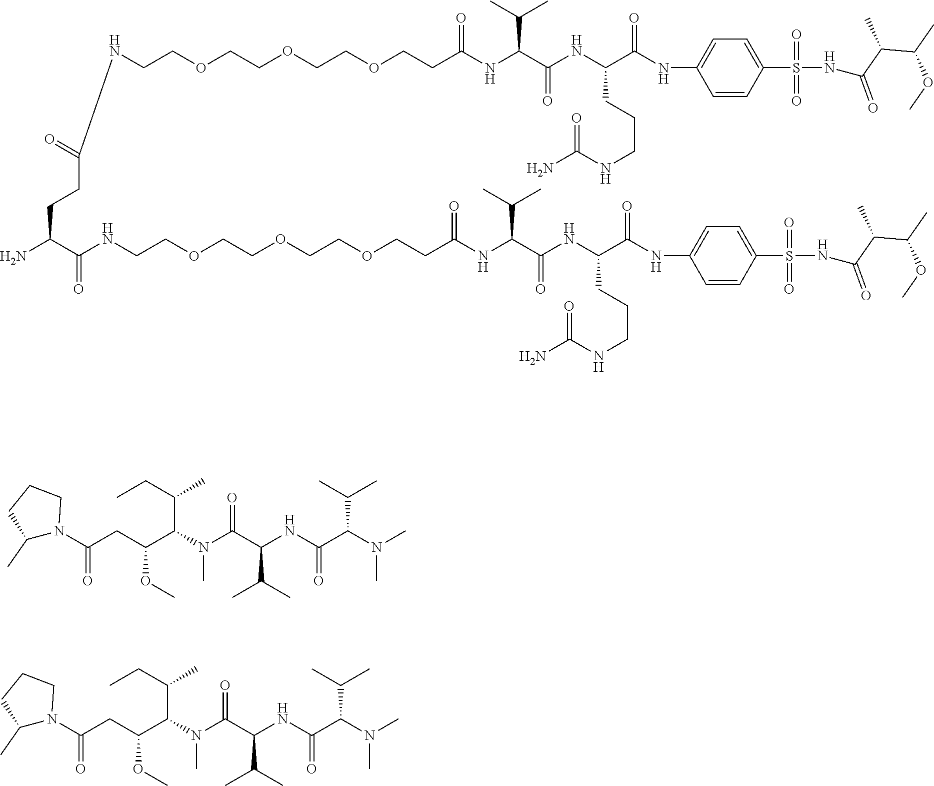

Compound 9 (850 mg, 0.322 mmol, 1 equiv.) was dissolved in 10% TFA/DCM (10 mL) and stirred at room temperature. Reaction was completed within 2 h as determined by LC/MS. The reaction was evaporated in vacuo to an oily residue, co-evaporated with ACN (10 mL), then redissolved into 10 mL of 2:1 H₂O/ACN and purified via reverse phase chromatography over a 10-50% ACN/H₂O+0.1% gradient. Product fractions were pooled and lyophilized to recover the title compound as a white solid. (0.845 g, 0.294 mmol, 91.1%).

LC/MS: calc'd m/z=2535.48 for C₁₁₉H₂₀₇N₂₃O₃₂S₂ detected [M+3H]³⁺=846.6 m/z. 1H NMR (400 MHZ, MeOD) δ 8.03-7.92 (m, 7H), 7.92 (s, 2H), 4.72 (t, J=8.4 Hz, 1H), 4.56 (dd, J=9.3, 4.8 Hz, 3H), 4.30-4.20 (m, 2H), 4.16-4.02 (m, 3H), 3.98 (d, J=14.4 Hz, 1H), 3.92-3.82 (m, 3H), 3.77 (qd, J=6.5, 3.2 Hz, 9H), 3.68-3.45 (m, 36H), 3.46-3.37 (m, 7H), 3.37-3.28 (m, 31H), 3.29-3.19 (m, 1H), 2.95 (d, J=15.1 Hz, 16H), 2.59 (dd, J=8.1, 3.8 Hz, 8H), 2.51 (d, J=7.7 Hz, 2H), 2.43 (dq, J=9.1, 6.7 Hz, 5H), 2.13 (dt, J=11.9, 6.7 Hz, 7H), 2.05-1.87 (m, 1H), 1.80 (ddd, J=20.2, 14.6, 9.2 Hz, 2H), 1.71-1.54 (m, 4H), 1.49-1.36 (m, 1H), 1.18-0.96 (m, 42H), 0.88 (q, J=7.1 Hz, 9H).

3.12 (S)-N¹,N⁵-bis((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl)-2-(3-(2-(2-(2-(2,5-dioxo-2,5-dihydro-1H-pyrrol-1-yl) ethoxy) ethoxy) ethoxy) propanamido) pentanediamide (Drug-Linker 003)

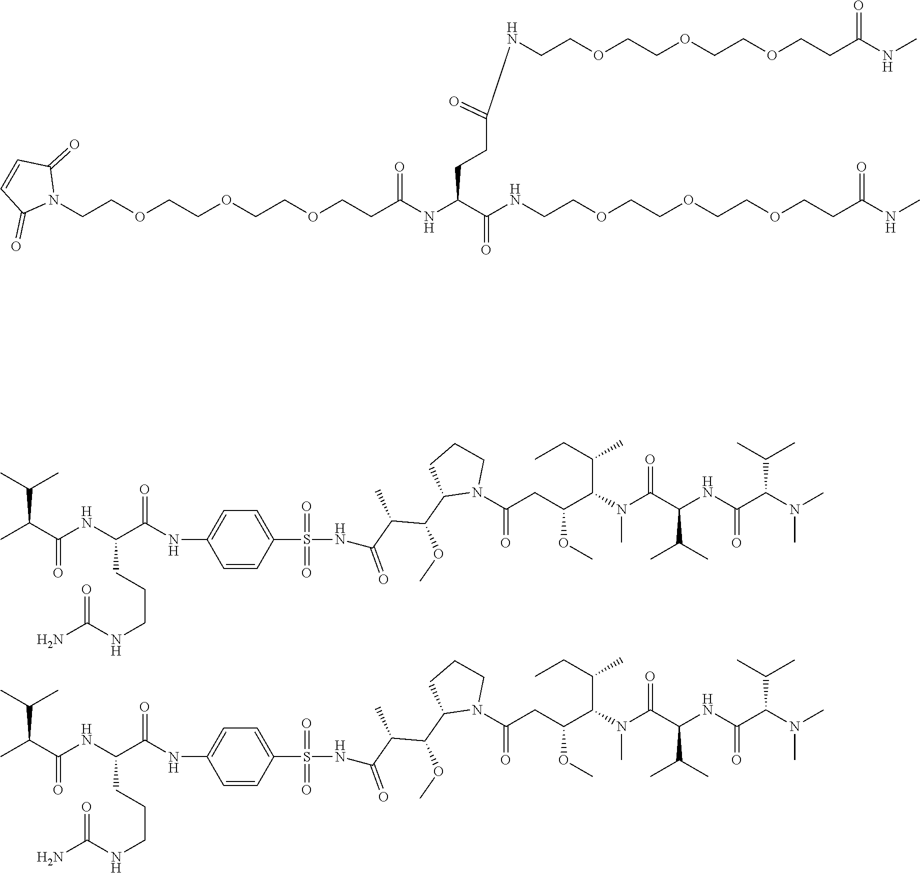

Compound 10 (840 mg, 0.292 mmol, 1 equiv.) and 2,3,5,6-tetrafluorophenyl 3-(2-{2-[2-(2,5-dioxopyrrol-1-yl) ethoxy]ethoxy}ethoxy)propanoate (0.144 g, 0.321 mmol, 1.1 equiv.) were dissolved in DMF (5 mL) in a 50 mL round bottom flask. N-ethyldiisopropylamine (0.189 g, 0.255 mL, 0.74 g/mL, 1.46 mmol, 5 equiv.) was added and stirred at room temperature. After 1 h, LC/MS indicated reaction was complete. The reaction was diluted with 1 M HCl (5 mL) and purified via reverse phase chromatography over a 10-60% ACN/H₂O+0.1% TFA gradient. Product fractions were pooled and lyophilized to recover the title compound as a white solid (0.660 g, 0.217 mmol, 74.2%).

LC/MS: calc'd m/z=2818.58 for C₁₃₂H₂₂₄N₂₄O₃₈S₂, detected [M+3H]³⁺=941.0 m/z. ¹H NMR (300 MHz, MeOD) δ 7.99-7.82 (m, 8H), 6.85 (s, 2H), 4.77-4.70 (m, 1H), 4.62-4.49 (m, 3H), 4.30-4.20 (m, 2H), 4.17-4.03 (m, 2H), 3.86 (d, J=7.3 Hz, 2H), 3.83-3.66 (m, 9H), 3.66-3.46 (m, 40H), 3.43-3.23 (m, 48H), 3.16 (s, 6H), 2.95 (d, J=10.9 Hz, 12H), 2.59 (d, J=6.8 Hz, 6H), 2.53 (s, 1H), 2.45 (d, J=5.8 Hz, 4H), 2.22 (d, J=9.1 Hz, 5H), 2.01 (s, 4H), 1.60 (d, J=6.9 Hz, 2H), 1.52-1.37 (m, 2H), 1.18-0.96 (m, 53H), 0.89 (q, J=6.8 Hz, 6H).

### 3.13 2,3,5,6-tetrafluorophenyl 2,2-dimethyl-4-oxo-3,8,11,14-tetraoxa-5-azaheptadecan-17-oate (Compound 11)

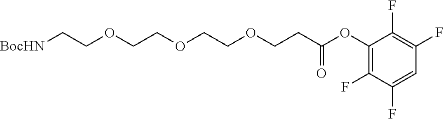 [SMILES: CC(C)(C)OC(=O)NCCOCCOCCOCCC(=O)Oc1c(F)c(F)cc(F)c1F]

2,2-dimethyl-4-oxo-3,8,11,14-tetraoxa-5-azaheptadecan-17-oic acid (1.412 g, 4.394 mmol, 1 equiv.), 2,3,5,6-tetrafluorophenol (0.803 g, 4.83 mmol, 1.1 equiv.) and 1-ethyl-3-(3-dimethylaminopropyl) carbodiimide hydrochloride (0.926 g, 4.83 mmol, 1.1 equiv.) were dissolved in ACN (15 mL) in a 250 mL round bottom flask. The reaction was stirred at room temperature for 18 h, at which point LC/MS indicated reaction was complete. The solvent was evaporated to recover crude product and redissolved into Et₂O (100 mL) and H₂O (20 mL). The organic layer was washed with saturated NaHCO₃ (3×20 mL), 1 M HCl (2×20 mL) and brine (20 mL), then dried over MgSO₄, filtered and evaporated filtrate to dryness in vacuo to recover the title compound as a clear/colourless oil (2.06 g, 4.39 mmol, 99%).

LC/MS: calc'd m/z=469.17 for C₂₀H₂₇F₄NO₇ detected [M+H-Boc]⁺=370.2 m/z. 1H NMR (300 MHz, CDCl₃) δ 7.03 (tt, J=9.9, 7.1 Hz, 1H), 5.05 (d, J=15.3 Hz, 1H), 3.92 (t, J=6.2 Hz, 2H), 3.77-3.62 (m, 8H), 3.60-3.51 (m, 2H), 3.34 (t, J=5.2 Hz, 2H), 2.99 (t, J=6.3 Hz, 2H), 1.47 (s, 9H).

3.14 tert-butyl ((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl) carbamate (Compound 12)

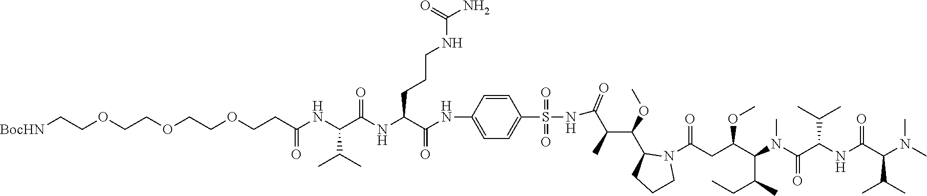 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)OC(C)(C)C)C(C)C)cc1)OC)N(C)C(=O)[C@@H](NC(=O)[C@H](C(C)C)N(C)C)C(C)C]

Compound 15 (3.00 g, 80 w/w %, 2.38 mmol, 1.1 equiv.) and Compound 11 (1.02 g, 2.16 mmol, 1 equiv.) were dissolved in DMF (10 mL) in a round bottom flask. N-ethyldiisopropylamine (0.838 g, 1.13 mL, 0.74 g/mL, 6.49 mmol, 3 equiv.) was added and stirred at room temperature. Reaction completed within 30 min as determined by LC/MS. The reaction was concentrated on a rotovap to ˜5 mL, diluted with a premixed solution of H₂O (7 mL) and TFA (1 mL), then purified via reverse phase chromatography over a 10-45% ACN/H₂O+0.1% TFA gradient. Product fractions were pooled and lyophilized to recover the title compound as a white solid (1.80 g, 1.26 mmol, 84.6%).

LC/MS: calc'd m/z=1311.77 for C₆₂H₁₀₉N₁₁O₁₇S detected [M+2H]⁺²=657.2 m/z. 1H NMR (300 MHz, MeOD) δ 8.02-7.91 (m, 2H), 7.90-7.81 (m, 1H), 4.73 (t, J=8.4 Hz, 1H), 4.56 (dt, J=8.7, 4.2 Hz, 1H), 4.23 (ddd, J=8.6, 5.8, 2.6 Hz, 1H), 4.16-4.01 (m, 1H), 3.94-3.82 (m, 1H), 3.82-3.69 (m, 3H), 3.62 (d, J=2.3 Hz, 7H), 3.57-3.47 (m, 2H), 3.38 (s, 1H), 3.35-3.27 (m, 1H), 3.23 (t, J=5.6 Hz, 2H), 3.16 (s, 2H), 2.99-2.90 (m, 6H), 2.65-2.55 (m, 2H), 2.53-2.33 (m, 1H), 2.19-2.08 (m, 1H), 2.05 (s, 1H), 2.02-1.84 (m, 1H), 1.84-1.71 (m, 1H), 1.68-1.55 (m, 1H), 1.45 (s, 9H), 1.18-0.94 (m, 22H), 0.89 (q, J=6.9 Hz, 3H)

3.15 (S)-2-((S)-1-amino-14-isopropyl-12-oxo-3,6,9-trioxa-13-azapentadecan-15-amido)-N-(4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl)-5-ureidopentanamide (Compound 13)

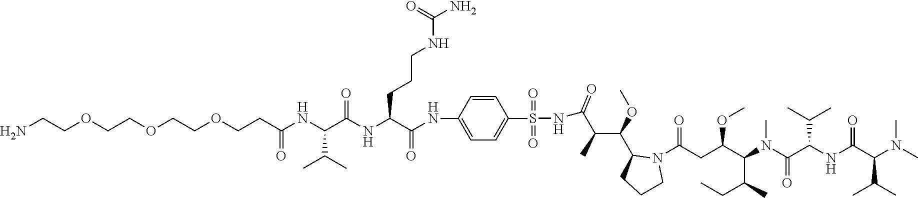 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCN)C(C)C)cc1)OC)N(C)C(=O)[C@@H](NC(=O)[C@H](C(C)C)N(C)C)C(C)C]

Compound 12 (1.80 g, 1.26 mmol, 1 equiv.) was dissolved in 10% TFA/DCM (15 mL) in a 250 mL round bottom flask and stirred at room temperature for 2 h at which point LC/MS indicated reaction was complete. The reaction was evaporated to dryness in vacuo, redissolved into 2:1 H₂O/ACN solution (8 mL) and lyophilized to recover the title compound as an oily white solid (1.81 g, 1.25 mmol, 99%).

LC/MS: calc'd m/z=1211.72 for C₅₇H₁₀₁N₁₁O₁₅S detected [M+2H-Boc]⁺²=607.2 m/z. ¹H NMR (300 MHZ, MeOD) δ 8.02-7.93 (m, 2H), 7.93-7.81 (m, 2H), 4.73 (d, J=8.7 Hz, 1H), 4.60-4.46 (m, 1H), 4.24 (d, J=6.9 Hz, 1H), 4.15-4.03 (m, 1H), 3.87 (d, J=5.7 Hz, 1H), 3.77 (dd, J=6.1, 2.8 Hz, 1H), 3.72 (t, J=5.2 Hz, 2H), 3.69-3.62 (m, 7H), 3.35-3.29 (m, 11H), 3.19-3.10 (m, 4H), 3.03-2.90 (m, 6H), 2.59 (t, J=6.2 Hz, 2H), 2.56-2.49 (m, 1H), 2.49-2.32 (m, 3H), 2.12 (dt, J=13.4, 6.7 Hz, 1H), 2.05 (s, 1H), 1.98 (d, J=4.8 Hz, 1H), 1.90 (t, J=6.8 Hz, 1H), 1.82-1.71 (m, 1H), 1.65-1.54 (m, 1H), 1.52-1.38 (m, 1H), 1.20-0.96 (m, 13H), 0.89 (q, J=7.0 Hz, 3H).

3.16 4-((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11,24-tetraoxo-14,17,20-trioxa-2,7,10,23-tetraazahexacosan-26-yl)-N¹,N⁷-bis((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl)-4-(3-(2-(2-(2-(2,5-dioxo-2,5-dihydro-1H-pyrrol-1-yl) ethoxy) ethoxy) ethoxy) propanamido) heptanediamide (Drug-Linker 004)

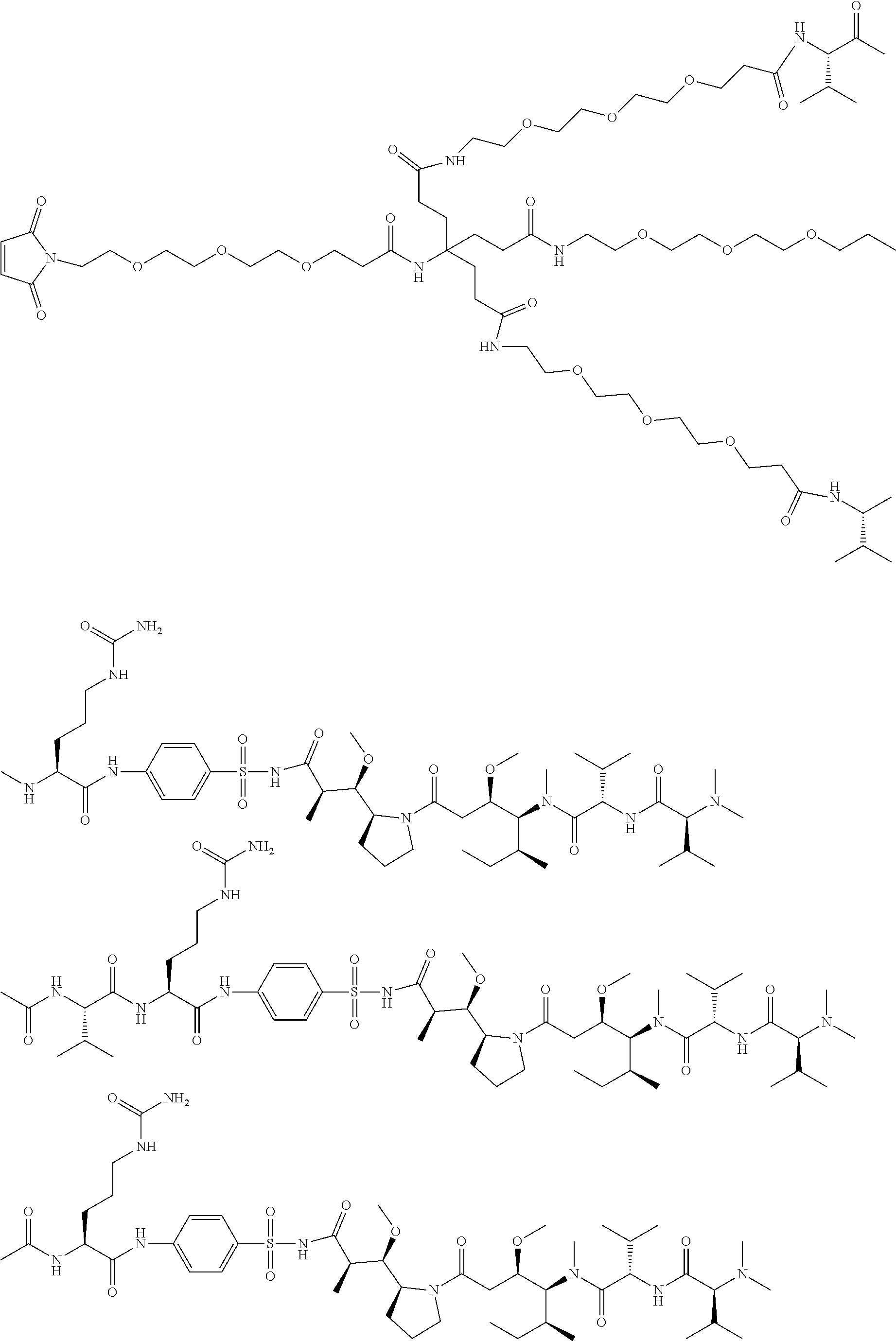

Compound 13 (3.104 g, 2.154 mmol, 4.2 equiv.) was dissolved in anhydrous DMF (8 mL). Compound 5 (500 mg, 0.513 mmol, 1 equiv.) was dissolved separately in DMF (1 mL). The two solutions were combined and rinsed with DMF (3 mL). N-ethyldiisopropylamine (0.796 g, 1.07 mL, 0.74 g/mL, 6.16 mmol, 12 equiv.) was added and stirred at room temperature. Reaction was complete within 3 h as determined by LC/MS. The reaction was neutralized with TFA (1 mL), diluted with 1 M HCl (3 mL) and H₂O (12 mL), then purified via reverse phase chromatography over a 10-50% ACN/H₂O+0.1% TFA gradient. Product fractions were isolated and lyophilized to recover the title compound as a white solid (1.07 g, 0.513 mmol, 46.6%).

LC/MS: calc'd m/z=4112.34 C₁₉₄H₃₃₁N₃₅O₅₄S₃, detected [M+4H]⁺⁴=1029.8 m/z. 1H NMR (300 MHZ, MeOD) δ 8.03-7.81 (m, 9H), 6.85 (s, 2H), 4.74 (d, J=8.8 Hz, 2H), 4.61-4.51 (m, 3H), 4.24 (t, J=6.4 Hz, 3H), 4.12-4.06 (m, 3H), 3.86 (d, J=7.6 Hz, 3H), 3.82-3.67 (m, 7H), 3.67-3.57 (m, 31H), 3.54 (t, J=5.5 Hz, 5H), 3.38 (s, 5H), 3.36 (s, 3H), 3.35-3.32 (m, 39H), 3.31 (d, J=3.8 Hz, 9H), 3.16 (d, J=7.8 Hz, 6H), 2.95 (d, J=10.8 Hz, 18H), 2.56 (d, J=15.7 Hz, 6H), 2.49-2.41 (m, 3H), 2.26-2.17 (m, 3H), 2.06-1.97 (m, 5H), 1.64-1.56 (m, 2H), 1.48-1.42 (m, 2H), 1.17-0.96 (m, 49H), 0.89 (q, J=6.8 Hz, 8H).

### 3.17 bis(2,3,5,6-tetrafluorophenyl) adipate (Compound 14)

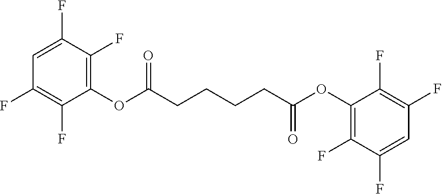 [SMILES: O=C(CCCCC(=O)Oc1c(F)c(F)cc(F)c1F)Oc1c(F)c(F)cc(F)c1F]

2,3,5,6-tetrafluorophenol (37.0 g, 222 mmol, 2.1 equiv.) and 1-ethyl-3-(3-dimethylaminopropyl) carbodiimide hydrochloride (44.7 g, 233 mmol, 2.2 equiv.) were dissolved in ACN (200 mL) at 0° C. Adipic acid (15.5 g, 106 mmol, 1 equiv.) was added in portions. Reaction became clear and colourless. The reaction was stirred for 18 h at room temperature at which point LC/MS indicated the reaction was complete. The solvent was evaporated in vacuo and the residue redissolved in Et₂O (200 mL) and 1 M HCl (50 mL). The organic layer was washed with 1 M HCl (2×50 mL) and mL brine (2×50 mL). Significant emulsions occurred, so the separating funnel was left to sit for 1 h between shaking. The organic layer was dried with MgSO₄, filtered and the solvent was evaporated in vacuo. A white solid was recovered which was dissolved in hot DCM (30 mL) and filtered, then allowed to stand in the fume hood, open to atmosphere, for 54 h, at which point crystal formation was significant. The solution was allowed to stand at −20° C. for a further 18 h, then filtered to recover crystals and rinsed with cold DCM (50 mL). 32.1 g of crystals were recovered from the first crop. The crystallization protocol was repeated on the mother liquor to recover additional 5.4 g of crystals. The two crops of crystals were combined to give the title compound as a crystalline solid (37.5 g, 84.8 mmol, 79.9%).

LC/MS: calc'd m/z=442.05 for C₁₈H₁₀F₈O₄, no m/z detected. ¹H NMR (300 MHZ, DMSO) δ 7.95 (tt, J=10.9, 7.4 Hz, 2H), 2.95-2.78 (m, 4H), 1.87-1.55 (m, 4H).

3.18 2,3,5,6-tetrafluorophenyl 6-(((S)-1-(((S)-1-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl)amino)-1-oxo-5-ureidopentan-2-yl)amino)-3-methyl-1-oxobutan-2-yl)amino)-6-oxohexanoate (Drug-Linker 002)

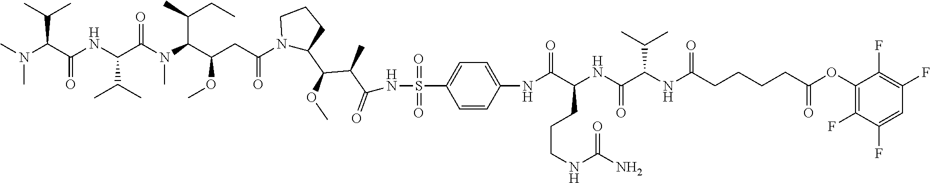 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCCCC(=O)Oc2c(F)c(F)cc(F)c2F)C(C)C)cc1)OC)N(C)C(=O)[C@@H](NC(=O)[C@H](C(C)C)N(C)C)C(C)C]

Compound 15 (2.35 g, 1.90 mmol, 1 equiv.) was dissolved in DMF (2 mL) in a 250 mL round bottom flask. Separately, Compound 14 (5.00 g, 11.4 mmol, 6 equiv.) was dissolved in DMF (10 mL), then added to the first solution. The reaction was stirred at room temperature and N,N-diisopropylethylamine (0.736 g, 0.992 mL, 5.70 mmol, 3 equiv.) was added. Reaction was complete within 30 min as indicated by LCMS. TFA (1 mL) was added and the reaction concentrated in vacuo to a 12 mL volume. The concentrate was purified via reverse phase chromatography over a 10-50% ACN/H₂O+0.1% TFA gradient. Product fractions were pooled and lyophilized to recover the title compound as a white solid powder. (2.17 g, 1.55 mmol, 81.5%).

LC/MS: calc'd m/z=1284.56 for C₆₀H₉₂F₄N₁₀O₁₄S, detected [M+H]⁺=1285.8 m/z. ¹H NMR (300 MHz, MeOD) δ 8.02-7.93 (m, 2H), 7.89 (d, J=9.0 Hz, 1H), 7.83 (d, J=9.0 Hz, 1H), 7.43 (tt, J=10.6, 7.3 Hz, 1H), 4.74 (d, J=8.8 Hz, 1H), 4.56 (dt, J=9.4, 4.8 Hz, 1H), 4.20 (dd, J=9.7, 7.5 Hz, 1H), 4.10 (s, 1H), 3.96-3.82 (m, 1H), 3.72 (t, J=4.7 Hz, 1H), 3.51 (d, J=9.3 Hz, 1H), 3.38 (s, 1H), 3.36-3.32 (m, 6H), 3.30 (d, J=5.4 Hz, 3H), 3.17 (s, 2H), 3.12 (d, J=6.9 Hz, 1H), 2.95 (d, J=10.2 Hz, 6H), 2.83-2.74 (m, 1H), 2.63-2.48 (m, 2H), 2.46 (d, J=6.9 Hz, 1H), 2.44-2.34 (m, 3H), 2.21-2.01 (m, 1H), 1.89 (dd, J=14.3, 7.7 Hz, 1H), 1.80 (q, J=3.5 Hz, 1H), 1.60 (d, J=7.4 Hz, 2H), 1.18-0.95 (m, 19H), 0.89 (q, J=6.9 Hz, 3H).

3.19 2,5-dioxopyrrolidin-1-yl (6S,9S,27S)-1-amino-27-(((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl) carbamoyl)-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11,24,29-pentaoxo-14,17,20-trioxa-2,7,10,23,28-pentaazatetratriacontan-34-oate (Drug-Linker 005)

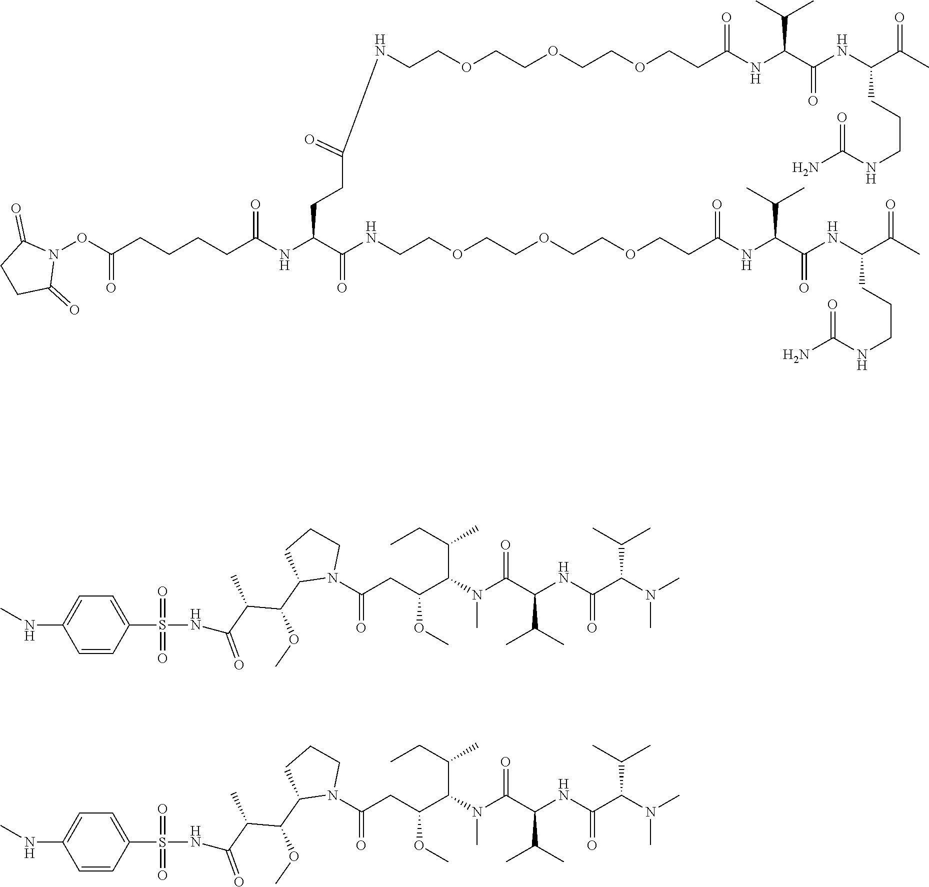

Compound 10 (104.0 mg, 0.0361 mmol, 1 eq) was dissolved in 2 mL DMF in a 50 mL round bottom flask. Bis(2,5-dioxopyrrolidin-1-yl) adipate (98.4 mg, 0.289 mmol, 8.0 eq) and N-ethyldiisopropylamine (23.3 mg, 0.0314 mL, 0.74 g/mL, 0.181 mmol, 5 equiv.) were added and stirred at room temperature. Reaction was determined to be complete after 30 min. by LC/MS. The reaction mixture was acidified with 0.5 mL of 1 M HCl and diluted with 4 mL of H₂O, then purified via reverse phase chromatography over a 10-50% ACN/H₂O+0.1% TFA gradient. Product-containing fractions were lyophilized to recover the title compound as a white solid (93.6 mg, 0.0313 mmol, 86.7%).

LC/MS: calc'd m/z=2760.54 for C₁₂₉H₂₁₈N₂₄O₃₇S₂, detected [M+2H]⁺²=1382.0 m/z. ¹H NMR (400 MHZ, MeOD) δ 8.01-7.94 (m, 3H), 7.91 (d, J=8.8 Hz, 1H), 7.86 (d, J=8.8 Hz, 1H), 4.74 (d, J=8.6 Hz, 1H), 4.60-4.51 (m, 2H), 4.43-4.33 (m, 1H), 4.24 (t, J=7.3 Hz, 1H), 4.11 (s, 1H), 3.93-3.82 (m, 1H), 3.81-3.68 (m, 4H), 3.61 (s, 10H), 3.56-3.52 (m, 2H), 3.52-3.49 (m, OH), 3.38 (s, 2H), 3.19-3.11 (m, 3H), 2.97 (s, 3H), 2.93 (s, 5H), 2.85 (t, J=1.7 Hz, 3H), 2.67 (s, 2H), 2.63-2.55 (m, 3H), 2.52 (d, J=8.0 Hz, 1H), 2.46 (t, J=6.6 Hz, 1H), 2.35-2.27 (m, 1H), 2.16-2.00 (m, 2H), 1.99-1.87 (m, 1H), 1.80-1.68 (m, 4H), 1.68-1.57 (m, 1H), 1.49-1.37 (m, 1H), 1.17-1.09 (m, 7H), 1.09-1.06 (m, 2H), 1.06-0.98 (m, 13H), 0.89 (q, J=7.4 Hz, 4H).

3.20 2,3,5,6-tetrafluorophenyl (6S,9S,27S)-1-amino-27-(((6S,9S)-1-amino-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11-trioxo-14,17,20-trioxa-2,7,10-triazadocosan-22-yl) carbamoyl)-6-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl) carbamoyl)-9-isopropyl-1,8,11,24,29-pentaoxo-14,17,20-trioxa-2,7,10,23,28-pentaazatetratriacontan-34-oate (Drug-Linker 006)

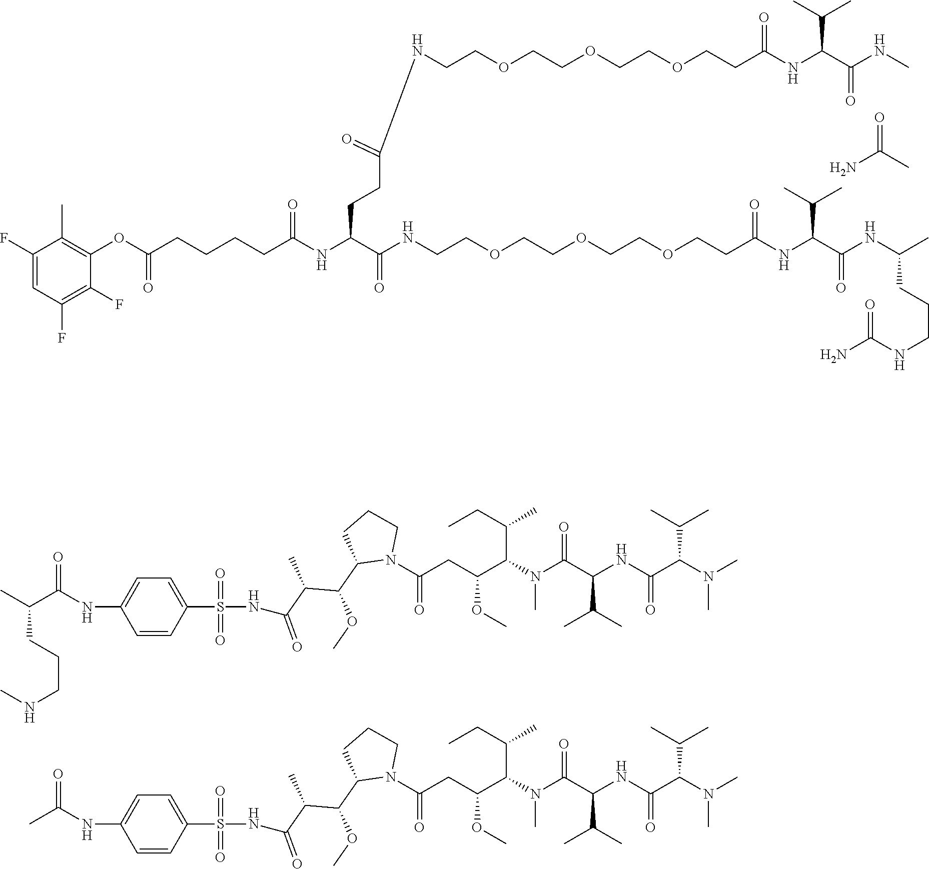

Drug-Linker 005 (17 mg, 0.00568 mmol, 1 equiv.) was dissolved in a 2:1 mixture of diH₂O:ACN (3 mL). The pH was raised to ˜11 with saturated NaHCO₃ (0.2 mL) and the reaction left to stir for 18 hr at room temperature. Hydrolysis of the NHS ester was determined to be complete by LC/MS. The reaction was acidified to pH 5 with 1 M NaH₂PO₄ (1 mL) and 2,3,5,6-tetrafluorophenol (4.72 mg, 0.0284 mmol. 5 equiv.) and 1-ethyl-3-(3-dimethylaminopropyl) carbodiimide hydrochloride (5.45 mg, 0.0284 mmol, 5 equiv.) were added. After 2 hr, reaction was determined to be complete by LC/MS. The reaction mixture was purified via reverse phase chromatography over a 10-50% ACN/H₂O+0.1% TFA gradient. Product-containing fractions were pooled and lyophilized to recover the title compound as a white solid (3.7 mg, 0.00122 mmol, 21.4%).

LC/MS: calc'd m/z=2811.52 for C₁₃₁H₂₁₅F₄N₂₃O₃₅S₂, detected [M+2H]⁺²=1407.0 m/z.

In the alternative, Drug-Linker 006 may be prepared via the same methods as Drug-Linker 005.

3.21 (S)—N-(4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl) pyrrolidin-2-yl)-3-methoxy-2-methylpropanoyl) sulfamoyl)phenyl)-2-((S)-1-(2,5-dioxo-2,5-dihydro-1H-pyrrol-1-yl)-14-isopropyl-12-oxo-3,6,9-trioxa-13-azapentadecan-15-amido)-5-ureidopentanamide (Drug-Linker 001)

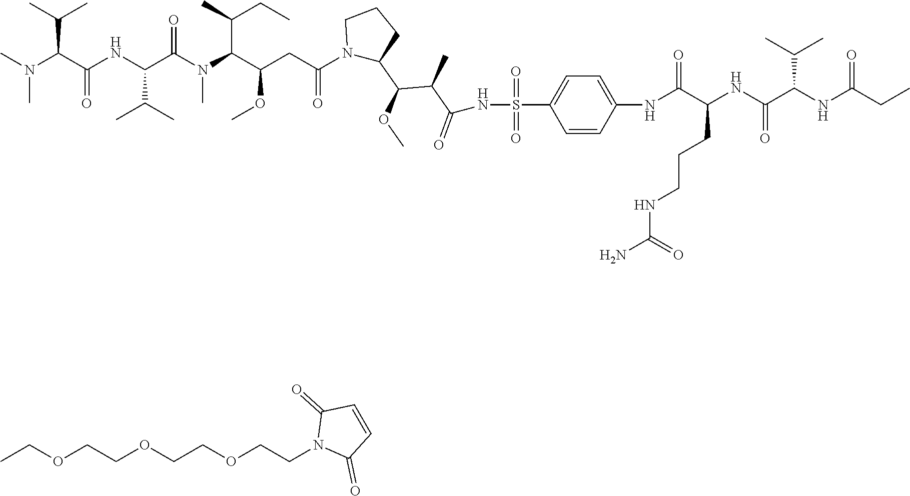

Prepared as described in International Publication No. WO 2019/173911.

### Example 4: Preparation of Additional Multivalent Drug-Linkers

### Drug-Linker 007 and Drug-Linker 008

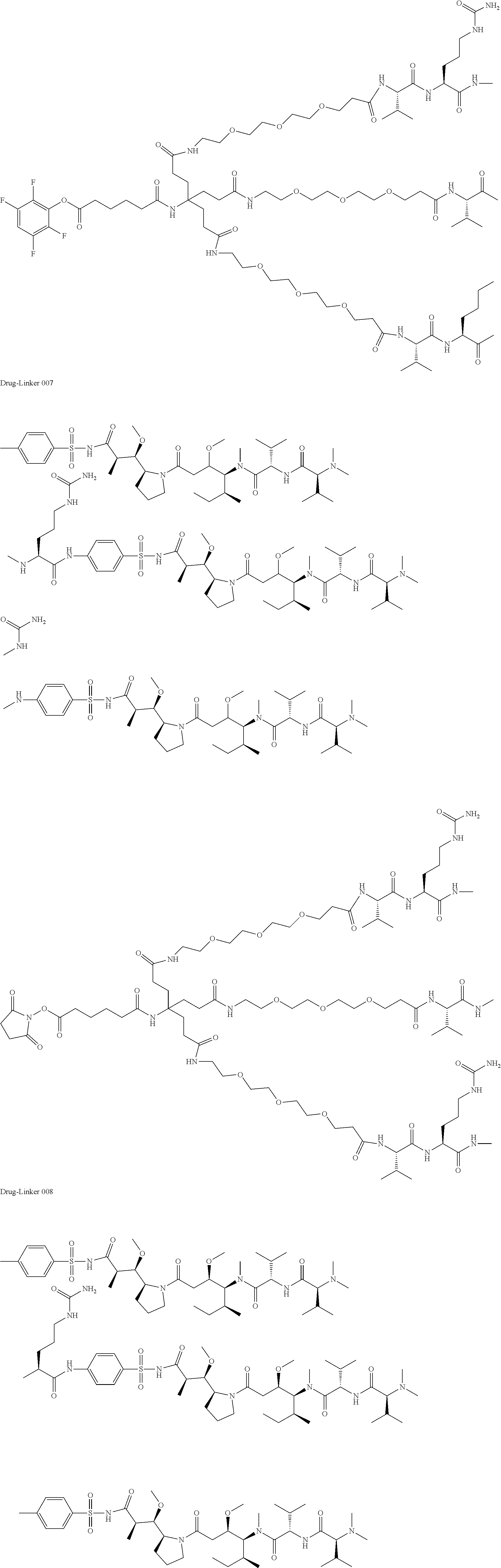

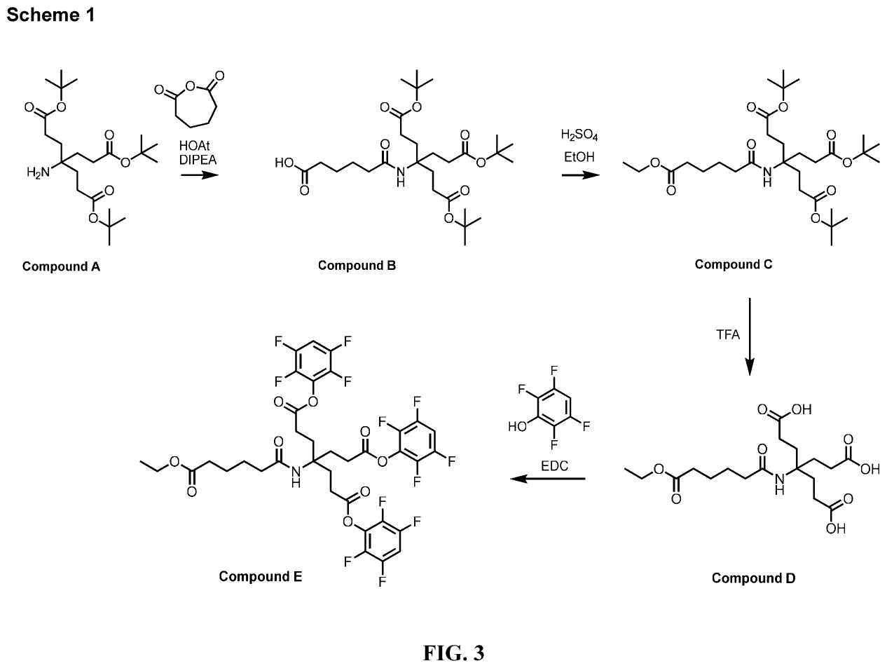

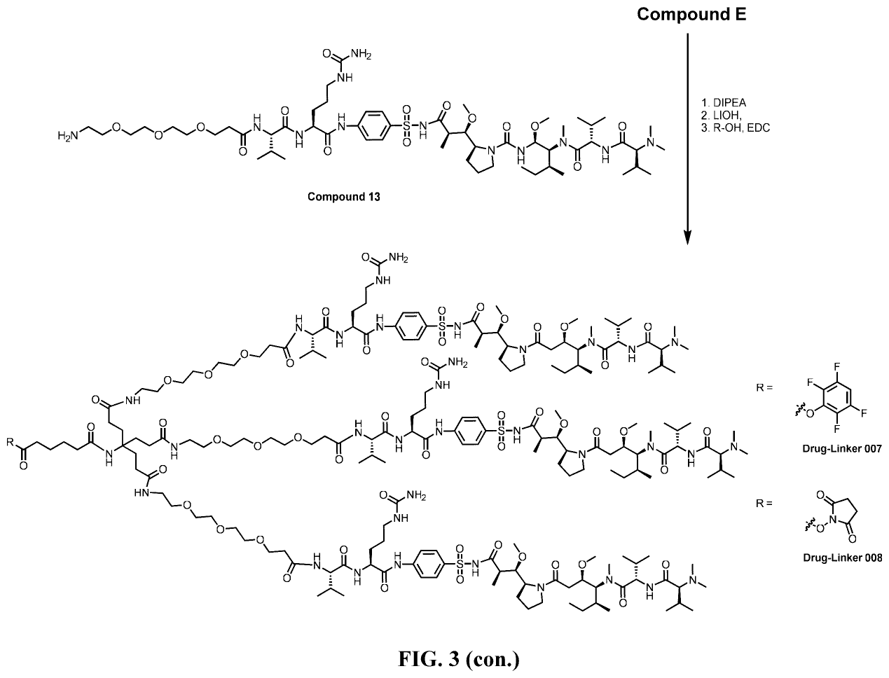

The trivalent drug-linker structure exemplified in Drug-Linker 004 (Example 3.16) can alternatively be adapted for conjugation to lysine residues by utilizing the 2,3,4,5-tetrafluorophenol or N-hydroxysuccinimide activated esters similar to those utilized in Drug-Linker 002 (Example 3.18) and Drug-Linker 006 (Example 3.20), and Drug-Linker 005 (Example 3.19), respectively. Such drug-linkers (Drug-Linker 007 and Drug-Linker 008) may be prepared through the reaction of adipic anhydride with 1,7-di-tert-butyl 4-amino-4-[3-(tert-butoxy)-3-oxopropyl]heptanedioate under amide bond formation conditions similar to those used for the generation of Compound 3 before being further elaborated into the final drug-linker. See Scheme 1 (FIG. 3).

### Example 5: Conjugation and Characterization of Anti-cMet Antibody-Drug Conjugates

The antibody-drug conjugates (ADCs) shown in Table 5.1 were prepared as described below.

### 5.1 Preparation of ADCs by Stochastic Lysine Conjugation

An exemplary protocol for the preparation of ADCs by stochastic lysine conjugation is provided below.

A solution (2.3 mL) of variant v17427 (25 mg) in PBS, pH 7.4 was reacted with 4-22 molar equivalents of Drug-Linker 002 (10-20 mM dissolved in DMSO) in the presence of 5-10% (v/v, final) DMSO at 5 mg/mL in PBS, pH 7.4. The reaction mixture was mixed by pipetting and then centrifuged at 400×g for 3 minutes. The resulting solution was incubated for 16-20 hours at room temperature prior to purification.

### 5.2 Preparation of ADCs by Stochastic Cysteine Conjugation

An exemplary protocol for the preparation of ADCs by stochastic cysteine conjugation is provided below.

A solution of variant v17427 in PBS, pH 7.4 was reduced by the addition of 5 mM diethylenetriamine pentaacetic acid (DTPA) (11.4 mL in PBS, pH adjusted to 7.4) and 10 mM of an aqueous tris(2-carboxyethyl) phosphine (TCEP) solution (591 uL, 2.2 eq.) at a final concentration of 5-10 mg/mL. The reduction reaction proceeded at 37° C. for 1-3 hours. The reduced protein was then reacted with excess Drug-Linker 001 (6-10 eq., 10-20 mM DMSO stock) for 1-2 hours on ice. An excess of N-acetyl-L-cysteine solution (6-10 eq.) from a 10 mM stock solution in water was added to quench the conjugation reaction. The quenched reaction was incubated on ice for 30 minutes prior to purification.

### 5.3 Preparation of ADCs by Site-Specific Conjugation to Cysteine Insertions

An exemplary protocol for the preparation of ADCs by site-specific conjugation to inserted cysteine residues is provided below. This protocol or a similar protocol has been applied to the site-specific conjugation of Drug-Linker 001, Drug-Linker 003 and Drug-Linker 004.

A solution (202 mL) of variant v29001 (2 g) in PBS, pH 7.4 was reduced by the addition of 10 mM DTPA (24 mL in PBS, pH adjusted to 7.4) and 25 mM of an aqueous TCEP solution (13.7 mL, 25 eq.). After 3-4 hours at 37° C., the reduced antibody was diluted to approximately 250 mL with PBS and purified using a Pellicon® XL Ultrafiltration Module (Ultracel 30 kDa 0.005 m²; MilliporeSigma, Burlington, MA; PXC030C50) with approximately 3 diavolumes of PBS, pH 7.4. The purified antibody was then re-oxidized with 25 molar equivalents of dehydroascorbic acid (DHAA) (50 mM DMSO stock) for 16-20 hours at 2-8° C. To the re-oxidized antibody (1.6 g, 220 mL) was added 5.9 mL of Drug-Linker 004 (5.5 molar equivalents) from a 10 mM DMSO stock solution. The conjugation reaction proceeded at room temperature with mixing for 3-4 hours. An excess of N-acetyl-L-cysteine solution (5.6 mL, 5.25 eq.) from a 10 mM stock solution (10% DMSO in water) was added to quench the conjugation reaction. The quenched reaction was incubated at room temperature with mixing for 30 minutes and then incubated at 2-8° C. for 16-20 hours prior to purification.

5.4 Purification and Characterization of ADCs

ADCs prepared at a scale greater than 50 mg were purified using a Pellicon® XL Ultrafiltration Module (MilliporeSigma, Burlington, MA). Briefly, the crude ADC solution was diluted or concentrated to approximately 5-10 mg/mL with 10 mM NaOAc, pH 5.5 and purified using a Pellicon® XL Ultrafiltration Module (Ultracel 30 kDa 0.005 m²; MilliporeSigma, Burlington, MA; PXC030C50) with 8-15 diavolumes of 10 mM NaOAc, pH 4.5. The purified ADC was then sterile filtered (0.2 um).

ADCs prepared at a scale less than 50 mg were purified by Zeba™ Spin desalting columns, 40 MWCO (ThermoFisher Scientific, Waltham, MA), pre-equilibrated with 10 mM NaOAc, pH 4.5 as per the manufacturer's instructions.

Control variant v17606 conjugated to the drug-linker MCvcPABC-MMAE was purified using column chromatography as described in International Publication No. WO 2017/201204. Briefly, crude ADC sample was applied to a HiTrap™ Butyl HP column (Cytiva Life Sciences, Marlborough, MA) using an ammonium sulfate/sodium phosphate buffer and eluted with a sodium phosphate buffer containing 20% isopropyl alcohol. ADC species with an average drug to antibody ratio (DAR) of 2 and 4 were enriched and combined in a 1:1 ratio to achieve an ADC with an average DAR of approximately 3. The purified ADC was formulated in a 10 mM histidine, pH 6.0 buffer and sterile filtered.

Following purification, the concentration of the ADCs was determined by a BCA assay with reference to a standard curve generated using the respective unconjugated parent antibody. Alternatively, concentrations were estimated by measurement of absorption at 280 nm using the calculated extinction coefficients of the antibody sequence. ADCs were also characterized by hydrophobic interaction chromatography (HIC) and size exclusion chromatography (SEC) as described below.

5.4.1 Hydrophobic Interaction Chromatography

ADCs were analyzed by HIC to estimate the drug-to-antibody ratio (DAR). Chromatography was performed on an Agilent Infinity II 1290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

5.4.2 Size Exclusion Chromatography

The extent of aggregation of ADCs (˜15-150 ug, 5 uL injection volume) was assessed by SEC on an Agilent Infinity II 1260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

Table 5.1 summarizes the properties assessed for each of the ADCs.

TABLE 5.1
Properties of ADCs

| Antibody | | | | ADC | | |
|---|---|---|---|---|---|---|
| Variant | Fc | | Conjugation | Variant | Nominal | % |
| No. | Region | Drug-Linker | Technology | No. | DAR | Monomer |
| v17427 | HetFc | Drug-Linker 002 | Lysine | v34293 | 2 | 95.0% |
| | | Drug-Linker 002 | Lysine | v35702 | 4 | 96.7% |
| | | Drug-Linker 002 | Lysine | v35703 | 6 | 96.3% |
| | | Drug-Linker 001 | Cysteine | v19873 | 4 | 99.8% |
| | | MCvcPABC-MMAE | Cysteine | v19876 | 4 | 97.9% |
| v32634 | HomoFc | Drug-Linker 002 | Lysine | v35221 | 4 | 98.9% |
| | | Drug-Linker 002 | Lysine | v35222 | 6 | 98.8% |
| v28983 | HetFc | Drug-Linker 001 | SS¹ Cysteine | v34280 | 2 | 95.3% |
| v29001 | HetFc | Drug-Linker 001 | SS Cysteine | v34279 | 2 | 96.4% |
| | | Drug-Linker 003 | SS Cysteine | v35530 | 4 | 94.4% |
| | | Drug-Linker 004 | SS Cysteine | v35531 | 6 | 93.5% |
| v35527 | HomoFc | Drug-Linker 003 | SS Cysteine | v35743 | 4 | 99.2% |
| | | Drug-Linker 004 | SS Cysteine | v35744 | 6 | 99.1% |
| v33967 | HetFc | Drug-Linker 001 | SS Cysteine | v34276 | 1 | 99.2% |
| v33968 | HetFc | Drug-Linker 001 | SS Cysteine | v34277 | 1 | 97.7% |
| v33970 | HetFc | Drug-Linker 001 | SS Cysteine | v34281 | 3 | 96.1% |
| v33971 | HetFc | Drug-Linker 001 | SS Cysteine | v34282 | 3 | 92.5% |
| v33979 | HetFc | Drug-Linker 001 | SS Cysteine | v34286 | 3 | 96.9% |

Control ADCs

| | | | | | | |
|---|---|---|---|---|---|---|
| v17606 | HomoFc | MCvcPABC-MMAE | Cysteine | v36198 | 3 (HIC | 99.4% |
| | | | | | purified) | |
| | | MCvcPABC-MMAE | Cysteine | v19875 | 3 | 99.3% |

¹SS = site-specific

### Example 6: In Vitro Cytotoxicity of Stochastic Cysteine-Conjugated Anti-cMet Antibody-Drug Conjugates (Dar 4)

The cell growth inhibition (cytotoxicity) capabilities of anti-cMet ADCs comprising variant v17427 (HetFc, unmodified hinge) conjugated to Drug-Linker 001 or MCvcPABC-MMAE by stochastic cysteine conjugation at an average DAR 4 were determined in a panel of four cMet-expressing cell lines as described below. The cell lines were EBC-1 (lung squamous cell carcinoma), H292 (lung carcinoma), BT-20 (breast cancer) and SW48 (colorectal cancer).

Briefly, cells were seeded in 384-well plates at 1,000-1,500 cells/well density and treated with a titration of test article, generated in complete cell growth medium. Treated cells were incubated for 4-5 days under standard culturing conditions (37° C./5% CO₂). After incubation, CellTiter-Glo® reagent (Promega Corporation, Madison, WI; Cat. No. G7570) was spiked into each well and luminescence corresponding to ATP present in each well was measured using a Synergy™ H1 microplate (BioTek Instruments, Winooski, VT). Based on blank wells (no test article added, only blank media), % cytotoxicity values were calculated using ATP measurement RLU values (Relative Light Units) and plotted against test article concentration using GraphPad Prism 8 software (GraphPad Software, San Diego, CA).

### Results

The results are summarized in Table 5.1. DAR-matched v17427-MCvcPABC-MMAE DAR 4 ADC showed lower in vitro cytotoxicity compared to v17427-Drug-Linker 001 ADC in the high and moderate cMet-expressing cells EBC-1, HT-29 and BT-20. The difference in cytotoxicity was less evident in the highest cMet-expressing cell lines tested EBC-1. Neither ADC displayed significant cytotoxicity in low cMet-expressing cell line SW48. Free payload from Drug-Linker 001 (Compound 1) showed cytotoxicity in all tumour cell lines as expected, but inferior to the v17427-Drug-Linker 001 ADC.

TABLE 6.1
In vitro Cytotoxicity - EC50 Summary

| | | EC50 (nM) - DAR corrected | | | |
|---|---|---|---|---|---|
| ADC | DAR | EBC-1 | H292 | BT-20 | SW48 |
| v17427-Drug-Linker 001 | 4.0 | 0.02 | 0.68 | 0.12 | >12 |
| v17427-MCvcPABC- | 4.1 | 0.08 | >12 | >12 | >12 |
| MMAE | | | | | |
| Free Compound 1 | — | 44.37 | 22.20 | 39.17 | 43.61 |

### Example 7: cMet Pathway Agonism by Stochastic Cysteine-Conjugated Anti-cMet Antibody-Drug Conjugates (Dar 4)

cMet pathway agonism of the anti-cMet antibody variant v17427 (HetFc, unmodified hinge) and ADCs comprising variant v17427 stochastically cysteine-conjugated to Drug-Linker 001 or MCvcPABC-MMAE at DAR 4 was assessed by cell proliferation and ELISA to measure for AKT phosphorylation as a downstream indicator of cMET activation.

Cell proliferation: Briefly, H596 lung cancer cells were serum starved overnight (37° C., 5% CO₂) by replacing the complete growth medium with serum-free RPMI-1640 (Thermo Fisher Scientific Inc., Waltham, MA; Cat. No. A1049101). Cells were then detached with Cell Dissociation Buffer (Thermo Fisher Scientific Inc., Cat. No. 13151014), resuspended in RPMI-1640 supplemented with 1% FBS (v/v) (Thermo Fisher Scientific Inc., Cat. No. 12483-020) and seeded in 384-well tissue culture plates at a density of 1,000 cells/well. Cells were treated with a titration of test articles prepared in RPMI-1640+1% FBS (v/v) and incubated for 6 days (37° C., 5% CO₂). After incubation, CellTiter-Glo® reagent (Promega Corporation, Madison, WI; Cat. No. G7570) was spiked in all wells and luminescence corresponding to ATP present in each well was measured using a Synergy™ H1 microplate (BioTek Instruments, Winooski, VT). Based on untreated cells (no test article added), % viability values were calculated using ATP measurement RLU values (Relative Light Units) and plotted against test article concentration using GraphPad Prism 8 software (GraphPad Software, San Diego, CA).

Phospho-AKT ELISA: Briefly, H441 or H596 lung cancer cells were detached with Cell Dissociation Buffer, seeded in 24-well tissue culture plates with RPMI-1640+10% FBS at a density of 50,000 cells/well, and incubated overnight (37° C., 5% CO₂). Cells were then serum starved overnight (37° C., 5% CO₂) by replacing the complete growth medium with serum-free RPMI-1640. Culture media was removed, and cells were then treated with test article prepared at 100 nM in serum-free RPMI-1640 and incubated (37° C., 5% CO₂) for the appropriate time points. Cell lysates were generated by addition of Cell Lysis Buffer (Cell Signaling Technology, Danvers, MA; Cat. No. 9803S)+1 mM PMSF (Cell Signaling Technology, Cat. No. 8553S). Lysate protein concentration was assessed by a BCA protein assay (Thermo Fisher Scientific Inc., Cat. No. 23223 and 23224). Phospho-AKT levels in 10 μg of lysate was assessed using the PathScan® Phospho-Akt1 (Ser473) Sandwich ELISA Kit (Cell Signaling Technology, Cat. No. 7160C) as per manufacturer's instructions. Absorbance at 450 nm was measured using a Synergy™ H1 microplate (BioTek Instruments, Winooski, VT). Fold AKT phosphorylation was calculated by subtracting background A450 nm signal from all wells using signal blank wells, and then normalizing to the A450 nm signal of untreated cells at each corresponding timepoint. Fold change was then plotted per timepoint using GraphPad Prism 8 software (GraphPad Software, San Diego, CA).

### Results

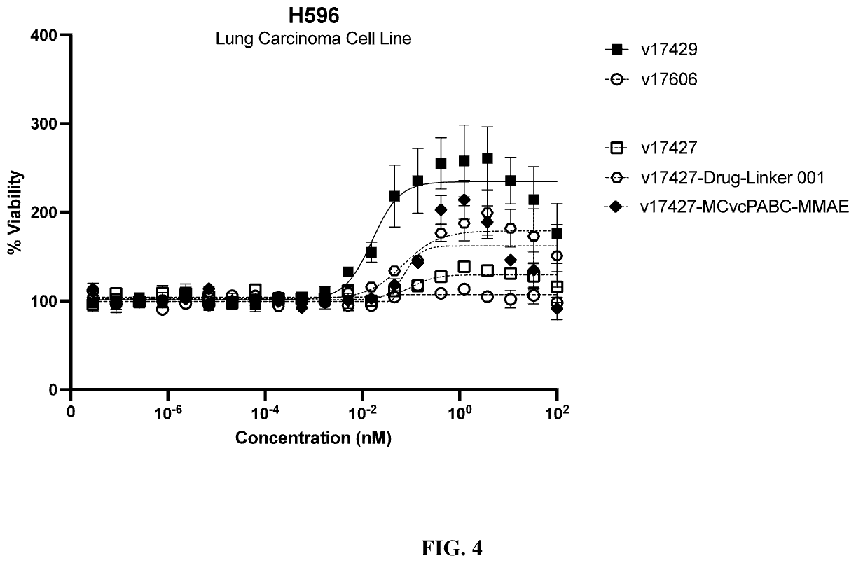

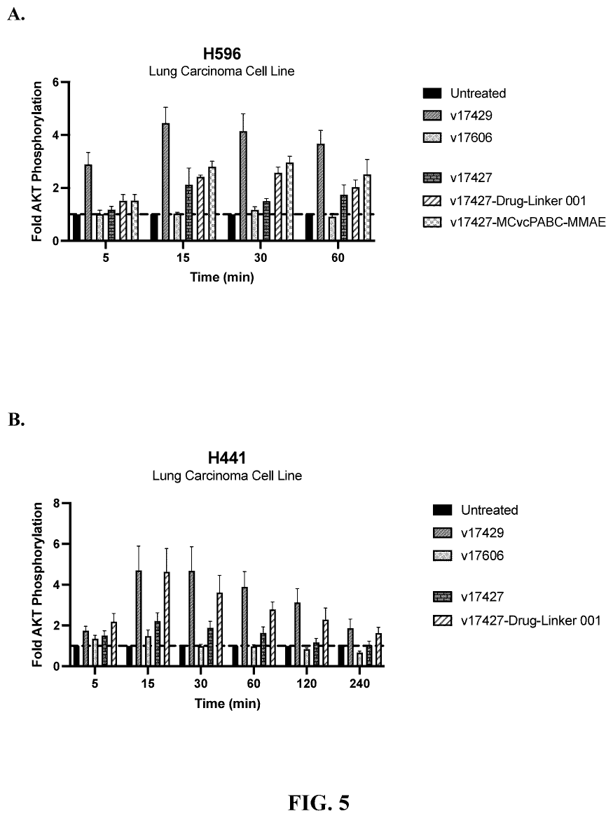

The results are shown in FIG. 4 and FIGS. 5A & B. As can be seen from FIG. 4, bivalent MetMab (v17429) induced a dose-dependent increase in H596 proliferation compared to untreated controls, whereas hinge-modified telisotuzumab (v17606) exhibited minimal effects, as expected. Unconjugated variant v17427 (HetFc, unmodified hinge) exhibited a slight but consistent increase in H596 proliferation after 6 days. Variant v17427 stochastically conjugated to Drug-Linker 001 or MCvcPABC-MMAE at DAR 4 demonstrated a noticeable dose-dependent increase in proliferation compared to untreated controls.

FIGS. 5A & B shows that bivalent MetMab (v17429) induced strong AKT phosphorylation in both H596 and H441 cells, which was sustained up to 60 minutes after treatment. In contrast, hinge-modified telisotuzumab (v17606) showed minimal effect on AKT phosphorylation in either cell line. Unconjugated variant v17427 induced a slight transient increase in AKT phosphorylation in both cell lines. Stochastic ADCs generated from variant v17427 appeared to show an increase in phosphorylation compared to the unconjugated parental antibody.

### Example 8: In Vitro Cytotoxicity of Lysine-Conjugated Anti-cMet Antibody-Drug Conjugates (Dar 2, 4 & 6)

The cell growth inhibition capabilities of anti-cMet ADCs comprising variant v17427 (HetFc, unmodified hinge) conjugated to Drug-Linker 002 by stochastic lysine conjugation at DARs ranging from DAR 2 to DAR 6 were determined in a panel of seven cMet-expressing cell lines. Variant v17427 conjugated to MCvcPABC-MMAE drug linker at DAR 4, and anti-RSV antibody, palivizumab (v22277), conjugated to Drug-Linker 002 or MCvcPABC-MMAE at DAR 4, were used as controls. Cell lines were SNU-5 (gastric carcinoma), EBC-1 (lung squamous cell carcinoma), HCC827 (lung cancer), H1975 (non-small cell lung cancer), HCT-116 (colorectal cancer), H292 (lung carcinoma) and BT-20 (breast cancer).

Cytotoxicity was determined as described in Example 6 with treated cells (1,000 cells/well) being incubated for 4 days under standard culturing conditions (37° C./5% CO₂).

### Results

The results are summarized in Table 8.1. Stochastic v17427-Drug Linker 002 ADCs with DARs ranging from DAR 1.9 to DAR 6.2 yielded a range of cytotoxic activity across the panel of 7 cMet-expressing cell lines, with higher DAR conjugates generally showing lower EC50 values in all cell lines tested. In high expression cell lines SNU-5 and EBC-1, the difference between DAR 1.9 to DAR 6.2 ADCs was minimal, with EC50s for the lowest DAR and highest DAR conjugates ranging from 0.043 to 0.021 nM, and 0.012 to 0.004 nM, respectively. In moderate to low expression cell lines HCC827, H1975, HCT-116, H292 and BT-20, the difference between 10 DAR 1.9 to DAR 6.2 ADCs was more pronounced. Stochastic palivizumab-Drug-Linker 002 and palivizumab-MCvcPABC-MMAE conjugates did not show cytotoxicity in cMET-expressing cell lines as expected.

TABLE 8.1
In Vitro Cytotoxicity - EC50 Summary

| | | EC50 (nM) | | | | | | |
|---|---|---|---|---|---|---|---|---|
| ADC | DAR | SNU-5 | EBC-1 | HCC827 | H1975 | HCT-116 | H292 | BT-20 |
| v17427-Drug- | 1.9 | 0.043 | 0.012 | 0.109 | 0.058 | >10 | 0.118 | 0.072 |
| Linker 002 | | | | | | | | |
| v17427-Drug- | 2.6 | 0.037 | 0.012 | 0.161 | 0.083 | 0.122 | >10 | 0.038 |
| Linker 002 | | | | | | | | |
| v17427-Drug- | 3.4 | 0.030 | 0.007 | 0.105 | 0.036 | 0.351 | 0.197 | 0.041 |
| Linker 002 | | | | | | | | |
| v17427-Drug- | 3.9 | 0.024 | 0.004 | 0.060 | 0.026 | 0.025 | 0.098 | 0.039 |
| Linker 002 | | | | | | | | |
| v17427-Drug- | 4.0 | 0.029 | 0.005 | 0.059 | 0.029 | 0.048 | 0.067 | 0.028 |
| Linker 002 | | | | | | | | |
| v17427-Drug- | 4.6 | 0.036 | 0.003 | 0.044 | 0.036 | 0.071 | 0.098 | 0.022 |
| Linker 002 | | | | | | | | |
| v17427-Drug- | 5.5 | 0.026 | 0.004 | 0.037 | 0.026 | 0.018 | 0.069 | 0.030 |
| Linker 002 | | | | | | | | |
| v17427-Drug- | 6.0 | 0.027 | 0.003 | 0.023 | 0.052 | 0.028 | 0.103 | 0.037 |
| Linker 002 | | | | | | | | |
| v17427-Drug- | 6.2 | 0.021 | 0.004 | 0.021 | 0.023 | 0.013 | 0.077 | 0.026 |
| Linker 002 | | | | | | | | |

Controls

| | | | | | | | | |
|---|---|---|---|---|---|---|---|---|
| v22277-Drug- | 4.0 | >10 | >10 | >10 | >10 | >10 | >10 | >10 |
| Linker 002 | | | | | | | | |
| v17427- | 4.0 | 0.036 | 0.003 | 0.088 | 0.054 | 1.844 | 1.099 | 0.263 |
| MCvcPABC- | | | | | | | | |
| MMAE | | | | | | | | |
| V22277- | 4.0 | >10 | >10 | >10 | >10 | >10 | >10 | >10 |
| MCvcPABC- | | | | | | | | |
| MMAE | | | | | | | | |

### Example 9: In Vitro Cytotoxicity of Anti-cMet Antibody-Drug Conjugates with HomoFc or HetFc Scaffolds

The cell growth inhibition capabilities of ADCs comprising anti-cMet antibodies with either a HomoFc or HetFc backbone (v32634 and v17427, respectively) conjugated to Drug-Linker 002 by stochastic lysine conjugation at approximately DAR 4 and DAR 6 were assessed in a panel of four cMet-expressing tumour cell lines and one cMet-negative cell line. The anti-RSV antibody, palivizumab (v22277), conjugated to Drug-Linker 002 at DAR 3.7, and free payload (Compound 1), were used as controls. Cytotoxicity was determined as described in Example 8. Cell lines were SNU-5 (gastric carcinoma), EBC-1 (lung squamous cell carcinoma), H1975 (non-small cell lung cancer), H292 (lung carcinoma) and cMet-negative T-47D (breast cancer).

### Results

The results are summarized in Table 9.1. DAR-matched ADCs comprising anti-cMet antibodies with HetFc or HomoFc backbones conjugated to Drug-Linker 002 yielded comparable cytotoxicity in cMet-expressing cell lines. None of the ADCs showed activity in cMet-negative cell line T-47D, as expected. In high expression cell lines, SNU-5 and EBC-1, differences in potency were discrete between DAR 3.9-4.0 and DAR 5.8 conjugates. In moderate expression cell lines, H1975 and H292, differences in potency between DAR 3.9-4.0 and DAR 5.8 conjugates were more evident. DAR 5.8 conjugates yielded 2.0 to 3.4-fold higher EC50s compared to DAR 4 conjugates in H1975 and H292 cell lines, respectively.

TABLE 9.1
In Vitro Cytotoxicity - EC50 Summary

| | Fc | | EC50 (nM) | | | | |
|---|---|---|---|---|---|---|---|
| ADC | Region | DAR | SNU-5 | EBC-1 | H1975 | H292 | T-47D |
| v17427-Drug-Linker 002 | HetFc | 4.0 | 0.04 | 0.01 | 0.08 | 0.12 | >20 |
| v17427-Drug-Linker 002 | HetFc | 5.8 | 0.01 | 0.01 | 0.04 | 0.06 | >20 |
| v32634-Drug-Linker 002 | HomoFc | 3.9 | 0.04 | 0.01 | 0.08 | 0.17 | >20 |
| v32634-Drug-Linker 002 | HomoFc | 5.8 | 0.03 | 0.01 | 0.04 | 0.05 | >20 |
| v22277-Drug-Linker 002 | HomoFc | 3.7 | >20 | >20 | >20 | >20 | >20 |
| Compound 1 | — | — | 40.05 | 26.29 | 22.71 | 7.42 | 44.83 |

### Example 10: Cellular Equilibrium Binding of Lysine-Conjugated Anti-cMet Antibody-Drug Conjugates

The on-cell binding capabilities of ADCs comprising anti-cMet antibodies with either a HomoFc or HetFc backbone (v32634 and v17427, respectively) conjugated to Drug-Linker 002 by stochastic lysine conjugation at approximately DAR 4 and DAR 6 were assessed for cMet binding using SNU-5 and H292 endogenous cMet-expressing cell lines by flow cytometry as described below. Unconjugated anti-RSV antibody palivizumab (v22277) was used as a control.

Briefly, cells were seeded at 50,000 cells/well in V-bottom 96-well plates and treated with antibody for 24 hours at 4° C. to prevent internalization. Following incubation, cells were washed and stained with anti-human IgG Fc AF647 conjugate (Jackson ImmunoResearch Laboratories, Inc., West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted using GraphPad Prism Version 8 (GraphPad Software, San Diego, CA).

### Results

The results are summarized in Table 10.1. Both unconjugated variants, v32634 and v17427 (HomoFc and HetFc regions, respectively), yielded comparable apparent Kd and Bmax values in both SNU-5 and H292 cell lines (high and moderate endogenous cMet expression, respectively). In SNU-5 and H292 cell lines, unconjugated HomoFc variant v32634 yielded 0.59 and 0.02 nM Kd values, respectively. Similarly, in SNU-5 and H292 cell lines, unconjugated HetFc variant v17427 yielded 0.33 and 0.03 nM Kd values, respectively. Binding affinities of unconjugated HomoFc and HetFc variants was comparable to lysine-conjugated ADC counterparts, DAR 4 and DAR 6, in both cMet-expressing cell lines. Unconjugated palivizumab antibody, v22277, did not show binding to cMet-expressing cell lines, as expected.

TABLE 10.1
Cellular binding - Kd and Bmax Summary

| | | Bmax | | Apparent Kd (nM) | |
|---|---|---|---|---|---|
| Test article | Fc Region | SNU-5 | H292 | SNU-5 | H292 |
| v32634 | HomoFc | 37244 | 2240 | 0.59 | 0.02 |
| v32634-Drug-Linker | HomoFc | 33736 | 2130 | 0.46 | 0.02 |
| 002 DAR4 | | | | | |
| v32634-Drug-Linker | HomoFc | 41080 | 2199 | 0.71 | 0.04 |
| 002 DAR6 | | | | | |
| v17427 | HetFc | 34831 | 2116 | 0.33 | 0.03 |
| v17427-Drug-Linker | HetFc | 34053 | 2120 | 0.43 | 0.04 |
| 002 DAR 4 | | | | | |
| v17427-Drug-Linker | HetFc | 33523 | 2148 | 0.43 | 0.05 |
| 002 DAR 6 | | | | | |
| v22277 | HomoFc | NA | NA | >50 | >50 |

### Example 11: In Vitro Cytotoxicity of Site-Specific Cysteine-Conjugated Anti-cMet Antibody-Drug Conjugates

The cell growth inhibition capabilities of site-specific cysteine-conjugated ADCs comprising various cysteine insertion antibody variants conjugated to Drug-Linker 001 at approximately DAR 1, 2 or 3, were assessed in a panel of four cMet-expressing tumour cell lines, SNU-5 (gastric carcinoma), EBC-1 (lung squamous cell carcinoma), H292 (lung carcinoma) and H1975 (non-small cell lung cancer), and one cMet-negative cell line, T-47D (breast carcinoma). An ADC comprising the anti-RSV antibody palivizumab (v22277) conjugated to Drug-Linker 001 at approximately DAR 4 was used as a non-targeted control. Actual DARs are shown in Table 11.1. “DAR 1” ADCs ranged from DAR 0.85-0.89, and “DAR 3” ADCs ranged from DAR 2.54-2.84. Cytotoxicity was determined as described in Example 7.

### Results

The results are summarized in Table 11.1. The cytotoxicity observed in cMet-expressing cell lines for site-specific ADCs with average DAR 1, 2 and 3 was drug-loading dependent. Generally, the three average DAR 1 site-specific ADCs showed lower cytotoxicity than the two average DAR 2 ADCs and five average DAR 3 ADCs. Stochastic ADCs with average DAR 4 exhibited higher cytotoxicity than DAR 3, 2 and 1 site-specific ADCs in all cell lines tested, yielding lower EC50 values and higher % maximum cytotoxicity. Two of the three DAR 1 site-specific ADCs yielded comparable cytotoxicity, these ADCs comprised variants v33967 and v33968 (see Table 1.3). The third DAR 1 ADC, comprising variant v33969, yielded higher cytotoxicity than the other DAR 1 ADCs in HT-29 and H441 cell lines. Both DAR 2 site-specific ADCs yielded comparable cytotoxicity in the cell lines tested. Finally, the five DAR 3 ADCs also yielded comparable cytotoxicity. The palivizumab stochastic DAR 4 ADC did not exhibit cytotoxicity in cMet-expressing cell lines, as expected.

TABLE 11.1
In vitro Cytotoxicity - EC50 Summary

| | No. of | | | | | |
|---|---|---|---|---|---|---|
| | Cysteine | | EC50 (nM) | | | |
| ADC | Insertions | DAR | EBC-1 | HT-29 | H292 | H441 |
| v17427-Drug- | — | 3.80 | 0.002 | 0.01 | 0.07 | 0.02 |
| Linker 001 | | | | | | |
| v17427-Drug- | — | 2.07 | 0.01 | 0.05 | 0.22 | 0.03 |
| Linker 002 | | | | | | |
| v33967-Drug- | 1 | 0.89 | 0.04 | 0.21 | >15 | 0.22 |
| Linker 001 | | | | | | |
| v33968-Drug- | 1 | 0.86 | 0.04 | 0.31 | >15 | IC* |
| Linker 001 | | | | | | |
| v33969-Drug- | 1 | 0.85 | 0.04 | 0.22 | >15 | 0.05 |
| Linker 001 | | | | | | |
| v29001-Drug- | 2 | 2.00 | 0.02 | 0.07 | 0.74 | 0.06 |
| Linker 001 | | | | | | |
| v28983-Drug- | 2 | 2.00 | 0.02 | 0.09 | 0.54 | 0.02 |
| Linker 001 | | | | | | |
| v33971-Drug- | 3 | 2.73 | 0.01 | 0.03 | 0.16 | 0.02 |
| Linker 001 | | | | | | |
| v33970-Drug- | 3 | 2.84 | 0.01 | 0.04 | 0.28 | 0.02 |
| Linker 001 | | | | | | |
| v33974-Drug- | 3 | 2.68 | 0.01 | 0.04 | 0.34 | 0.02 |
| Linker 001 | | | | | | |
| v33977-Drug- | 3 | 2.84 | 0.01 | 0.03 | 0.13 | 0.01 |
| Linker 001 | | | | | | |
| v33979-Drug- | 3 | 2.54 | 0.01 | 0.03 | 36.19 | 0.08 |
| Linker 001 | | | | | | |
| v22277-Drug- | — | 3.70 | >15 | >15 | >15 | >15 |
| Linker 001 | | | | | | |

*Incomplete curve

### Example 12: In Vitro Cytotoxicity of Site-Specific Anti-cMet Antibody-Drug Conjugates Comprising Multivalent Linkers

The cell growth inhibition capabilities of ADCs comprising variant v29001, having two cysteine insertions, conjugated to multivalent Drug-Linker 003 or multivalent Drug-Linker 004, were assessed in a panel of cMet-expressing tumour cell lines together with ADCs comprising parental variant v17427 conjugated to the monovalent Drug-Linker 002 at DAR 4 or DAR 6. Cell lines used were SNU-5 (gastric carcinoma), EBC-1 (lung squamous cell carcinoma), H292 (lung carcinoma), H1975 (non-small cell lung cancer) and T-47D (breast carcinoma, cMet-negative). ADCs comprising the anti-RSV antibody palivizumab (v22277) conjugated to Drug-Linker 002 were used as non-targeted controls. Free payload Compound 1 was also included as a control. Cytotoxicity was determined as described in Example 8.

### Results

The results are summarized in Table 12.1. In all cMet-expressing cell lines tested, average DAR 6 ADCs exhibited higher cytotoxicity than average DAR 4 ADCs, regardless of whether a monovalent or multivalent drug linker was used.

In high expression cMet cell lines, SNU-5 and EBC-1, monovalent drug-linker ADCs showed comparable cytotoxicity to DAR-matched multivalent drug-linker ADCs. In SNU-5 and EBC-1 cells, the monovalent drug-linker DAR 4 ADC yielded 38.5 pM and 9.6 pM EC50 values, respectively. In the same cell lines, multivalent drug-linker DAR 4 ADCs yielded 33.1 pM and 8.5 pM EC50 values, respectively. Similarly, in SNU-5 cells, monovalent and multivalent drug-linker DAR 6 ADCs yielded 13.6 pM and 18.7 pM EC50 values, respectively. In EBC-1 cells, monovalent and multivalent drug-linker DAR 6 ADCs yielded 6.2 pM and 4.1 pM EC50 values, respectively.

In moderate expression cMet cell lines, H1975 and H292, monovalent drug-linker ADCs showed comparable cytotoxicity to DAR-matched multivalent drug-linker ADCs. In H1975 and H292 cells, the monovalent drug-linker DAR 4 ADC yielded 83.6 pM and 118.0 pM EC50 values, respectively. In the same cell lines, multivalent drug-linker DAR 4 ADCs yielded 48.2 pM and 93.2 pM EC50 values, respectively. Similarly, in H1975 cells, monovalent and multivalent drug-linker DAR 6 ADCs yielded 43.7 pM and 27.7 pM EC50 values, respectively. In H292 cells, monovalent and multivalent drug-linker DAR 6 ADCs yielded 118.0 pM and 93.2 pM EC50 values, respectively. In the cMet-negative T-47D cell line, none of the monovalent and multivalent drug-linker DAR 4 and DAR 6 ADCs exhibited cytotoxicity, as expected. Palivizumab control ADC did not exhibit cytotoxicity in any of the cell lines tested, as expected.

TABLE 12.1
In vitro Cytotoxicity - EC50 Summary

| | | EC50 (pM) | | | | |
|---|---|---|---|---|---|---|
| ADC | DAR | SNU-5 | EBC-1 | H1975 | H292 | T-47D |
| v29001-Drug-Linker 003 | 4.0 | 33.1 | 8.5 | 48.2 | 93.2 | >20,000 |
| v29001-Drug-Linker 004 | 6.0 | 18.7 | 4.1 | 27.7 | 39.4 | >20,000 |
| v17427-Drug-Linker 002 | 4.0 | 38.5 | 9.6 | 83.6 | 118.0 | >20,000 |
| v17427-Drug-Linker 002 | 5.8 | 13.6 | 6.2 | 43.7 | 60.0 | >20,000 |
| v22277-Drug-Linker 002 | 3.7 | >20,000 | >20,000 | >20,000 | >20,000 | >20,000 |
| Compound 1 | — | 40050.0 | 26290.0 | 22710.0 | 7415.0 | 44830.0 |

### Example 13: Cellular Equilibrium Binding of Site-Specific Anti-cMet Antibody-Drug Conjugates

The on-cell binding capabilities of the following unconjugated antibodies and ADCs were assessed on SNU-5 and H292 endogenous cMet-expressing cell lines by flow cytometry: unconjugated variant v17427 (HetFc, unmodified hinge), and cysteine insertion variant v29001 (two cysteine insertions), lysine conjugated ADCs comprising variant v17427 and Drug-Linker 002 at DAR 4 or DAR 6, and cysteine conjugated ADCs comprising cysteine insertion variant insertion v29001 conjugated to Drug-Linker 001, Drug-Linker 003 or Drug-Linker 004 at DAR 2, 4 or 6, respectively. Unconjugated palivizumab v22277 was used as a non-targeted control. Cellular binding was determined as described in Example 10.

### Results

The results are summarized in Table 13.1. All unconjugated anti-cMet antibodies and ADCs exhibited comparable apparent Kd and Bmax values in both SNU-5 and H292 cell lines (high and moderate endogenous cMet expression, respectively). Unconjugated antibodies, v17427 and v29001, yielded 0.328 nM and 0.428 nM Kd values, respectively, in the high cMet expression cell line SNU-5. Similarly, variant v17427 DAR 4 and DAR 6 ADCs yielded 0.428 nM and 0.435 nM Kd values, respectively, in SNU-5 cells. Variant v29001 ADCs yielded ranges of Kd values between 0.584 nM and 1.131 nM in SNU-5 cells.

In moderate cMet expression cell line H292, unconjugated antibodies, v17427 and v29001, yielded 0.026 nM and 0.024 nM Kd values, respectively. Similarly, variant v17427 DAR 4 and DAR 6 ADCs yielded 0.040 nM and 0.047 nM Kd values, respectively, in H292 cells. Variant v29001 ADCs yielded ranges of Kd values between 0.028 nM and 0.064 nM in H292 cells. Palivizumab negative control did not exhibit cell binding to any of the cMet-expressing cell lines, as expected.

TABLE 13.1
Cellular Binding - Kd and Bmax Summary

| | | Bmax | | Apparent Kd (nM) | |
|---|---|---|---|---|---|
| Test Article | Antibody Backbone | SNU-5 | H292 | SNU-5 | H292 |
| v17427 | HetFc | 34831 | 2116 | 0.328 | 0.026 |
| | unmodified hinge | | | | |
| v29001 | HetFc | 32115 | 2039 | 0.428 | 0.024 |
| | unmodified hinge, | | | | |
| | 2 cysteine insertions | | | | |
| v17427-Drug- | HetFc | 34053 | 2120 | 0.428 | 0.040 |
| Linker 002 DAR 4 | unmodified hinge | | | | |
| v17427-Drug- | HetFc | 33523 | 2148 | 0.435 | 0.047 |
| Linker 002 DAR 6 | unmodified hinge | | | | |
| v29001-Drug- | HetFc | 35814 | 2069 | 0.584 | 0.064 |
| Linker 001 DAR 2 | unmodified hinge, | | | | |
| | 2 cysteine insertions | | | | |
| v29001-Drug- | HetFc | 38631 | 2048 | 1.131 | 0.030 |
| Linker 003 DAR 4 | unmodified hinge, | | | | |
| | 2 cysteine insertions | | | | |
| v29001-Drug- | HetFc | 37715 | 2008 | 0.615 | 0.028 |
| Linker 004 DAR 6 | unmodified hinge, | | | | |
| | 2 cysteine insertions | | | | |
| v22277 | Palivizumab | NA | NA | >50 | >50 |
| | Het Fc | | | | |

### Example 14: Internalization of Anti-cMet Antibody-Drug Conjugates

The receptor-mediated internalization capabilities of unconjugated variant v17427 (HetFc, unmodified hinge) and v17427-Drug-Linker 002 DAR 6.7 ADC were assessed in two cMet-expressing cell lines, IGROV-1 and OVCAR-3, using high content imaging as described below. Unconjugated anti-RSV antibody palivizumab (v22277) was used as a negative control.

Briefly, antibodies were fluorescently labeled by coupling to an anti-Human IgG Fc Fab fragment AF488 conjugate (Jackson Immuno Research Labs, West Grove, PA; Cat. No. 109-547-008) at a 1:1 molar ratio in PBS pH 7.4 (Thermo Fisher Scientific, Waltham, MA; Cat. No. 10010-023) for 24 hours at 4° C. Cells were seeded at 5,000 cells/well in 384-well plates and incubated overnight under standard culturing conditions (37° C./5% CO₂) to allow attachment. Coupled antibodies were added to cells the following day at various concentrations (70 to 0.3 nM) and incubated under standard culturing conditions for 5 hours to allow for internalization. Following incubation, cells were fixed using FluoroFix™ Buffer (BioLegend, San Diego, CA; Cat. No. 422101), 20 uL/well, for 30 min at room temperature. Nuclear stain, Hoechst 33342 (Thermo Fisher Scientific, Waltham, MA; Cat. No. 62249), was added to wells at 10 uM and the assay plate was incubated for 1 hour at 37° C./5% CO₂. Following incubation with nuclear stain, fluorescence images were captured using the Cytation 5 Cell Imaging Multimode Reader (BioTek Instruments, Winooski, VT) and analyzed using Gen 5 software (BioTek Instruments, Winooski, VT) to identify Mean Object Fluorescence per well. Mean Object Fluorescence (GFP channel) values were plotted using GraphPad Prism Version 9 (GraphPad Software, San Diego, CA).

### Results

The results are summarized in Table 14.1. Unconjugated variant v17427 showed comparable receptor-mediated internalization to the ADC, variant v17427-Drug-Linker 002 DAR 6.7, in EBC-1 and HT-29 (high and moderate cMet-expressing cell lines, respectively), suggesting conjugation of drug-linker does not affect the binding and internalization capabilities of the antibody. Both unconjugated antibody and ADC showed dose-dependent internalization with 70 nM to 0.3 nM treatments in both cMet-expressing cell lines. Palivizumab control (v22277) did not exhibit any internalization in EBC-1 and HT-29 cells, as expected.

TABLE 14.1
Receptor-Mediated Internalization

| | | Mean Object Fluorescence (Internalized) | |
|---|---|---|---|
| | | 5 Hours 37° C. | |
| Sample | Conc. (nM) | EBC-1 | HT-29 |
| v17427 | 70 | 8,387 | 4,588 |
| | 12 | 5,797 | 4,140 |
| | 1.9 | 4,930 | 3,761 |
| | 0.3 | 4,713 | NA |
| v17427-Drug- | 70 | 9,276 | 4,468 |
| Linker 002 | 12 | 9,514 | NA |
| DAR 6.7 | 1.9 | 7,172 | 3,541 |
| | 0.3 | 3,465 | 3,601 |
| v22277 | 70 | 2,605 | 2,290 |
| | 12 | 1,989 | 2,331 |
| | 1.9 | 3,054 | 2,363 |
| | 0.3 | 2,686 | 2,746 |
| Blank | NA | 2,346 | 2,503 |
| | | 1,984 | 2,318 |

### Example 15: cMet Pathway Agonism by Site-Specific Cysteine-Conjugated or Lysine-Conjugated Anti-cMet Antibody-Drug Conjugates

cMet pathway agonism of ADCs comprising various cysteine insertion variants conjugated to Drug-Linker 001 at DAR 2 was assessed by cell proliferation. cMet pathway agonism was also assessed by an ELISA to measure for AKT phosphorylation as a downstream indicator of cMet activation following the protocols described in Example 7 for ADCs comprising cysteine insertion variants conjugated to Drug-Linker 001 at DAR 1, 2 or 3, as well as cysteine-conjugated v17427-Drug-Linker 001 at DAR 4 and lysine-conjugated v17427-Drug-Linker 002 at DAR 2.

### Results

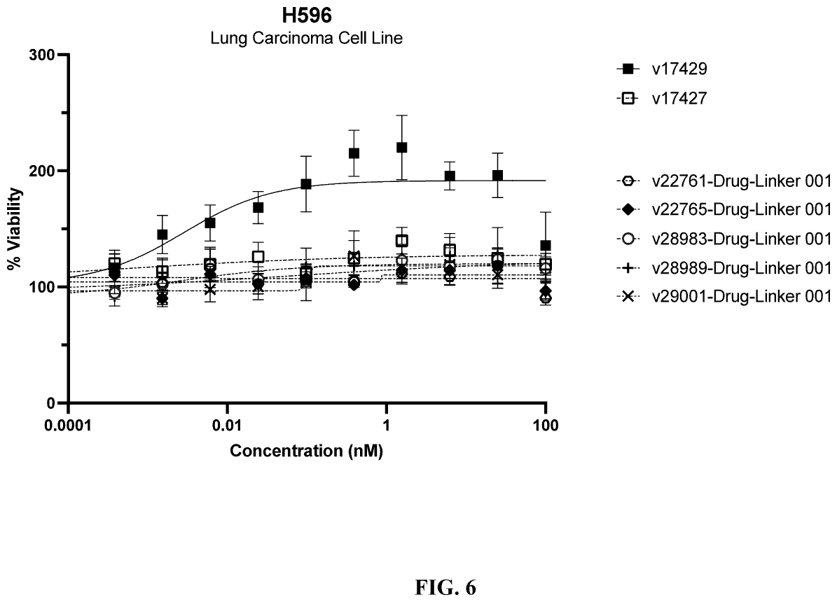

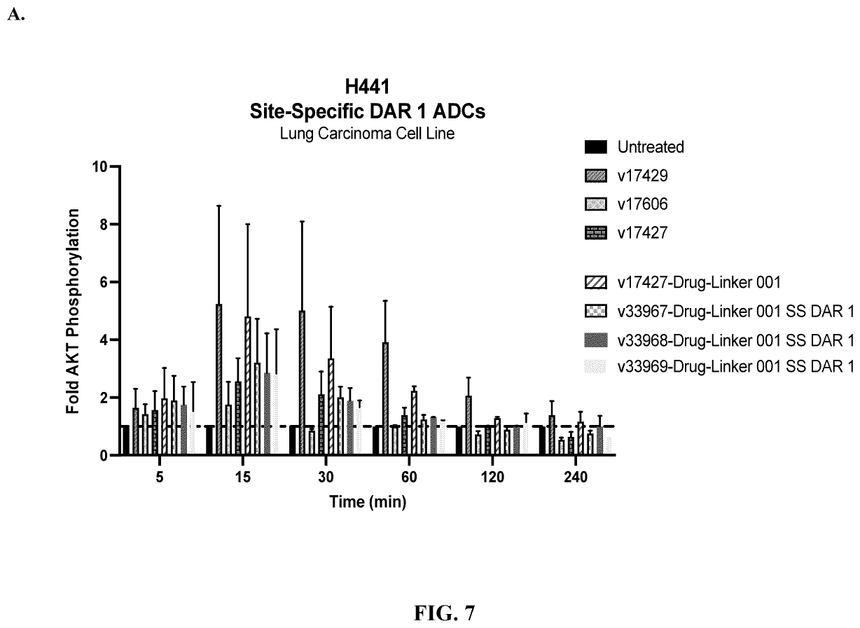

The results of the cell proliferation assessment are shown in FIG. 6. Bivalent MetMab (v17429) exhibited a strong dose-dependent increase in proliferation of H596 cells compared to untreated controls, whereas variant v17427 (HetFc, unmodified hinge) showed a slight increase in proliferation. Site-specific DAR 2 ADCs comprising variants v22761, v22765, v28983, v28989 or v29001 did not exhibit a noticeable increase in proliferation of H596 cells.

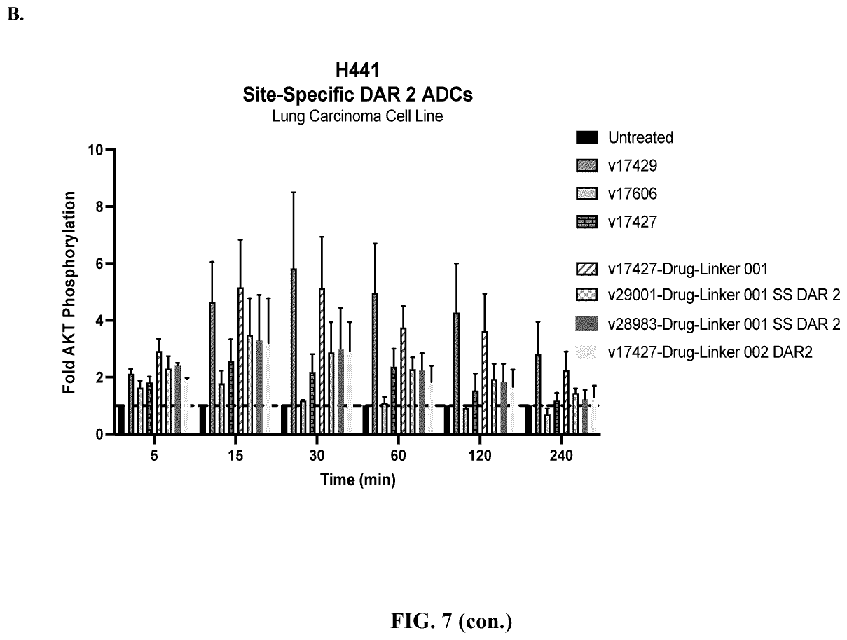

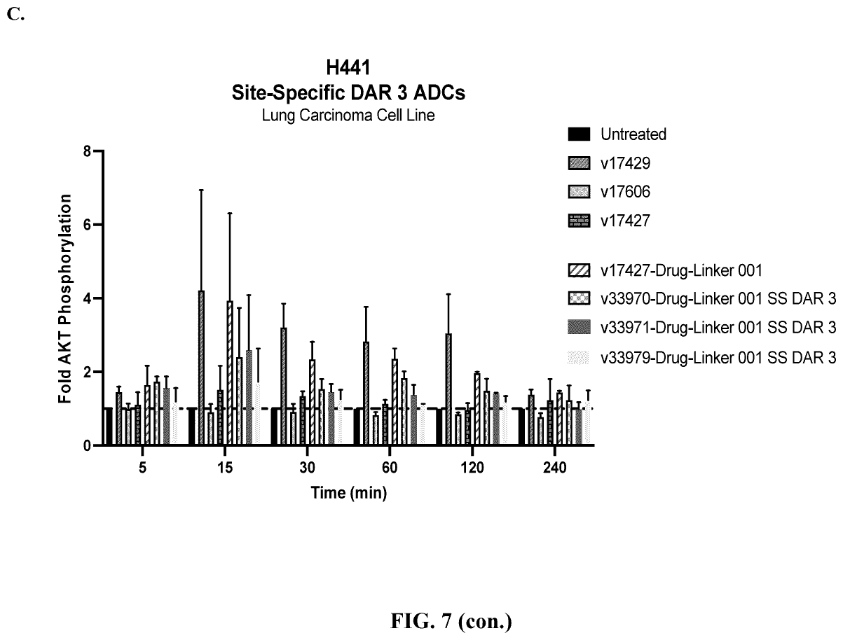

Results of the AKT phosphorylation assessment are shown in FIG. 7A-C. Bivalent MetMab (v17429) strongly induced phosphorylation in H441 cells whereas minimal effect was seen with hinge-modified telisotuzumab (v17606). Consistent with results described in Example 7, variant v17427 (HetFc, unmodified hinge) induced slight transient AKT phosphorylation, which was further increased with stochastic cysteine conjugation of Drug-Linker 001. In contrast, phosphorylation profiles for ADCs with site-specific cysteine conjugation of Drug-Linker 001 at various DARs did not differ from those of variant v17427. These observations suggest that a reduction in hinge cysteines during stochastic conjugation may increase cMET agonism, which can be circumvented through site-specific conjugation to inserted cysteine residues. The ADC with stochastic DAR 2 lysine conjugation of Drug-Linker 002 to variant v17427 also resulted in minimal change to AKT phosphorylation compared to the unconjugated parental antibody (FIG. 7B), further supporting the potential impact of stochastic cysteine conjugation on cMET agonism.

### Example 16: In Vivo Activity of Stochastic Cysteine-Conjugated Anti-cMet Antibody-Drug Conjugates (Dar 4)

In vivo anti-tumour activity of ADCs comprising variant v17427 (HetFc, unmodified hinge) stochastically cysteine-conjugated to Drug-Linker 001 or MCvcPABC-MMAE at DAR 4 was assessed in a number of cell-line derived xenograft (CDX) models expressing a range of cMet levels as described below. Cell-lines were as follows: cMet high HCC827 lung cancer, cMet high EBC1 lung cancer, cMet high H1975 lung cancer, cMet mid/high HT29 colorectal cancer, cMet low H292 lung cancer and cMet low SW48 colorectal cancer.

For the cMet high HCC827 lung cancer model, 5×10⁶ cells were implanted to BALB/c nude mice in 0.1 ml 1:1 PBS:Matrigel and upon tumour volume reaching approximately 125 mm³, mice were assigned to groups (n=5 per group) and treated as specified in Table 16.1 on Day 0, with a study duration of 60 days. For the cMet high EBC1 lung cancer model, 3×10⁶ cells in 0.1 ml PBS were implanted to female BALB/c nude mice and upon tumour volume reaching approximately 135 mm³, mice were assigned to groups (n=6 per group) and treated as specified in Table 16.1 on Day 0, with a study duration of 60 days. For the cMet high H1975 lung cancer model, 5×10⁶ cells in 0.1 ml PBS were implanted to BALB/c-Foxn1^{nu} mice and upon tumour volume reaching approximately 155 mm³, mice were assigned to groups (n=5 per group) and treated as specified in Table 16.1 on Day 0, with a study duration of 56 days. For the cMet mid/high HT29 colorectal model, 3×10⁶ cells in 0.1 ml PBS were implanted to BALB/c nude mice and upon tumour volume reaching approximately 150 mm³, mice were assigned to groups (n=5 per group) and treated as specified in Table 16.1 on Day 0, with a study duration of 60 days. For the cMet low H292 lung cancer model, 5×10⁶ cells in 0.1 ml PBS were implanted to SCID/beige mice and upon tumour volume reaching approximately 160 mm³, mice were assigned to groups (n=5 per group) and treated as specified in Table 16.1 on Day 0, with a study duration of 46 days. For the cMet low SW48 colorectal cancer model, 1×10⁷ cells in 0.1 ml PBS were implanted to BALB/c nude mice and upon tumour volume reaching approximately 120 mm³, mice were assigned to groups (n=5 per group) and treated as specified in Table 16.1 on Day 0, with a study duration of 42 days.

For all models, tumour volume and body weight were measured twice weekly. Tumour volume plots represent the mean and standard error of the mean. Mean data was plotted only if greater or equal to 80% of the mice remain on study for that timepoint. For statistical analyses, a linear mixed effects model was fit to log-transformed tumour volumes, followed by F-test for the null hypothesis that mean growth rates are equal and post-hoc pairwise comparisons.

TABLE 16.1
Test Articles and Dosing

| Study | Test Articles | DAR | Dose (mg/kg) |
|---|---|---|---|
| HCC827 | v17427-Drug-Linker 001 | 4 | 3, 10 |
| | v17427-MCvcPABC-MMAE | 4 | 3, 10 |
| EBC1 | v17427-Drug-Linker 001 | 4 | 5, 10 |
| | v17427-MCvcPABC-MMAE | 4 | 5, 10 |
| H1975 | v17427-Drug-Linker 001 | 4 | 2, 4, 8 |
| | v17427-MCvcPABC-MMAE | 4 | 2, 4, 8 |
| HT29 | v17427-Drug-Linker 001 | 4 | 3, 10 |
| | v17427-MCvcPABC-MMAE | 4 | 3, 10 |
| H292 | v17427-Drug-Linker 001 | 4 | 3, 10 |
| | v17427-MCvcPABC-MMAE | 4 | 3, 10 |
| SW48 | v17427-Drug-Linker 001 | 4 | 10, 20 |
| | v17427-MCvcPABC-MMAE | 4 | 5, 10 |

### Results

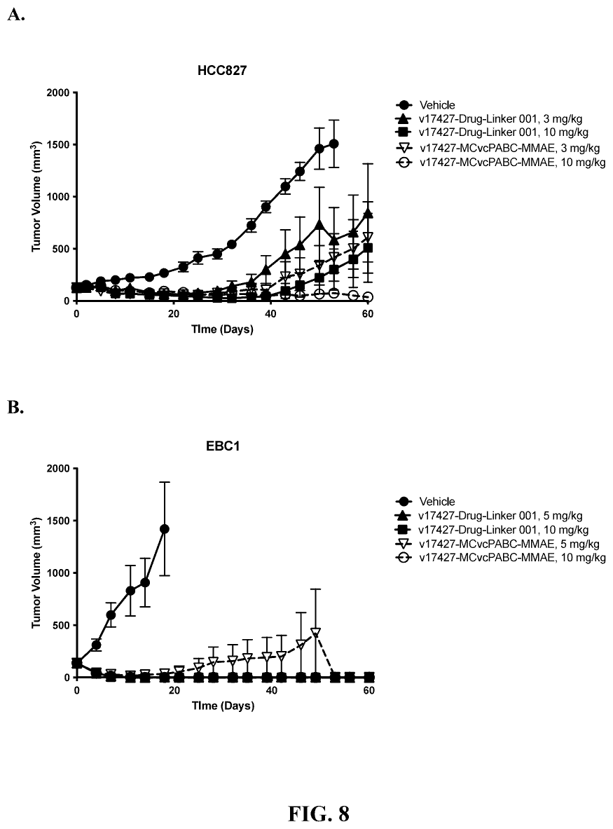

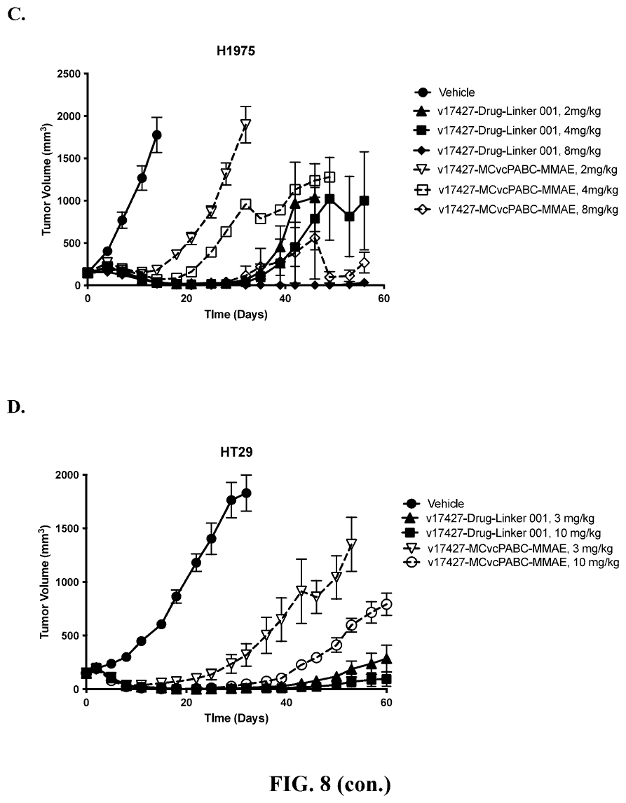

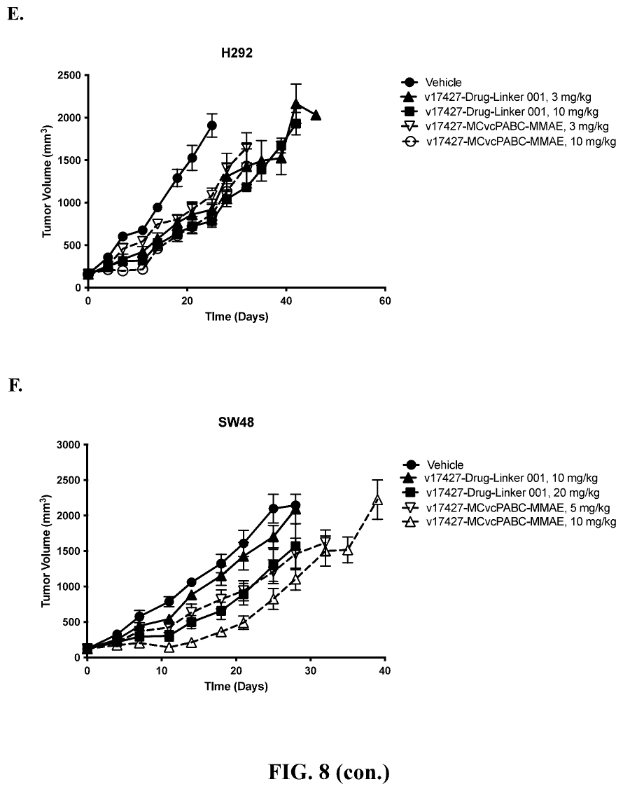

The results are shown in FIG. 8A-F and summarized below.

In the cMet high HCC827 lung cancer model (FIG. 8A) variant v17427 conjugated to Drug-Linker 001 or MCvcPABC-MMAE demonstrated strong inhibition of tumour growth at both 3 and 10 mg/kg dose levels compared to vehicle control (p<0.0001). Tumour growth inhibition trended to be more sustained following v17427-MCvcPABC-MMAE dosing than following v17427-Drug-Linker 001 dosing.

In the cMet high EBC1 lung cancer model (FIG. 8B) v17427-Drug-Linker 001 and v17427-MCvcPABC-MMAE demonstrated strong inhibition of tumour growth at both 5 and 10 mg/kg dose levels compared to vehicle control (p<0.005).

In the cMet high H1975 lung cancer model (FIG. 8C) v17427-Drug-Linker 001 and v17427-MCvcPABC-MMAE demonstrated inhibition of tumour growth at 2, 4 and 8 mg/kg dose levels compared to vehicle control (p<0.01), with a trend of dose responses for both ADCs. v17427-Drug-Linker 001 trended to demonstrate greater tumour growth inhibition than v17427-MCvcPABC-MMAE and this comparison was significant at the 2 mg/kg dose level (p<0.01).

In the cMet mid/high HT29 colorectal cancer model (FIG. 8D) v17427-Drug-Linker 001 and v17427-MCvcPABC-MMAE demonstrated strong inhibition of tumour growth at both 3 and 10 mg/kg dose levels compared to vehicle control (p<0.0001). Tumour growth inhibition trended to be more sustained following v17427-Drug-Linker 001 dosing than following v17427-MCvcPABC-MMAE dosing and the rate of regrowth was slower following v17427-Drug-Linker 001 dosing than v17427-MCvcPABC-MMAE dosing when each was dosed at 10 mg/kg (p<0.005).

In the cMet low H292 lung cancer model (FIG. 8E) v17427-Drug-Linker 001 and v17427-MCvcPABC-MMAE demonstrated moderate inhibition of tumour growth at both 3 and 10 mg/kg dose levels compared to vehicle control (p<0.05). No significant dose response was observed between 3 and 10 mg/kg dose levels or between v17427-Drug-Linker 001 and v17427-MCvcPABC-MMAE responses.

In the cMet low SW48 colorectal cancer model (FIG. 8F) neither v17427-Drug-Linker 001 nor v17427-MCvcPABC-MMAE demonstrated strong inhibition of tumour growth at the administered dose levels and there was no significant difference in the activities of v17427-Drug-Linker 001 and v17427-MCvcPABC-MMAE.

Together, these data demonstrate v17427-Drug-Linker 001 and v17427-MCvcPABC-MMAE ADCs to be active against cMet-expressing models in vivo. A trend was demonstrated between degree of response and cMet expression. v17427-Drug-Linker 001 and v17427-MCvcPABC-MMAE demonstrated largely comparable activities, with superior activity of v17427-Drug-Linker 001 observed in the HT29 and H1975 models.

### Example 17: In Vivo Activity of Anti-cMet Antibody-Drug Conjugates (DAR 1, 2, 3 and 4)

In vivo anti-tumour activity of cMet-targeting ADCs comprising stochastic lysine conjugated Drug-Linker 002 at DAR 2 and site-specific cysteine conjugated Drug-Linker 001 at DAR 1, 2, 3 and 4 was assessed in the H1975 (cMet high) lung cancer and HT29 (cMet mid/high) colorectal cancer CDX models. The ADCs and their dosing are listed in Table 17.1. Doses were selected in order to make comparisons at antibody-matched and toxin-matched dose levels. The studies using these models were performed following the methods as described in Example 16, except that the H1975 model used n=8 mice per group and a study duration of 41 days, and the HT29 model used n=8 mice per group and a study duration of 32 days. Mean data was plotted only if greater or equal to 80% of the mice remain on study for that timepoint.

TABLE 17.1
Test Articles and Dosing

| Study | Test Articles | DAR | Dose (mg/kg) |
|---|---|---|---|
| H1975 | v33967-Drug-Linker 001 | 1 | 4, 24 |
| | v33968-Drug-Linker 001 | 1 | 4, 24 |
| | v29001-Drug-Linker 001 | 2 | 2, 12 |
| | v28983-Drug-Linker 001 | 2 | 2, 12 |
| | v17427-Drug-Linker 002 | 2 | 2, 12 |
| | v33970-Drug-Linker 001 | 3 | 1.3, 8 |
| | v33971-Drug-Linker 001 | 3 | 1.3, 8 |
| | v33979-Drug-Linker 001 | 3 | 1.3, 8 |
| | v17427-Drug-Linker 001 | 4 | 1, 6 |
| | v17427-MCvcPABC-MMAE | 4 | 6 |
| HT29 | v33967-Drug-Linker 001 | 1 | 6, 12 |
| | v33968-Drug-Linker 001 | 1 | 6, 12 |
| | v29001-Drug-Linker 001 | 2 | 3, 6 |
| | v28983-Drug-Linker 001 | 2 | 3, 6 |
| | v17427-Drug-Linker 002 | 2 | 3, 6 |
| | v33970-Drug-Linker 001 | 3 | 2, 4 |
| | v33971-Drug-Linker 001 | 3 | 2, 4 |
| | v33979-Drug-Linker 001 | 3 | 2, 4 |
| | v17427-Drug-Linker 001 | 4 | 1.5, 3 |
| | v17427-MCvcPABC-MMAE | 4 | 1.5 |

### Results

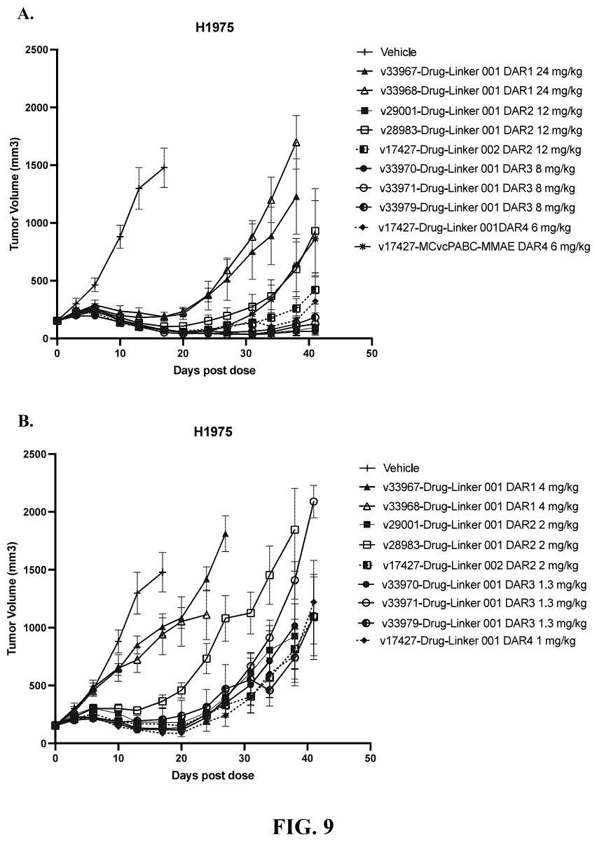

The results for the cMet high H1975 CDX lung cancer model are shown in FIGS. 9A & B. For the H1975 model, all DAR 1 Drug-Linker 001, DAR 2 Drug-Linker 001 and Drug-Linker 002, DAR 3 Drug-Linker 001 and DAR 4 Drug-Linker 001 ADCs significantly inhibited tumour growth compared to vehicle at the toxin-matched antibody doses of 24, 12, 8 and 6 mg/kg, respectively (FIG. 9A). All DAR 2 Drug-Linker 001 and Drug-Linker 002, DAR 3 Drug-Linker 001 and DAR 4 Drug-Linker 001 ADCs also significantly inhibited tumour growth compared to vehicle at the toxin-matched antibody doses of 2, 1.3 and 1 mg/kg, respectively (FIG. 9B). While DAR 2, DAR 3 and DAR 4 Drug-Linker 001 site-specific ADCs, and v17427-Drug-Linker 001 and v17427-Drug-Linker 002 ADCs showed comparable activity at toxin-matched doses, DAR 1 Drug-Linker 001 ADCs appeared less active at toxin-matched doses.

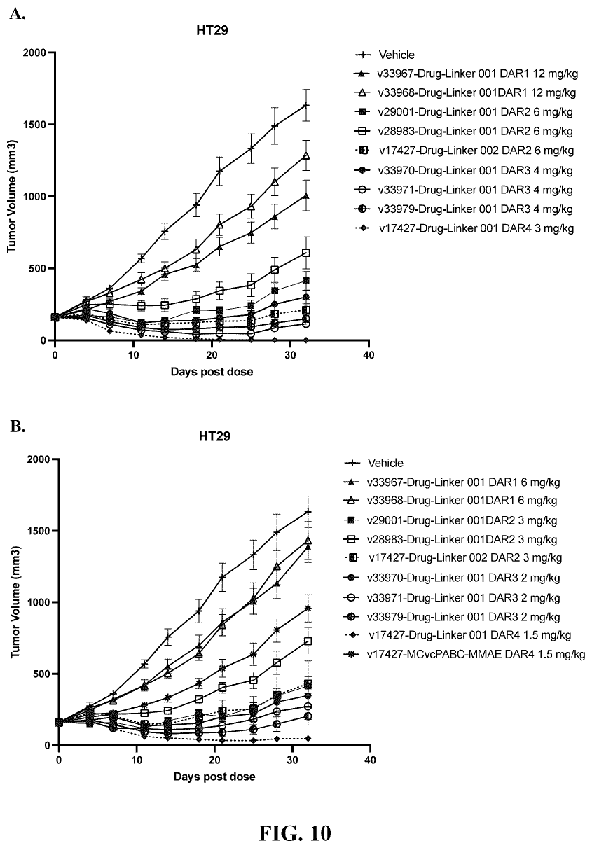

The results for the cMet mid/high HT29 CDX model of colorectal cancer are shown in FIGS. 10A & B. In the HT29 model, all DAR 2 Drug-Linker 001 and Drug-Linker 002, DAR 3 Drug-Linker 001 and DAR 4 Drug-Linker 001 ADCs significantly inhibited tumour growth compared to vehicle at the toxin matched antibody doses of 6, 4 and 3 mg/kg, respectively (p<0.05, mixed effects model for tumour growth rate) (FIG. 10A). DAR 1 Drug-Linker 001 ADCs did not inhibit tumour growth at the toxin-matched dose of 12 mg/kg. All DAR 2 Drug-Linker 001 and Drug-Linker 002, DAR 3 Drug-Linker 001 and DAR 4 Drug-Linker 001 ADCs, with the exception of v28983-Drug-Linker 001, also significantly inhibited tumour growth compared to vehicle at the toxin-matched antibody doses of 3, 2 and 1.5 mg/kg, respectively (FIG. 10B). By comparison, v17427-MCvcPABC-MMAE DAR 4 did not significantly inhibit tumour growth at the 1.5 mg/kg dose tested. There was a trend for a positive correlation of DAR with anti-tumour activity, when doses were toxin-matched. Activity of the site-specific cysteine-conjugated v29001-Drug-Linker 001 DAR 2 ADC was comparable to that of the stochastic lysine-conjugated v17427-Drug-Linker 002 DAR 2.

Together, these data demonstrate activity of stochastic and site-specific anti-cMet ADCs in cMet-expressing models in vivo and a trend for lower toxin-matched activity for low DAR ADCs.

### Example 18: In Vivo Activity of Anti-cMet Antibody-Drug Conjugates Comprising Multivalent Drug Linkers (CDX Models)

In vivo anti-tumour activities of ADCs comprising anti-cMet antibodies conjugated stochastically via lysine to Drug-Linker 002 or stochastically via cysteine to MCvcPABC-MMAE and ADCs comprising anti-cMet antibodies conjugated via site-specific cysteine insertions to multivalent Drug-Linker 003 or Drug-Linker 004 at DAR 4 or 6, respectively, were assessed in a number of CDX models expressing a range of cMet levels. ADCs were administered as specified in Table 18.1 in order to make comparisons at toxin-matched dose levels.

Studies using the H1975, HT29 and H292 models were performed following the methods as described in Example 16 with the following differences: the H1975 model used n=12 mice per group and a study duration of 28 days, and the HT29 model used n=12 mice per group and a study duration of 25 days. For the cMet mid/high Hs746t gastric cancer model, 5×10⁶ cells in 0.1 ml 1:1 PBS:matrigel were implanted to BALB/c nude mice and upon tumour volume reaching approximately 150 mm³, mice were assigned to groups (n=7 per group) and treated as specified in Table 18.1 for a study duration of 28 days. For the cMet mid HCT116 colorectal cancer model, 5×10⁵ cells in 0.1 ml PBS were implanted to BALB/c mice and upon tumour volume reaching approximately 150 mm³, mice were assigned to groups (n=7 per group) and treated as specified in Table 18.1 for a study duration of 27 days. Mean data was plotted only if greater or equal to 80% of the mice remain on study for that timepoint.

TABLE 18.1
Test Articles and Dosing

| Study | Test Articles | DAR | Dose (mg/kg) |
|---|---|---|---|
| H1975 | v17427-Drug-Linker 002 | 4 | 0.4, 0.8 |
| | v17427-Drug-Linker 002 | 6 | 0.27, 0.53 |
| | v29001-Drug-Linker 003 | 4 | 0.4, 0.8 |
| | v29001-Drug-Linker 004 | 6 | 0.27, 0.53 |
| | v17427-MCvcPABC-MMAE | 4 | 0.8 |
| HT29 | v17427-Drug-Linker 002 | 4 | 0.8 |
| | v17427-Drug-Linker 002 | 6 | 0.53 |
| | v29001-Drug-Linker 003 | 4 | 0.8 |
| | v29001-Drug-Linker 004 | 6 | 0.53 |
| | v17427-MCvcPABC-MMAE | 4 | 0.8 |
| H292 | v17427-Drug-Linker 002 | 4 | 6 |
| | v17427-Drug-Linker 002 | 6 | 4 |
| | v29001-Drug-Linker 004 | 6 | 4 |
| | v17427-MCvcPABC-MMAE | 4 | 6 |
| Hs746t | v32634-Drug-Linker 002 | 4 | 3 |
| | v35527-Drug-Linker 003 | 4 | 3 |
| | v35527-Drug-Linker 004 | 6 | 2 |
| | v17606-MCvcPABC-MMAE (HIC purified) | 3 | 3 |
| HCT116 | v32634-Drug-Linker 002 | 4 | 3 |
| | v35527-Drug-Linker 003 | 4 | 3 |
| | v35527-Drug-Linker 004 | 6 | 2 |
| | v17606-MCvcPABC-MMAE (HIC purified) | 3 | 3 |

### Results

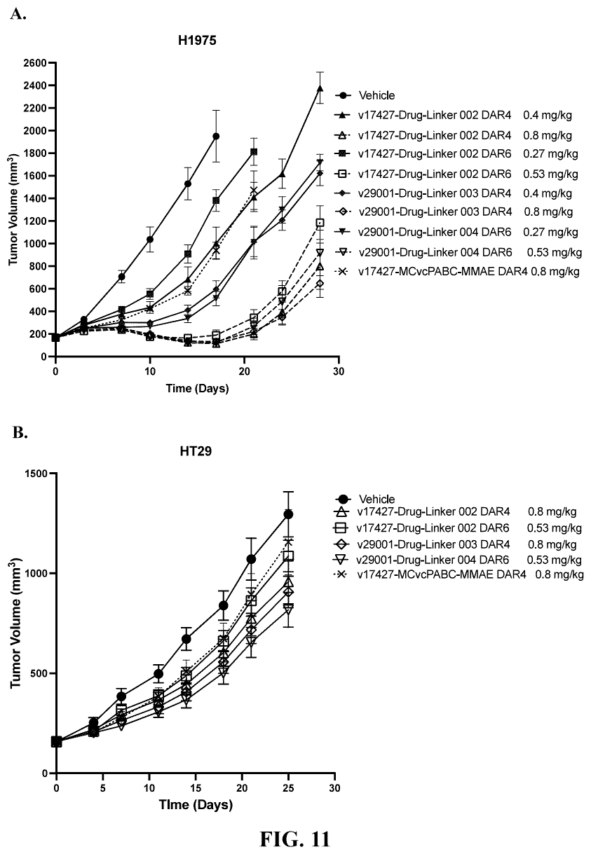

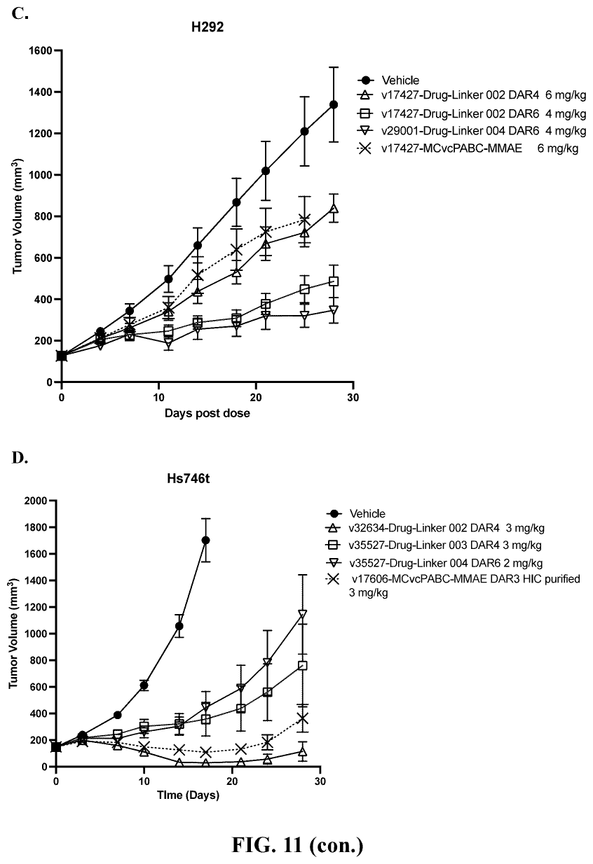

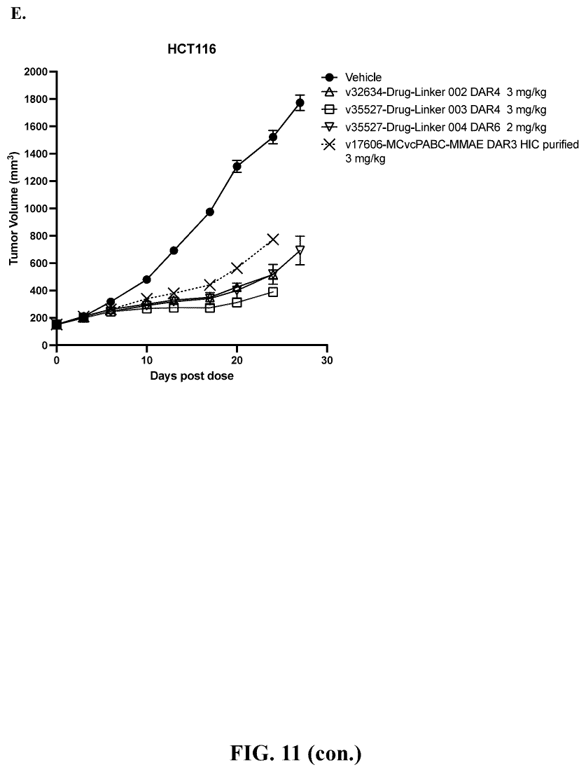

The results are shown in FIG. 11A-E and summarized below.

In the cMet high H1975 lung cancer model, tumour growth inhibition activity was assessed at 0.4 mg/kg and 0.8 mg/kg for DAR 4 ADCs and at 0.27 mg/kg and 0.53 mg/kg for DAR 6 ADCs. These relatively low doses were to allow for comparisons at toxin-matched doses. All ADCs resulted in a statistically significant inhibition of tumour growth compared to vehicle control (p<0.03) with the exceptions of v17427-MCvcPABC-MMAE DAR 4 at 0.8 mg/kg and lysine conjugated v17427-Drug-Linker 002 DAR 6 at 0.27 mg/kg (FIG. 11A). All Drug-Linker 002, 003 and 004 ADCs showed a statistically greater inhibition of tumour growth than v17427-MCvcPABC-MMAE at toxin-matched dose levels. At the 0.8 and 0.53 mg/kg dose levels of DAR 4 and DAR 6 ADCs, respectively, there was no differentiation of activity between any of the Drug-Linker 002, 003 and 004 ADCs. At the lower 0.4 and 0.27 mg/kg dose levels of DAR 4 and DAR 6 ADCs, respectively, the site-specific v29001-Drug-Linker 004 DAR 6 ADC demonstrated superior activity to the stochastic lysine conjugated v17427-Drug-Linker 002 DAR 4 and DAR 6 ADCs. The site-specific v29001-Drug-Linker 003 DAR 4 ADC demonstrated greater activity compared to the stochastic lysine v17427-Drug-Linker 002 DAR 4 ADC. Overall, these data indicate a superiority in activity of ADCs comprising drug-linkers conjugated via inserted cysteines over ADCs comprising drug-linkers conjugated by stochastic lysine conjugation, and a superiority in activity of all Compound 1-containing ADCs over the comparator MMAE ADC.

In the cMet mid/high HT29 colorectal cancer model, tumour growth inhibition activity was assessed at 0.8 and 0.53 mg/kg for DAR 4 and DAR 6 ADCs, respectively. These relatively low doses were to allow for comparisons at toxin-matched dose levels. Site-specific ADCs v29001-Drug-Linker 004 DAR 6 and v29001-Drug-Linker 003 DAR 4 and stochastic lysine conjugated v17427-Drug-Linker 002 DAR 4 all resulted in statistically significant inhibition of tumour growth compared to vehicle control and to the comparator ADC v17427-MCvcPABC-MMAE DAR 4 (FIG. 11B). Stochastic lysine conjugated v17427-Drug-Linker 002 DAR 6 did not differentiate from vehicle control or the comparator ADC v17427-MCvcPABC-MMAE DAR 4. Overall, these data indicate a superiority in activity of ADCs comprising drug-linkers conjugated via inserted cysteines over ADCs comprising drug-linkers conjugated by stochastic lysine conjugation and over the comparator MCvcPABC-MMAE ADC.

In the cMet low H292 lung cancer model, site-specific v29001-Drug-Linker 004 DAR 6 and stochastic lysine conjugated v32634-Drug-Linker 002 DAR 6 ADCs both demonstrated moderate inhibition of tumour volume compared to vehicle control and demonstrated greater activity compared to v17427-MCvcPABC-MMAE DAR 4 (FIG. 11C).

In the cMet mid/high Hs746t gastric cancer model, all ADCs demonstrated inhibition of tumour growth compared to vehicle control (FIG. 11D). In this model, the stochastic lysine conjugated ADC, v32634-Drug-Linker 002 DAR 4 demonstrated the greatest activity and was superior to the site-specific v35527 DAR 4 and DAR6 ADCs. v32634-Drug-Linker 002 DAR 4 trended superior to v17606-MCvcPABC-MMAE DAR 3.

In the cMet mid HCT116 colorectal cancer model, all tested ADCs demonstrated moderate inhibition of tumour growth compared to vehicle control (FIG. 11E). The site-specific DAR 4 and DAR 6 ADCs and stochastic DAR 4 ADC did not statistically differentiate from each other, but all demonstrated statistically superior activity to v17606-MCvcPABC-MMAE DAR 3 (p<0.01).

Together, these data demonstrate that ADCs comprising Compound 1 as a payload (i.e. Drug-Linkers 002, 003 and 004) conjugated by either stochastic lysine conjugation or site-specific cysteine conjugation at DAR 4 or DAR 6 are active against cMet-expressing in vivo xenograft models and are largely superior in activity to ADCs comprising MMAE as a payload (i.e. drug-linker MCvcPABC-MMAE).

### Example 19: In Vivo Activity of Anti-cMet Antibody-Drug Conjugates Comprising Multivalent Drug Linkers (PDX Models)

In vivo anti-tumour activities of lysine-conjugated DAR 4 ADCs (v32634-Drug-Linker 002) and site-specific cysteine-conjugated DAR 6 ADCs (v35527-Drug-Linker 004 or v29001-Drug-Linker 004) were compared to v17606-MCvcPABC-MMAE DAR 3 (either HIC purified v36198 ADC or non-HIC purified v19875 ADC-see Table 5.1) in a panel of lung cancer PDX models expressing a range of cMet levels. The site-specific DAR 6 ADC was dosed at 2 mg/kg to toxin-match to the 3 mg/kg dose of the lysine conjugated DAR 4 ADC. Variants v32634, v35527 and v29001 all comprise an unmodified hinge, whereas variant v17606 comprises a modified hinge (see Table 1.3). Variant v17606-MCvcPABC-MMAE is equivalent to telisotuzumab vedotin (ABBV399).

Mice (NudeFoxn1nu or BALB/c nude) were implanted with tumour fragments from stock mice and assigned to treatment groups when tumour volume reached approximately 150 to 300 mm³. ADCs were administered by single IV injection as specified in Table 19.1. Study durations ranged from 25 to 42 days. Tumors from non-treated mice were excised for formalin fixation and paraffin embedding and subjected to immunohistochemistry (IHC) analysis for relative cMet levels using antibody clone SP44 (Abcam) according to standard IHC methods.

TABLE 19.1
Test Articles and Dosing

| | Relative c-Met | | | |
|---|---|---|---|---|
| | Level (H-score, | | | Dose |
| PDX Model | IHC) | Test Articles | DAR | (mg/kg) |
| LU1235 | 81 | v32634-Drug-Linker 002 | 4 | 3 |
| | | v17606-MCvcPABC-MMAE1 | 3 | 3 |
| LU0387 | 3 | v32634-Drug-Linker 002 | 4 | 3 |
| | | v17606-MCvcPABC-MMAE1 | 3 | 3 |
| CTG0178 | 147 | v32634-Drug-Linker 002 | 4 | 3 |
| | | v17606-MCvcPABC-MMAE1 | 3 | 3 |
| LU2049 | 117 | v32634-Drug-Linker 002 | 4 | 3 |
| | | v29001-Drug-Linker 004 | 6 | 2 |
| | | v17606-MCvcPABC-MMAE1 | 3 | 3 |
| LU3075 | 69 | v32634-Drug-Linker 002 | 4 | 3 |
| | | v29001-Drug-Linker 004 | 6 | 2 |
| | | v17606-MCvcPABC-MMAE1 | 3 | 3 |
| CTG2533 | 126 | v32634-Drug-Linker 002 | 4 | 3 |
| | | v35527-Drug-Linker 004 | 6 | 2 |
| | | v17606-MCvcPABC-MMAE2 | 3 | 3 |
| CTG0159 | 171 | v32634-Drug-Linker 002 | 4 | 3 |
| | | v35527-Drug-Linker 004 | 6 | 2 |
| | | v17606-MCvcPABC-MMAE2 | 3 | 3 |
| CTG1766 | 74 | v32634-Drug-Linker 002 | 4 | 3 |
| | | v17606-MCvcPABC-MMAE1 | 3 | 3 |
| LU0858 | 177 | v32634-Drug-Linker 002 | 4 | 3 |
| | | v17606-MCvcPABC-MMAE1 | 3 | 3 |
| LU2529 | 140 | v32634-Drug-Linker 002 | 4 | 3 |
| | | v29001-Drug-Linker 004 | 6 | 2 |
| | | v17606-MCvcPABC-MMAE1 | 3 | 3 |
| CTG0165 | 204 | v32634-Drug-Linker 002 | 4 | 3 |
| | | v35527-Drug-Linker 004 | 6 | 2 |
| | | v17606-MCvcPABC-MMAE2 | 3 | 3 |
| LU5381 | 137 | v32634-Drug-Linker 002 | 4 | 3 |
| | | v29001-Drug-Linker 004 | 6 | 2 |
| | | v17606-MCvcPABC-MMAE1 | 3 | 3 |
| LU1868 | 66 | v32634-Drug-Linker 002 | 4 | 3 |
| | | v17606-MCvcPABC-MMAE1 | 3 | 3 |
| LU2503 | 194 | v32634-Drug-Linker 002 | 4 | 3 |
| | | v17606-MCvcPABC-MMAE1 | 3 | 3 |
| LU1901 | 136 | v32634-Drug-Linker 002 | 4 | 3 |
| | | v29001-Drug-Linker 004 | 6 | 2 |
| | | v17606-MCvcPABC-MMAE1 | 3 | 3 |

### Results

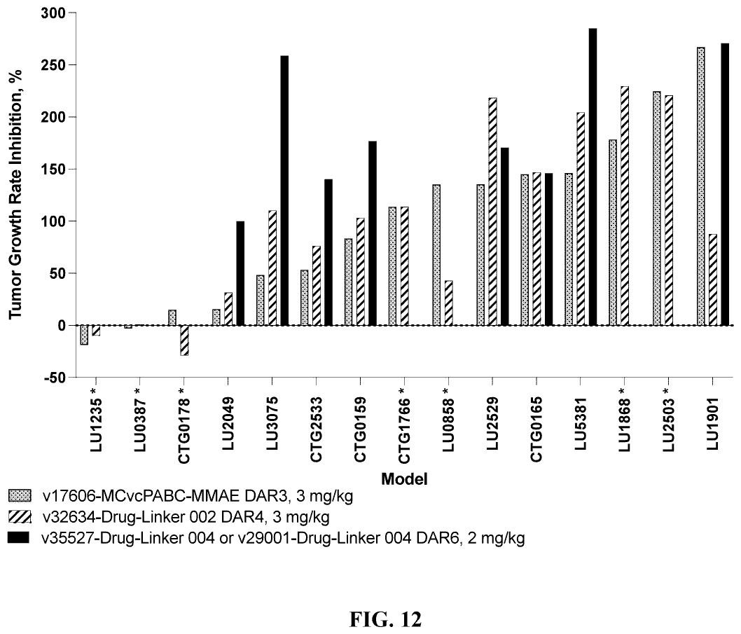

Tumour growth rate inhibition values are shown in FIG. 12. v32634-Drug-Linker 002 DAR 4 ADCs demonstrated similar or superior activity to v17606-MCvcPABC-MMAE DAR 3 ADCs in 12 of 16 models. Site-specific v29001-Drug-Linker 004 DAR 6 ADCs demonstrated superior activity to both v17606-MCvcPABC-MMAE DAR 3 ADCs and v32634-Drug-Linker 002 DAR 4 ADCs in 6 of 8 models where it was assessed, and non-inferior activity in the remaining two models.

Together, these data demonstrate that ADCs comprising site-specifically conjugated Compound 1 at DAR 6 are largely superior in activity to ADCs comprising lysine conjugated Compound 1 when administered at toxin-matched doses. Overall, ADCs comprising lysine conjugated Compound 1 at DAR 4 and ADCs comprising site-specifically conjugated Compound 1 at DAR 6 have superior activity to MCvcPABC-MMAE ADCs.

### Example 20: Pharmacokinetics of Anti-cMet Antibody-Drug Conjugates in Tg32 Mice

The pharmacokinetics of anti-cMet ADCs and corresponding free antibodies were assessed in humanized FcRn Tg32 mice as described below. Humanized FcRn Tg32 mice models were selected for this study as they are good predictors of the pharmacokinetics of a drug in humans. The ADCs and antibodies assessed were: v29001 (HetFc, unmodified hinge, two cysteine insertions), v17427 (HetFc, unmodified hinge), v29001-Drug-Linker 003 DAR 4, v29001-Drug-Linker 004 DAR 6, v17427-Drug-Linker 002 DAR 4 and v17427-Drug-Linker 002 DAR 6.

All test articles were administered at 5 mg/kg to hFcRn Tg32 mice (The Jackson Laboratory, Sacramento, CA; Stock #014565) by intravenous injection. For each test article, blood was collected from n=4 animals by retro-orbital or terminal bleed at 1, 4 and 8 hours and 1, 3, 7, 10, 14 and 21 days post-dose. Blood was processed to serum and stored frozen at −80° C. in 96-well storage plates prior to pharmacokinetics analysis.

Total IgG and total ADC concentrations of test articles in mouse serum were measured by sandwich ELISA utilizing an anti-human IgG1 Fc capture antibody (Jackson ImmunoResearch Labs, West Grove, PA; Cat. #709-005-098) or a rabbit anti-toxin capture antibody and an HRP-conjugated anti-IgG1 Fab detection antibody (Jackson ImmunoResearch Labs; Cat. #109-035-097). Absorbance at 450 nm was measured using a Synergy™ H1 Hybrid Multi-Mode Plate Reader (BioTek Instruments, Winooski, VT). Sample data were analyzed using SoftMax® Pro 7.1 (Molecular Devices, San Jose, CA). Pharmacokinetics parameters were calculated from non-compartmental analysis using Phoenix WinNonlin™ software (Certara, Princeton, NJ).

### Results

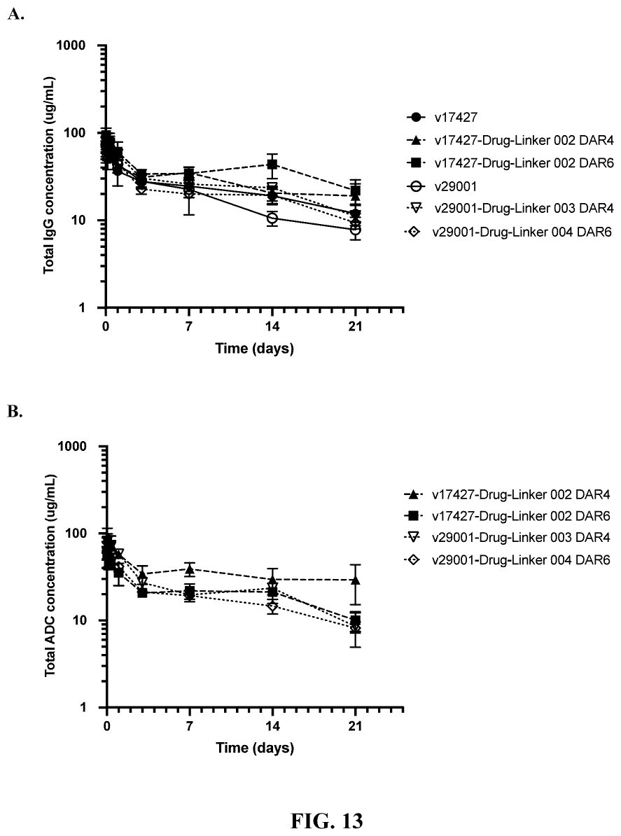

The results are shown in FIGS. 13A & B and Table 20.1. All mAbs and ADCs demonstrated typical antibody-like prolonged exposures. Variant v17427 and the cysteine insertion variant v29001 demonstrated minimal differences in their PK parameters. The ADCs v29001-Drug-Linker 003 DAR 4 and v29001-Drug-Linker 004 DAR 6, which utilize multivalent drug linkers, demonstrated comparable PK to their parent antibody, variant v29001.

TABLE 20.1
Test Articles and PK Values

| | | AUCinf | Clearance |
|---|---|---|---|
| Test Article | Half-life (days) | (day*ug/ml) | (ml/day/kg) |
| v17427 | 13.8 | 722.8 | 6.9 |
| v29001 | 9.2 | 509.9 | 9.8 |
| v29001-Drug-Linker 003 DAR4 | 8.1 | 687.1 | 7.3 |
| v29001-Drug-Linker 004 DAR6 | 9.4 | 565.1 | 8.8 |
| v17427-Drug-Linker 002 DAR4 | 18.0 | 1084.9 | 4.6 |
| v17427-Drug-Linker 002 DAR6 | 13.5 | 1219.5 | 4.1 |

### Example 21: In Vivo Stability of Anti-cMet Antibody-Drug Conjugates

The in vivo stability of the four ADCs described in Example 20 (v17427-Drug-Linker 002 DAR 4, v17427-Drug-Linker 002 DAR 6, v29001-Drug-Linker 003 DAR 4, and v29001-Drug-Linker 004 DAR 6) in Tg32 mice was assessed using immunoprecipitation/mass spectrometry as described below. Serum samples taken from Tg32 mice as noted in Example 20 at various time points in circulation (1 hr to 10 days) were employed. For all groups, mouse serum samples from each time point (1 hr to 21 days post-dose) were tested.

Briefly, biotinylated anti-human IgG F(ab′)2 antibody was coupled to magnetic beads coated with streptavidin (11 ug antibody per sample) for 30 min at room temperature. Following coupling, the beads were incubated with test sample for 1.5 hrs at room temperature to allow for immunocapture. Samples were washed with PBS pH 7.4 using DynaMag™-2 magnet (ThermoFisher Scientific Corporation, Waltham, MA). Immunocaptured samples were reduced (v29001-Drug-Linker 003 and v29001-Drug-Linker 004 only) using dithiothreitol (DTT) in PBS, pH 7.4, for 1 hr at room temperature. v17427-Drug-Linker 002 ADCs were not reduced. After additional washes with PBS pH 7.4, samples were eluted by incubating with pH 3.0 buffer (distilled water containing 20% acetonitrile and 1% formic acid) for 1 hr at room temperature. Isolated ADC samples were then analyzed by mass spectrometry to quantify DAR or drug loading or kept frozen at −80° C. until further analysis.

For LC-MS analysis, samples were injected into an Agilent™ PLRP-S 1000 Å 8 uM 50×2.1 mm column using a Agilent™ 1290 Infinity™ II LC System coupled with Agilent™ 6545 Quadrupole Time of Flight (Q-TOF) with a column temperature of 70° C. and a flow rate of 0.3 ml/min. Mobile phases consisted of A: LC-MS grade water with 0.1% v/v formic acid, 0.025 v/v trifluoroacetic acid and 10% v/v isopropyl alcohol, and B: acetonitrile with 0.1% v/v formic acid and 10% v/v isopropyl alcohol. The column was pre-equilibrated in 20% mobile phase B before sample injection. Then, a 20 min, 20 to 40% mobile phase B gradient was applied, followed by a 2 min, 40 to 90% mobile phase B gradient and a column wash of 2.5 min at 99% mobile phase B. The column was re-equilibrated to 10% mobile phase B for 2 minutes between runs. Electrospray Ionization (ESI) was performed in a Dual AJS™ ESI source (Agilent Technologies, Santa Clara, CA) in positive mode with 5000V of capillary voltage, 2000V nozzle voltage, 170V fragmentor voltage, 65V skimmer, 750V Octople RFPeak, 300° C. gas temperature, 13 L/min gas flow, 45 psig nebulizer, 400° C. sheath gas temperature. Data was acquired at a scan rate of 1 spectra/see with a m/z range from 500 to 7000.

Peak integration, MS deconvolution and mass assignments were performed in Protein Metrics Byos™ v4.0 (Protein Metrics Inc., Cupertino, CA) using a deconvolution window of 50000-170000 Da (for v17427-Drug-Linker 002 ADCs) or 20000-60000 Da (for v29001-Drug-Linker 003 and v29001-Drug-Linker 004), with an m/z range of 600-6000 (v17427-Drug-Linker 002 ADCs) or 850-4000 (v29001-Drug-Linker 003 and v29001-Drug-Linker 004). For reduced analysis of v29001-Drug-Linker 003 and v29001-Drug-Linker 004, the reference masses were defined as the average masses of each heavy chain of the parental antibody v29001 with one 2-acetamido-2-deoxy-beta-D-glucopyranose-(1-4)-[alpha-L-fucopyranose-(1-6)] stub arising from EndoS activity on N-glycans and the formation of pyroglutamic acid if a glutamine residue was present at the N-terminus in the protein sequence. For intact analysis of the v17427-Drug-Linker 002 ADCs, reference masses were defined as the average mass of the parental antibody v17427 with two 2-acetamido-2-deoxy-beta-D-glucopyranose-(1-4)-[alpha-L-fucopyranose-(1-6)] stubs, 16 disulfide bonds and the formation of pyroglutamic acid(s) if glutamine residue(s) were present at the N-terminus in the protein sequence. Drug loading was evaluated based on mass shifts equal to the mass of x linker-drugs relative to the reference masses, and weighted average DAR was calculated using deconvoluted MS peak intensities. For the expected drug-linker attached to each variant, see Example 3. Thiosuccinimide ring opening was defined as a deconvoluted MS peak with a mass shift of 18 Da relative to a drug-loaded deconvoluted MS peak. Mass assignments had a mass tolerance of +10 Da. Extent of drug loading and % of thiosuccinimide ring opening at each time point was graphed using GraphPad Prism software (GraphPad Software, San Diego, CA).

### Results

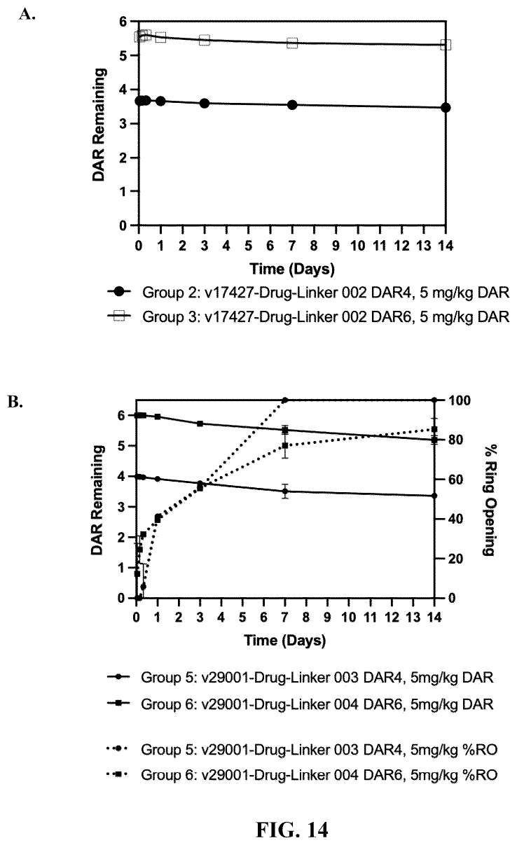

The results are summarized in FIG. 14A-B and Table 21.1. All four ADCs showed >84% DAR remaining after 21 days. v29001-Drug-Linker 003 and v29001-Drug-Linker 004 showed thiosuccinimide ring opening. For the v17427-Drug-Linker 002 ADCs, minimal drug-linker decomposition was observed at day 21 (observed as a loss of ˜582 Da from drug-loaded species). No linker drug decomposition was observed for v29001-Drug-Linker 003 and v29001-Drug-Linker 004 after 21 days.

TABLE 21.1
DAR Remaining and % Thiosuccinimide Ring Opening*

| | % DAR Remaining | % Thiosuccinimide Ring |
|---|---|---|
| ADC | (mean, SD) | Opening (mean, SD) |
| v17427-Drug-Linker 002 DAR4 | 93.0, 0.81 | Not applicable |
| v17427-Drug-Linker 002 DAR6 | 95.5, 1.73 | Not applicable |
| v29001-Drug-Linker 003 DAR4 | 84.2, 2.06 | 100, 0.00 |
| v29001-Drug-Linker 004 DAR6 | 84.0, 2.45 | 88.5, 0.58 |

*changes over 21 days

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

### Sequence Tables

TABLE A
Clone Numbers for Variants

| | |
|---|---|
| Variant | Clone Numbers |

| ID | H1 | H2 | L1 | L2 |
|---|---|---|---|---|
| 17606 | 11509 | 11509 | 11462 | 11462 |
| 17427 | 11466 | 11467 | 11462 | 11462 |
| 32634 | 23993 | 23993 | 23998 | 23998 |
| 33967 | 21292 | 11467 | 11462 | 11462 |
| 33968 | 17430 | 11467 | 11462 | 11462 |
| 33969 | 21233 | 11467 | 11462 | 11462 |
| 22761 | 11466 | 11467 | 17423 | 17423 |
| 22765 | 17430 | 17431 | 11462 | 11462 |
| 28983 | 11466 | 11467 | 21226 | 21226 |
| 28989 | 21233 | 21234 | 11462 | 11462 |
| 29001 | 21292 | 21293 | 11462 | 11462 |
| 35527 | 26704 | 26704 | 23998 | 23998 |
| 33970 | 21292 | 11467 | 21226 | 21226 |
| 33971 | 25169 | 21293 | 11462 | 11462 |
| 33974 | 25165 | 21293 | 11462 | 11462 |
| 33977 | 21233 | 11467 | 21226 | 21226 |
| 33979 | 25161 | 21234 | 11462 | 11462 |

TABLE B
Clone Sequences

| | | | SEQ |
|---|---|---|---|
| Clone | | | ID |
| ID | Region | Sequence | NO |
| 11462 | Full | DIVMTQSPDSLAVSLGERATINCKSSESVDSYANSFLHWYQQKPGQPP | 38 |
| and | | KLLIYRASTRESGVPDRFSGSGSGTDFTLTISSLQAEDVAVYYCQQSKE | |
| 23998 | | DPLTFGGGTKVEIKRTVAAPSVFIFPPSDEQLKSGTASVVCLLNNFYPR | |
| | | EAKVQWKVDNALQSGNSQESVTEQDSKDSTYSLSSTLTLSKADYEKH | |
| | | KVYACEVTHQGLSSPVTKSFNRGEC | |
| | Full | GACATCGTGATGACCCAGTCCCCTGATTCTCTGGCCGTGTCCCTGGG | 39 |
| | | CGAGAGGGCAACAATCAACTGCAAGAGCTCCGAGAGCGTGGACTC | |
| | | CTACGCCAATTCTTTCCTGCACTGGTATCAGCAGAAGCCAGGCCAG | |
| | | CCCCCTAAGCTGCTGATCTACAGGGCCTCTACCAGAGAGAGCGGAG | |
| | | TGCCAGACCGCTTCTCCGGCTCTGGCAGCGGCACAGACTTCACCCT | |
| | | GACAATCTCTAGCCTGCAGGCCGAGGACGTGGCCGTGTACTATTGT | |
| | | CAGCAGTCCAAGGAGGACCCCCTGACCTTTGGCGGAGGAACAAAG | |
| | | GTGGAGATCAAGCGGACCGTGGCGGCGCCCAGTGTCTTCATTTTTC | |
| | | CCCCTAGCGACGAACAGCTGAAGTCTGGGACAGCCAGTGTGGTCTG | |
| | | TCTGCTGAACAACTTCTACCCTAGAGAGGCTAAAGTGCAGTGGAAG | |
| | | GTCGATAACGCACTGCAGTCCGGAAATTCTCAGGAGAGTGTGACTG | |
| | | AACAGGACTCAAAAGATAGCACCTATTCCCTGTCAAGCACACTGAC | |
| | | TCTGAGCAAGGCCGACTACGAGAAGCATAAAGTGTATGCTTGTGAA | |
| | | GTCACCCACCAGGGGCTGAGTTCACCAGTCACAAAATCATTCAACA | |
| | | GAGGGGAGTGC | |
| | VL | DIVMTQSPDSLAVSLGERATINCKSSESVDSYANSFLHWYQQKPGQPP | 40 |
| | | KLLIYRASTRESGVPDRFSGSGSGTDFTLTISSLQAEDVAVYYCQQSKE | |
| | | DPLTFGGGTKVEIK | |
| 11466 | Full | QVQLVQSGAEVKKPGASVKVSCKASGYIFTAYTMHWVRQAPGQGLE | 41 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPI | |
| | | EKTISKAKGQPREPQVYVYPPSRDELTKNQVSLTCLVKGFYPSDIAVE | |
| | | WESNGQPENNYKTTPPVLDSDGSFALVSKLTVDKSRWQQGNVFSCSV | |
| | | MHEALHNHYTQKSLSLSPG | |
| | Full | CAGGTGCAGCTGGTGCAGAGCGGAGCCGAGGTGAAGAAGCCAGGG | 42 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCAGAACTGCTGGGAGGACCAAGCGTGTTCCTGTTTCCAC | |
| | | CCAAGCCTAAAGACACCCTGATGATTTCCCGGACTCCTGAGGTCAC | |
| | | CTGCGTGGTCGTGGACGTGTCTCACGAGGACCCCGAAGTCAAGTTC | |
| | | AACTGGTACGTGGATGGCGTCGAAGTGCATAATGCCAAGACCAAA | |
| | | CCCCGGGAGGAACAGTACAACTCTACCTATAGAGTCGTGAGTGTCC | |
| | | TGACAGTGCTGCACCAGGACTGGCTGAATGGGAAGGAGTATAAGT | |
| | | GTAAAGTGAGCAACAAAGCCCTGCCCGCCCCAATCGAAAAAACAA | |
| | | TCTCTAAAGCAAAAGGACAGCCTCGCGAACCACAGGTCTACGTCTA | |
| | | CCCCCCATCAAGAGATGAACTGACAAAAAATCAGGTCTCTCTGACA | |
| | | TGCCTGGTCAAAGGATTCTACCCTTCCGACATCGCCGTGGAGTGGG | |
| | | AAAGTAACGGCCAGCCCGAGAACAATTACAAGACCACACCCCCTG | |
| | | TCCTGGACTCTGATGGGAGTTTCGCTCTGGTGTCAAAGCTGACCGTC | |
| | | GATAAAAGCCGGTGGCAGCAGGGCAATGTGTTTAGCTGCTCCGTCA | |
| | | TGCACGAAGCCCTGCACAATCACTACACACAGAAGTCCCTGAGCCT | |
| | | GAGCCCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 11467 | Full | QVQLVQSGAEVKKPGASVKVSCKASGYIFTAYTMHWVRQAPGQGLE | 44 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPI | |
| | | EKTISKAKGQPREPQVYVLPPSRDELTKNQVSLLCLVKGFYPSDIAVE | |
| | | WESNGQPENNYLTWPPVLDSDGSFFLYSKLTVDKSRWQQGNVFSCSV | |
| | | MHEALHNHYTQKSLSLSPG | |
| | Full | CAGGTGCAGCTGGTGCAGAGCGGAGCCGAGGTGAAGAAGCCAGGG | 45 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCAGAACTGCTGGGAGGACCAAGCGTGTTCCTGTTTCCAC | |
| | | CCAAGCCTAAAGACACCCTGATGATTTCCCGGACTCCTGAGGTCAC | |
| | | CTGCGTGGTCGTGGACGTGTCTCACGAGGACCCCGAAGTCAAGTTC | |
| | | AACTGGTACGTGGATGGCGTCGAAGTGCATAATGCCAAGACCAAA | |
| | | CCCCGGGAGGAACAGTACAACTCTACCTATAGAGTCGTGAGTGTCC | |
| | | TGACAGTGCTGCACCAGGACTGGCTGAATGGGAAGGAGTATAAGT | |
| | | GTAAAGTGAGCAACAAAGCCCTGCCCGCCCCAATCGAAAAAACAA | |
| | | TCTCTAAAGCAAAAGGACAGCCTCGCGAACCACAGGTCTACGTGCT | |
| | | GCCCCCTAGCCGCGACGAACTGACTAAAAATCAGGTCTCTCTGCTG | |
| | | TGTCTGGTCAAAGGATTCTACCCTTCCGACATCGCCGTGGAGTGGG | |
| | | AAAGTAACGGCCAGCCCGAGAACAATTACCTGACCTGGCCCCCTGT | |
| | | GCTGGACTCTGATGGGAGTTTCTTTCTGTATTCAAAGCTGACAGTCG | |
| | | ATAAAAGCCGGTGGCAGCAGGGCAATGTGTTCAGCTGCTCCGTCAT | |
| | | GCACGAAGCACTGCACAACCATTACACTCAGAAGTCCCTGTCCCTG | |
| | | TCACCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 11509 | Full | QVQLVQSGAEVKKPGASVKVSCKASGYIFTAYTMHWVRQAPGQGLE | 46 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKRVEPKSCDCHCPPCPAPELLGGPS | |
| | | VFLFPPKPKDTLMISRTPEVTCVVVDVSHEDPEVKFNWYVDGVEVHN | |
| | | AKTKPREEQYNSTYRVVSVLTVLHQDWLNGKEYKCKVSNKALPAPIE | |
| | | KTISKAKGQPREPQVYTLPPSREEMTKNQVSLTCLVKGFYPSDIAVEW | |
| | | ESNGQPENNYKTTPPVLDSDGSFFLYSKLTVDKSRWQQGNVFSCSVM | |
| | | HEALHNHYTQKSLSLSPG | |
| | Full | CAGGTCCAGCTGGTCCAGAGCGGAGCAGAGGTGAAGAAACCCGGA | 47 |
| | | GCAAGCGTCAAGGTCTCATGCAAAGCAAGCGGCTACATTTTCACCG | |
| | | CCTACACAATGCACTGGGTGCGGCAGGCCCCTGGACAGGGCCTGGA | |
| | | GTGGATGGGCTGGATCAAGCCAAACAATGGCCTGGCCAACTACGCC | |
| | | CAGAAGTTCCAGGGCAGGGTGACCATGACACGCGACACCTCTATCA | |
| | | GCACAGCCTATATGGAGCTGTCTCGGCTGAGAAGCGACGATACCGC | |
| | | CGTGTACTATTGCGCCAGGTCTGAGATCACCACAGAGTTCGATTAC | |
| | | TGGGGCCAGGGCACCCTGGTGACAGTGAGCTCCGCTAGCACAAAG | |
| | | GGACCAAGCGTGTTCCCACTGGCCCCCTCTAGCAAGTCCACCTCTG | |
| | | GAGGAACAGCCGCCCTGGGCTGTCTGGTGAAGGACTATTTCCCCGA | |
| | | GCCTGTGACCGTGAGCTGGAACAGCGGGGCCCTGACCAGCGGAGT | |
| | | GCACACATTTCCCGCCGTGCTGCAGTCCTCTGGCCTGTACTCCCTGA | |
| | | GCTCCGTGGTGACCGTGCCCTCTAGCTCCCTGGGCACCCAGACATA | |
| | | TATCTGCAACGTGAATCACAAGCCTAGCAATACTAAGGTCGACAAA | |
| | | AGAGTGGAGCCCAAGTCCTGCGATTGTCATTGCCCCCCCTGCCCGG | |
| | | CGCCAGAGCTGCTGGGCGGACCATCCGTGTTCCTGTTTCCACCCAA | |
| | | GCCTAAGGACACCCTGATGATCTCCAGGACCCCAGAGGTGACATGC | |
| | | GTGGTGGTGGACGTGTCTCACGAGGACCCCGAGGTGAAGTTTAACT | |
| | | GGTACGTGGATGGCGTGGAGGTGCACAATGCCAAGACCAAGCCCA | |
| | | GGGAGGAGCAGTACAACAGCACCTATCGCGTGGTGTCCGTGCTGAC | |
| | | AGTGCTGCACCAGGACTGGCTGAACGGCAAGGAGTATAAGTGCAA | |
| | | GGTGAGCAATAAGGCCCTGCCCGCCCCTATCGAGAAGACCATCTCC | |
| | | AAGGCCAAGGGCCAGCCTCGCGAACCACAGGTGTACACACTGCCTC | |
| | | CAAGCAGAGAGGAGATGACCAAGAACCAGGTGTCCCTGACATGTC | |
| | | TGGTGAAGGGCTTCTATCCTTCCGACATCGCCGTGGAGTGGGAGTC | |
| | | TAATGGCCAGCCAGAGAACAATTACAAGACCACACCCCCTGTGCTG | |
| | | GACTCTGATGGCAGCTTCTTTCTGTATTCTAAGCTGACCGTGGATAA | |
| | | GAGCAGATGGCAGCAGGGCAACGTGTTCAGCTGTTCTGTGATGCAC | |
| | | GAAGCACTGCACAATCACTACACTCAGAAAAGCCTGAGCTTAAGCC | |
| | | CTGGG | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 17423 | Full | DIVMTQSPDSLAVSLGERATINCKSSESVDSYANSFLHWYQQKPGQPP | 48 |
| | | KLLIYRASTRESGVPDRFSGSGSGTDFTLTISSLQAEDVAVYYCQQSKE | |
| | | DPLTFGGGTKVEIKRTVAAPSVFIFPPSDEQLKCSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGECESSCDVKLV | |
| | Full | GACATCGTGATGACCCAGTCCCCTGATTCTCTGGCCGTGTCCCTGGG | 49 |
| | | CGAGAGGGCAACAATCAACTGCAAGAGCTCCGAGAGCGTGGACTC | |
| | | CTACGCCAATTCTTTCCTGCACTGGTATCAGCAGAAGCCAGGCCAG | |
| | | CCCCCTAAGCTGCTGATCTACAGGGCCTCTACCAGAGAGAGCGGAG | |
| | | TGCCAGACCGCTTCTCCGGCTCTGGCAGCGGCACAGACTTCACCCT | |
| | | GACAATCTCTAGCCTGCAGGCCGAGGACGTGGCCGTGTACTATTGT | |
| | | CAGCAGTCCAAGGAGGACCCCCTGACCTTTGGCGGAGGAACAAAG | |
| | | GTGGAGATCAAGCGGACCGTGGCGGCGCCCAGCGTGTTCATCTTTC | |
| | | CCCCTAGCGACGAGCAGCTGAAGTGCAGCGGAACCGCCTCCGTGGT | |
| | | GTGCCTGCTGAACAATTTCTACCCACGGGAGGCCAAGGTGCAGTGG | |
| | | AAGGTGGATAACGCCCTGCAGAGCGGCAATTCCCAGGAGTCTGTGA | |
| | | CCGAGCAGGACAGCAAGGATTCCACATATTCTCTGAGCTCCACCCT | |
| | | GACACTGTCCAAGGCCGACTACGAGAAGCACAAGGTGTATGCCTGC | |
| | | GAGGTGACCCACCAGGGCCTGTCTAGCCCCGTGACAAAGTCTTTTA | |
| | | ACAGAGGCGAGTGCGAGTCCTCTTGTGATGTGAAGCTGGTG | |
| | VL | DIVMTQSPDSLAVSLGERATINCKSSESVDSYANSFLHWYQQKPGQPP | 40 |
| | | KLLIYRASTRESGVPDRFSGSGSGTDFTLTISSLQAEDVAVYYCQQSKE | |
| | | DPLTFGGGTKVEIK | |
| 17430 | Full | QVQLVQSGAEVKKPGASVKVSCKASGYIFTAYTMHWVRQAPGQGLE | 50 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | CPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPA | |
| | | PIEKTISKAKGQPREPQVYVYPPSRDELTKNQVSLTCLVKGFYPSDIAV | |
| | | EWESNGQPENNYKTTPPVLDSDGSFALVSKLTVDKSRWQQGNVFSCS | |
| | | VMHEALHNHYTQKSLSLSPG | |
| | Full | CAGGTGCAGCTGGTGCAGAGCGGAGCCGAGGTGAAGAAGCCAGGG | 51 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCCGAGCTGCTGGGCGGCTGCCCTAGCGTGTTCCTGTTTCC | |
| | | CCCTAAGCCAAAGGACACCCTGATGATCTCTCGGACCCCCGAGGTG | |
| | | ACATGCGTGGTGGTGGACGTGAGCCACGAGGACCCCGAGGTGAAG | |
| | | TTCAACTGGTACGTGGATGGCGTGGAGGTGCACAATGCCAAGACAA | |
| | | AGCCCCGGGAGGAGCAGTACAACAGCACCTATAGAGTGGTGTCCG | |
| | | TGCTGACAGTGCTGCACCAGGACTGGCTGAACGGCAAGGAGTATA | |
| | | AGTGCAAGGTGTCCAATAAGGCCCTGCCAGCCCCCATCGAGAAGAC | |
| | | CATCTCTAAGGCCAAGGGCCAGCCTCGCGAACCACAGGTCTACGTC | |
| | | TACCCCCCATCAAGAGATGAACTGACAAAAAATCAGGTCTCTCTGA | |
| | | CATGCCTGGTCAAAGGATTCTACCCTTCCGACATCGCCGTGGAGTG | |
| | | GGAAAGTAACGGCCAGCCCGAGAACAATTACAAGACCACACCCCC | |
| | | TGTCCTGGACTCTGATGGGAGTTTCGCTCTGGTGTCAAAGCTGACC | |
| | | GTCGATAAAAGCCGGTGGCAGCAGGGCAATGTGTTTAGCTGCTCCG | |
| | | TCATGCACGAAGCCCTGCACAATCACTACACACAGAAGTCCCTGAG | |
| | | CCTGAGCCCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 17431 | Full | QVQLVQSGAEVKKPGASVKVSCKASGYIFTAYTMHWVRQAPGQGLE | 52 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | CPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPA | |
| | | PIEKTISKAKGQPREPQVYVLPPSRDELTKNQVSLLCLVKGFYPSDIAVE | |
| | | WESNGQPENNYLTWPPVLDSDGSFFLYSKLTVDKSRWQQGNVFSCSV | |
| | | MHEALHNHYTQKSLSLSPG | |
| | Full | CAGGTGCAGCTGGTGCAGAGCGGAGCCGAGGTGAAGAAGCCAGGG | 53 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCCGAGCTGCTGGGCGGCTGCCCTAGCGTGTTCCTGTTTCC | |
| | | CCCTAAGCCAAAGGACACCCTGATGATCTCTCGGACCCCCGAGGTG | |
| | | ACATGCGTGGTGGTGGACGTGAGCCACGAGGACCCCGAGGTGAAG | |
| | | TTCAACTGGTACGTGGATGGCGTGGAGGTGCACAATGCCAAGACAA | |
| | | AGCCCCGGGAGGAGCAGTACAACAGCACCTATAGAGTGGTGTCCG | |
| | | TGCTGACAGTGCTGCACCAGGACTGGCTGAACGGCAAGGAGTATA | |
| | | AGTGCAAGGTGTCCAATAAGGCCCTGCCAGCCCCCATCGAGAAGAC | |
| | | CATCTCTAAGGCCAAGGGCCAGCCTCGCGAACCACAGGTCTACGTG | |
| | | CTGCCCCCTAGCCGCGACGAACTGACTAAAAATCAGGTCTCTCTGC | |
| | | TGTGTCTGGTCAAAGGATTCTACCCTTCCGACATCGCCGTGGAGTG | |
| | | GGAAAGTAACGGCCAGCCCGAGAACAATTACCTGACCTGGCCCCCT | |
| | | GTGCTGGACTCTGATGGGAGTTTCTTTCTGTATTCAAAGCTGACAGT | |
| | | CGATAAAAGCCGGTGGCAGCAGGGCAATGTGTTCAGCTGCTCCGTC | |
| | | ATGCACGAAGCACTGCACAACCATTACACTCAGAAGTCCCTGTCCC | |
| | | TGTCACCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 21226 | Full | DIVMTQSPDSLAVSLGERATINCKSSESVDSYANSFLHWYQQKPCGQP | 54 |
| | | PKLLIYRASTRESGVPDRFSGSGSGTDFTLTISSLQAEDVAVYYCQQSK | |
| | | EDPLTFGGGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| | Full | GACATCGTGATGACCCAGTCCCCTGATTCTCTGGCCGTGTCCCTGGG | 55 |
| | | CGAGAGGGCAACAATCAACTGCAAGAGCTCCGAGAGCGTGGACTC | |
| | | CTACGCCAATTCTTTCCTGCACTGGTATCAGCAGAAGCCATGTGGC | |
| | | CAGCCCCCTAAGCTGCTGATCTACAGGGCCTCTACCAGAGAGAGCG | |
| | | GAGTGCCAGACAGGTTCTCCGGCTCTGGCAGCGGCACAGACTTCAC | |
| | | CCTGACAATCTCTAGCCTGCAGGCCGAGGACGTGGCCGTGTACTAT | |
| | | TGCCAGCAGTCCAAGGAGGACCCCCTGACCTTTGGCGGAGGAACA | |
| | | AAGGTGGAGATCAAGCGCACCGTGGCGGCGCCCAGTGTCTTCATTT | |
| | | TTCCCCCTAGCGACGAACAGCTGAAGTCTGGGACAGCCAGTGTGGT | |
| | | CTGTCTGCTGAACAACTTCTACCCTAGAGAGGCTAAAGTGCAGTGG | |
| | | AAGGTCGATAACGCACTGCAGTCCGGAAATTCTCAGGAGAGTGTGA | |
| | | CTGAACAGGACTCAAAAGATAGCACCTATTCCCTGTCAAGCACACT | |
| | | GACTCTGAGCAAGGCCGACTACGAGAAGCATAAAGTGTATGCTTGT | |
| | | GAAGTCACCCACCAGGGGCTGAGTTCACCAGTCACAAAATCATTCA | |
| | | ACAGAGGGGAGTGC | |
| | VL | DIVMTQSPDSLAVSLGERATINCKSSESVDSYANSFLHWYQQKPCGQP | 56 |
| | | PKLLIYRASTRESGVPDRFSGSGSGTDFTLTISSLQAEDVAVYYCQQSK | |
| | | EDPLTFGGGTKVEIK | |
| 21233 | Full | QVQLVQSGACEVKKPGASVKVSCKASGYIFTAYTMHWVRQAPGQGL | 57 |
| | | EWMGWIKPNNGLANYAQKFQGRVTMTRDTSISTAYMELSRLRSDDT | |
| | | AVYYCARSEITTEFDYWGQGTLVTVSSASTKGPSVFPLAPSSKSTSGGT | |
| | | AALGCLVKDYFPEPVTVSWNSGALTSGVHTFPAVLQSSGLYSLSSVVT | |
| | | VPSSSLGTQTYICNVNHKPSNTKVDKKVEPKSCDKTHTCPPCPAPELLG | |
| | | GPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPA | |
| | | PIEKTISKAKGQPREPQVYVYPPSRDELTKNQVSLTCLVKGFYPSDIAV | |
| | | EWESNGQPENNYKTTPPVLDSDGSFALVSKLTVDKSRWQQGNVFSCS | |
| | | VMHEALHNHYTQKSLSLSPG | |
| | Full | CAGGTGCAGCTGGTGCAGAGCGGAGCCTGCGAGGTGAAGAAGCCA | 58 |
| | | GGGGCCAGCGTGAAGGTGTCTTGTAAGGCCAGCGGCTACATCTTCA | |
| | | CCGCCTATACAATGCACTGGGTGCGGCAGGCCCCCGGACAGGGACT | |
| | | GGAGTGGATGGGCTGGATCAAGCCTAACAATGGCCTGGCCAACTAC | |
| | | GCCCAGAAGTTTCAGGGCAGGGTGACCATGACACGCGACACCTCTA | |
| | | TCAGCACAGCCTATATGGAGCTGTCCCGGCTGAGATCTGACGATAC | |
| | | CGCCGTGTACTATTGCGCCAGGTCCGAGATCACCACAGAGTTCGAT | |
| | | TACTGGGGCCAGGGCACCCTGGTGACAGTGAGCTCCGCTAGCACTA | |
| | | AGGGGCCTTCCGTGTTTCCACTGGCTCCCTCTAGTAAATCCACCTCT | |
| | | GGAGGCACAGCTGCACTGGGATGTCTGGTGAAGGATTACTTCCCTG | |
| | | AACCAGTCACAGTGAGTTGGAACTCAGGGGCTCTGACAAGTGGAGT | |
| | | CCATACTTTTCCCGCAGTGCTGCAGTCAAGCGGACTGTACTCCCTGT | |
| | | CCTCTGTGGTCACCGTGCCTAGTTCAAGCCTGGGCACCCAGACATA | |
| | | TATCTGCAACGTGAATCACAAGCCATCAAATACAAAAGTCGACAAG | |
| | | AAAGTGGAGCCCAAGAGCTGTGATAAAACTCATACCTGCCCACCTT | |
| | | GTCCGGCGCCAGAACTGCTGGGAGGACCAAGCGTGTTCCTGTTTCC | |
| | | ACCCAAGCCTAAAGACACCCTGATGATTTCCCGGACTCCTGAGGTC | |
| | | ACCTGCGTGGTCGTGGACGTGTCTCACGAGGACCCCGAAGTCAAGT | |
| | | TCAACTGGTACGTGGATGGCGTCGAAGTGCATAATGCCAAGACCAA | |
| | | ACCCCGGGAGGAACAGTACAACTCTACCTATAGAGTCGTGAGTGTC | |
| | | CTGACAGTGCTGCACCAGGACTGGCTGAATGGGAAGGAGTATAAG | |
| | | TGTAAAGTGAGCAACAAAGCCCTGCCCGCCCCAATCGAAAAAACA | |
| | | ATCTCTAAAGCAAAAGGACAGCCTCGCGAACCACAGGTCTACGTCT | |
| | | ACCCCCCATCAAGAGATGAACTGACAAAAAATCAGGTCTCTCTGAC | |
| | | ATGCCTGGTCAAAGGATTCTACCCTTCCGACATCGCCGTGGAGTGG | |
| | | GAAAGTAACGGCCAGCCCGAGAACAATTACAAGACCACACCCCCT | |
| | | GTCCTGGACTCTGATGGGAGTTTCGCTCTGGTGTCAAAGCTGACCG | |
| | | TCGATAAAAGCCGGTGGCAGCAGGGCAATGTGTTTAGCTGCTCCGT | |
| | | CATGCACGAAGCCCTGCACAATCACTACACACAGAAGTCCCTGAGC | |
| | | CTGAGCCCTGGC | |
| | VH | QVQLVQSGACEVKKPGASVKVSCKASGYIFTAYTMHWVRQAPGQGL | 59 |
| | | EWMGWIKPNNGLANYAQKFQGRVTMTRDTSISTAYMELSRLRSDDT | |
| | | AVYYCARSEITTEFDYWGQGTLVTVSS | |
| 21234 | Full | QVQLVQSGACEVKKPGASVKVSCKASGYIFTAYTMHWVRQAPGQGL | 60 |
| | | EWMGWIKPNNGLANYAQKFQGRVTMTRDTSISTAYMELSRLRSDDT | |
| | | AVYYCARSEITTEFDYWGQGTLVTVSSASTKGPSVFPLAPSSKSTSGGT | |
| | | AALGCLVKDYFPEPVTVSWNSGALTSGVHTFPAVLQSSGLYSLSSVVT | |
| | | VPSSSLGTQTYICNVNHKPSNTKVDKKVEPKSCDKTHTCPPCPAPELLG | |
| | | GPSVFLFPPKPKDTLMISRTPEVTCVVVDVSHEDPEVKFNWYVDGVEV | |
| | | HNAKTKPREEQYNSTYRVVSVLTVLHQDWLNGKEYKCKVSNKALPA | |
| | | PIEKTISKAKGQPREPQVYVLPPSRDELTKNQVSLLCLVKGFYPSDIAVE | |
| | | WESNGQPENNYLTWPPVLDSDGSFFLYSKLTVDKSRWQQGNVFSCSV | |
| | | MHEALHNHYTQKSLSLSPG | |
| | Full | CAGGTGCAGCTGGTGCAGAGCGGAGCCTGCGAGGTGAAGAAGCCA | 61 |
| | | GGGGCCAGCGTGAAGGTGTCTTGTAAGGCCAGCGGCTACATCTTCA | |
| | | CCGCCTATACAATGCACTGGGTGCGGCAGGCCCCCGGACAGGGACT | |
| | | GGAGTGGATGGGCTGGATCAAGCCTAACAATGGCCTGGCCAACTAC | |
| | | GCCCAGAAGTTTCAGGGCAGGGTGACCATGACACGCGACACCTCTA | |
| | | TCAGCACAGCCTATATGGAGCTGTCCCGGCTGAGATCTGACGATAC | |
| | | CGCCGTGTACTATTGCGCCAGGTCCGAGATCACCACAGAGTTCGAT | |
| | | TACTGGGGCCAGGGCACCCTGGTGACAGTGAGCTCCGCTAGCACTA | |
| | | AGGGGCCTTCCGTGTTTCCACTGGCTCCCTCTAGTAAATCCACCTCT | |
| | | GGAGGCACAGCTGCACTGGGATGTCTGGTGAAGGATTACTTCCCTG | |
| | | AACCAGTCACAGTGAGTTGGAACTCAGGGGCTCTGACAAGTGGAGT | |
| | | CCATACTTTTCCCGCAGTGCTGCAGTCAAGCGGACTGTACTCCCTGT | |
| | | CCTCTGTGGTCACCGTGCCTAGTTCAAGCCTGGGCACCCAGACATA | |
| | | TATCTGCAACGTGAATCACAAGCCATCAAATACAAAAGTCGACAAG | |
| | | AAAGTGGAGCCCAAGAGCTGTGATAAAACTCATACCTGCCCACCTT | |
| | | GTCCGGCGCCAGAACTGCTGGGAGGACCAAGCGTGTTCCTGTTTCC | |
| | | ACCCAAGCCTAAAGACACCCTGATGATTTCCCGGACTCCTGAGGTC | |
| | | ACCTGCGTGGTCGTGGACGTGTCTCACGAGGACCCCGAAGTCAAGT | |
| | | TCAACTGGTACGTGGATGGCGTCGAAGTGCATAATGCCAAGACCAA | |
| | | ACCCCGGGAGGAACAGTACAACTCTACCTATAGAGTCGTGAGTGTC | |
| | | CTGACAGTGCTGCACCAGGACTGGCTGAATGGGAAGGAGTATAAG | |
| | | TGTAAAGTGAGCAACAAAGCCCTGCCCGCCCCAATCGAAAAAACA | |
| | | ATCTCTAAAGCAAAAGGACAGCCTCGCGAACCACAGGTCTACGTGC | |
| | | TGCCCCCTAGCCGCGACGAACTGACTAAAAATCAGGTCTCTCTGCT | |
| | | GTGTCTGGTCAAAGGATTCTACCCTTCCGACATCGCCGTGGAGTGG | |
| | | GAAAGTAACGGCCAGCCCGAGAACAATTACCTGACCTGGCCCCCTG | |
| | | TGCTGGACTCTGATGGGAGTTTCTTTCTGTATTCAAAGCTGACAGTC | |
| | | GATAAAAGCCGGTGGCAGCAGGGCAATGTGTTCAGCTGCTCCGTCA | |
| | | TGCACGAAGCACTGCACAACCATTACACTCAGAAGTCCCTGTCCCT | |
| | | GTCACCTGGC | |
| | VH | QVQLVQSGACEVKKPGASVKVSCKASGYIFTAYTMHWVRQAPGQGL | 59 |
| | | EWMGWIKPNNGLANYAQKFQGRVTMTRDTSISTAYMELSRLRSDDT | |
| | | AVYYCARSEITTEFDYWGQGTLVTVSS | |
| 21292 | Full | QVQLVQSGAEVKKPGASVKVSCKASGYIFTAYTMHWVRQAPGQGLE | 62 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTCYRVVSVLTVLHQDWLNGKEYKCKVSNKALPA | |
| | | PIEKTISKAKGQPREPQVYVYPPSRDELTKNQVSLTCLVKGFYPSDIAV | |
| | | EWESNGQPENNYKTTPPVLDSDGSFALVSKLTVDKSRWQQGNVFSCS | |
| | | VMHEALHNHYTQKSLSLSPG | |
| | Full | CAGGTGCAGCTGGTGCAGAGCGGAGCCGAGGTGAAGAAGCCAGGG | 63 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCCGAGCTGCTGGGCGGCCCTAGCGTGTTCCTGTTTCCCCC | |
| | | TAAGCCAAAGGACACCCTGATGATCTCTCGGACCCCCGAGGTGACA | |
| | | TGCGTGGTGGTGGACGTGAGCCACGAGGACCCCGAGGTGAAGTTC | |
| | | AACTGGTACGTGGATGGCGTGGAGGTGCACAATGCCAAGACAAAG | |
| | | CCCCGGGAGGAGCAGTACAACAGCACCTGCTATAGAGTGGTGTCCG | |
| | | TGCTGACAGTGCTGCACCAGGACTGGCTGAACGGCAAGGAGTATA | |
| | | AGTGTAAGGTGTCCAATAAGGCCCTGCCAGCCCCCATCGAGAAGAC | |
| | | CATCTCTAAGGCCAAGGGCCAGCCTCGCGAACCACAGGTCTACGTC | |
| | | TACCCCCCATCAAGAGATGAACTGACAAAAAATCAGGTCTCTCTGA | |
| | | CATGCCTGGTCAAAGGATTCTACCCTTCCGACATCGCCGTGGAGTG | |
| | | GGAAAGTAACGGCCAGCCCGAGAACAATTACAAGACCACACCCCC | |
| | | TGTCCTGGACTCTGATGGGAGTTTCGCTCTGGTGTCAAAGCTGACC | |
| | | GTCGATAAAAGCCGGTGGCAGCAGGGCAATGTGTTTAGCTGCTCCG | |
| | | TCATGCACGAAGCCCTGCACAATCACTACACACAGAAGTCCCTGAG | |
| | | CCTGAGCCCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 21293 | Full | QVQLVQSGAEVKKPGASVKVSCKASGYIFTAYTMHWVRQAPGQGLE | 64 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | Full | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTCYRVVSVLTVLHQDWLNGKEYKCKVSNKALPA | |
| | | PIEKTISKAKGQPREPQVYVLPPSRDELTKNQVSLLCLVKGFYPSDIAVE | |
| | | WESNGQPENNYLTWPPVLDSDGSFFLYSKLTVDKSRWQQGNVFSCSV | |
| | | MHEALHNHYTQKSLSLSPG | |
| | | CAGGTGCAGCTGGTGCAGAGCGGAGCCGAGGTGAAGAAGCCAGGG | 65 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCCGAGCTGCTGGGCGGCCCTAGCGTGTTCCTGTTTCCCCC | |
| | | TAAGCCAAAGGACACCCTGATGATCTCTCGGACCCCCGAGGTGACA | |
| | | TGCGTGGTGGTGGACGTGAGCCACGAGGACCCCGAGGTGAAGTTC | |
| | | AACTGGTACGTGGATGGCGTGGAGGTGCACAATGCCAAGACAAAG | |
| | | CCCCGGGAGGAGCAGTACAACAGCACCTGCTATAGAGTGGTGTCCG | |
| | | TGCTGACAGTGCTGCACCAGGACTGGCTGAACGGCAAGGAGTATA | |
| | | AGTGTAAGGTGTCCAATAAGGCCCTGCCAGCCCCCATCGAGAAGAC | |
| | | CATCTCTAAGGCCAAGGGCCAGCCTCGCGAACCACAGGTCTACGTG | |
| | | CTGCCCCCTAGCCGCGACGAACTGACTAAAAATCAGGTCTCTCTGC | |
| | | TGTGTCTGGTCAAAGGATTCTACCCTTCCGACATCGCCGTGGAGTG | |
| | | GGAAAGTAACGGCCAGCCCGAGAACAATTACCTGACCTGGCCCCCT | |
| | | GTGCTGGACTCTGATGGGAGTTTCTTTCTGTATTCAAAGCTGACAGT | |
| | | CGATAAAAGCCGGTGGCAGCAGGGCAATGTGTTCAGCTGCTCCGTC | |
| | | ATGCACGAAGCACTGCACAACCATTACACTCAGAAGTCCCTGTCCC | |
| | | TGTCACCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 23993 | Full | QVQLVQSGAEVKKPGASVKVSCKASGYIFTAYTMHWVRQAPGQGLE | 66 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTYRVVSVLTVLHQDWLNGKEYKCKVSNKALPAPI | |
| | | EKTISKAKGQPREPQVYTLPPSRDELTKNQVSLTCLVKGFYPSDIAVEW | |
| | Full | ESNGQPENNYKTTPPVLDSDGSFFLYSKLTVDKSRWQQGNVFSCSVM | |
| | | HEALHNHYTQKSLSLSPG | |
| | | CAGGTGCAGCTGGTGCAGAGCGGAGCCGAGGTGAAGAAGCCAGGG | 67 |
| | | GCCAGCGTGAAGGTGTCTTGCAAGGCCAGCGGCTACATCTTCACCG | |
| | | CCTATACAATGCACTGGGTGAGGCAGGCCCCCGGACAGGGACTGG | |
| | | AGTGGATGGGCTGGATCAAGCCTAACAATGGCCTGGCCAACTACGC | |
| | | CCAGAAGTTTCAGGGCAGGGTGACCATGACACGCGACACCTCTATC | |
| | | AGCACAGCCTATATGGAGCTGTCCCGGCTGAGATCTGACGATACAG | |
| | | CCGTGTACTATTGTGCCAGATCCGAGATCACCACAGAGTTTGATTA | |
| | | CTGGGGCCAGGGCACCCTGGTGACAGTGAGCTCCGCTAGCACTAAG | |
| | | GGGCCTTCCGTGTTTCCACTGGCTCCCTCTAGTAAATCCACCTCTGG | |
| | | AGGCACAGCTGCACTGGGATGTCTGGTGAAGGATTACTTCCCTGAA | |
| | | CCAGTCACAGTGAGTTGGAACTCAGGGGCTCTGACAAGTGGAGTCC | |
| | | ATACTTTTCCCGCAGTGCTGCAGTCAAGCGGACTGTACTCCCTGTCC | |
| | | TCTGTGGTCACCGTGCCTAGTTCAAGCCTGGGCACCCAGACATATA | |
| | | TCTGCAACGTGAATCACAAGCCATCAAATACAAAAGTCGACAAGA | |
| | | AAGTGGAGCCCAAGAGCTGTGATAAAACTCATACCTGCCCACCTTG | |
| | | TCCGGCGCCAGAACTGCTGGGAGGACCAAGCGTGTTCCTGTTTCCA | |
| | | CCCAAGCCTAAAGACACCCTGATGATTTCCCGGACTCCTGAGGTCA | |
| | | CCTGCGTGGTCGTGGACGTGTCTCACGAGGACCCCGAAGTCAAGTT | |
| | | CAACTGGTACGTGGATGGCGTCGAAGTGCATAATGCCAAGACCAA | |
| | | ACCCCGGGAGGAACAGTACAACTCTACCTATAGAGTCGTGAGTGTC | |
| | | CTGACAGTGCTGCACCAGGACTGGCTGAATGGGAAGGAGTATAAG | |
| | | TGTAAAGTGAGCAACAAAGCCCTGCCCGCCCCAATCGAAAAAACA | |
| | | ATCTCTAAAGCAAAAGGACAGCCTCGCGAACCACAGGTGTACACTC | |
| | | TGCCTCCATCTCGGGACGAGCTGACTAAGAACCAGGTCAGTCTGAC | |
| | | CTGTCTGGTGAAAGGATTCTATCCCAGCGATATCGCTGTGGAGTGG | |
| | | GAATCCAATGGCCAGCCTGAGAACAATTACAAGACCACACCCCCTG | |
| | | TGCTGGACTCTGATGGCAGTTTCTTTCTGTATAGTAAGCTGACCGTC | |
| | | GATAAATCACGATGGCAGCAGGGGAACGTGTTCAGCTGTTCAGTGA | |
| | | TGCACGAAGCCCTGCACAACCATTACACCCAGAAGAGCCTGAGCCT | |
| | | GTCTCCCGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 25161 | Full | QVQLVQSGACEVKKPGASVKVSCKASGYIFTAYTMHWVRQAPGQGL | 68 |
| | | EWMGWIKPNNGLANYAQKFQGRVTMTRDTSISTAYMELSRLRSDDT | |
| | | AVYYCARSEITTEFDYWGQGTLVTVSSASTKGPSVFPLAPSSKSTSGGT | |
| | | AALGCLVKDYFPEPVTVSWNSGALTSGVHTFPAVLQSSGLYSLSSVVT | |
| | | VPSSSLGTQTYICNVNHKPSNTKVDKKVEPKSCDKTHTCPPCPAPELLG | |
| | | GCPSVFLFPPKPKDTLMISRTPEVTCVVVDVSHEDPEVKFNWYVDGVE | |
| | | VHNAKTKPREEQYNSTYRVVSVLTVLHQDWLNGKEYKCKVSNKALP | |
| | | APIEKTISKAKGQPREPQVYVYPPSRDELTKNQVSLTCLVKGFYPSDIA | |
| | | VEWESNGQPENNYKTTPPVLDSDGSFALVSKLTVDKSRWQQGNVFSC | |
| | | SVMHEALHNHYTQKSLSLSPG | |
| | Full | CAGGTGCAGCTGGTGCAGAGCGGAGCCTGCGAGGTGAAGAAGCCA | 69 |
| | | GGGGCCAGCGTGAAGGTGTCTTGCAAGGCCAGCGGCTACATCTTCA | |
| | | CCGCCTATACAATGCACTGGGTGCGGCAGGCCCCCGGACAGGGCCT | |
| | | GGAGTGGATGGGCTGGATCAAGCCTAACAATGGCCTGGCCAACTAC | |
| | | GCCCAGAAGTTTCAGGGCAGGGTGACCATGACACGCGACACCTCTA | |
| | | TCAGCACAGCCTATATGGAGCTGTCCCGGCTGAGATCTGACGATAC | |
| | | CGCCGTGTACTATTGTGCCAGGTCCGAGATCACCACAGAGTTCGAT | |
| | | TACTGGGGCCAGGGCACCCTGGTGACAGTGAGCTCCGCTAGCACTA | |
| | | AGGGGCCTTCCGTGTTTCCACTGGCTCCCTCTAGTAAATCCACCTCT | |
| | | GGAGGCACAGCTGCACTGGGATGTCTGGTGAAGGATTACTTCCCTG | |
| | | AACCAGTCACAGTGAGTTGGAACTCAGGGGCTCTGACAAGTGGAGT | |
| | | CCATACTTTTCCCGCAGTGCTGCAGTCAAGCGGACTGTACTCCCTGT | |
| | | CCTCTGTGGTCACCGTGCCTAGTTCAAGCCTGGGCACCCAGACATA | |
| | | TATCTGCAACGTGAATCACAAGCCATCAAATACAAAAGTCGACAAG | |
| | | AAAGTGGAGCCCAAGAGCTGTGATAAAACTCATACCTGCCCACCTT | |
| | | GTCCGGCGCCCGAGCTGCTGGGCGGCTGCCCTAGCGTGTTCCTGTTT | |
| | | CCCCCTAAGCCAAAGGACACCCTGATGATCTCTCGGACCCCCGAGG | |
| | | TGACATGCGTGGTGGTGGACGTGAGCCACGAGGACCCCGAGGTGA | |
| | | AGTTCAACTGGTACGTGGATGGCGTGGAGGTGCACAATGCCAAGAC | |
| | | AAAGCCCCGGGAGGAGCAGTACAACAGCACCTATAGAGTGGTGTC | |
| | | CGTGCTGACAGTGCTGCACCAGGACTGGCTGAACGGCAAGGAGTAT | |
| | | AAGTGCAAGGTGTCCAATAAGGCCCTGCCAGCCCCCATCGAGAAG | |
| | | ACCATCTCTAAGGCCAAGGGCCAGCCTCGCGAACCACAGGTCTACG | |
| | | TCTACCCCCCATCAAGAGATGAACTGACAAAAAATCAGGTCTCTCT | |
| | | GACATGCCTGGTCAAAGGATTCTACCCTTCCGACATCGCCGTGGAG | |
| | | TGGGAAAGTAACGGCCAGCCCGAGAACAATTACAAGACCACACCC | |
| | | CCTGTCCTGGACTCTGATGGGAGTTTCGCTCTGGTGTCAAAGCTGAC | |
| | | CGTCGATAAAAGCCGGTGGCAGCAGGGCAATGTGTTTAGCTGCTCC | |
| | | GTCATGCACGAAGCCCTGCACAATCACTACACACAGAAGTCCCTGA | |
| | | GCCTGAGCCCTGGC | |
| | VH | QVQLVQSGACEVKKPGASVKVSCKASGYIFTAYTMHWVRQAPGQGL | 59 |
| | | EWMGWIKPNNGLANYAQKFQGRVTMTRDTSISTAYMELSRLRSDDT | |
| | | AVYYCARSEITTEFDYWGQGTLVTVSS | |
| 25165 | Full | QVQLVQSGACEVKKPGASVKVSCKASGYIFTAYTMHWVRQAPGQGL | 70 |
| | | EWMGWIKPNNGLANYAQKFQGRVTMTRDTSISTAYMELSRLRSDDT | |
| | | AVYYCARSEITTEFDYWGQGTLVTVSSASTKGPSVFPLAPSSKSTSGGT | |
| | | AALGCLVKDYFPEPVTVSWNSGALTSGVHTFPAVLQSSGLYSLSSVVT | |
| | | VPSSSLGTQTYICNVNHKPSNTKVDKKVEPKSCDKTHTCPPCPAPELLG | |
| | | GPSVFLFPPKPKDTLMISRTPEVTCVVVDVSHEDPEVKFNWYVDGVEV | |
| | | HNAKTKPREEQYNSTCYRVVSVLTVLHQDWLNGKEYKCKVSNKALP | |
| | | APIEKTISKAKGQPREPQVYVYPPSRDELTKNQVSLTCLVKGFYPSDIA | |
| | | VEWESNGQPENNYKTTPPVLDSDGSFALVSKLTVDKSRWQQGNVFSC | |
| | | SVMHEALHNHYTQKSLSLSPG | |
| | Full | CAGGTGCAGCTGGTGCAGAGCGGAGCCTGCGAGGTGAAGAAGCCA | 71 |
| | | GGGGCCAGCGTGAAGGTGTCTTGCAAGGCCAGCGGCTACATCTTCA | |
| | | CCGCCTATACAATGCACTGGGTGCGGCAGGCCCCCGGACAGGGCCT | |
| | | GGAGTGGATGGGCTGGATCAAGCCTAACAATGGCCTGGCCAACTAC | |
| | | GCCCAGAAGTTTCAGGGCAGGGTGACCATGACACGCGACACCTCTA | |
| | | TCAGCACAGCCTATATGGAGCTGTCCCGGCTGAGATCTGACGATAC | |
| | | CGCCGTGTACTATTGTGCCAGGTCCGAGATCACCACAGAGTTCGAT | |
| | | TACTGGGGCCAGGGCACCCTGGTGACAGTGAGCTCCGCTAGCACTA | |
| | | AGGGGCCTTCCGTGTTTCCACTGGCTCCCTCTAGTAAATCCACCTCT | |
| | | GGAGGCACAGCTGCACTGGGATGTCTGGTGAAGGATTACTTCCCTG | |
| | | AACCAGTCACAGTGAGTTGGAACTCAGGGGCTCTGACAAGTGGAGT | |
| | | CCATACTTTTCCCGCAGTGCTGCAGTCAAGCGGACTGTACTCCCTGT | |
| | | CCTCTGTGGTCACCGTGCCTAGTTCAAGCCTGGGCACCCAGACATA | |
| | | TATCTGCAACGTGAATCACAAGCCATCAAATACAAAAGTCGACAAG | |
| | | AAAGTGGAGCCCAAGAGCTGTGATAAAACTCATACCTGCCCACCTT | |
| | | GTCCGGCGCCAGAACTGCTGGGAGGACCAAGCGTGTTCCTGTTTCC | |
| | | ACCCAAGCCTAAAGACACCCTGATGATTTCCCGGACTCCTGAGGTC | |
| | | ACCTGCGTGGTCGTGGACGTGTCTCACGAGGACCCCGAAGTCAAGT | |
| | | TCAACTGGTACGTGGATGGCGTCGAAGTGCATAATGCCAAGACCAA | |
| | | ACCCCGGGAGGAACAGTACAACTCTACCTGCTATAGAGTCGTGAGT | |
| | | GTCCTGACAGTGCTGCACCAGGACTGGCTGAATGGGAAGGAGTATA | |
| | | AGTGTAAAGTGAGCAACAAAGCCCTGCCCGCCCCAATCGAAAAAA | |
| | | CAATCTCTAAAGCAAAAGGACAGCCTCGCGAACCACAGGTCTACGT | |
| | | CTACCCCCCATCAAGAGATGAACTGACAAAAAATCAGGTCTCTCTG | |
| | | ACATGCCTGGTCAAAGGATTCTACCCTTCCGACATCGCCGTGGAGT | |
| | | GGGAAAGTAACGGCCAGCCCGAGAACAATTACAAGACCACACCCC | |
| | | CTGTCCTGGACTCTGATGGGAGTTTCGCTCTGGTGTCAAAGCTGACC | |
| | | GTCGATAAAAGCCGGTGGCAGCAGGGCAATGTGTTTAGCTGCTCCG | |
| | | TCATGCACGAAGCCCTGCACAATCACTACACACAGAAGTCCCTGAG | |
| | | CCTGAGCCCTGGC | |
| | VH | QVQLVQSGACEVKKPGASVKVSCKASGYIFTAYTMHWVRQAPGQGL | 59 |
| | | EWMGWIKPNNGLANYAQKFQGRVTMTRDTSISTAYMELSRLRSDDT | |
| | | AVYYCARSEITTEFDYWGQGTLVTVSS | |
| 25169 | Full | ASTKGPSVFPLAPSSKSTSGGTAALGCLVKDYFPEPVTVSWNSGALTS | 72 |
| | | GVHTFPAVLQSSGLYSLSSVVTVPSSSLGTQTYICNVNHKPSNTKVDK | |
| | | KVEPKSCDKTHTCPPCPAPELLGGCPSVFLFPPKPKDTLMISRTPEVTCV | |
| | | VVDVSHEDPEVKFNWYVDGVEVHNAKTKPREEQYNSTCYRVVSVLT | |
| | | VLHQDWLNGKEYKCKVSNKALPAPIEKTISKAKGQPREPQVYVYPPSR | |
| | | DELTKNQVSLTCLVKGFYPSDIAVEWESNGQPENNYKTTPPVLDSDGS | |
| | | FALVSKLTVDKSRWQQGNVFSCSVMHEALHNHYTQKSLSLSPG | |
| | Full | CAGGTGCAGCTGGTGCAGAGCGGAGCCGAGGTGAAGAAGCCAGGG | 73 |
| | | GCCAGCGTGAAGGTGTCTTGCAAGGCCAGCGGCTACATCTTCACCG | |
| | | CCTATACAATGCACTGGGTGCGGCAGGCCCCCGGACAGGGCCTGGA | |
| | | GTGGATGGGCTGGATCAAGCCTAACAATGGCCTGGCCAACTACGCC | |
| | | CAGAAGTTTCAGGGCAGGGTGACCATGACACGCGACACCTCTATCA | |
| | | GCACAGCCTATATGGAGCTGTCCCGGCTGAGATCTGACGATACCGC | |
| | | CGTGTACTATTGTGCCAGGTCCGAGATCACCACAGAGTTCGATTAC | |
| | | TGGGGCCAGGGCACCCTGGTGACAGTGAGCTCCGCTAGCACTAAGG | |
| | | GGCCTTCCGTGTTTCCACTGGCTCCCTCTAGTAAATCCACCTCTGGA | |
| | | GGCACAGCTGCACTGGGATGTCTGGTGAAGGATTACTTCCCTGAAC | |
| | | CAGTCACAGTGAGTTGGAACTCAGGGGCTCTGACAAGTGGAGTCCA | |
| | | TACTTTTCCCGCAGTGCTGCAGTCAAGCGGACTGTACTCCCTGTCCT | |
| | | CTGTGGTCACCGTGCCTAGTTCAAGCCTGGGCACCCAGACATATAT | |
| | | CTGCAACGTGAATCACAAGCCATCAAATACAAAAGTCGACAAGAA | |
| | | AGTGGAGCCCAAGAGCTGTGATAAAACTCATACCTGCCCACCTTGT | |
| | | CCGGCGCCCGAGCTGCTGGGCGGCTGCCCTAGCGTGTTCCTGTTTCC | |
| | | CCCTAAGCCAAAGGACACCCTGATGATCTCTCGGACCCCCGAGGTG | |
| | | ACATGCGTGGTGGTGGACGTGAGCCACGAGGACCCCGAGGTGAAG | |
| | | TTCAACTGGTACGTGGATGGCGTGGAGGTGCACAATGCCAAGACAA | |
| | | AGCCCCGGGAGGAGCAGTACAACAGCACCTGCTATAGAGTGGTGT | |
| | | CCGTGCTGACAGTGCTGCACCAGGACTGGCTGAACGGCAAGGAGT | |
| | | ATAAGTGCAAGGTGTCCAATAAGGCCCTGCCAGCCCCCATCGAGAA | |
| | | GACCATCTCTAAGGCCAAGGGCCAGCCTCGCGAACCACAGGTCTAC | |
| | | GTCTACCCCCCATCAAGAGATGAACTGACAAAAAATCAGGTCTCTC | |
| | | TGACATGCCTGGTCAAAGGATTCTACCCTTCCGACATCGCCGTGGA | |
| | | GTGGGAAAGTAACGGCCAGCCCGAGAACAATTACAAGACCACACC | |
| | | CCCTGTCCTGGACTCTGATGGGAGTTTCGCTCTGGTGTCAAAGCTGA | |
| | | CCGTCGATAAAAGCCGGTGGCAGCAGGGCAATGTGTTTAGCTGCTC | |
| | | CGTCATGCACGAAGCCCTGCACAATCACTACACACAGAAGTCCCTG | |
| | | AGCCTGAGCCCTGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |
| 26704 | Full | QVQLVQSGAEVKKPGASVKVSCKASGYIFTAYTMHWVRQAPGQGLE | 74 |
| | | WMGWIKPNNGLANYAQKFQGRVTMTRDTSISTAYMELSRLRSDDTA | |
| | | VYYCARSEITTEFDYWGQGTLVTVSSASTKGPSVFPLAPSSKSTSGGTA | |
| | | ALGCLVKDYFPEPVTVSWNSGALTSGVHTFPAVLQSSGLYSLSSVVTV | |
| | | PSSSLGTQTYICNVNHKPSNTKVDKKVEPKSCDKTHTCPPCPAPELLGG | |
| | | PSVFLFPPKPKDTLMISRTPEVTCVVVDVSHEDPEVKFNWYVDGVEVH | |
| | | NAKTKPREEQYNSTCYRVVSVLTVLHQDWLNGKEYKCKVSNKALPA | |
| | | PIEKTISKAKGQPREPQVYTLPPSRDELTKNQVSLTCLVKGFYPSDIAVE | |
| | | WESNGQPENNYKTTPPVLDSDGSFFLYSKLTVDKSRWQQGNVFSCSV | |
| | | MHEALHNHYTQKSLSLSPG | |
| | Full | CAGGTGCAGCTGGTGCAGAGCGGAGCCGAGGTGAAGAAGCCAGGG | 75 |
| | | GCCAGCGTGAAGGTGTCTTGCAAGGCCAGCGGCTACATCTTCACCG | |
| | | CCTATACAATGCACTGGGTGAGGCAGGCCCCCGGACAGGGACTGG | |
| | | AGTGGATGGGCTGGATCAAGCCTAACAATGGCCTGGCCAACTACGC | |
| | | CCAGAAGTTTCAGGGCAGGGTGACCATGACACGCGACACCTCTATC | |
| | | AGCACAGCCTATATGGAGCTGTCCCGGCTGAGATCTGACGATACAG | |
| | | CCGTGTACTATTGTGCCAGATCCGAGATCACCACAGAGTTTGATTA | |
| | | CTGGGGCCAGGGCACCCTGGTGACAGTGAGCTCCGCTAGCACTAAG | |
| | | GGGCCTTCCGTGTTTCCACTGGCTCCCTCTAGTAAATCCACCTCTGG | |
| | | AGGCACAGCTGCACTGGGATGTCTGGTGAAGGATTACTTCCCTGAA | |
| | | CCAGTCACAGTGAGTTGGAACTCAGGGGCTCTGACAAGTGGAGTCC | |
| | | ATACTTTTCCCGCAGTGCTGCAGTCAAGCGGACTGTACTCCCTGTCC | |
| | | TCTGTGGTCACCGTGCCTAGTTCAAGCCTGGGCACCCAGACATATA | |
| | | TCTGCAACGTGAATCACAAGCCATCAAATACAAAAGTCGACAAGA | |
| | | AAGTGGAGCCCAAGAGCTGTGATAAAACTCATACCTGCCCACCTTG | |
| | | TCCGGCGCCCGAGCTGCTGGGCGGCCCTAGCGTGTTCCTGTTTCCCC | |
| | | CTAAGCCAAAGGACACCCTGATGATCTCTCGGACCCCCGAGGTGAC | |
| | | ATGCGTGGTGGTGGACGTGAGCCACGAGGACCCCGAGGTGAAGTT | |
| | | CAACTGGTACGTGGATGGCGTGGAGGTGCACAATGCCAAGACAAA | |
| | | GCCCCGGGAGGAGCAGTACAACAGCACCTGCTATAGAGTGGTGTCC | |
| | | GTGCTGACAGTGCTGCACCAGGACTGGCTGAACGGCAAGGAGTAT | |
| | | AAGTGTAAGGTGTCCAATAAGGCCCTGCCAGCCCCCATCGAGAAGA | |
| | | CCATCTCTAAGGCCAAGGGCCAGCCTCGCGAACCACAGGTGTACAC | |
| | | TCTGCCTCCATCTCGGGACGAGCTGACTAAGAACCAGGTCAGTCTG | |
| | | ACCTGTCTGGTGAAAGGATTCTATCCCAGCGATATCGCTGTGGAGT | |
| | | GGGAATCCAATGGCCAGCCTGAGAACAATTACAAGACCACACCCC | |
| | | CTGTGCTGGACTCTGATGGCAGTTTCTTTCTGTATAGTAAGCTGACC | |
| | | GTCGATAAATCACGATGGCAGCAGGGGAACGTGTTCAGCTGTTCAG | |
| | | TGATGCACGAAGCCCTGCACAACCATTACACCCAGAAGAGCCTGAG | |
| | | CCTGTCTCCCGGC | |
| | VH | QVQLVQSGAEVKKPGASVKVSCKASGYIFTAYTMHWVRQAPGQGLE | 43 |
| | | WMGWIKPNNGLANYAQKFQGRVTMTRDTSISTAYMELSRLRSDDTA | |
| | | VYYCARSEITTEFDYWGQGTLVTVSS | |

TABLE C
Additional Sequences for Cysteine Insertion Variants

| | Position(s) | | |
|---|---|---|---|
| | of | | SEQ |
| | Cysteine | | ID |
| Domain | Insertion | Sequence | NO |
| CH2 Domain | Between | APELLGGPSVFLFPPKPKDTLMISRTPEVTCVVVDVSHEDP | 76 |
| | T299 and | EVKFNWYVDGVEVHNAKTKPREEQYNSTCYRVVSVLTV | |
| | Y300 | LHQDWLNGKEYKCKVSNKALPAPIEKTISKAK | |
| CH2 Domain | Between | APELLGGCPSVFLFPPKPKDTLMISRTPEVTCVVVDVSHED | 77 |
| | G237 and | PEVKFNWYVDGVEVHNAKTKPREEQYNSTYRVVSVLTVL | |
| | P238 | HQDWLNGKEYKCKVSNKALPAPIEKTISKAK | |
| CH2 Domain | Between | APELLGGCPSVFLFPPKPKDTLMISRTPEVTCVVVDVSHED | 78 |
| | T299 and | PEVKFNWYVDGVEVHNAKTKPREEQYNSTCYRVVSVLTV | |
| | Y300, and | LHQDWLNGKEYKCKVSNKALPAPIEKTISKAK | |
| | between | | |
| | G237 and | | |
| | P238 | | |
| CL Domain | Between | RTVAAPSVFIFPPSDEQLKCSGTASVVCLLNNFYPREAKVQ | 79 |
| | K126 and | WKVDNALQSGNSQESVTEQDSKDSTYSLSSTLTLSKADYE | |
| | S127 | KHKVYACEVTHQGLSSPVTKSFNRGEC | |
| VH Domain | Between | QVQLVQSGACEVKKPGASVKVSCKASGYIFTAYTMHWV | 59 |
| | A9 and | RQAPGQGLEWMGWIKPNNGLANYAQKFQGRVTMTRDTS | |
| | E10 | ISTAYMELSRLRSDDTAVYYCARSEITTEFDYWGQGTLVT | |
| | | VSS | |
| VL Domain | Between | DIVMTQSPDSLAVSLGERATINCKSSESVDSYANSFLHWY | 56 |
| | P40 and | QQKPCGQPPKLLIYRASTRESGVPDRFSGSGSGTDFTLTISS | |
| | G41 | LQAEDVAVYYCQQSKEDPLTFGGGTKVEIK | |

We claim:

1. An antibody-drug conjugate having Formula I:
A-(L-(D)ₙ)ₚ (I)
wherein:
A is an antibody construct comprising an antigen-binding domain and an immunoglobulin (Ig) hinge region, the antigen-binding domain specifically binding to c-Met and comprising the heavy chain CDR sequences (HCDR1, HCDR2 and HCDR3) of the VH domain sequence set forth in SEQ ID NO:1, and the light chain CDR sequences (LCDR1, LCDR2 and LCDR3) of the VL domain sequence set forth in SEQ ID NO:2, and the Ig hinge region comprising an upper hinge sequence having an amino acid sequence of a native IgG1, IgG2 or IgG4 upper hinge sequence;
L is a cleavable linker;
D is: 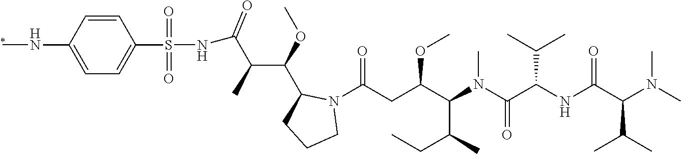 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]
where * is the point of attachment to L,
n is between 1 and 4, and
p is between 1 and 8.

2. The antibody-drug conjugate according to claim 1, wherein the antigen-binding domain comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23; an HCDR3 sequence selected from the sequences as set forth in SEQ ID NOs: 5, 11 and 18; an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21.

3. The antibody-drug conjugate according to claim 1 or 2, wherein the antibody construct comprises a VH domain sequence having the amino acid sequence as set forth in SEQ ID NO:1 and a VL domain sequence having the amino acid sequence as set forth in SEQ ID NO:2.

4. The antibody-drug conjugate according to any one of claims 1 to 3, wherein the Ig hinge region comprises an upper hinge sequence having the amino acid sequence of a native IgG1 upper hinge sequence.

5. The antibody-drug conjugate according to any one of claims 1 to 3, wherein the Ig hinge region comprises an upper hinge sequence having the amino acid sequence as set forth in SEQ ID NO: 25.

6. The antibody-drug conjugate according to any one of claims 1 to 5, wherein the antibody construct further comprises a scaffold, wherein the scaffold is based on an immunoglobulin Fc region.

7. The antibody-drug conjugate according to claim 6, wherein the immunoglobulin Fc region is an IgG1 Fc region.

8. The antibody-drug conjugate according to claim 6 or 7, wherein the Fc region is a heterodimeric Fc comprising a modified CH3 domain comprising one or more amino acid modifications that promote formation of the heterodimeric Fc over formation of a homodimeric Fc.

9. The antibody-drug conjugate according to claim 8, wherein the heterodimeric Fc comprises a first Fc polypeptide and a second Fc polypeptide, wherein:
a) the first Fc polypeptide comprises the amino acid modifications L351Y, F405A and Y407V, and the second Fc polypeptide comprises the amino acid modifications T366L, K392M and T394W, or
b) the first Fc polypeptide comprises the amino acid modifications L351Y, F405A and Y407V, and the second Fc polypeptide comprises the amino acid modifications T366L, K392L and T394W, or
c) the first Fc polypeptide comprises the amino acid modifications T350V, L351Y, F405A and Y407V, and the second Fc polypeptide comprises the amino acid modifications T350V, T366L, K392M and T394W, or
d) the first Fc polypeptide comprises the amino acid modifications T350V, L351Y, F405A and Y407V, and the second Fc polypeptide comprises the amino acid modifications T350V, T366L, K392L and T394W, or
e) the first Fc polypeptide comprises the amino acid modifications T350V, L351Y, S400E, F405A and Y407V, and the second Fc polypeptide comprises the amino acid modifications T350V, T366L, N390R, K392M and T394W.

10. The antibody-drug conjugate according to any one of claims 1 to 9, wherein the antibody construct is a bivalent antibody comprising two antigen-binding domains, wherein both antigen-binding domains specifically bind to c-Met.

11. The antibody-drug conjugate according to any one of claims 1 to 10, wherein L is a protease-cleavable linker.

12. The antibody-drug conjugate according to any one of claims 1 to 11, wherein each L is conjugated to a sulfhydryl group of a cysteine residue of the antibody construct.

13. The antibody-drug conjugate according to claim 12, wherein each cysteine residue is a native cysteine residue.

14. The antibody-drug conjugate according to claim 13, wherein p is 2 or 4.

15. The antibody-drug conjugate according to claim 12, wherein each cysteine residue is a non-native cysteine residue.

16. The antibody-drug conjugate according to claim 15, wherein each non-native cysteine residue is a cysteine insertion mutation or a cysteine substitution mutation.

17. The antibody-drug conjugate according to claim 15, wherein each non-native cysteine residue is a cysteine insertion mutation independently selected from:
(a) a cysteine residue inserted between positions 40 and 41 in the VL domain;
(b) a cysteine residue inserted between positions 126 and 127 in the CL domain;
(c) a cysteine residue inserted between positions 9 and 10 in the VH domain;
(d) a cysteine residue inserted between positions 237 and 238 in the CH2 domain, and
(e) a cysteine residue inserted between positions 299 and 300 in the CH2 domain,
wherein the numbering of amino acids in the VL, CL and VH domains is Kabat numbering and the numbering of amino acids in the CH2 domain is EU numbering.

18. The antibody-drug conjugate according to any one of claims 15 to 17, wherein p is 1, 2, 3 or 4.

19. The antibody-drug conjugate according to any one of claims 1 to 11, wherein each L is conjugated to an amino group of a lysine residue of the antibody construct.

20. The antibody-drug conjugate according to claim 19, wherein p is 2, 4 or 6.

21. The antibody-drug conjugate according to any one of claims 1 to 20, wherein L-D has one of the following structures:
a) Formula IV 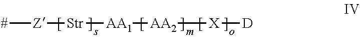
wherein:
Z′ is a linking group that joins the linker to a target group on the antibody construct, A;
Str is a stretcher;
AA₁ and AA₂ are each independently an amino acid, wherein AA₁-[AA₂]ₘ forms a protease cleavage site;
X is a self-immolative group;
s is 0 or 1;
m is 1, 2 or 3;
o is 0, 1 or 2, and #is the point of attachment to the antibody construct, A, or
b) Formula XII 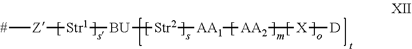
wherein:
Z′ is a linking group that joins the linker to a target group on the antibody construct, A;
Str¹ and Str² are each independently a stretcher;
BU is a branch unit;
AA₁ and AA₂ are each independently an amino acid, wherein AA₁-[AA₂]ₘ forms a protease cleavage site;
X is a self-immolative group;
s and s′ are each independently 0 or 1;
m is 1, 2 or 3;
o is 0, 1 or 2;
#is 2 or 3, and #is the point of attachment to the antibody construct, A.

22. The antibody-drug conjugate according to claim 21, wherein L-D has structure IV, and wherein:
Z′ is a carbonyl group (—C(O)—) or 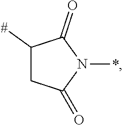
where #is the point of attachment to the anti-cMet antibody construct, A, and * is the point of attachment to the remainder of the linker.

23. The antibody-drug conjugate according to claim 21 or 22, wherein L-D has structure IV, and wherein:
s is 1, and
Str is 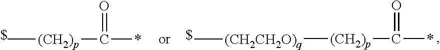
where $ is the point of attachment to Z′, * is the point of attachment to the remainder of the linker, p is an integer between 2 and 6, and q is an integer between 2 and 8.

24. The antibody-drug conjugate according to any one of claims 21 to 23, wherein L-D has structure IV, and wherein:
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit, and
o is 0.

25. The antibody-drug conjugate according to claim 21, wherein L-D has structure XII, and wherein:
Z′ is a carbonyl group (—C(O)—) or 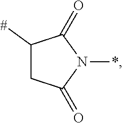
where #is the point of attachment to the anti-cMet antibody construct, A, and * is the point of attachment to the remainder of the linker.

26. The antibody-drug conjugate according to claim 21 or 25, wherein L-D has structure XII, and wherein:
s is 1;
s′ is 1;
Str¹ is 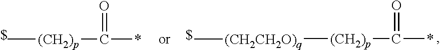
where $ is the point of attachment to Z′, * is the point of attachment to the remainder of the linker, p is an integer between 2 and 6, and q is an integer between 2 and 8, and
Str² is 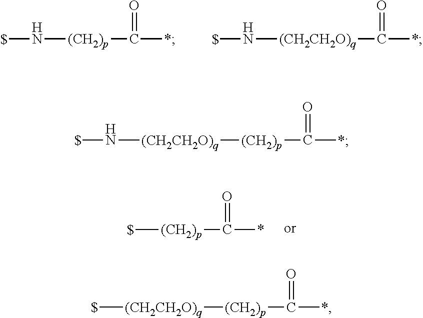
where $ is the point of attachment to BU, * is the point of attachment to the remainder of the linker, p is an integer between 2 and 6, and q is an integer between 2 and 8.

27. The antibody-drug conjugate according to any one of claims 21, 25 and 26, wherein L-D has structure XII, and wherein:
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit, and
o is 0.

28. The antibody-drug conjugate according to any one of claims 21 and 25 to 27, wherein L-D has structure XII, and wherein:
BU is an amino acid or Behera's amine.

29. The antibody-drug conjugate according to claim 1, having one of the following structures:

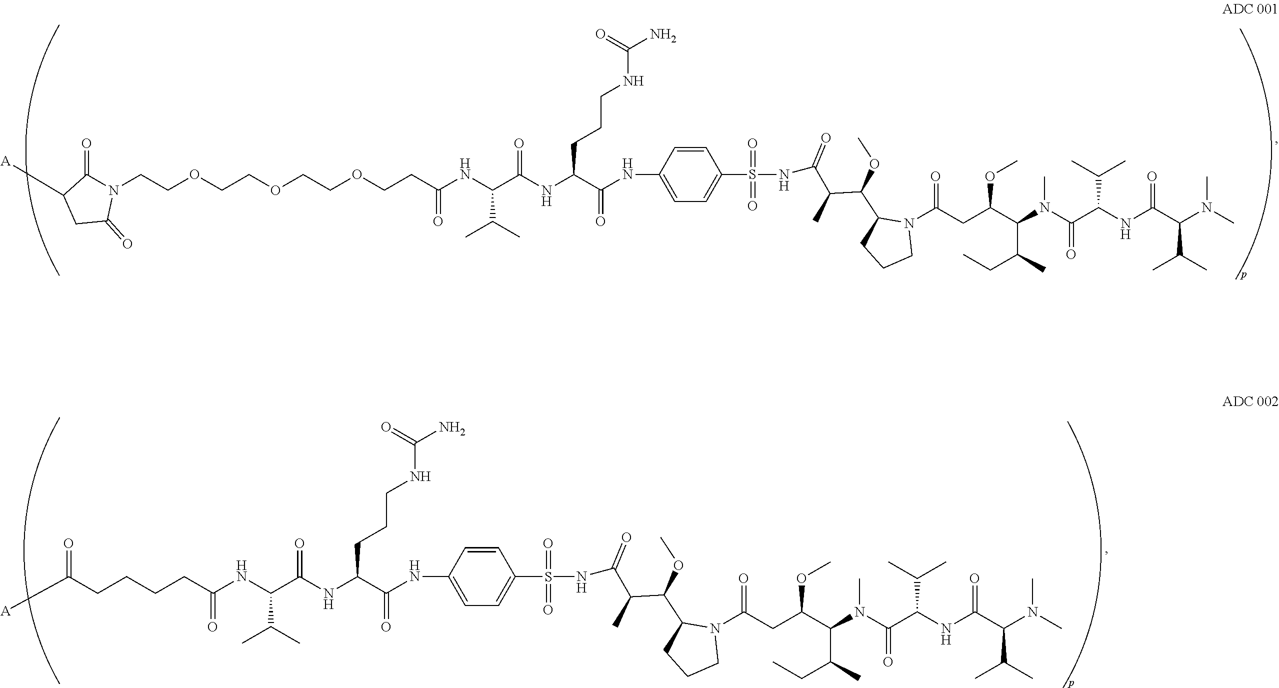

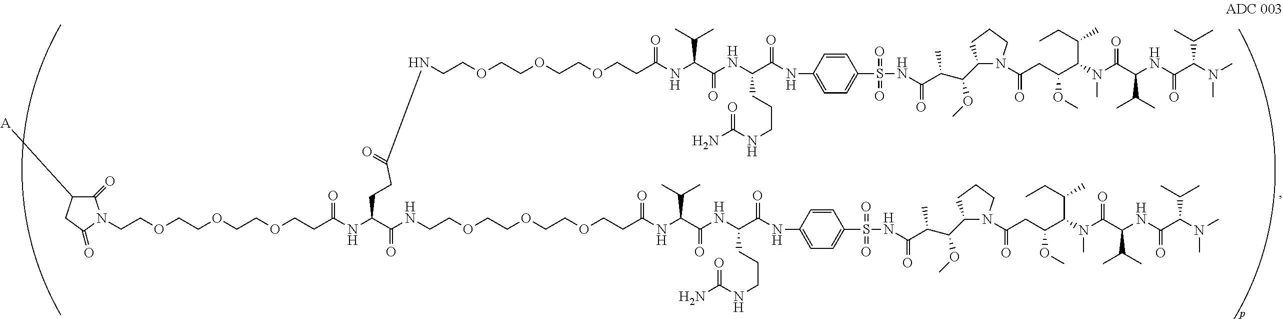

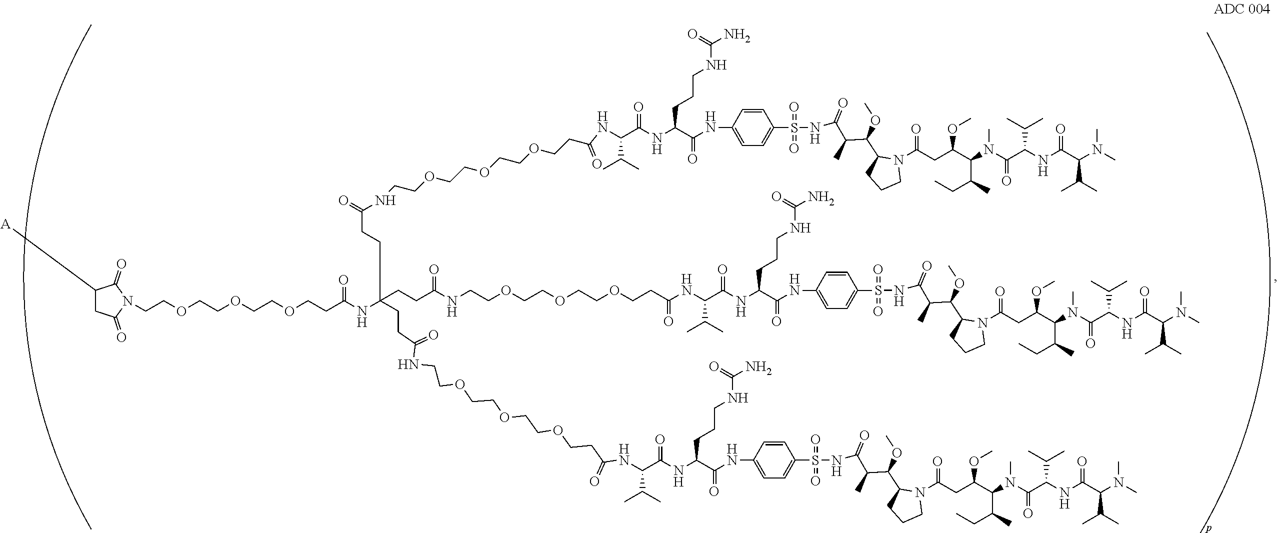

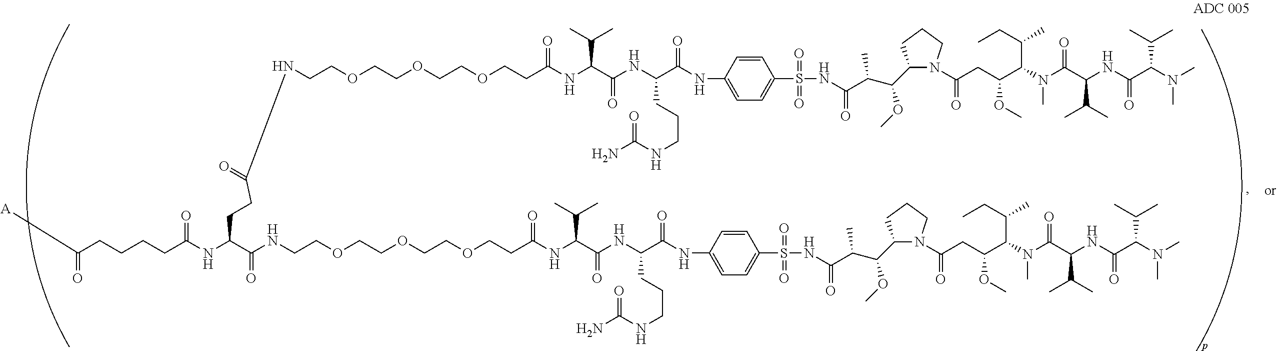

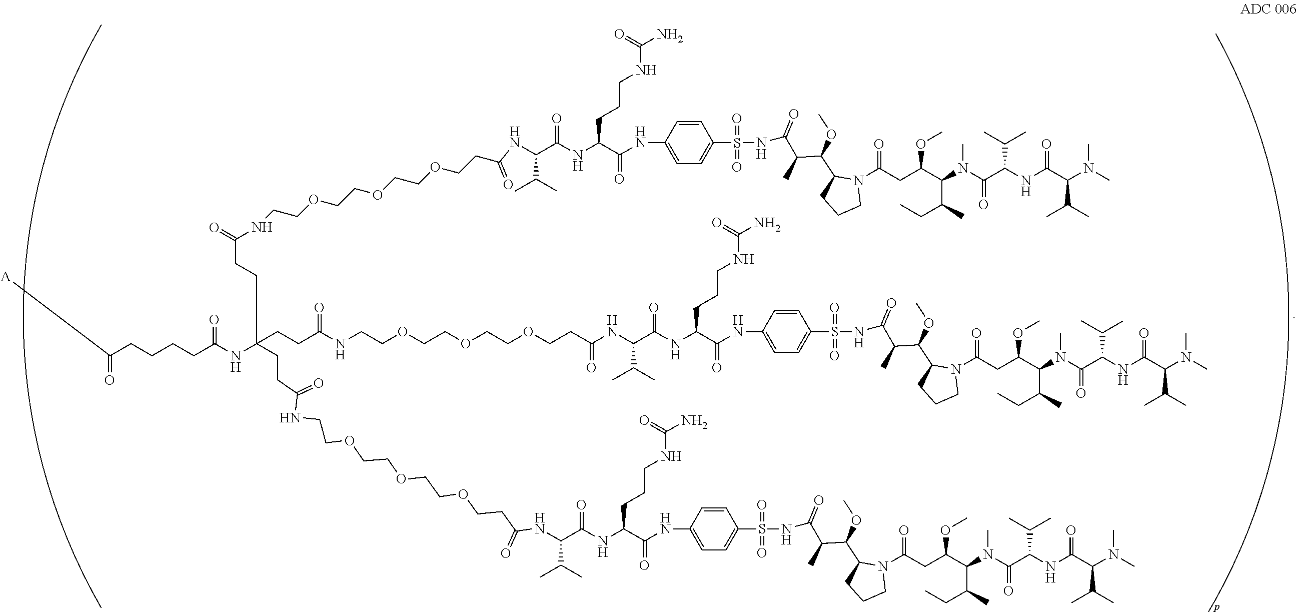

30. The antibody-drug conjugate according to claim 1 having the structure: 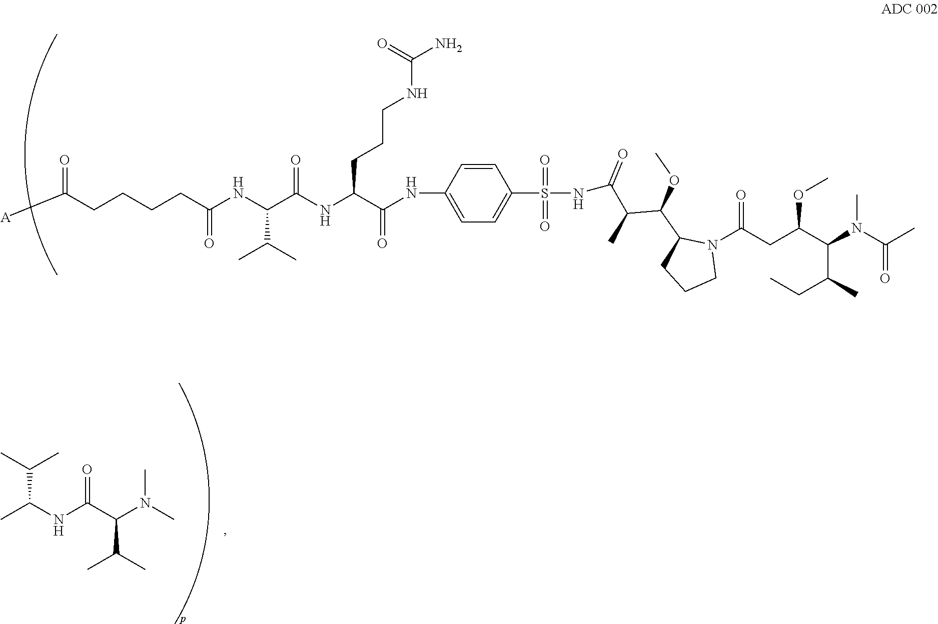
wherein A is the antibody construct that specifically binds to c-Met, and p is 6.

31. The antibody-drug conjugate according to claim 1 having the structure: 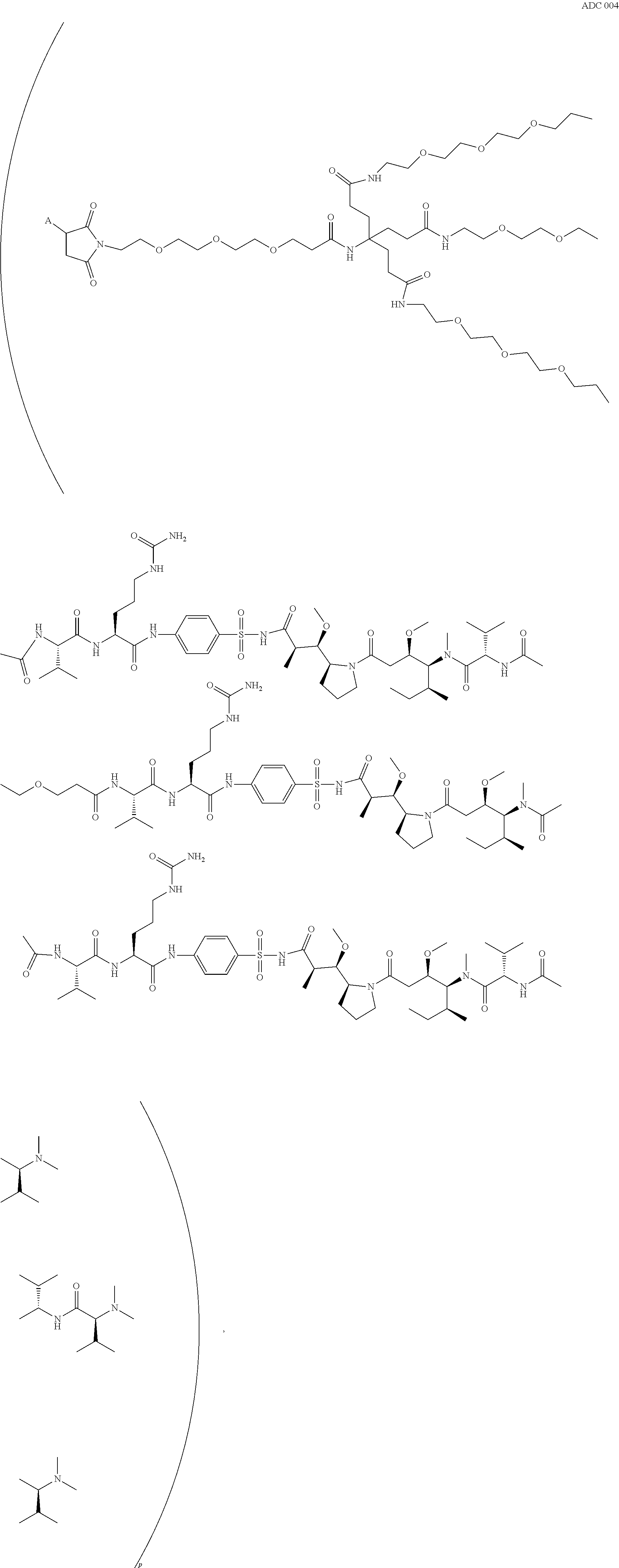
wherein A is the antibody construct that specifically binds to c-Met, and p is 2.

32. An antibody construct comprising:
an antigen-binding domain comprising a VL domain and a VH domain, and optionally a CH1 domain and a CL domain, wherein the antigen-binding domain specifically binds c-Met,
an Fc region comprising a CH2 domain having two CH2 domain sequences and a CH3 domain having two CH3 domain sequences,
wherein the antigen-binding domain comprises the heavy chain CDR sequences (HCDR1, HCDR2 and HCDR3) of the VH domain sequence set forth in SEQ ID NO:1, and the light chain CDR sequences (LCDR1, LCDR2 and LCDR3) of the VL domain sequence set forth in SEQ ID NO: 2, and
wherein the antibody construct comprises one or more cysteine insertion mutations independently selected from:
(a) a cysteine residue inserted between positions 40 and 41 in the VL domain;
(b) a cysteine residue inserted between positions 126 and 127 in the CL domain;
(c) a cysteine residue inserted between positions 9 and 10 in the VH domain;
(d) a cysteine residue inserted between positions 237 and 238 in a CH2 domain sequence, and
(e) a cysteine residue inserted between positions 299 and 300 in a CH2 domain sequence,
wherein the numbering of amino acids in the VL, CL and VH domains is Kabat numbering and the numbering of amino acids in the CH2 domain is EU numbering.

33. The antibody construct according to claim 32, further comprising an immunoglobulin (Ig) hinge region, wherein the Ig hinge region comprises an upper hinge sequence having an amino acid sequence of a native IgG1, IgG2 or IgG4 upper hinge sequence.

34. The antibody construct according to claim 33, wherein the Ig hinge region comprises an upper hinge sequence having the amino acid sequence of a native IgG1 upper hinge sequence.

35. The antibody construct according to claim 33, wherein the Ig hinge region comprises an upper hinge sequence having the amino acid sequence as set forth in SEQ ID NO: 25.

36. The antibody construct according to any one of claims 32 to 35, wherein the antigen-binding domain comprises an HCDR1 sequence selected from the sequences as set forth in SEQ ID NOs: 3, 9, 14, 16 and 22; an HCDR2 sequence selected from the sequences as set forth in SEQ ID NOs: 4, 10, 15, 17 and 23; an HCDR3 sequence selected from the sequences as set forth in SEQ ID NOs: 5, 11 and 18; an LCDR1 sequence selected from the sequences as set forth in SEQ ID NOs: 6, 12 and 19; an LCDR2 sequence selected from the sequences as set forth in SEQ ID NOs: 7, 13 and 20, and an LCDR3 sequence selected from the sequences as set forth in SEQ ID NOs: 8 and 21.

37. The antibody construct according to any one of claims 32 to 36, wherein the Fc region is an IgG1 Fc region.

38. The antibody construct according to any one of claims 32 to 37, wherein the Fc region is a heterodimeric Fc comprising a modified CH3 domain comprising one or more amino acid modifications that promote formation of the heterodimeric Fc over formation of a homodimeric Fc.

39. The antibody construct according to claim 38, wherein the heterodimeric Fc comprises a first Fc polypeptide and a second Fc polypeptide, wherein:
a) the first Fc polypeptide comprises the amino acid modifications L351Y, F405A and Y407V, and the second Fc polypeptide comprises the amino acid modifications T366L, K392M and T394W, or
b) the first Fc polypeptide comprises the amino acid modifications L351Y, F405A and Y407V, and the second Fc polypeptide comprises the amino acid modifications T366L, K392L and T394W, or
c) the first Fc polypeptide comprises the amino acid modifications T350V, L351Y, F405A and Y407V, and the second Fc polypeptide comprises the amino acid modifications T350V, T366L, K392M and T394W, or
d) the first Fc polypeptide comprises the amino acid modifications T350V, L351Y, F405A and Y407V, and the second Fc polypeptide comprises the amino acid modifications T350V, T366L, K392L and T394W, or
e) the first Fc polypeptide comprises the amino acid modifications T350V, L351Y, S400E, F405A and Y407V, and the second Fc polypeptide comprises the amino acid modifications T350V, T366L, N390R, K392M and T394W.

40. The antibody construct according to any one of claims 32 to 39, wherein the antibody construct is a bivalent antibody comprising two antigen-binding domains, wherein both antigen-binding domains specifically bind to c-Met.

41. The antibody construct according to any one of claims 32 to 40, wherein the antibody construct comprises a combination of cysteine insertions, the combination comprising:
(a) a cysteine residue inserted between positions 299 and 300 and between positions 237 and 238 in one or both CH2 domain sequences, or
(b) a cysteine residue inserted between positions 299 and 300 in one or both CH2 domain sequences, and a cysteine residue inserted between positions 9 and 10 in a VH domain, or
(c) a cysteine residue inserted between positions 299 and 300 in one or both CH2 domain sequences, and a cysteine residue inserted between positions 40 and 41 in a VL domain, or
(d) a cysteine residue inserted between positions 237 and 238 in one or both CH2 domain sequences, and a cysteine residue inserted between positions 9 and 10 in a VH domain, or
(e) a cysteine residue inserted between positions 9 and 10 in a VH domain, and a cysteine residue inserted between positions 40 and 41 in a VL domain.

42. The antibody construct according to claim 40, wherein the antibody construct comprises:
(i) a cysteine residue inserted between positions 299 and 300 in one CH2 domain sequence;
(ii) a cysteine residue inserted between positions 299 and 300 in each CH2 domain sequence;
(iii) a cysteine residue inserted between positions 237 and 238 in one CH2 domain sequence;
(iv) a cysteine residue inserted between positions 237 and 238 in each CH2 domain sequence;
(v) a cysteine residue inserted between positions 9 and 10 in one VH domain;
(vi) a cysteine residue inserted between positions 9 and 10 in each VH domain;
(vii) a cysteine residue inserted between positions 40 and 41 in each VL domain;
(viii) a cysteine residue inserted between positions 126 and 127 in each CL domain;
(ix) a cysteine insertion between positions 299 and 300 in a first CH2 domain sequence, a cysteine residue inserted between positions 299 and 300 in a second CH2 domain sequence, and a cysteine residue inserted between positions 237 and 238 in the second CH2 domain sequence;
(x) a cysteine residue inserted between positions 9 and 10 in one VH domain, and a cysteine insertion between positions 299 and 300 in each CH2 domain sequence;
(xi) a cysteine residue inserted between positions 40 and 41 in each VL domain, and a cysteine residue inserted between positions 299 and 300 in one CH2 domain sequence;
(xii) a cysteine residue inserted between positions 40 and 41 in each VL domain, and a cysteine residue inserted between positions 9 and 10 in one VH domain, or
(xiii) a cysteine residue inserted between positions 9 and 10 in each VH domain, and a cysteine insertion between positions 237 and 238 in one CH2 domain sequence.

43. The antibody construct according to claim 40, wherein the antibody construct comprises a cysteine residue inserted between positions 299 and 300 in each CH2 domain sequence.

44. The antibody construct according to any one of claims 32 to 40, wherein the antibody construct comprises a VH domain sequence having the amino acid sequence as set forth in SEQ ID NO: 1 or SEQ ID NO:59 and a VL domain sequence having the amino acid sequence as set forth in SEQ ID NO:2 or SEQ ID NO: 56.

45. The antibody construct according to any one of claims 32 to 40 and 44, wherein the anti-cMet antibody construct comprises a first heavy chain and a second heavy chain, each of the first and second heavy chains comprising a CH2 domain, and wherein one or both of the CH2 domains comprise an amino acid sequence selected from the sequences set forth in SEQ ID NOs: 76, 77 and 78.

46. The antibody construct according to any one of claims 32 to 40, 44 and 45, wherein the anti-cMet antibody construct comprises a first light chain and a light chain, each of the first and second light chains comprising a CL domain, wherein one or both of the CL domains comprises an amino acid sequence as set forth in SEQ ID NO. 79.

47. Use of the antibody construct according to any one of claims 32 to 46 to prepare an antibody drug conjugate.

48. An antibody-drug conjugate comprising the antibody construct according to any one of claims 32 to 46, conjugated to a cytotoxin via a linker.

49. The antibody-drug conjugate according to claim 48 having Formula I:
A-(L-(D)ₙ)ₚ (I)
wherein:
A is the antibody construct according to any one of claims 30 to 44;
L is a cleavable linker;
D is: 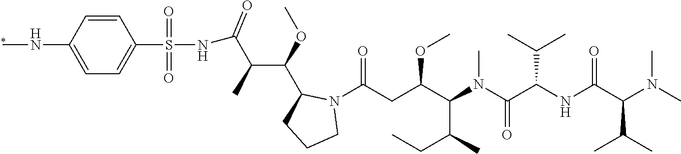 [SMILES: *Nc1ccc(S(=O)(=O)NC(=O)[C@H](C)[C@@H](OC)[C@@H]2CCCN2C(=O)C[C@@H](OC)[C@H]([C@@H](C)CC)N(C)C(=O)[C@@H](NC(=O)[C@H](C(C)C)N(C)C)C(C)C)cc1]
where * is the point of attachment to L,
n is between 1 and 4, and
p is between 1 and 8, and
wherein each L is conjugated to a sulfhydryl group of an inserted cysteine residue.

50. The antibody-drug conjugate according to claim 49, wherein L is a protease-cleavable linker.

51. The antibody-drug conjugate according to claim 49 or 50, wherein L-D has one of the following structures:
a) Formula IV 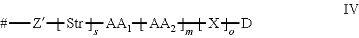
wherein:
Z′ is a linking group that joins the linker to a target group on the antibody construct, A;
Str is a stretcher;
AA₁ and AA₂ are each independently an amino acid, wherein AA₁-[AA₂]ₘ forms a protease cleavage site;
X is a self-immolative group;
s is 0 or 1;
m is 1, 2 or 3;
o is 0, 1 or 2, and #is the point of attachment to the antibody construct, A, or
b) Formula XII 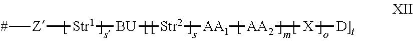
wherein:
Z′ is a linking group that joins the linker to a target group on the antibody construct, A;
Str¹ and Str² are each independently a stretcher;
BU is a branch unit;
AA₁ and AA₂ are each independently an amino acid, wherein AA₁-[AA₂]ₘ forms a protease cleavage site;
X is a self-immolative group;
s and s′ are each independently 0 or 1;
m is 1, 2 or 3;
o is 0, 1 or 2;
#is 2 or 3, and #is the point of attachment to the antibody construct, A.

52. The antibody-drug conjugate according to claim 51, wherein L-D has structure IV, and wherein:
Z′ is a carbonyl group (—C(O)—) or 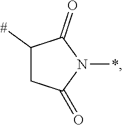
where #is the point of attachment to the antibody construct, A, and * is the point of attachment to the remainder of the linker.

53. The antibody-drug conjugate according to claim 51 or 52, wherein L-D has structure IV, and wherein:
s is 1, and
Str is 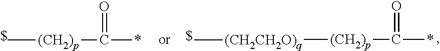
where $ is the point of attachment to Z′, * is the point of attachment to the remainder of the linker, p is an integer between 2 and 6, and q is an integer between 2 and 8.

54. The antibody-drug conjugate according to any one of claims 51 to 53, wherein L-D has structure IV, and wherein:
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit, and
o is 0.

55. The antibody-drug conjugate according to claim 51, wherein L-D has structure XII, and wherein:
Z′ is a carbonyl group (—C(O)—) or 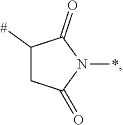
where #is the point of attachment to the antibody construct, A, and * is the point of attachment to the remainder of the linker.

56. The antibody-drug conjugate according to claim 51 or 55, wherein L-D has structure XII, and wherein:
s is 1;
s′ is 1;
Str¹ is 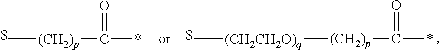
where $ is the point of attachment to Z′, * is the point of attachment to the remainder of the linker, p is an integer between 2 and 6, and q is an integer between 2 and 8, and
Str² is 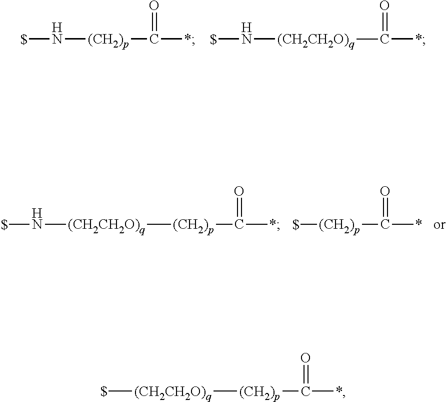
where $ is the point of attachment to BU, * is the point of attachment to the remainder of the linker, p is an integer between 2 and 6, and q is an integer between 2 and 8.

57. The antibody-drug conjugate according to any one of claims 51, 55 and 56, wherein L-D has structure XII, and wherein:
m is 1 and AA₁-[AA₂]ₘ is a dipeptide selected from Val-Lys, Ala-Lys, Phe-Lys, Val-Cit, Phe-Cit, Leu-Cit, Ile-Cit and Trp-Cit, and
o is 0.

58. The antibody-drug conjugate according to any one of claims 51 and 55 to 57, wherein L-D has structure XII, and wherein:
BU is an amino acid or Behera's amine.

59. The antibody-drug conjugate according to any one of claims 49 to 58, wherein p is 1, 2, 3 or 4.

60. The antibody-drug conjugate according to claim 49, having one of the following structures:

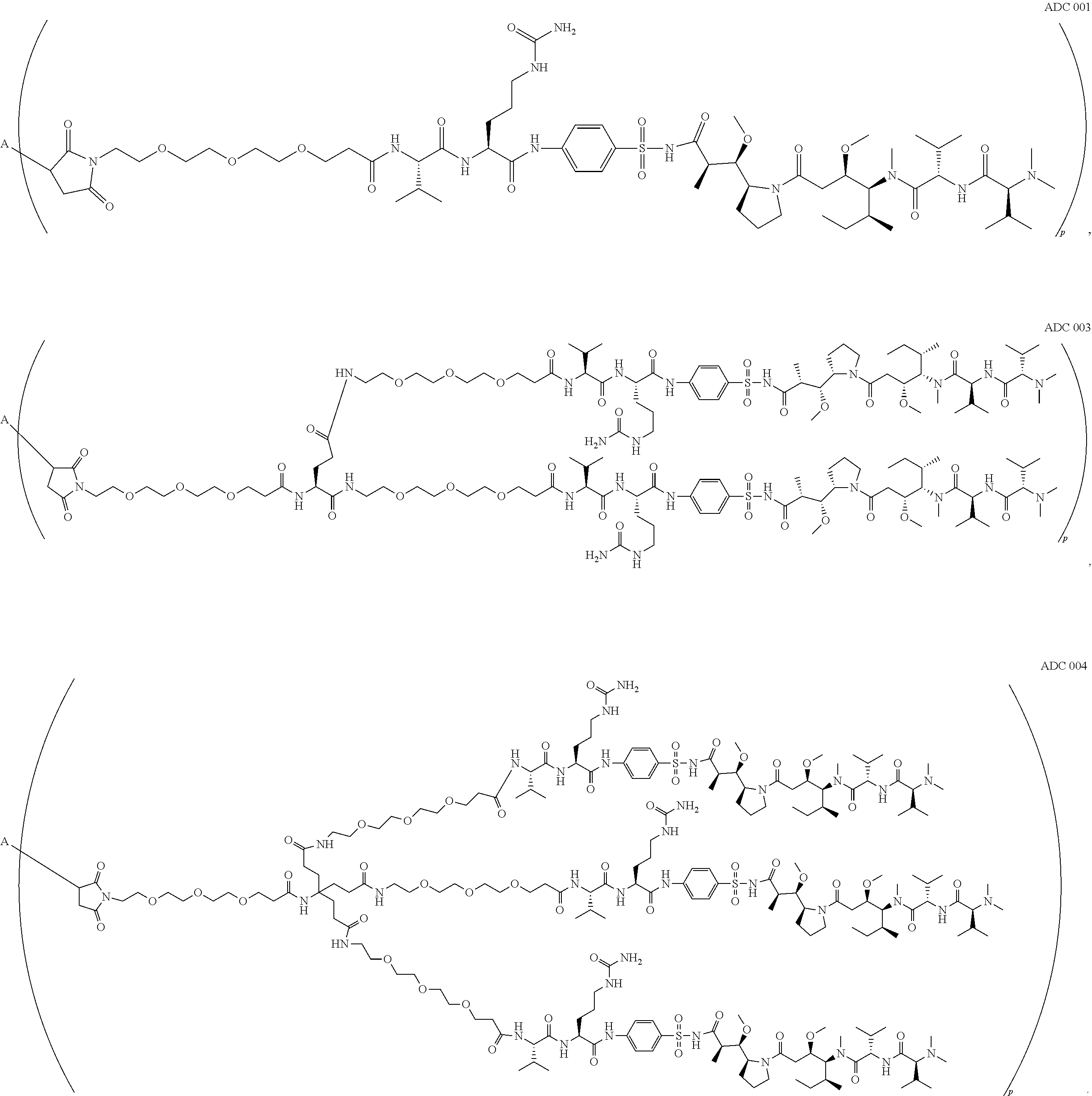

61. The antibody-drug conjugate according to claim 49, having the structure: 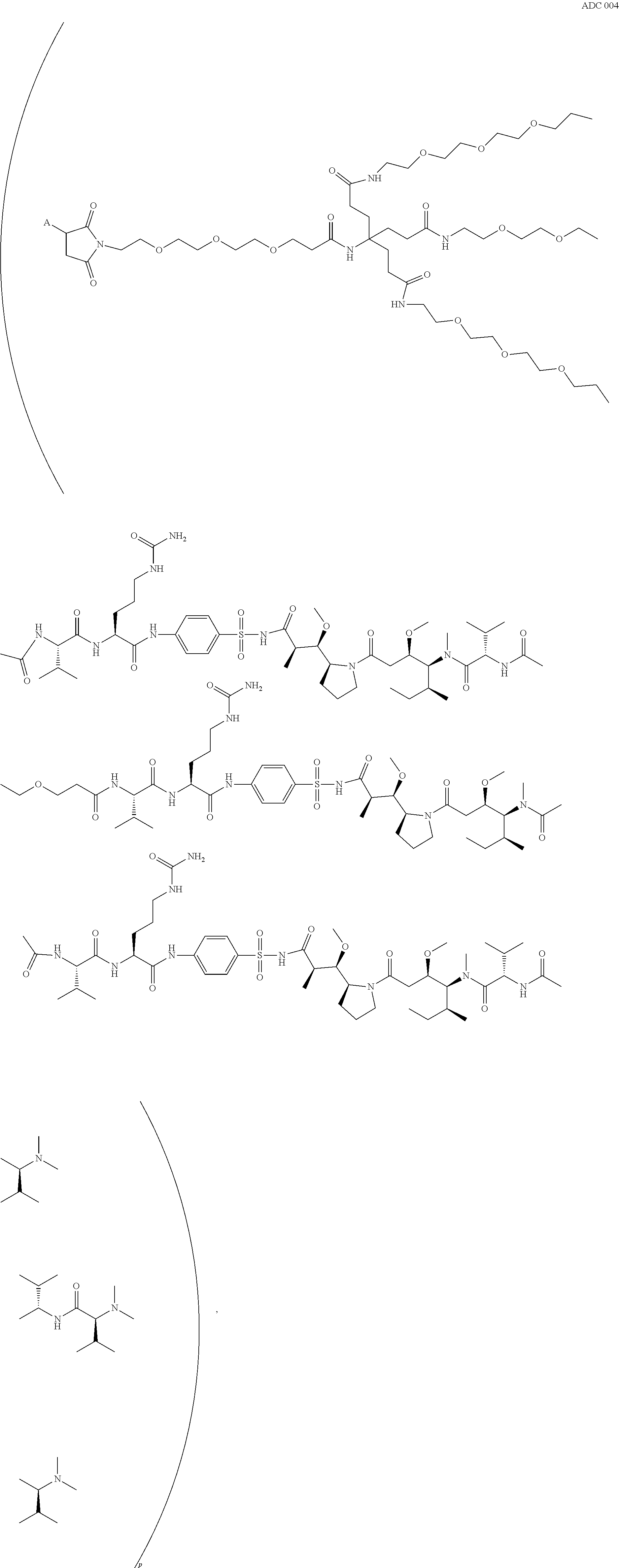
wherein A is the antibody construct according to claim 43, and p is 2.

62. A pharmaceutical composition comprising the antibody-drug conjugate according to any one of claims 1 to 31 and 48 to 61 and a pharmaceutically acceptable carrier or diluent.

63. A method of treating cancer in a subject comprising administering to the subject an effective amount of the antibody-drug conjugate according to any one of claims 1 to 31 and 48 to 61.

64. An antibody-drug conjugate according to any one of claims 1 to 31 and 48 to 61 for use in therapy.

65. The antibody-drug conjugate for use according to claim 64, wherein the therapy comprises treating cancer in a subject in need thereof.

66. Use of an antibody-drug conjugate according to any one of claims 1 to 31 and 48 to 61 in the manufacture of a medicament for the treatment of cancer.

67. A polynucleotide or set of polynucleotides encoding the antibody construct according to any one of claims 32 to 46.

68. A vector or set of vectors comprising the polynucleotide or set of polynucleotides according to claim 67.

69. A host cell comprising the vector or set of vectors according to claim 68.

70. A multivalent drug-linker selected from: 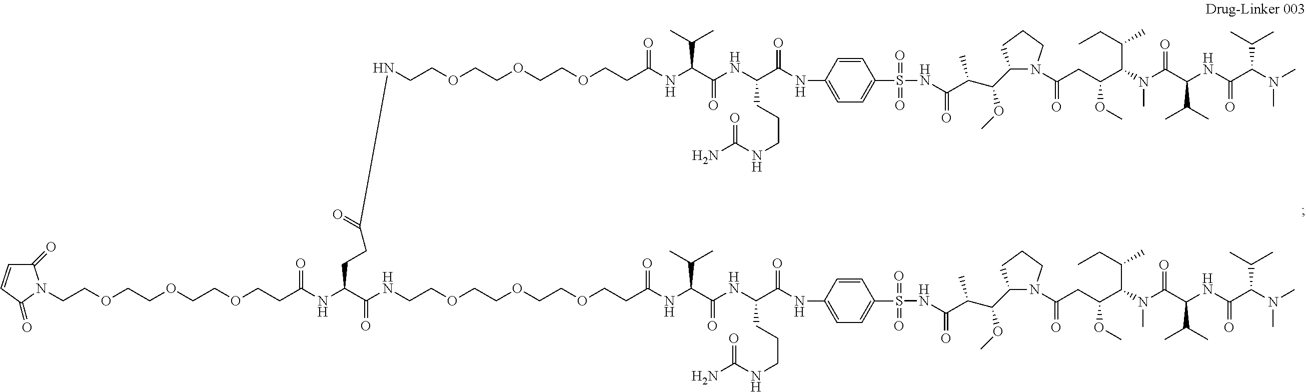 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCOCCOCCOCCN2C(=O)C=CC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] 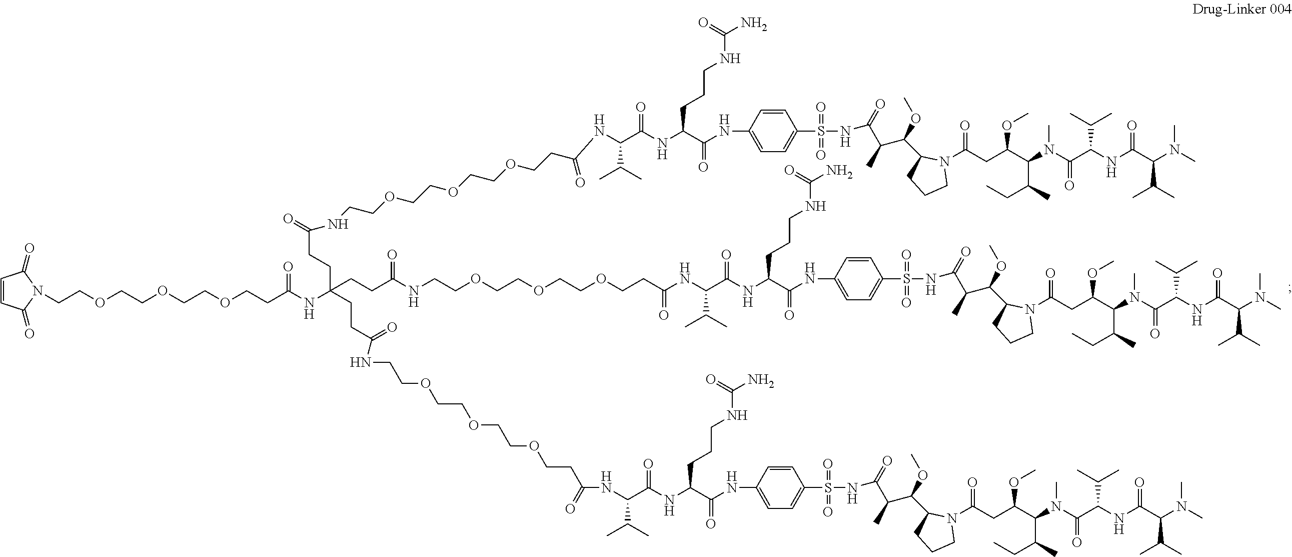 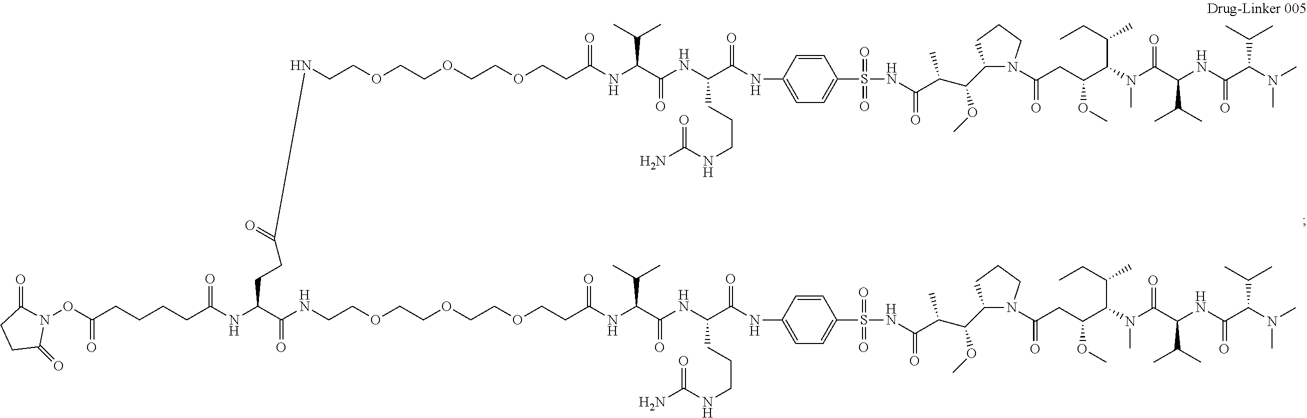 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)ON2C(=O)CCC2=O)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] 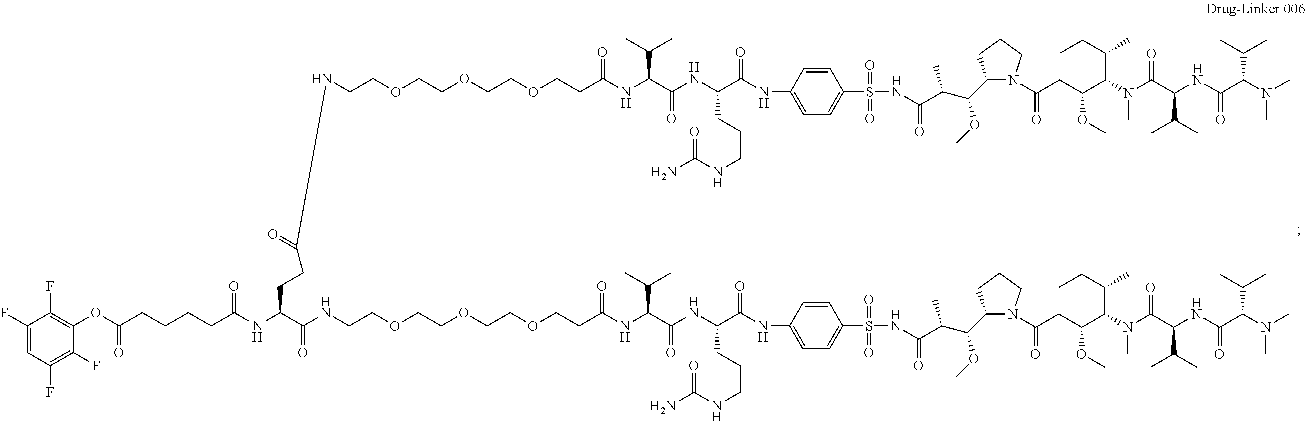 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)CCOCCOCCOCCNC(=O)CC[C@H](NC(=O)CCCCC(=O)Oc2c(F)c(F)cc(F)c2F)C(=O)NCCOCCOCCOCCC(=O)N[C@H](C(=O)N[C@@H](CCCNC(N)=O)C(=O)Nc2ccc(S(=O)(=O)NC(=O)[C@H](C)[C@@H](OC)[C@@H]3CCCN3C(=O)C[C@@H](OC)[C@H]([C@@H](C)CC)N(C)C(=O)[C@@H](NC(=O)[C@H](C(C)C)N(C)C)C(C)C)cc2)C(C)C)C(C)C)cc1)OC)N(C)C(=O)[C@@H](NC(=O)[C@H](C(C)C)N(C)C)C(C)C] 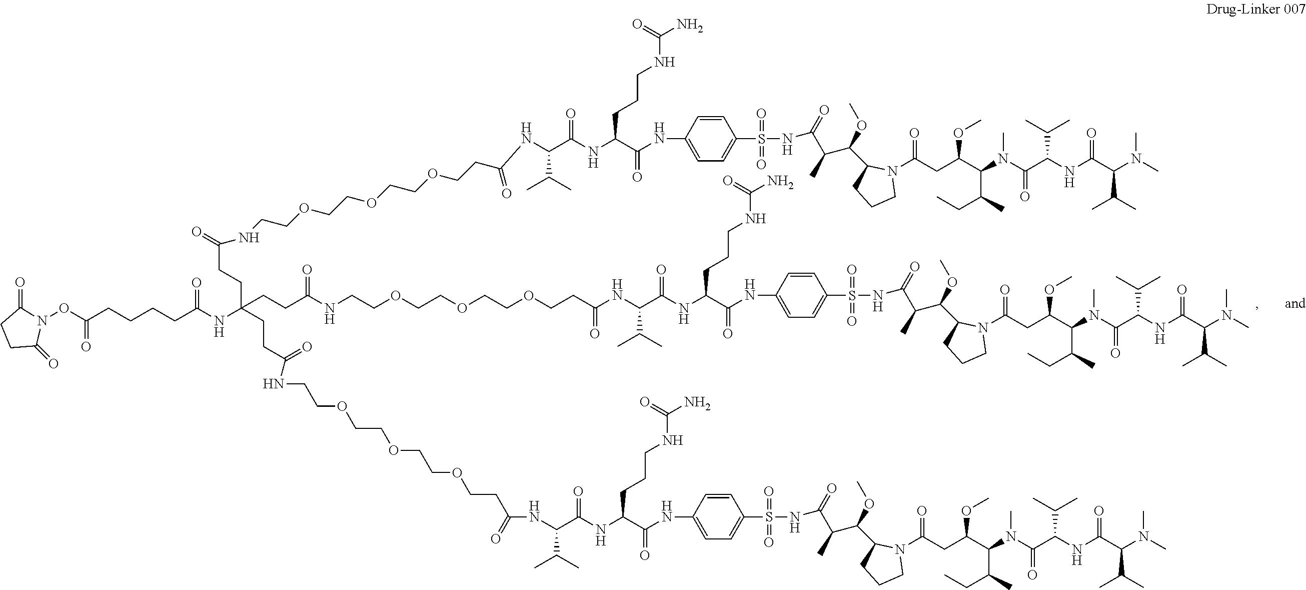 [chemical structure]
